(19) United States
(12) Patent Application Publication
(10) Pub. No.: US 2026/0199483 A1
(43) Pub. Date: Jul. 16, 2026

(54) CONJUGATES COMPRISING ANTIFUNGALS AND CASEIN KINASE (CK1) INHIBITORS AND METHODS OF USE THEREOF

(71) Applicant: Bright Angel Therapeutics Inc., Toronto (CA)

(72) Inventors: Dominic Jaikaran, Toronto (CA); Abdelmalik Slassi, Toronto (CA); Gennady Poda, Toronto (CA); Luke Whitesell, Toronto (CA); Kejia Ding, Shanghai (CN); Zeren Zhang, Shanghai (CN)

(73) Assignee: Bright Angel Therapeutics Inc., Toronto, ON (CA)

(21) Appl. No.: 19/138,251

(22) PCT Filed: Dec. 20, 2023
(86) PCT No.: PCT/CA2023/051712

§ 371 Date: Jun. 12, 2025

Related U.S. Application Data
(60) Provisional application No. 63/434,607, filed on Dec. 22, 2022.

(51) Int. Cl. A61K 47/54; A61K 47/55; A61P 31/10

(52) U.S. Cl. CPC A61K 47/545; A61K 47/55; A61P 31/10

(57) ABSTRACT

The present application relates conjugate compounds of Formula (I): A-L1-B(I) wherein: A is a moiety that increases fungal cell uptake and/or fungal cell permeability; B is a CK1 inhibiting moiety; and L1 is a linker; or pharmaceutically acceptable salts, solvates and/or prodrugs thereof, to compositions comprising these compounds or pharmaceutically acceptable salts, solvates and/or prodrugs thereof, to processes for their preparation, and their use in therapy such as in the treatment or prevention of fungal-related diseases, disorders or conditions.

### CROSS-REFERENCE TO RELATED APPLICATION

The present application claims the benefit of priority of co-pending U.S. Provisional Patent Application No. 63/434,607, which was filed Dec. 22, 2022, the contents of which are incorporated herein by reference in their entirety.

### FIELD

The present application relates to conjugate compounds, to processes for their preparation, to compositions comprising them, and to their use in therapy. More particularly, it relates to conjugates comprising a moiety that increases fungal cell uptake and/or fungal cell permeability and a type 1 casein kinase inhibitor moiety, useful in the treatment or prevention of fungal-related diseases, disorders or conditions.

### BACKGROUND

Fungal pathogens have an enormous impact on human health worldwide (Fisher et al., Science 360, 739-742 (2018)). Poor clinical outcome for most invasive fungal infections is attributable to the very limited number of effective antifungals available for systemic infections and the emergence of clinical resistance to each of the three main modes of action they target (Brown et al., Sci Transl Med B, 165rv13 (2012)). A rapidly escalating need exists for new, more effective and resistance-aversive antifungals (Banumathy et al., Science 425, 407-410 (2012)).

Protein kinases have emerged as a particularly promising, but to date, clinically unexploited target space for new antifungal drug development. Protein kinases are core signaling molecules central to the regulation of proliferation, survival, and stress responses. As a result, they have been widely studied as potential targets for diverse therapeutic indications, most prominently cancer (Elkins et al., Nat Biotechnol 34, 95-103 (2016); Zhang, et al., Nat Rev Cancer 9, 28-39 (2009)). Kinase inhibitors, however, also possess considerable promise as antimicrobials against diverse pathogenic organisms, including those causing tuberculosis (Carette et al., mBio 9, e02333-17 (2018)), malaria (Derbyshire et al., Chembiochem 15, 1920-30 (2014)) and the most common systemic mycoses (Mattos et al., mBio 11, e02962-19 (2020); LaFayette et al., PLoS Pathog 6, e1001069 (2010); Lee et al., Nat Commun 11(1):1521 (2020)). In fungi, kinases have been implicated in the regulation of diverse biological processes required for the major pathogenic species to cause life-threatening systemic disease (Mattos et al., mBio 11, e02962-19 (2020); LaFayette et al., PLoS Pathog 6, e1001069 (2010); Lee et al., Nat Commun 11, 1521 (2020); Fu et al., Nat Commun 12, 6497 (2021); O'Meara et al., mBio 9, e01581-18 (2018)), but no kinase inhibitors have been approved as antifungals for clinical use to date (Perfect JR., Nat Rev Drug Discov 16, 603-616 (2017)).

Members of the Type 1 casein kinase (CK1) family of serine/threonine kinases are expressed in most eukaryotes (Cheong et al., Int. J. Biochem. Cell Biol 43, 465-469 (2011); Knippschild et al., Cell Signal 17, 675-89 (2005)), but considerable opportunity exists for the development of isoform-specific inhibitors (Fulcher et al., Biochem J 477, 4603-4621 (2020); Monastyrskyi et al., Bioorg Med Chem 26, 590-602 (2018); Wager et al., ACS Chem Neurosci 5, 1253-65 (2014)). In the model fungus Saccharomyces cerevisiae, CK1-family members have been implicated in cell cycle progression, cell morphology, and cell wall integrity as well as endocytosis, cell morphogenesis, mRNA localization, and nutrient sensing (Robinson et al., Mol Biol Cell 10, 1077-1092 (1999); Robinson et al., Mol Cell Biol 13, 2870-2881 (1993); Snowdon et al., Mol Biol Cell 27, 3369-3375 (2016); Hoekstra et al., Science 253, 1031-4 (1991); Ho et al., Proc Natl Acad Sci USA, 94, 581-6 (1997)).

In diverse human fungal pathogens, the CK1-family members Yck2 and Hrr25 are known to play key roles in cell wall integrity, genome maintenance, virulence and drug resistance (Wang et al., Eukaryot Cell 10, 1455-1464 (2011); Caplan et al., Cell Chem Biol 27, 1-14 (2020)). In Candida albicans, the most common cause of invasive fungal infection in humans, the kinase domains of Yck2 and Hrr25 are very highly conserved, but the proteins themselves play very different biological roles. Yck2 is non-essential under rich laboratory growth conditions, but is required for virulence and antifungal drug-resistance (Park et al., Eukaryot Cell 8, 1498-510 (2009); Jung et al., Plos one 12, e0187721 (2017); Liboro et al., Front Cell Infect Microbiol 11, 636834 (2021)).

In contrast, Hrr25 has been shown essential for fungal viability even under rich laboratory culture conditions (Segal et al., mBio 9, e02048-18 (2018)). The role of Yck2 in virulence has been investigated most extensively in C. albicans using both genetical techniques and small molecule inhibitors (Caplan et al., Cell Chem Biol 27, 1-14 (2020)). Genetic-depletion of YCK2 or treatment with inhibitors disrupts the C. albicans morphogenetic program, induces a cell-wall stress response and sensitizes drug-resistant isolates to conventional antifungals in culture. As proof-of-concept, suppression of YCK2 expression using genetic techniques has also been shown to dramatically reduce fungal burden and prolong survival of mice infected with C. albicans (Caplan et al., Cell Chem Biol 27, 1-14 (2020)).

There is a need for new therapeutics for abrogating fungal virulence and enhancing the efficacy of conventional antifungals.

### SUMMARY

The present application includes a conjugate compound of Formula (I), or a pharmaceutically acceptable salt, solvate and/or prodrug thereof,

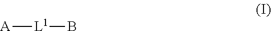

wherein: A is a moiety that increases fungal cell uptake and/or fungal cell permeability; B is a type 1 casein kinase (CK1) inhibiting moiety; and L¹ is a linker comprising at least one first complimentary functional group to covalently link to a functional group in A and at least one second complimentary functional group to covalently link to a functional group in B, to form the conjugate of Formula (I).

In some embodiments, the compound of Formula I optionally comprises one or more atoms that are isotopic variants. In some embodiments, the isotopic variant is deuterium and the compound of Formula I comprises one or more deuterium atoms.

Also included is a method of treating or preventing a fungal-related disease, disorder or condition comprising administering a therapeutically effective amount of one or more conjugate compounds of the present application to a subject in need thereof.

In some embodiments, the present application includes a method of inhibiting or preventing fungal growth comprising administering a therapeutically effective amount of one or more conjugate compounds of the present application to a subject in need thereof.

In some embodiments, the application includes a method of inhibiting fungal CK1 activity comprising administering a therapeutically effective amount of one or more conjugate compounds of the present application to a subject in need thereof.

In some embodiments, the application includes a method of selectively inhibiting fungal CK1 activity comprising administering a therapeutically effective amount of one or more conjugate compounds of the present application to a subject in need thereof.

In some embodiments, the application includes a method of treating or preventing mycosis comprising administering a therapeutically effective amount of one or more conjugate compounds of the present application to a subject in need thereof.

In some embodiments, the application includes a method of treating or preventing a fungal-related disease, disorder or condition comprising administering a therapeutically effective amount of one or more conjugate compounds of the present application in combination with another known agent useful for treatment or prevention of a fungal-related disease, disorder or condition to a subject in need thereof.

Also included in the present application is a pharmaceutical composition comprising one or more conjugate compounds of the present application, or a pharmaceutically acceptable salt, and/or solvate thereof, and a pharmaceutically acceptable carrier and/or diluent.

In some embodiments, the application includes is an agricultural composition comprising one or more conjugate compounds of the present application, or a salt, and/or solvate thereof, and an agriculturally acceptable carrier and/or diluent.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component or effect, such as an additional or second compound, the second compound as used herein is different from the other compounds or first compound. A “third” compound is different from the other, first, and second compounds, and further enumerated or “additional” compounds are similarly different.

The term “and/or” as used herein means that the listed items are present, or used, individually or in combination. In effect, this term means that “at least one of” or “one or more” of the listed items is used or present. The term “and/or” with respect to enantiomers, prodrugs, salts and/or solvates thereof means that the compounds of the application exist as individual enantiomers, prodrugs, salts and hydrates, as well as a combination of, for example, a salt of a solvate of a compound of the application.

The term “compound of the application” or “conjugate compound of the application” and the like as used herein refers to a conjugate compound of Formula (I), or pharmaceutically acceptable salts, solvates and/or prodrugs thereof.

The term “composition of the application” or “composition of the present application” and the like as used herein refers to a composition comprising one or more conjugate compounds of the application.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “inert organic solvent” as used herein refers to a solvent that is generally considered as non-reactive with the functional groups that are present in the compounds to be combined together in any given reaction so that it does not interfere with or inhibit the desired synthetic transformation. Organic solvents are typically non-polar and dissolve compounds that are non soluble in aqueous solutions.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₁₋₆alkylene means an alkylene group having 1, 2, 3, 4, 5 or 6 carbon atoms.

The term “alkenyl” as used herein, whether it is used alone or as part of another group, means straight or branched chain, unsaturated alkyl groups containing at least one double bond. The number of carbon atoms that are possible in the referenced alkylene group are indicated by the prefix “Cₙ₁₋ₙ₂”. For example, the term C₂₋₆alkenyl means an alkenyl group having 2, 3, 4, 5 or 6 carbon atoms and at least one double bond.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆alkynyl means an alkynyl group having 2, 3, 4, 5 or 6 carbon atoms.

The term “cycloalkyl,” as used herein, whether it is used alone or as part of another group, means a saturated carbocyclic group containing from 3 to 20 carbon atoms and one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms.

The term “aryl” as used herein, whether it is used alone or as part of another group, refers to carbocyclic groups containing at least one aromatic ring and contains 6 to 20 carbon atoms.

The term “heterocycloalkyl” as used herein, whether it is used alone or as part of another group, refers to cyclic groups containing at least one non-aromatic ring containing from 3 to 20 atoms in which one or more of the atoms are a heteroatom selected from O, S and N and the remaining atoms are C. Heterocycloalkyl groups are either saturated or unsaturated (i.e. contain one or more double bonds). When a heterocycloalkyl group contains the prefix Cₙ₁₋ₙ₂ this prefix indicates the number of carbon atoms in the corresponding carbocyclic group, in which one or more, suitably 1 to 5, of the ring atoms is replaced with a heteroatom as selected from O, S and N and the remaining atoms are C. Heterocycloalkyl groups are optionally benzofused.

The term “heteroaryl” as used herein, whether it is used alone or as part of another group, refers to cyclic groups containing at least one heteroaromatic ring containing 5-20 atoms in which one or more of the atoms are a heteroatom selected from O, S and N and the remaining atoms are C. When a heteroaryl group contains the prefix Cₙ₁₋ₙ₂ this prefix indicates the number of carbon atoms in the corresponding carbocyclic group, in which one or more, suitably 1 to 5, of the ring atoms is replaced with a heteroatom as defined above. Heteroaryl groups are optionally benzofused.

All cyclic groups, including aryl, heteroaryl, heterocycloalkyl and cycloalkyl groups, contain one or more than one ring (i.e. are polycyclic). When a cyclic group contains more than one ring, the rings may be fused, bridged, spirofused or linked by a bond.

The term “benzofused” as used herein refers to a polycyclic group in which a benzene ring is fused with another ring.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The term “fluorosubstituted” refers to the substitution of one or more, including all, available hydrogens in a referenced group with fluoro.

The terms “halo” or “halogen” as used herein, whether it is used alone or as part of another group, refers to a halogen atom and includes fluoro, chloro, bromo and iodo.

The term “isotopic variant” as used herein refers to an atom that has the same atomic number as the isotope predominantly found in nature, but has an atomic mass or mass number that is different from the atomic mass or mass number of the isotope predominantly found in nature.

The term “available”, as in “available hydrogen atoms” or “available atoms” refers to atoms that would be known to a person skilled in the art to be capable of replacement by a substituent.

The term “cross-coupling” as used herein refers to chemical reactions in which two different starting materials, each of which is usually endowed with an activating group, are reacted together with the aid of a metal catalyst. The result is the loss of the two activating groups and the formation of a new covalent bond between the remaining fragments.

The term “cell” as used herein refers to a single cell or a plurality of cells and includes a cell either in a cell culture or in a subject.

The term “subject” as used herein includes all members of the animal kingdom including mammals and the plant kingdom. Thus the methods and uses of the present application are applicable to human therapy, veterinary and agricultural applications.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with the treatment of subjects.

The term “solvate” as used herein means a compound, or a salt and/or prodrug of a compound, wherein molecules of a suitable solvent are incorporated in the crystal lattice. A suitable solvent is physiologically tolerable at the dosage administered.

The term “prodrug” as used herein means a compound, or salt and/or solvate of a compound, that, after administration, is converted into an active drug.

The term “moiety” as used herein refers to a part of a molecule that is given a name, typically to describe the larger and characteristic parts of organic molecules.

The term “treating” or “treatment” as used herein and as is well understood in the art, means an approach for obtaining beneficial or desired results, including clinical results. Beneficial or desired clinical results can include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alternatively comprise a series of administrations.

“Palliating” a disease or disorder means that the extent and/or undesirable clinical manifestations of a disorder or a disease state are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a fungal-related disease, disorder or condition, or manifesting a symptom associated with a fungal-related disease, disorder or condition.

As used herein, the term “effective amount” or “therapeutically effective amount” means an amount of a compound, or one or more compounds, of the application that is effective, at dosages and for periods of time necessary to achieve the desired result.

The expression “inhibiting CK1” as used herein refers to inhibiting, blocking and/or disrupting CK1 activity in a fungal cell, whether direct or indirect. The inhibiting, blocking and/or disrupting causes a therapeutic effect in the cell.

By “inhibiting, blocking and/or disrupting” it is meant any detectable inhibition, block and/or disruption in the presence of a compound compared to otherwise the same conditions, except for in the absence in the compound.

The term “fungal-related disease, disorder or condition” means that the disease, disorder or condition to be treated is affected by, modulated by and/or has some biological basis, either direct or indirect, that includes fungal activity. These diseases respond favourably when fungal activity associated with the disease, disorder or condition is inhibited by one or more of the compounds or compositions of the application.

The term “CK1” as used herein refers to type 1 casein kinase.

The term “fungal cell uptake” or “fungal cell permeability” as used herein refers to the regulation or controlling exchanges of molecules between the cell and its environment, for example an increased cell permeability would mean an increased ability for a molecule to internalize into the cell.

The term “administered” as used herein means administration of a therapeutically effective amount of a compound, or one or more compounds, or a composition of the application to a cell either in cell culture or in a subject.

The term “linker moiety” as used herein refers to any molecular structure that joins two or more other molecular structures together.

The term “complementary functional group” as used herein refers to a group of atoms or a single atom that will react with another group of atoms or a single atom to form a covalent bond between the two groups or atoms.

The term “reacts with” as used herein generally means that there is a flow of electrons or a transfer of electrostatic charge resulting in the formation of a covalent bond.

The term “conjugating” as used herein means to bind two molecules together via a covalent bond.

The present description refers to a number of chemical terms and abbreviations used by those skilled in the art. Nevertheless, definitions of selected terms are provided for clarity and consistency.

The term “aq.” as used herein refers to aqueous.

The term “rt” as used herein refers to room temperature.

The term “Me” as used herein refers to methyl.

The term “Et” as used herein refers to ethyl.

The term “Pr” as used herein refers to propyl.

The term “Bu” as used herein refers to butyl.

The term “Ac” as used herein refers to acetyl.

The term “Ph” as used herein refers to phenyl.

The term “Ts” as used herein refers to tosyl.

The term “Ms” as used herein refers to mesyl.

The term “THF” as used herein refers to tetrahydrofuran.

The term “HATU” as used herein refers to hexafluorophosphate azabenzotriazole tetramethyl uronium.

The terms “DIPEA” or “DIEA” as used herein refer to N,N-diisopropylethylamine.

The term “TEA” as used herein refers to triethylamine.

The terms “TBME” or “MTBE” as used herein refer to methyl tert-butyl ether.

The term “DMF” as used herein refers to dimethylformamide.

The term “DCM” as used herein refers to dichloromethane.

The term “DME” as used herein refers to dichloroethane.

The term “DMAP” as used herein refers to 4-dimethylaminopyridine.

The term “DMA” as used herein refers to dimethylacetamide.

The term “DIB” as used herein refers to diisobutylene.

The term “DEA” as used herein refers to diethanolamine.

The term “DBU” as used herein refers to 1,8-diazabicyclo[5.4.0]undec-7-ene

The term “TFA” as used herein refers to trifluoroacetic acid.

The term “ACN” as used herein refers to acetonitrile.

The term “TMS” as used herein refers to trimethylsilyl.

The term “SEM” as used herein refers to trimethylsilylethoxymethyl.

The term “BOC” as used herein refers to tert-butyloxycarbonyl.

The term “DMSO” as used herein refers to dimethylsulfoxide.

The term “NBS” as used herein refers to N-bromosuccinimide.

The term “IPA” as used herein refers to isopropyl alcohol.

The term “DMP” as used herein refers to Dess-Martin periodinane.

The term “CDI” as used herein refers to 1,1′-carbonyldiimidazole.

The term “HBPIN” as used herein refers to pinacolborane or 4,4,5,5-tetramethyl-1,3,2-dioxaborolanee.

The term “TEMPO” as used herein refers to (2,2,6,6-Tetramethylpiperidin-1-yl)oxyl or (2,2,6,6-tetramethylpiperidin-1-yl)oxidanyl.

The term “CTAB” as used herein refers to cetrimonium bromide or cetyltrimethylammonium bromide or hexadecyltrimethylammonium bromide.

The term “MW” as used herein refers to microwave.

The term “ee” as used herein refers to enantiomeric excess.

The term “HPLC” as used herein refers to high-performance liquid chromatography.

The term “NMR” as used herein refers to nuclear magnetic resonance.

The term “MS” as used herein refers to mass spectrometry.

The term “LCMS” as used herein refers to liquid chromatography-mass spectrometry.

The term “IC₅₀” as used herein refers to half maximal inhibitory concentration.

The term “ATP” as used herein refers to adenosine 5′-triphosphate.

The term “PCR” as used herein refers to polymerase chain reaction.

The term “MIC” as used herein refers to minimum inhibitory concentrations.

The term “TEV” as used herein refers to tobacco etch virus.

### II. Compounds and Compositions

The present application describes a novel class of conjugate compounds.

Accordingly, the application includes a conjugate compound of Formula (I), or a pharmaceutically acceptable salt, solvate and/or prodrug thereof,

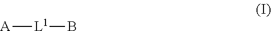

wherein:
A is a moiety that increases fungal cell uptake and/or fungal cell permeability;
B is a CK1 inhibiting moiety; and
L¹ is a linker comprising at least one first complimentary functional group to covalently link to a functional group in A and at least one second complimentary functional group to covalently link to a functional group in B.

In some embodiments, the compound of Formula I optionally comprises one or more atoms that are isotopic variants. In some embodiments, the isotopic variant is deuterium and the compound of Formula I comprises one or more deuterium atoms.

In some embodiments, A is an antifungal moiety. In some embodiments, A comprises one or more of: an alkylene moiety, an azole moiety, an indole moiety, a phenyl moiety, a carbazole moiety, pyridoindole moiety, a tetrahydropyridoindole moiety, a polyamine moiety, a fatty acid alkyl chain moiety, a flucytosine (5-FC) moiety and a triphenylphosphonium moiety, each of which are optionally substituted with one or more groups independently selected from halo, OH, C₁₋₄alkyl, C₁₋₄haloalkyl, OC₁₋₄alkyl, C(O)OH, C(O)NH₂, C(O)C₁₋₄alkyl, C(O)OC₁₋₄alkyl, C(O)NHC₁₋₄alkyl, C(O)N(C₁₋₄alkyl)C₁₋₄alkyl), C(NH)NH₂, C(NC₁₋₄alkyl)NH₂, C(NC₁₋₄alkyl)NHC₁₋₄alkyl, C(NC₁₋₄alkyl)N(C₁₋₄alkyl)(C₁₋₄alkyl), C(NH)NHC₁₋₄alkyl and C(NH)N(C₁₋₄alkyl)(C₁₋₄alkyl), wherein each alkyl group is optionally halosubstituted, suitably fluorosubstituted. In some embodiments, A optionally comprises one or more atoms that are isotopic variants. In some embodiments, the isotopic variant is deuterium and A comprises one or more deuterium atoms. In some embodiments, an azole moiety refers to a five-member heterocyclic rings containing a nitrogen and at least one other non-carbon atom of general structure:

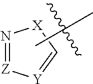

Y where X, Y and Z are independently selected from CH, N, O and S, and optionally their reduced analogs, and/or optionally substituted versions thereof wherein the optional substituents are as defined above. The point of attachment indicated by [symbol] can be at any atom in the ring that has a valency which permits a covalent attachment. In some embodiments, the azole moiety is selected from an imidazole moiety, a triazole moiety, tetrazole moiety and a thiazole moiety.

In some embodiments, the indole moiety has the general structure:

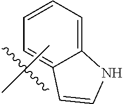

and optionally substituted versions thereof wherein the optional substituents are as defined above. The point of attachment indicated by [symbol] can be at any atom in the two rings that has a valency which permits a covalent attachment.

In some embodiments, the carbazole moiety has the general structure:

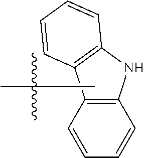

and optionally substituted versions thereof wherein the optional substituents are as defined above. The point of attachment indicated by [symbol] can be at any atom in the three rings that has a valency which permits a covalent attachment.

In some embodiments, the pyridoindole moiety has the general structure:

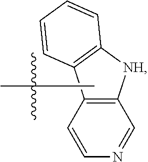

and optionally substituted versions thereof wherein the optional substituents are as defined above. The point of attachment indicated by [symbol] can be at any atom in the three rings that has a valency which permits a covalent attachment.

In some embodiments, the tetrahydropyridoindole moiety has the general structure:

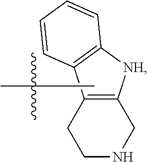

and optionally substituted versions thereof wherein the optional substituents are as defined above. The point of attachment indicated by [symbol] can be at any atom in the three rings that has a valency which permits a covalent attachment.

In some embodiments, the polyamine moiety is an alkyl chain that is saturated or unsaturated, branched or linear, and comprising from 4 to 20 carbons and two or more amino groups of the formula NR^{y}, and optionally one terminal amino group of the formula N(R^{y})₂, wherein R^{y} is selected from H, C₁₋₆alkyl and C₁₋₆haloalkyl.

In some embodiments, the fatty acid alkyl chain moiety refers to an aliphatic chain comprising from 4 to 28 carbons, which is either saturated or unsaturated, branched or linear, and optionally halosubstituted, suitably fluorosubstituted. In some embodiments, when the fatty acid is unsaturated, said fatty acid is monounsaturated or polyunsaturated containing two or more carbon-carbon double bonds. In some embodiments, the fatty acid contains two, three or four double bonds. In some embodiments, the fatty acid contains three double bonds.

In some embodiments, a flucytosine (5-FC) moiety refers to a compound of formula

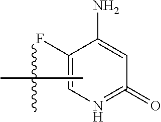

and analogs thereof, wherein point of attachment indicated by [symbol] can be at any atom in the moiety that has a valency which permits a covalent attachment.

In some embodiments, a triphenylphosphonium moiety refers to a moiety of general formula:

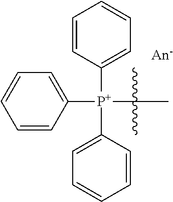

where An⁻ is an associated anion.

In some embodiments, A is any antifungal agent known in the art. In some embodiments, A is selected from fluconazole, itraconazole, clotrimazole, ketoconazole, voriconazole, posaconazole, isavuconazonium, miconazole, flucytosine, olorofim, manogepix, ibrexafungerp, caspofungin, micafungin, anidulafungin, rezafungin, amphotericin B, and VT-1161, each of which is bound to L¹ via any reactive functional group or atom in the molecule.

In some embodiments, A is selected from:

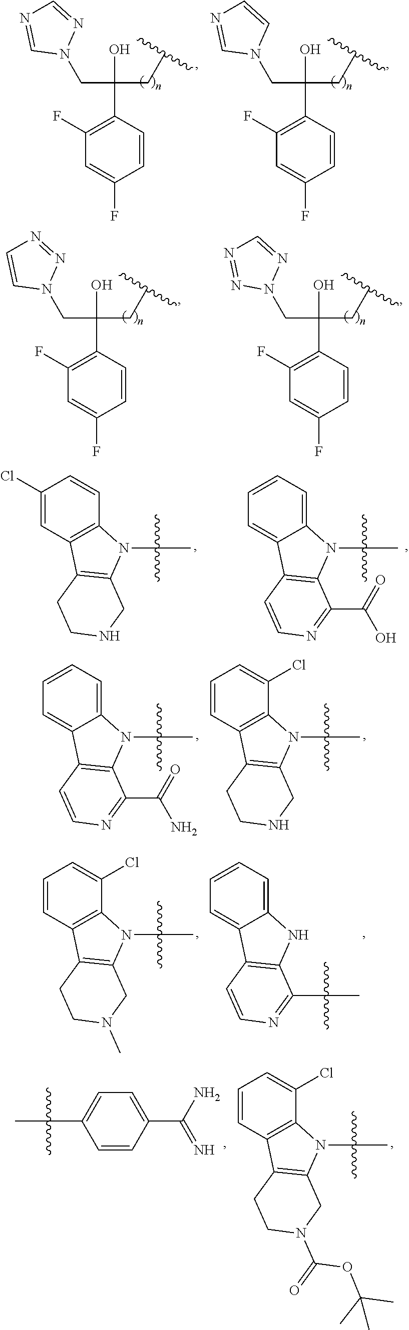

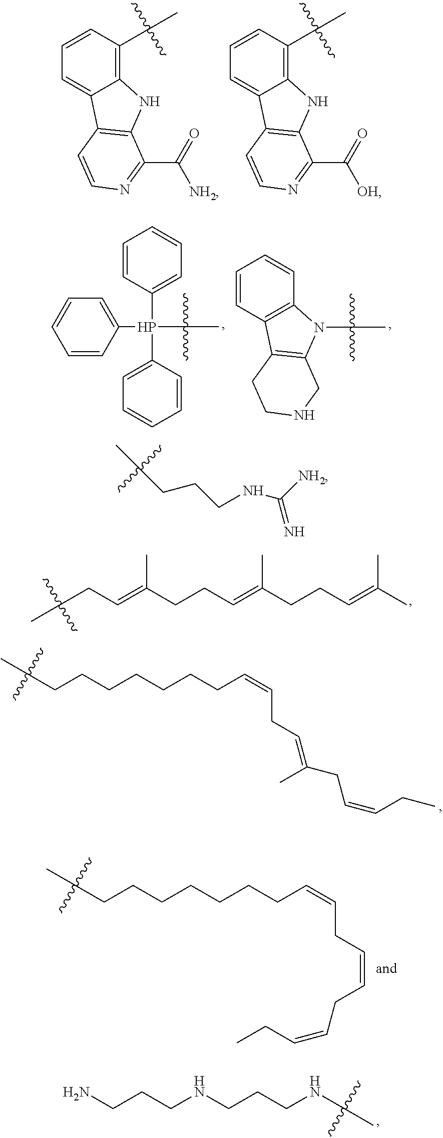

wherein each n is independently an integer from 0 to 4, and

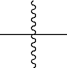

represents the point of attachment to L¹, including all stereoisomers thereof.

In some embodiments, A is selected from:

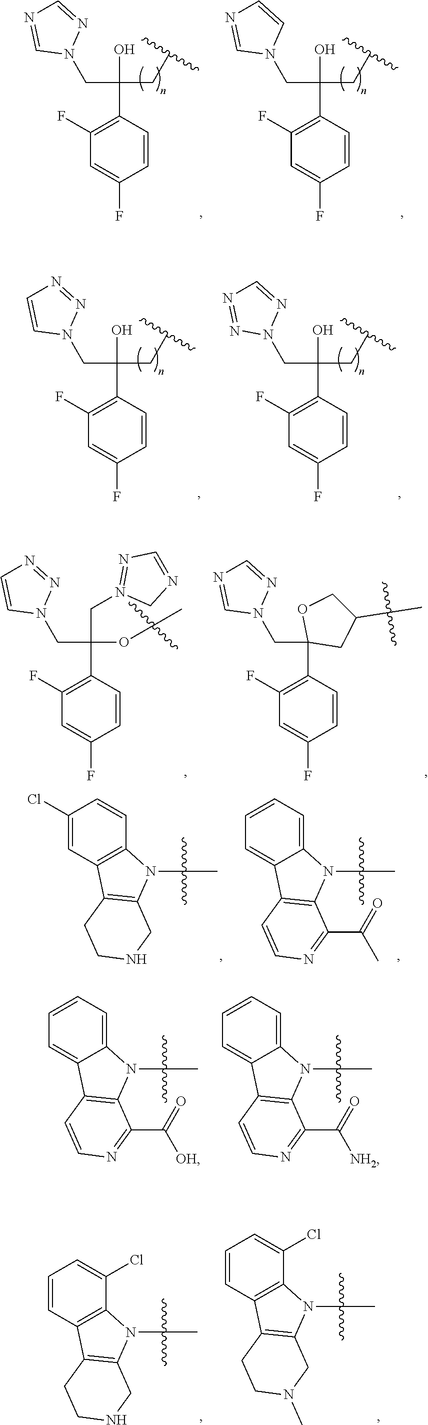

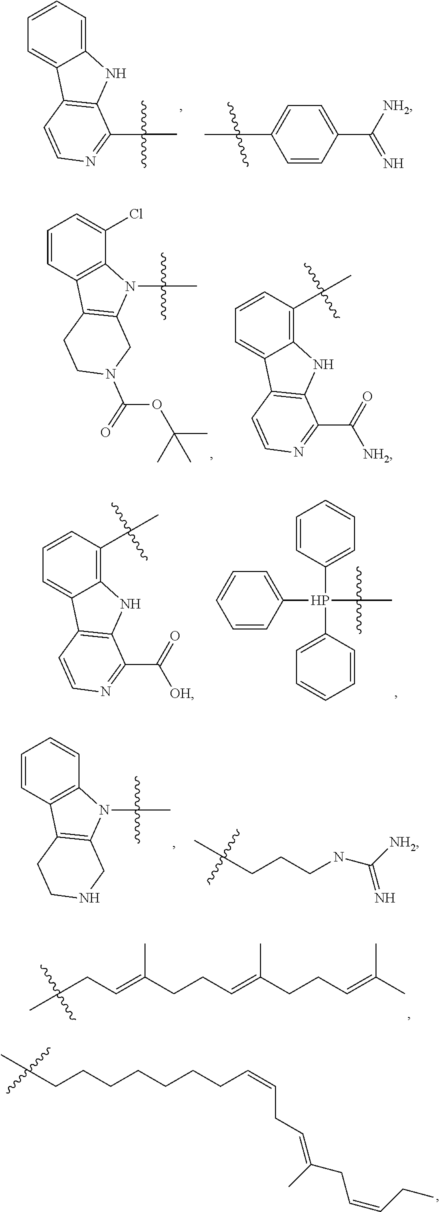

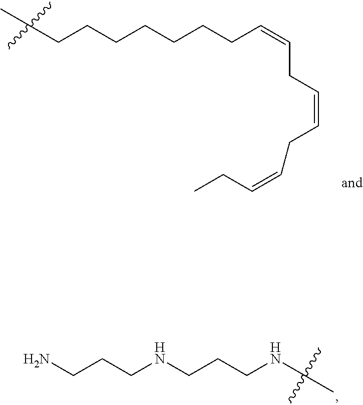

wherein each n is independently an integer from 0 to 4, and

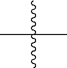

represents the point of attachment to L¹, including all stereoisomers thereof; and
wherein A optionally comprises one or more atoms that are isotopic variants, for example, wherein the isotopic variant is deuterium and A comprises one or more deuterium atoms.

In some embodiments, L¹ comprises one or more independently selected groups selected from C(O), C(═NR¹), O, NR¹, C₁₋₁₂alkylene, C₆₋₂₀arylene, C₃₋₁₀cycloalkylene, C₂₋₂₀heteroarylene and C₃₋₁₀heterocycloalkylene, wherein each of the alkylene, arylene, cycloalkylene, heteroarylene and heterocycloalkylene is optionally substituted with one or more R², and each R² is independently selected from C₁₋₁₂alkyl, NR³C(O)R⁴, NR³R⁴, halo, C(O)NR³R⁴, and OR³; and R¹, R³ and R⁴ are independently selected from H, C₁₋₁₂alkyl and C₁₋₁₂haloalkyl. In some embodiments, L¹ optionally comprises one or more atoms that are isotopic variants. In some embodiments, the isotopic variant is deuterium and L¹ comprises one or more deuterium atoms.

In some embodiments, L¹ comprises 6 groups independently selected from C(O), C(═NR¹), O, NR¹, C₁₋₁₂alkylene, C₆₋₂₀arylene, C₃₋₁₀cycloalkylene, C₂₋₂₀heteroarylene and C₃₋₁₀heterocycloalkylene. In some embodiments, L¹ comprises 5 groups independently selected from C(O), C(═HR¹), O, NR¹, C₁₋₁₂alkylene, C₆₋₂₀arylene, C₃₋₁₀cycloalkylene, C₂₋₂₀heteroarylene and C₃₋₁₀heterocycloalkylene. In some embodiments, L¹ comprises 4 groups independently selected from C(O), C(═R¹), O, NR¹, C₁₋₁₂alkylene, C₆₋₂₀arylene, C₃₋₁₀cycloalkylene, C₂₋₂₀heteroarylene and C₃₋₁₀heterocycloalkylene. In some embodiments, L¹ comprises 3 groups independently selected from C(O), C(═NR¹), O, NR¹, C₁₋₁₂alkylene, C₆₋₂₀arylene, C₃₋₁₀cycloalkylene, C₂₋₂₀heteroarylene and C₃₋₁₀heterocycloalkylene. In some embodiments, L¹ comprises 2 groups selected from C(O), C(═NR¹), O, NR², C₁₋₁₂alkylene, C₆₋₂₀arylene, C₃₋₁₀cycloalkylene, C₂₋₂₀heteroarylene and C₃₋₁₀heterocycloalkylene. In some embodiments, each of the alkylene, arylene, cycloalkylene, heteroarylene and heterocycloalkylene is optionally and independently substituted with one or more, one to six, one to five, one to four, one to three, one or two, or one R². In some embodiments, L¹ comprises one or more, one to six, one to five, one to four, one to three, one or two, or one of C₁₋₆alkylene, C₆₋₁₀arylene, C₃₋₈cycloalkylene, C₂₋₁₀heteroarylene and/or C₃₋₈heterocycloalkylene, each of which is optionally and independently substituted with one or more, one to six, one to five, one to four, one to three, one or two, or one R².

In some embodiments, L¹ is selected from C₁₋₁₂alkylene-NR¹—C(O), C₁₋₆alkylene-C₆₋₁₀arylene-NR¹—C(O), C₁₋₆alkylene-NR¹—C(O)—C₂₋₁₀heteroarylene, C₁₋₆alkylene-C₂₋₁₀heteroarylene-NR¹—C(O), C₂₋₁₀heteroarylene-NR¹—C(O), C₁₋₆alkylene-C₃₋₁₀heterocycloalkylene-NR¹—C(O)—C₂₋₁₀heteroarylene, C₁₋₆alkylene-C₃₋₁₀heterocycloalkylene-C₂₋₁₀heteroarylene, C₁₋₆alkylene-C₃₋₁₀heterocycloalkylene-NR¹—C(O), C₁₋₆alkylene-C₃₋₁₀heterocycloalkylene-C₁₋₆alkylene-NR¹—C(O), C₁₋₆alkylene-C₃₋₁₀heterocycloalkylene-C₆₋₁₀arylene-NR¹—C(O), C₁₋₆alkylene-C₃₋₁₀heterocycloalkylene-C₁₋₆alkylene-C₆₋₁₀arylene-NR¹—C(O), C₁₋₆alkylene-C(O)—C₃₋₁₀heterocycloalkylene-NR¹—C(O), C₁₋₆alkylene-C(O)—C₃₋₁₀heterocycloalkylene-NR¹—C₁₋₆alkylene, C₁₋₆alkylene-O—C(O)—C₁₋₆alkylene-C₃₋₁₀heterocycloalkylene-NR¹—C(O), C₁₋₆alkylene-C₃₋₁₀heterocycloalkylene-NR¹—C₁₋₆alkylene, C₁₋₆alkylene-C₃₋₁₀heterocycloalkylene-C₁₋₆alkylene-NR¹—C₁₋₆alkylene, C₁₋₆alkylene-C₃₋₁₀heterocycloalkylene-C₁₋₆alkylene, C₁₋₆alkylene-NR¹—C₁₋₆alkylene, 0-C₁₋₁₂alkylene, C₁₋₆alkylene-C₃₋₁₀heterocycloalkylene-O—C₁₋₆alkylene-C₆₋₁₀arylene-NR¹—C(O), C₁₋₆alkylene-C₃₋₁₀heterocycloalkylene-O—C₁₋₆alkylene-O—C₆₋₁₀arylene-NR¹—C(O), and NR¹—C₁₋₆alkylene-O—C₁₋₆ alkylene-O—C₁₋₆alkylene-NR¹. Each L¹ defined above is optionally substituted with one or more R². A person skilled in the art will appreciate that each of A and B are covalently bonded to either end of L¹ as defined above.

In some embodiments, each R² is independently selected from C₁₋₆alkyl, NR³C(O)R⁴, NR³R⁴, C(O)NR³R⁴, and OR³; and R³ and R⁴ are independently selected from H, C₁₋₆alkyl and C₁₋₆fluoroalkyl. In some embodiments, each R² is independently selected from methyl, ethyl, propyl, NH₂, NHC(O)C₁₋₆alkyl, C(O)NH₂ and OH. In some embodiments, each R² is independently selected from methyl, ethyl, C(O)NH₂ and OH. In some embodiments, R³ and R⁴ are independently selected from H, methyl, ethyl, and propyl.

In some embodiments, L¹ is selected from:

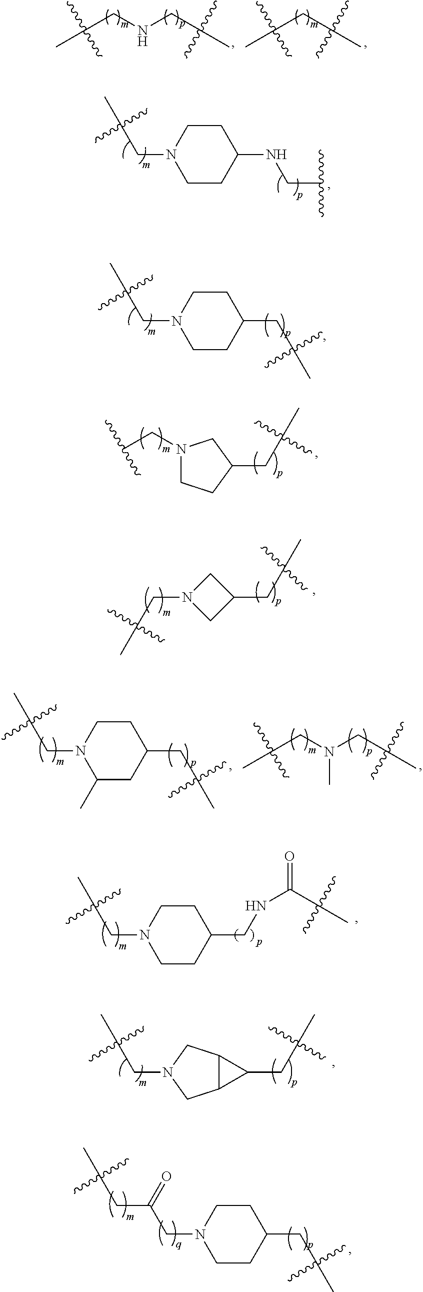

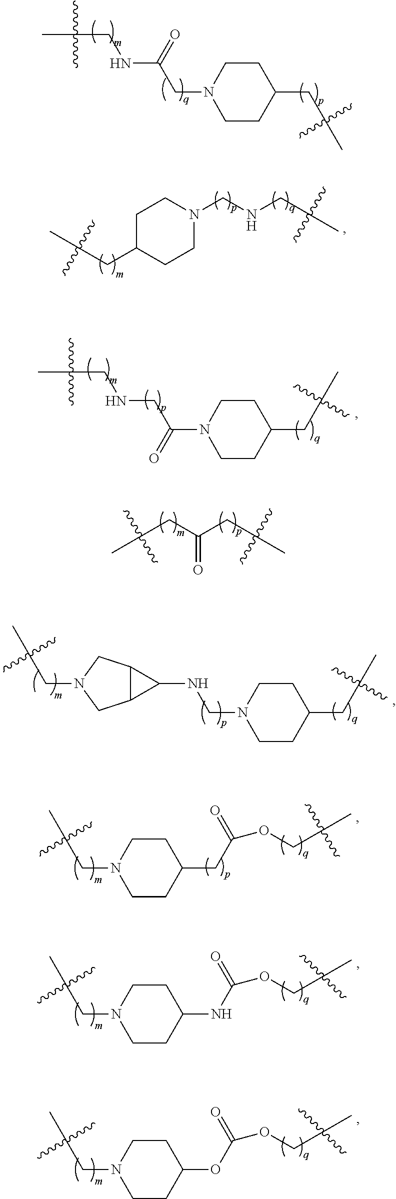

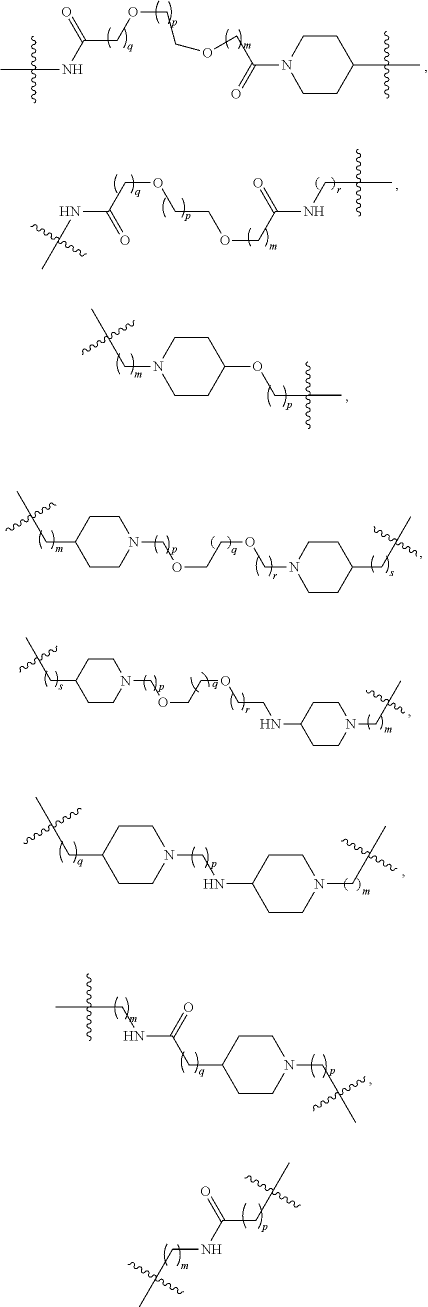

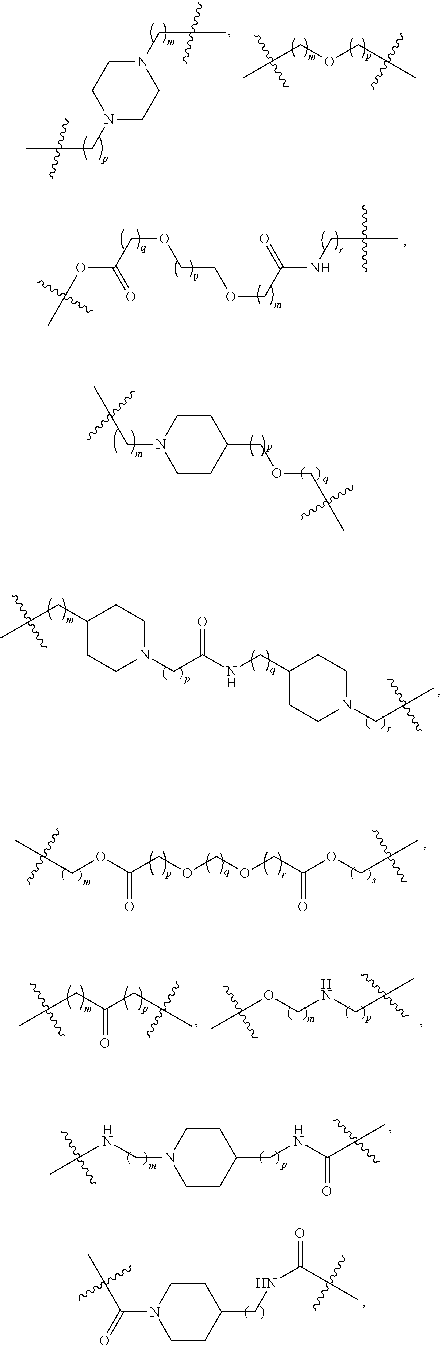

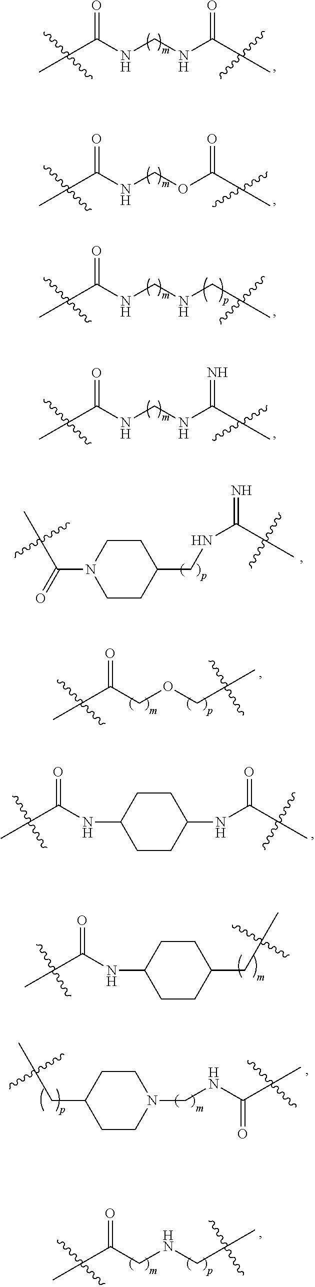

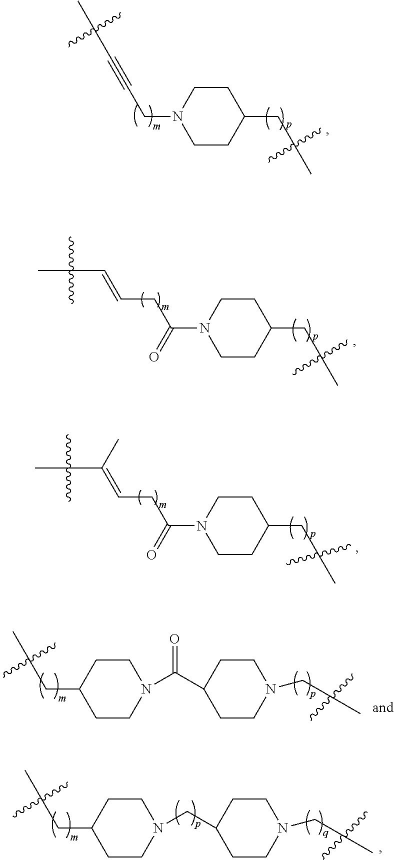

wherein each m, p, q, r and s is independently an integer from 0 to 6, and

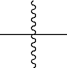

represents the points of attachment to either A or B, including all stereoisomers thereof.

In some embodiments, L¹ is selected from:

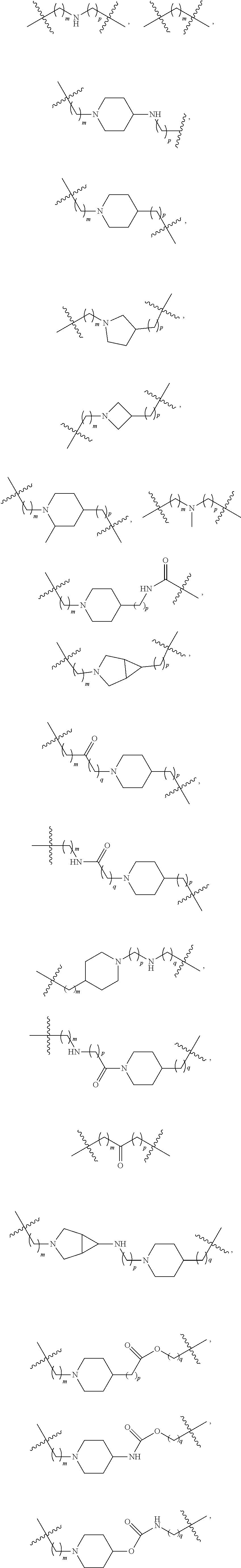

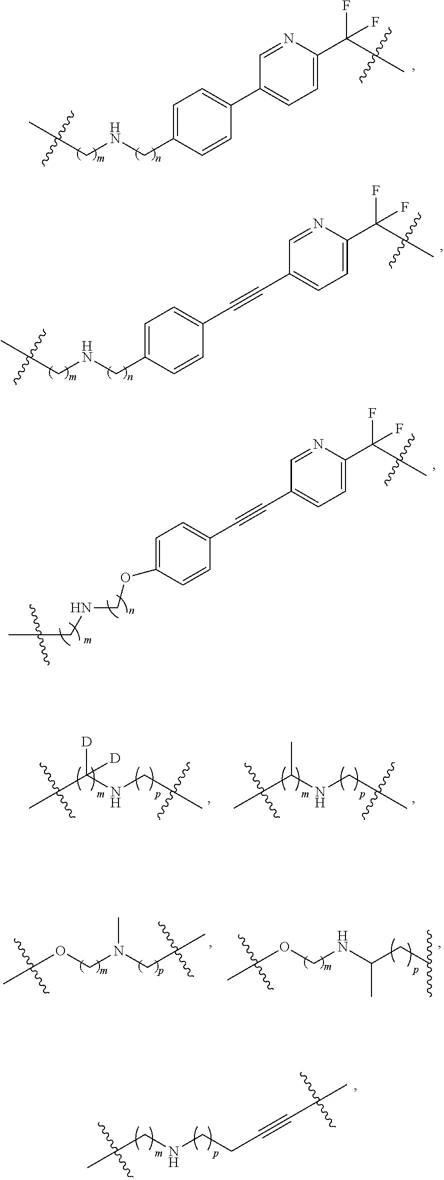

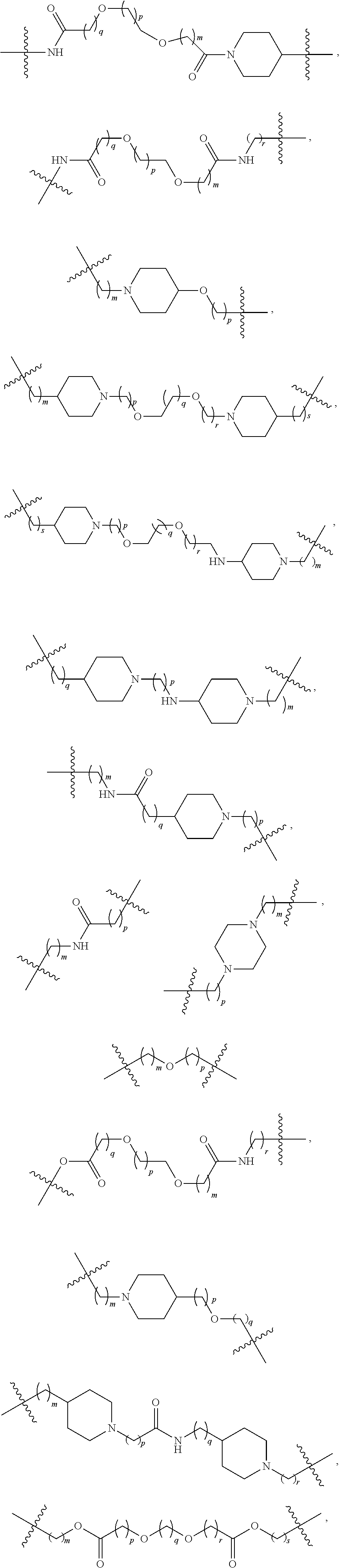

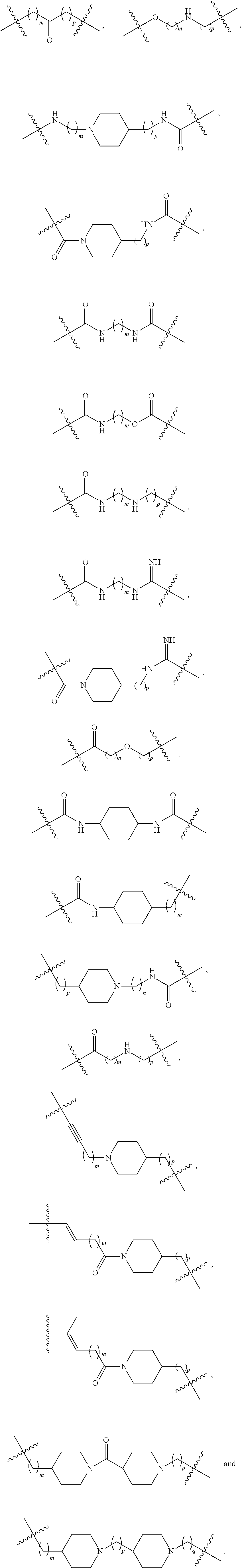

wherein each m, p, q, r and s is independently an integer from 0 to 6, and

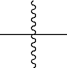

represents the points of attachment to either A or B, including all stereoisomers thereof, and wherein L¹ optionally comprises one or more atoms that are isotopic variants, for example, wherein the isotopic variant is deuterium and L¹ comprises one or more deuterium atoms.

In some embodiments, B is a C₄₋₂₀heteroaryl moiety comprising between 2 and 6 N heteroatoms. In some embodiments, B comprises at least one fluorophenyl. In some embodiments, B comprises one or more of: a pyridine moiety, a pyrimidine moiety, a pyrazole moiety, a piperazine moiety and an imidazole moiety. In some embodiments, B comprises a pyrazolopyridine moiety or a pyrrolopyridinone moiety. In some embodiments. B comprises any compound known to inhibit CK1, such as the compounds listed in Table 1 below, including all stereoisomers thereof. The compounds shown for B may be bonded to L¹ via any reactive functional group present in the molecule, and this would be well within the purview of a skilled person in the art. In some embodiments, B optionally comprises one or more atoms that are isotopic variants. In some embodiments, the isotopic variant is deuterium and B comprises one or more deuterium atoms.

TABLE 1
CK1 Inhibitors

| Name | IUPAC Chemical Name |
|---|---|
| CK1-7 | N-(2-aminoethyl)-5-chloroisoquinoline- |
| | 8-sulfonamide |
| D4476 | 4-(4-(2,3-dihydrobenzo[b][1,4]dioxin-6-yl)-5- |
| | (pyridin-2-yl)-1H-imidazol-2-yl)benzamide |
| IC261 | (E)-3-(2,4,5-trimethoxybenzylidene)indolin-2-one |
| PF-670462 | 4-(1-cyclohexyl-4-(4-fluorophenyl)-1H-imidazol- |
| | 5-yl)pyrimidin-2-amine |
| PF5006739 | 4-(4-(4-fluorophenyl)-1-(1-(isoxazol- |
| | 3-ylmethyl)piperidin-4-yl)-1H- |
| | imidazol-5-yl)pyrimidin-2-amine |
| PF4800567 | 3-((3-chlorophenoxy)methyl)-1- |
| | (tetrahydro-2H-pyran-4-yl)-1H- |
| | pyrazolo[3,4-d]pyrimidin-4-amine |
| (R)- | (R)-2-((9-isopropyl-6-((3-(pyridin-2- |
| DRF053 | yl)phenyl)amino)-9H-purin-2- |
| | yl)amino)butan-1-ol |
| SR-2890 | N-((6-chloro-1H-benzo[d]imidazol- |
| | 2-yl)methyl)-9-(3-fluorophenyl)-2- |
| | (piperazin-1-yl)-9H-purin-6-amine |
| SR-3029 | N-((6,7-difluoro-1H-benzo[d]imidazol- |
| | 2-yl)methyl)-9-(3-fluorophenyl)-2- |
| | morpholino-9H-purin-6-amine |
| SR-653234 | N-((1H-benzo[d]imidazol-2-yl)methyl)- |
| | 2-morpholino-9-(thiophen-3- |
| | yl)-9H-purin-6-amine |
| IGS-2.7 | 2-(3-chlorophenyl)-N-(6- |
| | (trifluoromethyl)benzo[d]thiazol-2- |
| | yl)acetamide |
| NCC007 | 9-(1-cyclopropylethyl)-N2-(2- |
| | (dimethylamino)ethyl)-N6-(3- |
| | (trifluoromethyl)benzyl)-9H-purine-2,6-diamine |
| SR-2890 | N-((6-chloro-1H-benzo[d]imidazol-2- |
| | yl)methyl)-9-(3-fluorophenyl)-2- |
| | (piperazin-1-yl)-9H-purin-6-amine |
| IG-2.7 | 2-(3-chlorophenyl)-N-(6- |
| | (trifluoromethyl)benzo[d]thiazol-2- |
| | yl)acetamide |
| K687 | 1-(benzo[d]thiazol-2-yl)-3-(3-chloro- |
| | 4-hydroxyphenyl)urea |
| IWP-2 | N-(5-methylbenzo[d]thiazol-2-yl)-2- |
| | ((4-oxo-3-phenyl-3,4,6,7-tetrahydrothieno[3,2- |
| | d]pyrimidin-2-yl)thio)acetamide |
| Prodigiosin | (Z)-4-methoxy-5-((5-methyl-4-pentyl- |
| | 2H-pyrrol-2-ylidene)methyl)- |
| | 1H, 1′H-2,2′-bipyrrole |
| Ningalin C | 3-(3,4-dimethoxyphenethyl)-1,4- |
| | bis(3,4-dimethoxyphenyl)-7,8- |
| | dimethoxy-2H-benzo[e]indole-2,5(3H)-dione |
| TP | 5-amino-N-(5-aminopentyl)- |
| | 2-(3,5-dihydroxyphenyl)-[1,2,4]triazolo[1,5- |
| | c]pyrimidine-8-carboxamide |
| TT | 5-(7-amino-5-(benzylamino)- |
| | [1,2,4]triazolo[1,5-a][1,3,5]triazin-2- |
| | yl)benzene-1,3-diol |

In some embodiments, B is selected from:

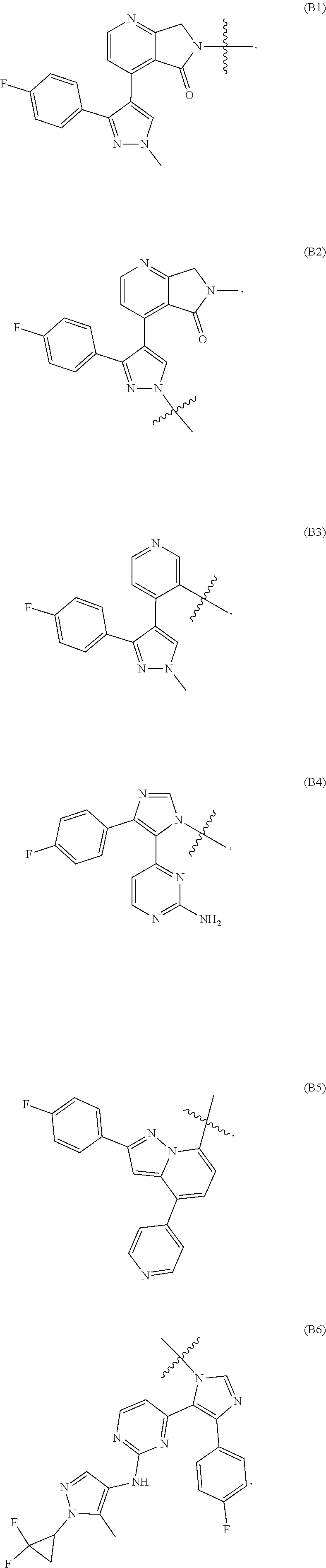 [SMILES: *N1Cc2nccc(-c3cn(C)nc3-c3ccc(F)cc3)c2C1=O ; *n1cc(-c2ccnc3c2C(=O)N(C)C3)c(-c2ccc(F)cc2)n1 ; *c1cnccc1-c1cn(C)nc1-c1ccc(F)cc1 ; *n1cnc(-c2ccc(F)cc2)c1-c1ccnc(N)n1 ; *c1ccc(-c2ccncc2)c2cc(-c3ccc(F)cc3)nn12 ; *n1cnc(-c2ccc(F)cc2)c1-c1ccnc(Nc2cnn(C3CC3(F)F)c2C)n1]

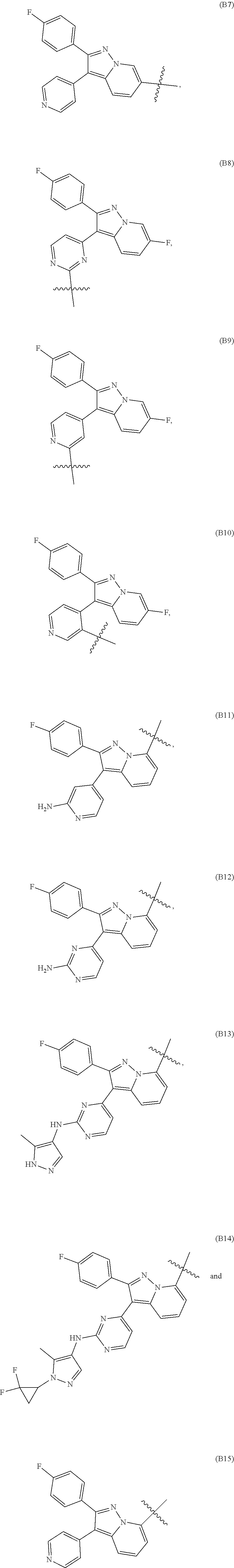

wherein

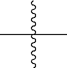

represents the point of attachment to L¹.

In some embodiments, B optionally comprises one or more atoms that are isotopic variants, for example, wherein the isotopic variant is deuterium and B comprises one or more deuterium atoms.

In some embodiments, the conjugate compound of Formula (I) is selected from the compounds listed in Table 2:

TABLE 2

| Compound | | |
|---|---|---|
| ID# | IUPAC Name | Structure |
| I-1 | 6-(2-(2,4-difluorophenyl)-2- hydroxy-3-(1H-imidazol-1- yl)propyl)-4-(3-(4- fluorophenyl)-1-methyl-1H- pyrazol-4-yl)-6,7-dihydro-5H- pyrrolo[3,4-b]pyridin-5-one | 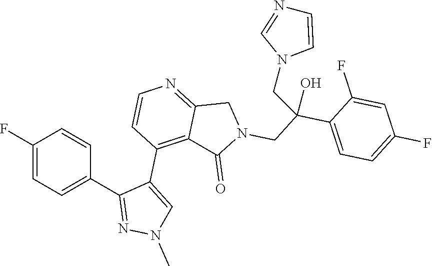 [SMILES: Cn1cc(-c2ccnc3c2C(=O)N(CC(O)(Cn2ccnc2)c2ccc(F)cc2F)C3)c(-c2ccc(F)cc2)n1] |
| I-2 | 4-(1-(3-(2,4-difluorophenyl)-3- hydroxy-4-(1H-1,2,4-triazol-1- yl)butyl)-3-(4-fluorophenyl)-1H- pyrazol-4-yl)-6-methyl-6,7- dihydro-5H-pyrrolo[3,4- b]pyridin-5-one | 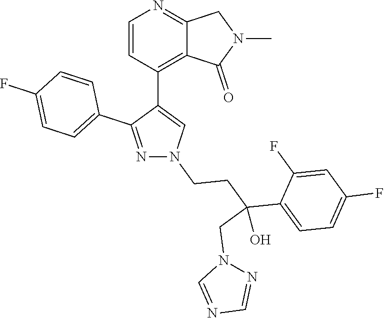 [SMILES: CN1Cc2nccc(-c3cn(CCC(O)(Cn4cncn4)c4ccc(F)cc4F)nc3-c3ccc(F)cc3)c2C1=O] |
| I-3 | 2-(2,4-difluorophenyl)-4-(((4- (3-(4-fluorophenyl)-1-methyl- 1H-pyrazol-4-yl)pyridin-3- yl)methyl)amino)-1-(1H-1,2,4- triazol-1-yl)butan-2-ol | 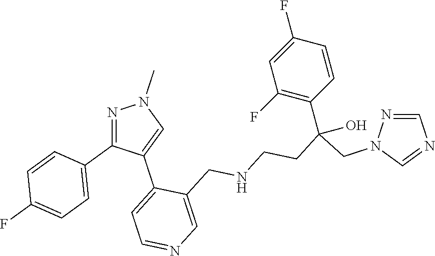 [SMILES: Cn1cc(-c2ccncc2CNCCC(O)(Cn2cncn2)c2ccc(F)cc2F)c(-c2ccc(F)cc2)n1] |
| I-4 | 2-(2,4-difluorophenyl)- 1-(4- (((4-(3-(4-fluorophenyl)-1- methyl-1H-pyrazol-4-yl)pyridin- 3-yl)methyl)amino)piperidin-1- yl)-3-(1H-1,2,4-triazol-1- yl)propan-2-ol | 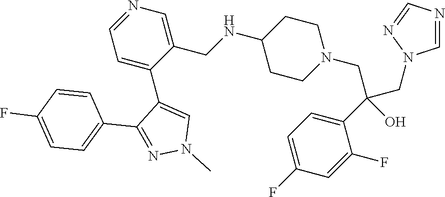 [SMILES: Cn1cc(-c2ccncc2CNC2CCN(CC(O)(Cn3cncn3)c3ccc(F)cc3F)CC2)c(-c2ccc(F)cc2)n1] |
| I-5 | 2-(2,4-difluorophenyl)- 1-(((4- (3-(4-fluorophenyl)-1-methyl- 1H-pyrazol-4-yl)pyridin-3- yl)methyl)amino)-3-(1H-1,2,4- triazol-1-yl)propan-2-ol | 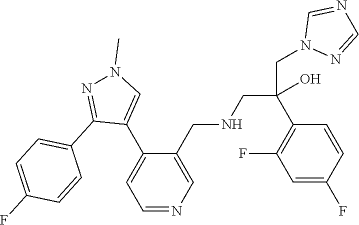 [SMILES: Cn1cc(-c2ccncc2CNCC(O)(Cn2cncn2)c2ccc(F)cc2F)c(-c2ccc(F)cc2)n1] |
| I-6 | 6-(2-(2,4-difluorophenyl)-2- hydroxy-3-(1H-1,2,4-triazol-1- yl)propyl)-4-(3-(4- fluorophenyl)-1-methyl-1H- pyrazol-4-yl)-6,7-dihydro-5H- pyrrolo[3,4-b]pyridin-5-one | 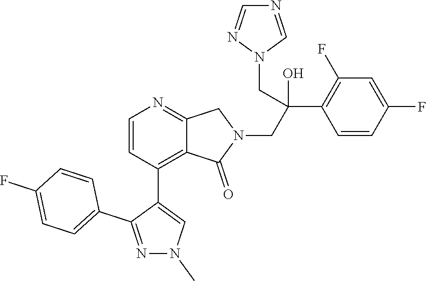 [SMILES: Cn1cc(-c2ccnc3c2C(=O)N(CC(O)(Cn2cncn2)c2ccc(F)cc2F)C3)c(-c2ccc(F)cc2)n1] |
| I-7 | (S)-6-(2-(2,4-difluorophenyl)-2- hydroxy-3-(1H-1,2,4-triazol-1- yl)propyl)-4-(3-(4- fluorophenyl)-1-methyl-1H- pyrazol-4-yl)-6,7-dihydro-5H- pyrrolo[3,4-b]pyridin-5-one | 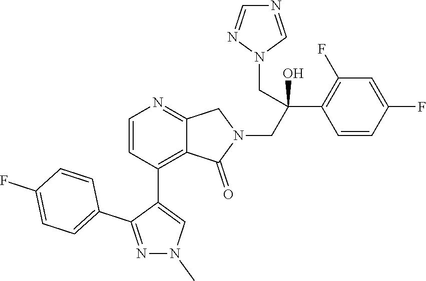 [SMILES: Cn1cc(-c2ccnc3c2C(=O)N(C[C@](O)(Cn2cncn2)c2ccc(F)cc2F)C3)c(-c2ccc(F)cc2)n1] |
| I-8 | (R)-6-(2-(2,4-difluorophenyl)-2- hydroxy-3-(1H-1,2,4-triazol-1- yl)propyl)-4-(3-(4- fluorophenyl)-1-methyl-1H- pyrazol-4-yl)-6,7-dihydro-5H- pyrrolo[3,4-b]pyridin-5-one | 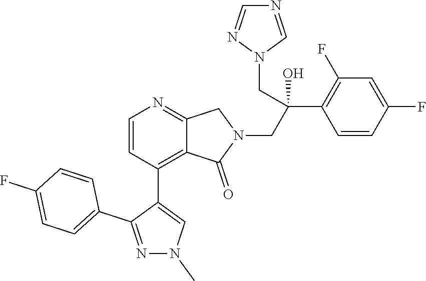 [SMILES: Cn1cc(-c2ccnc3c2C(=O)N(C[C@@](O)(Cn2cncn2)c2ccc(F)cc2F)C3)c(-c2ccc(F)cc2)n1] |
| I-9 | 6-(3-(2,4-difluorophenyl)-3- hydroxy-4-(1H-1,2,4-triazol-1- yl)butyl)-4-(3-(4-fluorophenyl)- 1-methyl-1H-pyrazol-4-yl)-6,7- dihydro-5H-pyrrolo[3,4- b]pyridin-5-one | 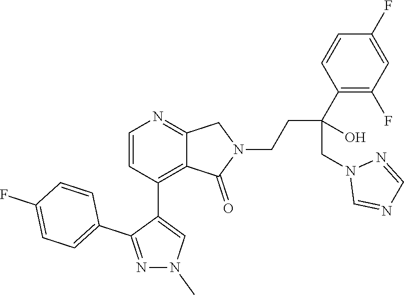 [SMILES: Cn1cc(-c2ccnc3c2C(=O)N(CCC(O)(Cn2cncn2)c2ccc(F)cc2F)C3)c(-c2ccc(F)cc2)n1] |
| I-10 | (R)-6-(3-(2,4-difluorophenyl)-3- hydroxy-4-(1H-1,2,4-triazol-1- yl)butyl)-4-(3-(4-fluorophenyl)- 1-methyl-1H-pyrazol-4-yl)-6,7- dihydro-5H-pyrrolo[3,4- b]pyridin-5-one | 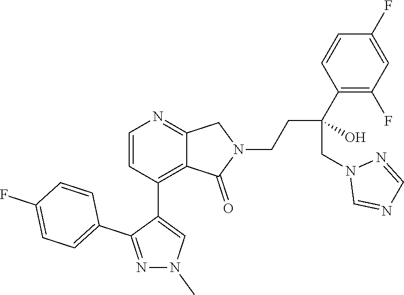 [SMILES: Cn1cc(-c2ccnc3c2C(=O)N(CC[C@](O)(Cn2cncn2)c2ccc(F)cc2F)C3)c(-c2ccc(F)cc2)n1] |
| I-11 | (S)-6-(3-(2,4-difluorophenyl)-3- hydroxy-4-(1H-1,2,4-triazol-1- yl)butyl)-4-(3-(4-fluorophenyl)- 1-methyl-1H-pyrazol-4-yl)-6,7- dihydro-5H-pyrrolo[3,4- b]pyridin-5-one | 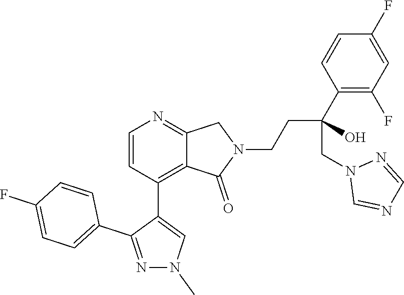 [SMILES: Cn1cc(-c2ccnc3c2C(=O)N(CC[C@@](O)(Cn2cncn2)c2ccc(F)cc2F)C3)c(-c2ccc(F)cc2)n1] |
| I-12 | (R)-6-(2-(2,4-difluorophenyl)-2- hydroxy-3-(1H-imidazol-1- yl)propyl)-4-(3-(4- fluorophenyl)-1-methyl-1H- pyrazol-4-yl)-6,7-dihydro-5H- pyrrolo[3,4-b]pyridin-5-one | 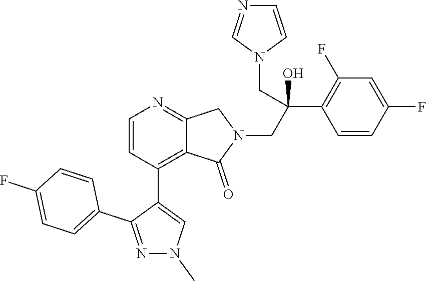 [SMILES: Cn1cc(-c2ccnc3c2C(=O)N(C[C@](O)(Cn2ccnc2)c2ccc(F)cc2F)C3)c(-c2ccc(F)cc2)n1] |
| I-13 | (S)-6-(2-(2,4-difluorophenyl)-2- hydroxy-3-(1H-imidazol-1- yl)propyl)-4-(3-(4- fluorophenyl)-1-methyl-1H- pyrazol-4-yl)-6,7-dihydro-5H- pyrrolo[3,4-b]pyridin-5-one | 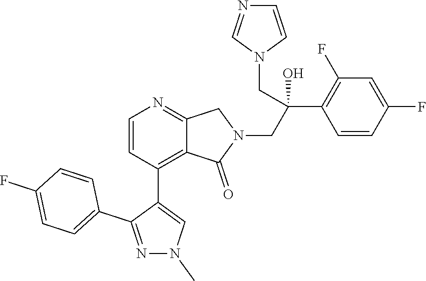 [SMILES: Cn1cc(-c2ccnc3c2C(=O)N(C[C@@](O)(Cn2ccnc2)c2ccc(F)cc2F)C3)c(-c2ccc(F)cc2)n1] |
| I-14 | (S)-2-(2,4-difluorophenyl)-1- (((4-(3-(4-fluorophenyl)-1- methyl-1H-pyrazol-4-yl)pyridin- 3-yl)methyl)amino)-3-(1H- 1,2,4-triazol-1-yl)propan-2-ol | 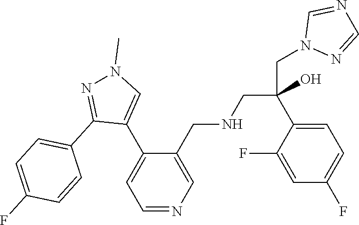 [SMILES: Cn1cc(-c2ccncc2CNC[C@](O)(Cn2cncn2)c2ccc(F)cc2F)c(-c2ccc(F)cc2)n1] |
| I-15 | (R)-2-(2,4-difluorophenyl)-1- (((4-(3-(4-fluorophenyl)-1- methyl-1H-pyrazol-4-yl)pyridin- 3-yl)methyl)amino)-3-(1H- 1,2,4-triazol-1-yl)propan-2-ol | 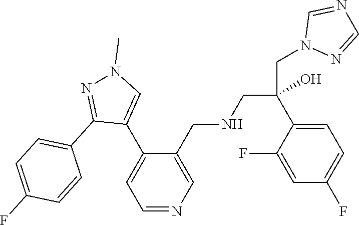 [SMILES: Cn1cc(-c2ccncc2CNC[C@@](O)(Cn2cncn2)c2ccc(F)cc2F)c(-c2ccc(F)cc2)n1] |
| I-16 | (R)-2-(2,4-difluorophenyl)-4- (((4-(3-(4-fluorophenyl)-1- methyl-1H-pyrazol-4-yl)pyridin- 3-yl)methyl)amino)-1-(1H- 1,2,4-triazol-1-yl)butan-2-ol | 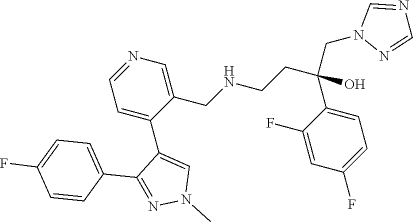 [SMILES: Cn1cc(-c2ccncc2CNCC[C@](O)(Cn2cncn2)c2ccc(F)cc2F)c(-c2ccc(F)cc2)n1] |
| I-17 | (S)-2-(2,4-difluorophenyl)-4- (((4-(3-(4-fluorophenyl)-1- methyl-1H-pyrazol-4-yl)pyridin- 3-yl)methyl)amino)-1-(1H- 1,2,4-triazol-1-yl)butan-2-ol | 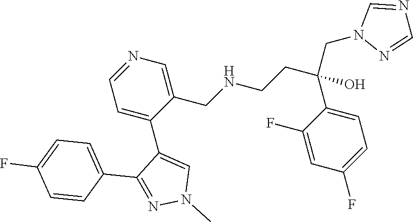 [SMILES: Cn1cc(-c2ccncc2CNCC[C@@](O)(Cn2cncn2)c2ccc(F)cc2F)c(-c2ccc(F)cc2)n1] |
| I-18 | 4-(1-(2-(2,4-difluorophenyl)-2- hydroxy-3-(1H-1,2,4-triazol-1- yl)propyl)-3-(4-fluorophenyl)- 1H-pyrazol-4-yl)-6-methyl-6,7- dihydro-5H-pyrrolo[3,4- b]pyridin-5-one | 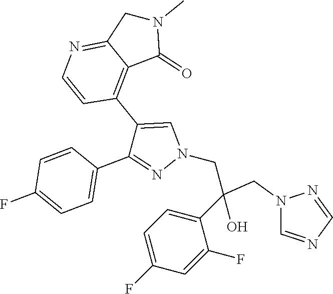 [SMILES: CN1Cc2nccc(-c3cn(CC(O)(Cn4cncn4)c4ccc(F)cc4F)nc3-c3ccc(F)cc3)c2C1=O] |
| I-19 | (S)-4-(1-(2-(2,4- difluorophenyl)-2-hydroxy-3- (1H-1,2,4-triazol-1-yl)propyl)-3- (4-fluorophenyl)-1H-pyrazol-4- yl)-6-methyl-6,7-dihydro-5H- pyrrolo[3,4-b]pyridin-5-one | 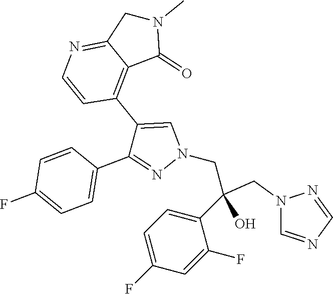 [SMILES: CN1Cc2nccc(-c3cn(C[C@](O)(Cn4cncn4)c4ccc(F)cc4F)nc3-c3ccc(F)cc3)c2C1=O] |
| I-20 | (R)-4-(1-(2-(2,4- difluorophenyl)-2-hydroxy-3- (1H-1,2,4-triazol-1-yl)propyl)-3- (4-fluorophenyl)-1H-pyrazol-4- yl)-6-methyl-6,7-dihydro-5H- pyrrolo[3,4-b]pyridin-5-one | 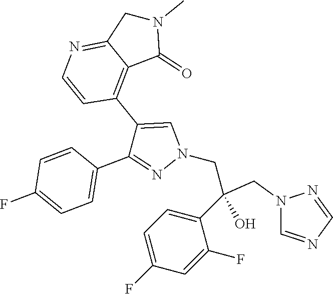 [SMILES: CN1Cc2nccc(-c3cn(C[C@@](O)(Cn4cncn4)c4ccc(F)cc4F)nc3-c3ccc(F)cc3)c2C1=O] |
| I-21 | (R)-4-(1-(3-(2,4- difluorophenyl)-3-hydroxy-4- (1H-1,2,4-triazol-1-yl)butyl)-3- (4-fluorophenyl)-1H-pyrazol-4- yl)-6-methyl-6,7-dihydro-5H- pyrrolo[3,4-b]pyridin-5-one | 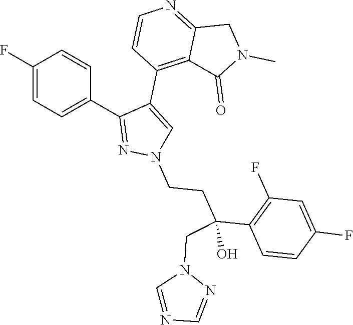 [SMILES: CN1Cc2nccc(-c3cn(CC[C@](O)(Cn4cncn4)c4ccc(F)cc4F)nc3-c3ccc(F)cc3)c2C1=O] |
| I-22 | (S)-4-(1-(3-(2,4- difluorophenyl)-3-hydroxy-4- (1H-1,2,4-triazol-1-yl)butyl)-3- (4-fluorophenyl)-1H-pyrazol-4- yl)-6-methyl-6,7-dihydro-5H- pyrrolo[3,4-b]pyridin-5-one | 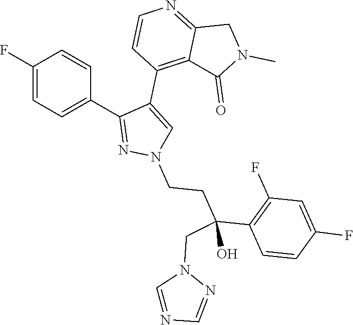 [SMILES: CN1Cc2nccc(-c3cn(CC[C@@](O)(Cn4cncn4)c4ccc(F)cc4F)nc3-c3ccc(F)cc3)c2C1=O] |
| I-23 | (S)-2-(2,4-difluorophenyl)-1-(4- (((4-(3-(4-fluorophenyl)-1- methyl-1H-pyrazol-4-yl)pyridin- 3-yl)methyl)amino)piperidin-1- yl)-3-(1H-1,2,4-triazol-1- yl)propan-2-ol | 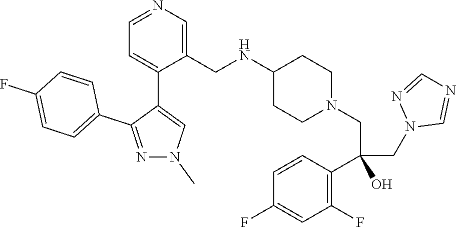 [SMILES: Cn1cc(-c2ccncc2CNC2CCN(C[C@](O)(Cn3cncn3)c3ccc(F)cc3F)CC2)c(-c2ccc(F)cc2)n1] |
| I-24 | (R)-2-(2,4-difluorophenyl)-1-(4- (((4-(3-(4-fluorophenyl)-1- methyl-1H-pyrazol-4-yl)pyridin- 3-yl)methyl)amino)piperidin-1- yl)-3-(1H-1,2,4-triazol-1- yl)propan-2-ol | 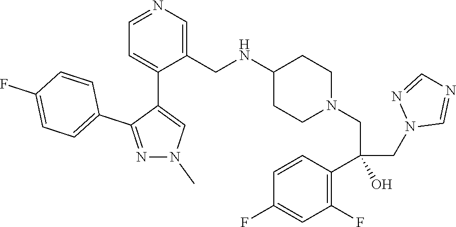 [SMILES: Cn1cc(-c2ccncc2CNC2CCN(C[C@@](O)(Cn3cncn3)c3ccc(F)cc3F)CC2)c(-c2ccc(F)cc2)n1] |
| I-25 | (S)-6-(2-((2-(2,4- difluorophenyl)-2-hydroxy-3- (1H-imidazol-1- yl)propyl)amino)ethyl)-4-(3-(4- fluorophenyl)-1-methyl-1H- pyrazol-4-yl)-6,7-dihydro-5H- pyrrolo[3,4-b]pyridin-5-one | 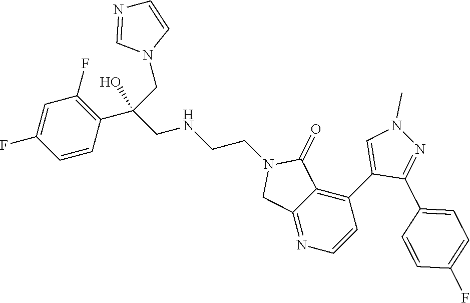 [SMILES: Cn1cc(-c2ccnc3c2C(=O)N(CCNC[C@](O)(Cn2ccnc2)c2ccc(F)cc2F)C3)c(-c2ccc(F)cc2)n1] |
| I-26 | 4-(1-(2-((2-(2,4- difluorophenyl)-2-hydroxy-3- (1H-1,2,4-triazol-1- yl)propyl)amino)ethyl)-3-(4- fluorophenyl)-1H-pyrazol-4-yl)- 6-methyl-6,7-dihydro-5H- pyrrolo[3,4-b]pyridin-5-one | 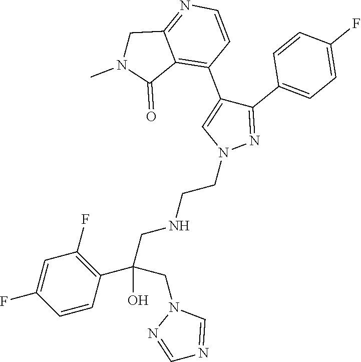 [SMILES: CN1Cc2nccc(-c3cn(CCNCC(O)(Cn4cncn4)c4ccc(F)cc4F)nc3-c3ccc(F)cc3)c2C1=O] |
| I-27 | (R)-4-(1-(2-((2-(2,4- difluorophenyl)-2-hydroxy-3- (1H-1,2,4-triazol-1- yl)propyl)amino)ethyl)-3-(4- fluorophenyl)-1H-pyrazol-4-yl)- 6-methyl-6,7-dihydro-5H- pyrrolo[3,4-b]pyridin-5-one | 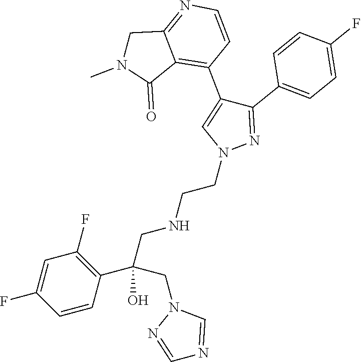 [SMILES: CN1Cc2nccc(-c3cn(CCNC[C@@](O)(Cn4cncn4)c4ccc(F)cc4F)nc3-c3ccc(F)cc3)c2C1=O] |
| I-28 | (S)-4-(1-(2-((2-(2,4- difluorophenyl)-2-hydroxy-3- (1H-1,2,4-triazol-1- yl)propyl)amino)ethyl)-3-(4- fluorophenyl)-1H-pyrazol-4-yl)- 6-methyl-6,7-dihydro-5H- pyrrolo[3,4-b]pyridin-5-one | 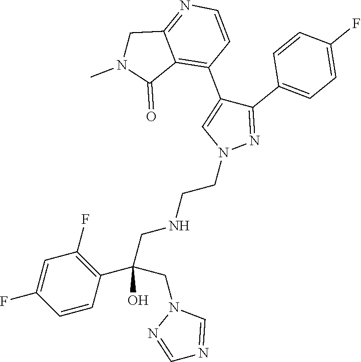 [SMILES: CN1Cc2nccc(-c3cn(CCNC[C@](O)(Cn4cncn4)c4ccc(F)cc4F)nc3-c3ccc(F)cc3)c2C1=O] |
| I-29 | 4-(1-(1-(2-(2,4-difluorophenyl)- 2-hydroxy-3-(1H-1,2,4-triazol- 1-yl)propyl)piperidin-4-yl)-3-(4- fluorophenyl)-1H-pyrazol-4-yl)- 6-methyl-6,7-dihydro-5H- pyrrolo[3,4-b]pyridin-5-one | 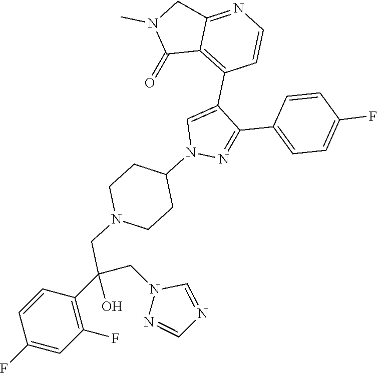 [SMILES: CN1Cc2nccc(-c3cn(C4CCN(CC(O)(Cn5cncn5)c5ccc(F)cc5F)CC4)nc3-c3ccc(F)cc3)c2C1=O] |
| I-30 | 4-(1-(1-(2-(2,4-difluorophenyl)- 2-hydroxy-3-(1H-1,2,4-triazol- 1-yl)propyl)pyrrolidin-3-yl)-3- (4-fluorophenyl)-1H-pyrazol-4- yl)-6-methyl-6,7-dihydro-5H- pyrrolo[3,4-b]pyridin-5-one | 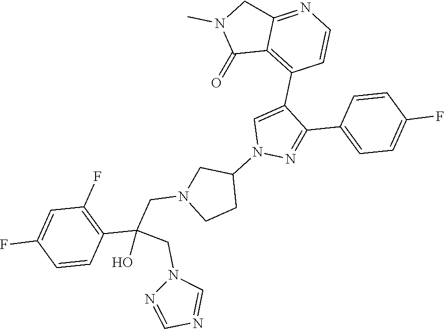 [SMILES: CN1Cc2nccc(-c3cn(C4CCN(CC(O)(Cn5cncn5)c5ccc(F)cc5F)C4)nc3-c3ccc(F)cc3)c2C1=O] |
| I-31 | 4-(1-(1-(2-(2,4-difluorophenyl)- 2-hydroxy-3-(1H-1,2,4-triazol- 1-yl)propyl)azetidin-3-yl)-3-(4- fluorophenyl)-1H-pyrazol-4-yl)- 6-methyl-6,7-dihydro-5H- pyrrolo[3,4-b]pyridin-5-one | 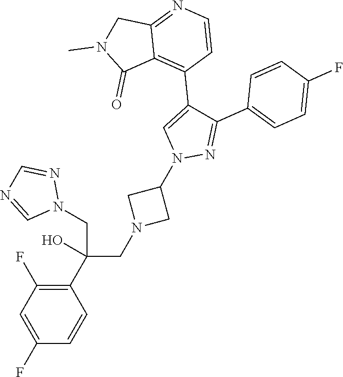 [SMILES: CN1Cc2nccc(-c3cn(C4CN(CC(O)(Cn5cncn5)c5ccc(F)cc5F)C4)nc3-c3ccc(F)cc3)c2C1=O] |
| I-32 | 1-(4-(5-(2-aminopyrimidin-4- yl)-4-(4-fluorophenyl)-1H- imidazol-1-yl)piperidin-1-yl)-2- (2,4-difluorophenyl)-3-(1H- 1,2,4-triazol-1-yl)propan-2-ol | 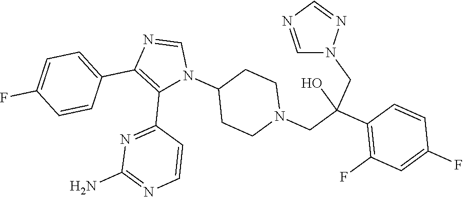 [SMILES: Nc1nccc(-c2c(-c3ccc(F)cc3)ncn2C2CCN(CC(O)(Cn3cncn3)c3ccc(F)cc3F)CC2)n1] |
| I-33 | (S)-1-(4-(5-(2-aminopyrimidin- 4-yl)-4-(4-fluorophenyl)-1H- imidazol-1-yl)piperidin-1-yl)-2- (2,4-difluorophenyl)-3-(1H- 1,2,4-triazol-1-yl)propan-2-ol | 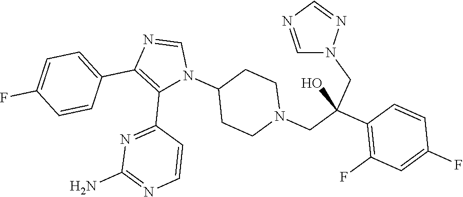 [SMILES: Nc1nccc(-c2c(-c3ccc(F)cc3)ncn2C2CCN(C[C@](O)(Cn3cncn3)c3ccc(F)cc3F)CC2)n1] |
| I-34 | (R)-1-(4-(5-(2-aminopyrimidin- 4-yl)-4-(4-fluorophenyl)-1H- imidazol-1-yl)piperidin-1-yl)-2- (2,4-difluorophenyl)-3-(1H- 1,2,4-triazol-1-yl)propan-2-ol | 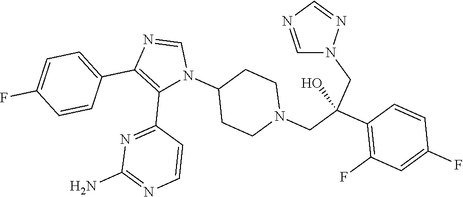 [SMILES: Nc1nccc(-c2c(-c3ccc(F)cc3)ncn2C2CCN(C[C@@](O)(Cn3cncn3)c3ccc(F)cc3F)CC2)n1] |
| I-35 | 1-(4-(5-(2-aminopyrimidin-4- yl)-4-(4-fluorophenyl)-1H- imidazol-1-yl)piperidin-1-yl)-2- (2,4-difluorophenyl)-3-(1H- 1,2,3-triazol-1-yl)propan-2-ol | 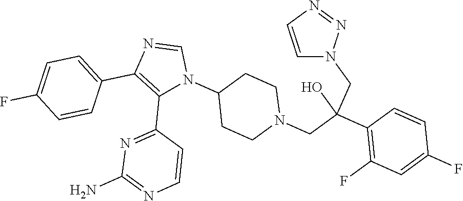 [SMILES: Nc1nccc(-c2c(-c3ccc(F)cc3)ncn2C2CCN(CC(O)(Cn3ccnn3)c3ccc(F)cc3F)CC2)n1] |
| I-36 | 1-(3-(5-(2-aminopyrimidin-4- yl)-4-(4-fluorophenyl)-1H- imidazol-1-yl)pyrrolidin-1-yl)-2- (2,4-difluorophenyl)-3-(1H- 1,2,4-triazol-1-yl)propan-2-ol | 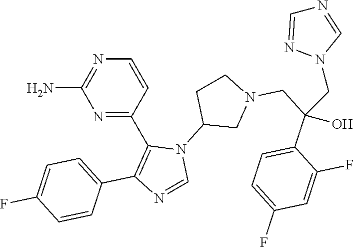 [SMILES: Nc1nccc(-c2c(-c3ccc(F)cc3)ncn2C2CCN(CC(O)(Cn3cncn3)c3ccc(F)cc3F)C2)n1] |
| I-37 | (2S)-1-(3-(5-(2- aminopyrimidin-4-yl)-4-(4- fluorophenyl)-1H-imidazol-1- yl)pyrrolidin-1-yl)-2-(2,4- difluorophenyl)-3-(1H-1,2,4- triazol-1-yl)propan-2-ol | 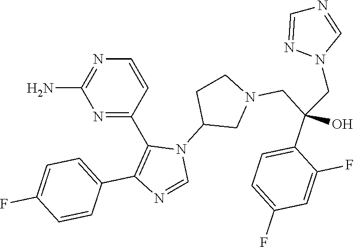 [SMILES: Nc1nccc(-c2c(-c3ccc(F)cc3)ncn2C2CCN(C[C@](O)(Cn3cncn3)c3ccc(F)cc3F)C2)n1] |
| I-38 | (2R)-1-(3-(5-(2- aminopyrimidin-4-yl)-4-(4- fluorophenyl)-1H-imidazol-1- yl)pyrrolidin-1-yl)-2-(2,4- difluorophenyl)-3-(1H-1,2,4- triazol-1-yl)propan-2-ol | 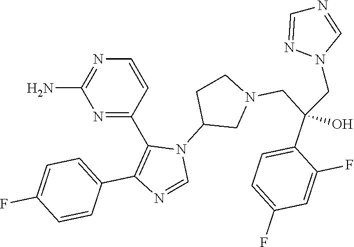 [SMILES: Nc1nccc(-c2c(-c3ccc(F)cc3)ncn2C2CCN(C[C@@](O)(Cn3cncn3)c3ccc(F)cc3F)C2)n1] |
| I-39 | 1-((S)-3-(5-(2-aminopyrimidin- 4-yl)-4-(4-fluorophenyl)-1H- imidazol-1-yl)pyrrolidin-1-yl)-2- (2,4-difluorophenyl)-3-(1H- 1,2,4-triazol-1-yl)propan-2-ol | 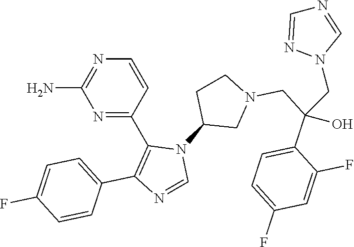 [SMILES: Nc1nccc(-c2c(-c3ccc(F)cc3)ncn2[C@H]2CCN(CC(O)(Cn3cncn3)c3ccc(F)cc3F)C2)n1] |
| I-40 | (S)-1-((S)-3-(5-(2- aminopyrimidin-4-yl)-4-(4- fluorophenyl)-1H-imidazol-1- yl)pyrrolidin-1-yl)-2-(2,4- difluorophenyl)-3-(1H-1,2,4- triazol-1-yl)propan-2-ol | 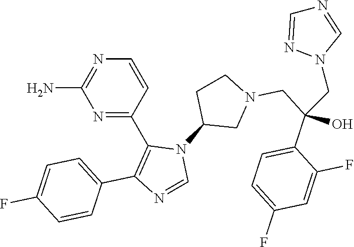 [SMILES: Nc1nccc(-c2c(-c3ccc(F)cc3)ncn2[C@H]2CCN(C[C@](O)(Cn3cncn3)c3ccc(F)cc3F)C2)n1] |
| I-41 | (R)-1-((S)-3-(5-(2- aminopyrimidin-4-yl)-4-(4- fluorophenyl)-1H-imidazol-1- yl)pyrrolidin-1-yl)-2-(2,4- difluorophenyl)-3-(1H-1,2,4- triazol-1-yl)propan-2-ol | 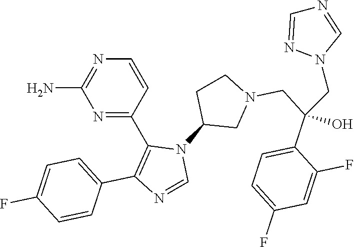 [SMILES: Nc1nccc(-c2c(-c3ccc(F)cc3)ncn2[C@H]2CCN(C[C@@](O)(Cn3cncn3)c3ccc(F)cc3F)C2)n1] |
| I-42 | (R)-1-((R)-3-(5-(2- aminopyrimidin-4-yl)-4-(4- fluorophenyl)-1H-imidazol-1- yl)pyrrolidin-1-yl)-2-(2,4- difluorophenyl)-3-(1H-1,2,4- triazol-1-yl)propan-2-ol | 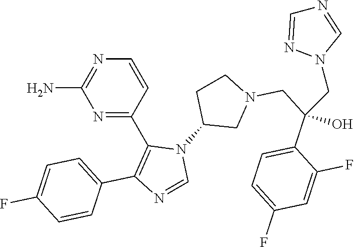 [SMILES: Nc1nccc(-c2c(-c3ccc(F)cc3)ncn2[C@@H]2CCN(C[C@@](O)(Cn3cncn3)c3ccc(F)cc3F)C2)n1] |
| I-43 | (S)-1-((R)-3-(5-(2- aminopyrimidin-4-yl)-4-(4- fluorophenyl)-1H-imidazol-1- yl)pyrrolidin-1-yl)-2-(2,4- difluorophenyl)-3-(1H-1,2,4- triazol-1-yl)propan-2-ol | 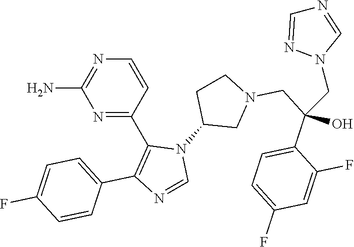 [SMILES: Nc1nccc(-c2c(-c3ccc(F)cc3)ncn2[C@@H]2CCN(C[C@](O)(Cn3cncn3)c3ccc(F)cc3F)C2)n1] |
| I-44 | 1-((R)-3-(5-(2-aminopyrimidin- 4-yl)-4-(4-fluorophenyl)-1H- imidazol-1-yl)pyrrolidin-1-yl)-2- (2,4-difluorophenyl)-3-(1H- 1,2,4-triazol-1-yl)propan-2-ol | 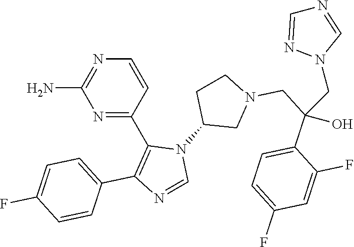 [SMILES: Nc1nccc(-c2c(-c3ccc(F)cc3)ncn2[C@@H]2CCN(CC(O)(Cn3cncn3)c3ccc(F)cc3F)C2)n1] |
| I-45 | (2S)-1-((2R)-4-(5-(2- aminopyrimidin-4-yl)-4-(4- fluorophenyl)-1H-imidazol-1- yl)-2-methylpiperidin-1-yl)-2- (2,4-difluorophenyl)-3-(1H- 1,2,4-triazol-1-yl)propan-2-ol | 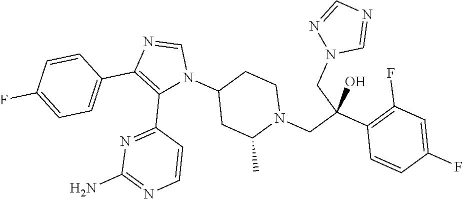 [SMILES: C[C@@H]1CC(n2cnc(-c3ccc(F)cc3)c2-c2ccnc(N)n2)CCN1C[C@](O)(Cn1cncn1)c1ccc(F)cc1F] |
| I-46 | 1-(4-(5-(2-((1-(2,2- difluorocyclopropyl)-5-methyl- 1H-pyrazol-4- yl)amino)pyrimidin-4-yl)-4-(4- fluorophenyl)-1H-imidazol-1- yl)piperidin-1-yl)-2-(2,4- difluorophenyl)-3-(1H-1,2,4- triazol-1-yl)propan-2-ol | 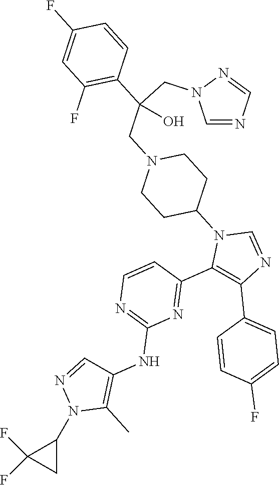 [SMILES: Cc1c(Nc2nccc(-c3c(-c4ccc(F)cc4)ncn3C3CCN(CC(O)(Cn4cncn4)c4ccc(F)cc4F)CC3)n2)cnn1C1CC1(F)F] |
| I-47 | (R)-1-(4-(5-(2-aminopyrimidin- 4-yl)-4-(4-fluorophenyl)-1H- imidazol-1-yl)piperidin-1-yl)-2- (2,4-difluorophenyl)-3-(1H- 1,2,3-triazol-1-yl)propan-2-ol | 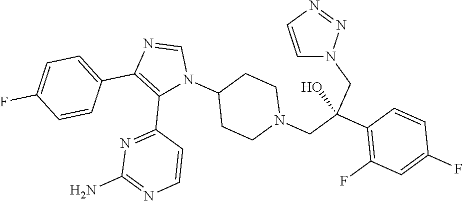 [SMILES: Nc1nccc(-c2c(-c3ccc(F)cc3)ncn2C2CCN(C[C@@](O)(Cn3ccnn3)c3ccc(F)cc3F)CC2)n1] |
| I-48 | (S)-1-(4-(5-(2-aminopyrimidin- 4-yl)-4-(4-fluorophenyl)-1H- imidazol-1-yl)piperidin-1-yl)-2- (2,4-difluorophenyl)-3-(1H- 1,2,3-triazol-1-yl)propan-2-ol | 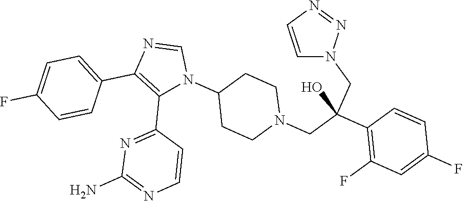 [SMILES: Nc1nccc(-c2c(-c3ccc(F)cc3)ncn2C2CCN(C[C@](O)(Cn3ccnn3)c3ccc(F)cc3F)CC2)n1] |
| I-49 | 1-(4-(((5-(2-aminopyrimidin-4- yl)-4-(4-fluorophenyl)-1H- imidazol-1- yl)methyl)amino)piperidin-1- yl)-2-(2,4-difluorophenyl)-3- (1H-1,2,4-triazol-1-yl)propan- 2-ol | 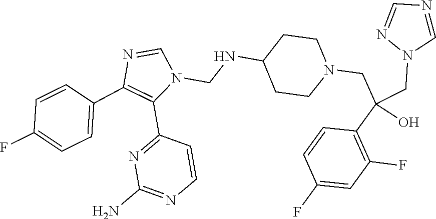 [SMILES: Nc1nccc(-c2c(-c3ccc(F)cc3)ncn2CNC2CCN(CC(O)(Cn3cncn3)c3ccc(F)cc3F)CC2)n1] |
| I-50 | 1-((3-(5-(2-aminopyrimidin-4- yl)-4-(4-fluorophenyl)-1H- imidazol-1-yl)propyl)amino)-2- (2,4-difluorophenyl)-3-(1H- 1,2,4-triazol-1-yl)propan-2-ol | 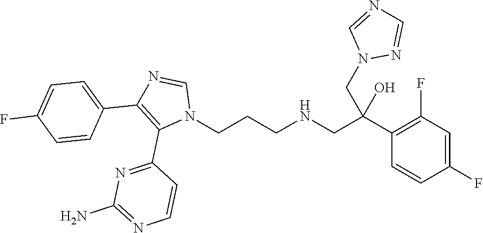 [SMILES: Nc1nccc(-c2c(-c3ccc(F)cc3)ncn2CCCNCC(O)(Cn2cncn2)c2ccc(F)cc2F)n1] |
| I-51 | 1-((2-(5-(2-aminopyrimidin-4- yl)-4-(4-fluorophenyl)-1H- imidazol-1-yl)ethyl)amino)-2- (2,4-difluorophenyl)-3-(1H- 1,2,4-triazol-1-yl)propan-2-ol | 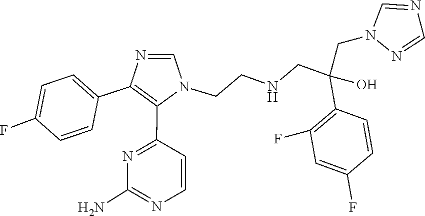 [SMILES: Nc1nccc(-c2c(-c3ccc(F)cc3)ncn2CCNCC(O)(Cn2cncn2)c2ccc(F)cc2F)n1] |
| I-52 | 1-(4-((2-(5-(2-aminopyrimidin- 4-yl)-4-(4-fluorophenyl)-1H- imidazol-1- yl)ethyl)amino)piperidin-1-yl)- 2-(2,4-difluorophenyl)-3-(1H- 1,2,4-triazol-1-yl)propan-2-ol | 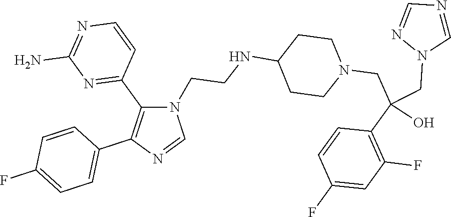 [SMILES: Nc1nccc(-c2c(-c3ccc(F)cc3)ncn2CCNC2CCN(CC(O)(Cn3cncn3)c3ccc(F)cc3F)CC2)n1] |
| I-53 | (R)-4-(4-(5-(2-aminopyrimidin- 4-yl)-4-(4-fluorophenyl)-1H- imidazol-1-yl)piperidin-1-yl)-2- (2,4-difluorophenyl)-1-(1H- 1,2,4-triazol-1-yl)butan-2-ol | 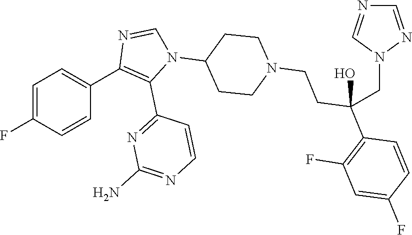 [SMILES: Nc1nccc(-c2c(-c3ccc(F)cc3)ncn2C2CCN(CC[C@](O)(Cn3cncn3)c3ccc(F)cc3F)CC2)n1] |
| I-54 | 4-(4-(5-(2-aminopyrimidin-4- yl)-4-(4-fluorophenyl)-1H- imidazol-1-yl)piperidin-1-yl)-2- (2,4-difluorophenyl)-1-(1H- 1,2,4-triazol-1-yl)butan-2-ol | 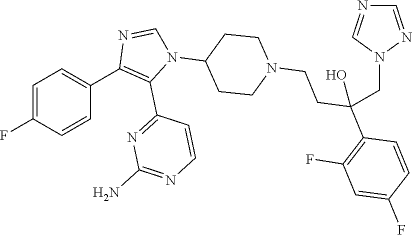 [SMILES: Nc1nccc(-c2c(-c3ccc(F)cc3)ncn2C2CCN(CCC(O)(Cn3cncn3)c3ccc(F)cc3F)CC2)n1] |
| I-55 | (S)-4-(4-(5-(2-aminopyrimidin- 4-yl)-4-(4-fluorophenyl)-1H- imidazol-1-yl)piperidin-1-yl)-2- (2,4-difluorophenyl)-1-(1H- 1,2,4-triazol-1-yl)butan-2-ol | 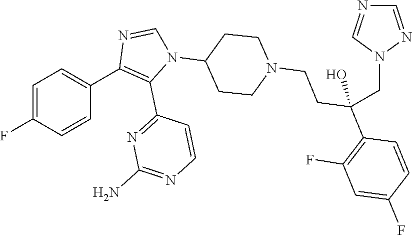 [SMILES: Nc1nccc(-c2c(-c3ccc(F)cc3)ncn2C2CCN(CC[C@@](O)(Cn3cncn3)c3ccc(F)cc3F)CC2)n1] |
| I-56 | (S)-5-(4-(5-(2-aminopyrimidin- 4-yl)-4-(4-fluorophenyl)-1H- imidazol-1-yl)piperidin-1-yl)-2- (2,4-difluorophenyl)-1-(1H- 1,2,4-triazol-1-yl)pentan-2-ol | 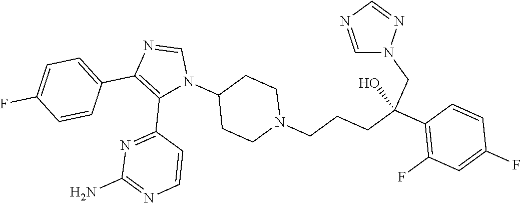 [SMILES: Nc1nccc(-c2c(-c3ccc(F)cc3)ncn2C2CCN(CCC[C@@](O)(Cn3cncn3)c3ccc(F)cc3F)CC2)n1] |
| I-57 | (R)-5-(4-(5-(2-aminopyrimidin- 4-yl)-4-(4-fluorophenyl)-1H- imidazol-1-yl)piperidin-1-yl)-2- (2,4-difluorophenyl)-1-(1H- 1,2,4-triazol-1-yl)pentan-2-ol | 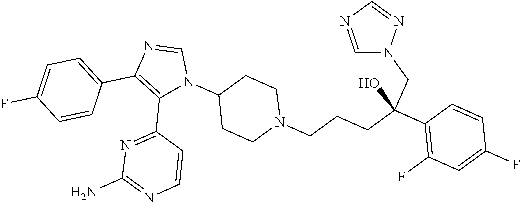 [SMILES: Nc1nccc(-c2c(-c3ccc(F)cc3)ncn2C2CCN(CCC[C@](O)(Cn3cncn3)c3ccc(F)cc3F)CC2)n1] |
| I-58 | 5-(4-(5-(2-aminopyrimidin-4- yl)-4-(4-fluorophenyl)-1H- imidazol-1-yl)piperidin-1-yl)-2- (2,4-difluorophenyl)-1-(1H- 1,2,4-triazol-1-yl)pentan-2-ol | 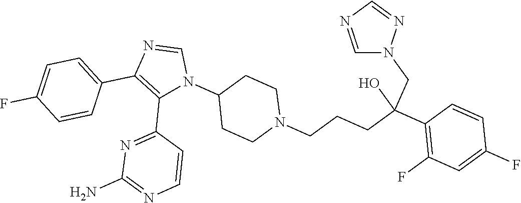 [SMILES: Nc1nccc(-c2c(-c3ccc(F)cc3)ncn2C2CCN(CCCC(O)(Cn3cncn3)c3ccc(F)cc3F)CC2)n1] |
| I-59 | (S)-1-((3-(5-(2-aminopyrimidin- 4-yl)-4-(4-fluorophenyl)-1H- imidazol-1- yl)propyl)(methyl)amino)-2- (2,4-difluorophenyl)-3-(1H- 1,2,4-triazol-1-yl)propan-2-ol | 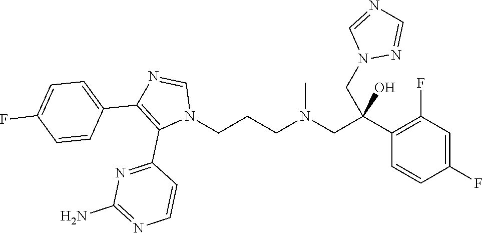 [SMILES: CN(CCCn1cnc(-c2ccc(F)cc2)c1-c1ccnc(N)n1)C[C@](O)(Cn1cncn1)c1ccc(F)cc1F] |
| I-60 | 1-((3-(5-(2-aminopyrimidin-4- yl)-4-(4-fluorophenyl)-1H- imidazol-1- yl)propyl)(methyl)amino)-2- (2,4-difluorophenyl)-3-(1H- 1,2,4-triazol-1-yl)propan-2-ol | 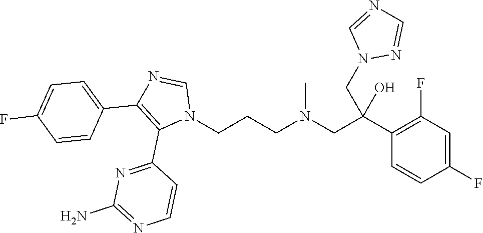 [SMILES: CN(CCCn1cnc(-c2ccc(F)cc2)c1-c1ccnc(N)n1)CC(O)(Cn1cncn1)c1ccc(F)cc1F] |
| I-61 | 5-(2-aminopyrimidin-4-yl)-N-(1- (2-(2,4-difluorophenyl)-2- hydroxy-3-(1H-1,2,4-triazol-1- yl)propyl)piperidin-4-yl)-4-(4- fluorophenyl)-1H-imidazole-1- carboxamide | 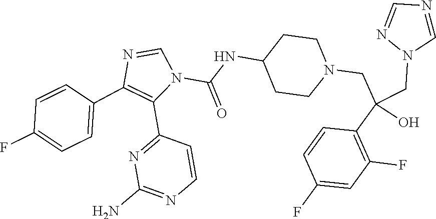 [SMILES: Nc1nccc(-c2c(-c3ccc(F)cc3)ncn2C(=O)NC2CCN(CC(O)(Cn3cncn3)c3ccc(F)cc3F)CC2)n1] |
| I-62 | 1-((1R,5S)-6-(5-(2- aminopyrimidin-4-yl)-4-(4- fluorophenyl)-1H-imidazol-1- yl)-3-azabicyclo[3.1.0]hexan-3- yl)-2-(2,4-difluorocyclohexyl)- 3-(1,2,4-triazolidin-1-yl)propan- 2-ol | 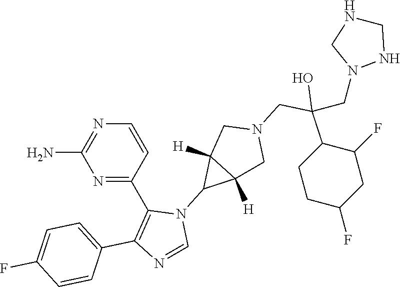 [SMILES: Nc1nccc(-c2c(-c3ccc(F)cc3)ncn2C2[C@H]3CN(CC(O)(CN4CNCN4)C4CCC(F)CC4F)C[C@@H]23)n1] |
| I-63 | (2R)-1-((1R,5S)-6-(5-(2- aminopyrimidin-4-yl)-4-(4- fluorophenyl)-1H-imidazol-1- yl)-3-azabicyclo[3.1.0]hexan-3- yl)-2-(2,4-difluorocyclohexyl)- 3-(1,2,4-triazolidin-1-yl)propan- 2-ol | 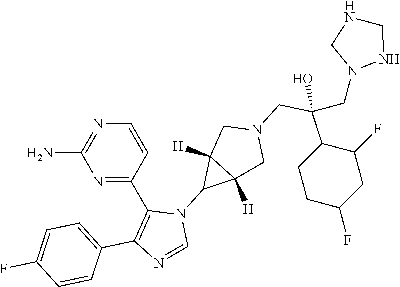 [SMILES: Nc1nccc(-c2c(-c3ccc(F)cc3)ncn2C2[C@H]3CN(C[C@@](O)(CN4CNCN4)C4CCC(F)CC4F)C[C@@H]23)n1] |
| I-64 | (2S)-1-((1R,5S)-6-(5-(2- aminopyrimidin-4-yl)-4-(4- fluorophenyl)-1H-imidazol-1- yl)-3-azabicyclo[3.1.0]hexan-3- yl)-2-(2,4-difluorocyclohexyl)- 3-(1,2,4-triazolidin-1-yl)propan- 2-ol | 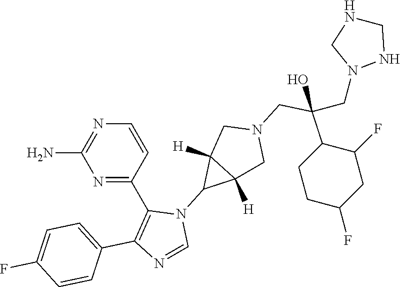 [SMILES: Nc1nccc(-c2c(-c3ccc(F)cc3)ncn2C2[C@H]3CN(C[C@](O)(CN4CNCN4)C4CCC(F)CC4F)C[C@@H]23)n1] |
| I-65 | (2S)-1-((1R,5S)-6-(5-(2- aminopyrimidin-4-yl)-4-(4- fluorophenyl)-1H-imidazol-1- yl)-3-azabicyclo[3.1.0]hexan-3- yl)-2-(2,4-difluorophenyl)-3- (1H-1,2,4-triazol-1-yl)propan- 2-ol | 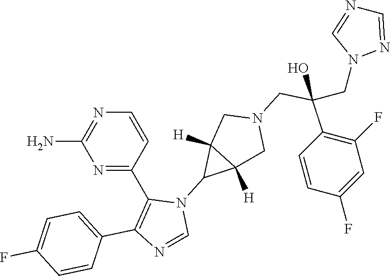 [SMILES: Nc1nccc(-c2c(-c3ccc(F)cc3)ncn2C2[C@H]3CN(C[C@](O)(Cn4cncn4)c4ccc(F)cc4F)C[C@@H]23)n1] |
| I-66 | (2R)-1-((1R,5S)-6-(5-(2- aminopyrimidin-4-yl)-4-(4- fluorophenyl)-1H-imidazol-1- yl)-3-azabicyclo[3.1.0]hexan-3- yl)-2-(2,4-difluorophenyl)-3- (1H-1,2,4-triazol-1-yl)propan- 2-ol | 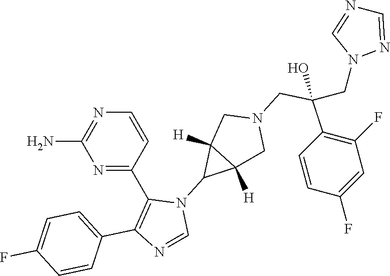 [SMILES: Nc1nccc(-c2c(-c3ccc(F)cc3)ncn2C2[C@H]3CN(C[C@@](O)(Cn4cncn4)c4ccc(F)cc4F)C[C@@H]23)n1] |
| I-67 | 1-((1R,5S)-6-(5-(2- aminopyrimidin-4-yl)-4-(4- fluorophenyl)-1H-imidazol-1- yl)-3-azabicyclo[3.1.0]hexan-3- yl)-2-(2,4-difluorophenyl)-3- (1H-1,2,4-triazol-1-yl)propan- 2-ol | 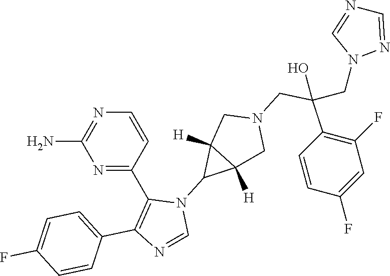 [SMILES: Nc1nccc(-c2c(-c3ccc(F)cc3)ncn2C2[C@H]3CN(CC(O)(Cn4cncn4)c4ccc(F)cc4F)C[C@@H]23)n1] |
| I-68 | (R)-1-(4-(5-(2-aminopyrimidin- 4-yl)-4-(4-fluorophenyl)-1H- imidazol-1-yl)piperidin-1-yl)-5- (2,4-difluorophenyl)-5-hydroxy- 6-(1H-1,2,4-triazol-1-yl)hexan- 2-one | 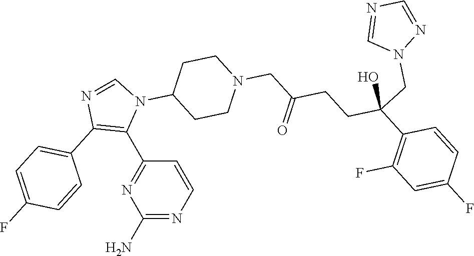 [SMILES: Nc1nccc(-c2c(-c3ccc(F)cc3)ncn2C2CCN(CC(=O)CC[C@](O)(Cn3cncn3)c3ccc(F)cc3F)CC2)n1] |
| I-69 | (S)-2-(4-(5-(2-aminopyrimidin- 4-yl)-4-(4-fluorophenyl)-1H- imidazol-1-yl)piperidin-1-yl)-N- (2-(2,4-difluorophenyl)-2- hydroxy-3-(1H-1,2,4-triazol-1- yl)propyl)acetamide | 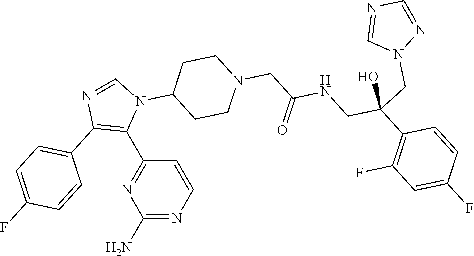 [SMILES: Nc1nccc(-c2c(-c3ccc(F)cc3)ncn2C2CCN(CC(=O)NC[C@](O)(Cn3cncn3)c3ccc(F)cc3F)CC2)n1] |
| I-70 | (R)-2-(4-(5-(2-aminopyrimidin- 4-yl)-4-(4-fluorophenyl)-1H- imidazol-1-yl)piperidin-1-yl)-N- (2-(2,4-difluorophenyl)-2- hydroxy-3-(1H-1,2,4-triazol-1- yl)propyl)acetamide | 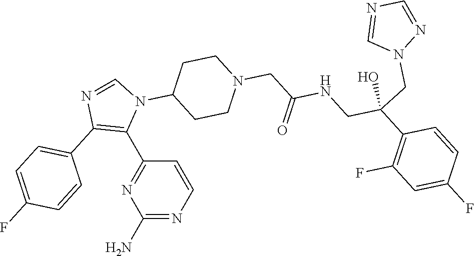 [SMILES: Nc1nccc(-c2c(-c3ccc(F)cc3)ncn2C2CCN(CC(=O)NC[C@@](O)(Cn3cncn3)c3ccc(F)cc3F)CC2)n1] |
| I-71 | 2-(4-(5-(2-aminopyrimidin-4- yl)-4-(4-fluorophenyl)-1H- imidazol-1-yl)piperidin-1-yl)-N- (2-(2,4-difluorophenyl)-2- hydroxy-3-(1H-1,2,4-triazol-1- yl)propyl)acetamide | 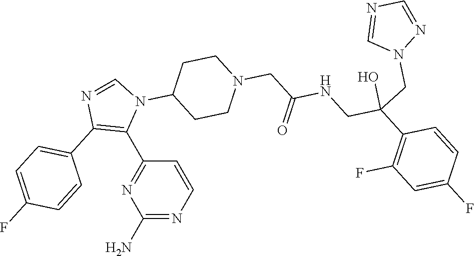 [SMILES: Nc1nccc(-c2c(-c3ccc(F)cc3)ncn2C2CCN(CC(=O)NCC(O)(Cn3cncn3)c3ccc(F)cc3F)CC2)n1] |
| I-72 | 1-((2-(4-(5-(2-aminopyrimidin- 4-yl)-4-(4-fluorophenyl)-1H- imidazol-1-yl)piperidin-1- yl)ethyl)amino)-2-(2,4- difluorophenyl)-3-(1H-1,2,4- triazol-1-yl)propan-2-ol | 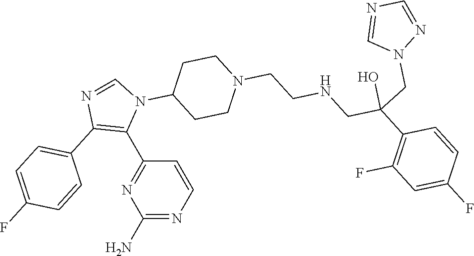 [SMILES: Nc1nccc(-c2c(-c3ccc(F)cc3)ncn2C2CCN(CCNCC(O)(Cn3cncn3)c3ccc(F)cc3F)CC2)n1] |
| I-73 | (S)-1-((2-(4-(5-(2- aminopyrimidin-4-yl)-4-(4- fluorophenyl)-1H-imidazol-1- yl)piperidin-1-yl)ethyl)amino)- 2-(2,4-difluorophenyl)-3-(1H- 1,2,4-triazol-1-yl)propan-2-ol | 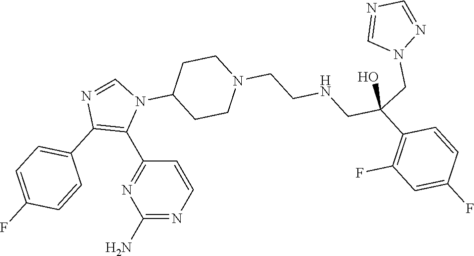 [SMILES: Nc1nccc(-c2c(-c3ccc(F)cc3)ncn2C2CCN(CCNC[C@](O)(Cn3cncn3)c3ccc(F)cc3F)CC2)n1] |
| I-74 | (R)-1-((2-(4-(5-(2- aminopyrimidin-4-yl)-4-(4- fluorophenyl)-1H-imidazol-1- yl)piperidin-1-yl)ethyl)amino)- 2-(2,4-difluorophenyl)-3-(1H- 1,2,4-triazol-1-yl)propan-2-ol | 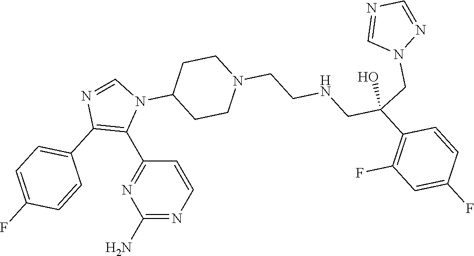 [SMILES: Nc1nccc(-c2c(-c3ccc(F)cc3)ncn2C2CCN(CCNC[C@@](O)(Cn3cncn3)c3ccc(F)cc3F)CC2)n1] |
| I-75 | (S)-1-(4-(5-(2-aminopyrimidin- 4-yl)-4-(4-fluorophenyl)-1H- imidazol-1-yl)piperidin-1-yl)-3- ((2-(2,4-difluorophenyl)-2- hydroxy-3-(1H-1,2,4-triazol-1- yl)propyl)amino)propan-1-one | 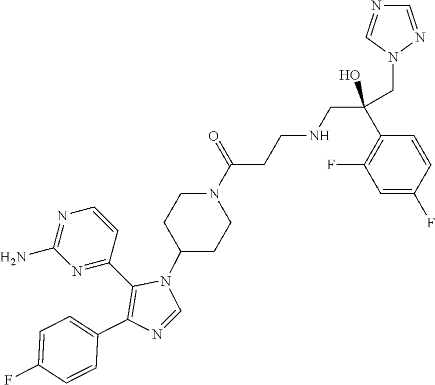 [SMILES: Nc1nccc(-c2c(-c3ccc(F)cc3)ncn2C2CCN(C(=O)CCNC[C@](O)(Cn3cncn3)c3ccc(F)cc3F)CC2)n1] |
| I-76 | (R)-1-(4-(5-(2-aminopyrimidin- 4-yl)-4-(4-fluorophenyl)-1H- imidazol-1-yl)piperidin-1-yl)-3- ((2-(2,4-difluorophenyl)-2- hydroxy-3-(1H-1,2,4-triazol-1- yl)propyl)amino)propan-1-one | 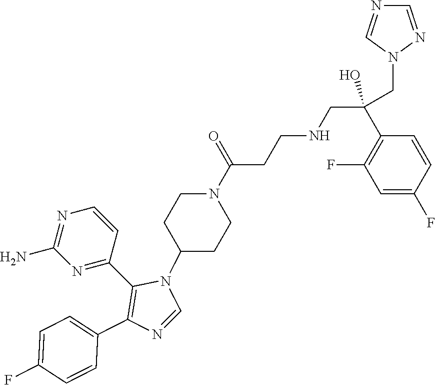 [SMILES: Nc1nccc(-c2c(-c3ccc(F)cc3)ncn2C2CCN(C(=O)CCNC[C@@](O)(Cn3cncn3)c3ccc(F)cc3F)CC2)n1] |
| I-77 | 1-(4-(5-(2-aminopyrimidin-4- yl)-4-(4-fluorophenyl)-1H- imidazol-1-yl)piperidin-1-yl)-3- ((2-(2,4-difluorophenyl)-2- hydroxy-3-(1H-1,2,4-triazol-1- yl)propyl)amino)propan-1-one | 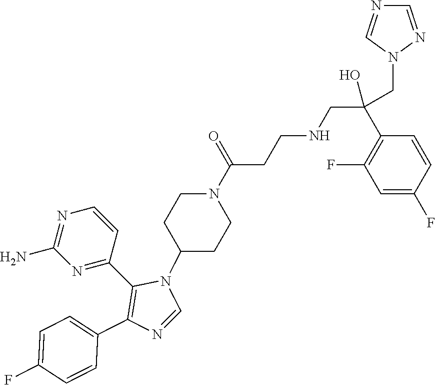 [SMILES: Nc1nccc(-c2c(-c3ccc(F)cc3)ncn2C2CCN(C(=O)CCNCC(O)(Cn3cncn3)c3ccc(F)cc3F)CC2)n1] |
| I-78 | (S)-1-((3-(4-(5-(2- aminopyrimidin-4-yl)-4-(4- fluorophenyl)-1H-imidazol-1- yl)piperidin-1-yl)propyl)amino)- 2-(2,4-difluorophenyl)-3-(1H- 1,2,4-triazol-1-yl)propan-2-ol | 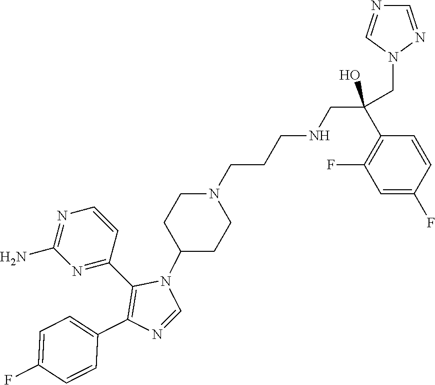 [SMILES: Nc1nccc(-c2c(-c3ccc(F)cc3)ncn2C2CCN(CCCNC[C@](O)(Cn3cncn3)c3ccc(F)cc3F)CC2)n1] |
| I-79 | (R)-1-((3-(4-(5-(2- aminopyrimidin-4-yl)-4-(4- fluorophenyl)-1H-imidazol-1- yl)piperidin-1-yl)propyl)amino)- 2-(2,4-difluorophenyl)-3-(1H- 1,2,4-triazol-1-yl)propan-2-ol | 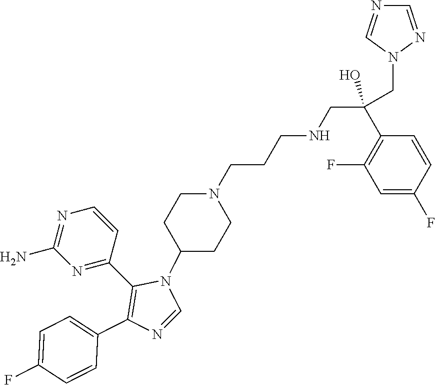 [SMILES: Nc1nccc(-c2c(-c3ccc(F)cc3)ncn2C2CCN(CCCNC[C@@](O)(Cn3cncn3)c3ccc(F)cc3F)CC2)n1] |
| I-80 | 1-((3-(4-(5-(2-aminopyrimidin- 4-yl)-4-(4-fluorophenyl)-1H- imidazol-1-yl)piperidin-1- yl)propyl)amino)-2-(2,4- difluorophenyl)-3-(1H-1,2,4- triazol-1-yl)propan-2-ol | 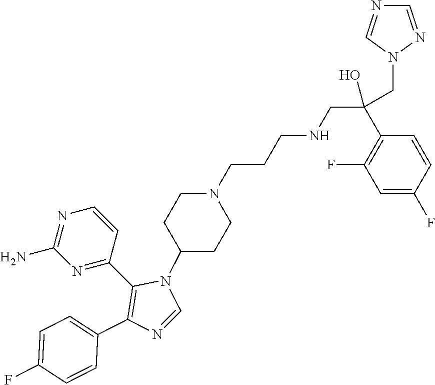 [SMILES: Nc1nccc(-c2c(-c3ccc(F)cc3)ncn2C2CCN(CCCNCC(O)(Cn3cncn3)c3ccc(F)cc3F)CC2)n1] |
| I-81 | (2R)-1-((1R,5S)-6-((3-(4-(5-(2- aminopyrimidin-4-yl)-4-(4- fluorophenyl)-1H-imidazol-1- yl)piperidin-1-yl)propyl)amino)- 3-azabicyclo[3.1.0]hexan-3-yl)- 2-(2,4-difluorophenyl)-3-(1H- 1,2,4-triazol-1-yl)propan-2-ol | 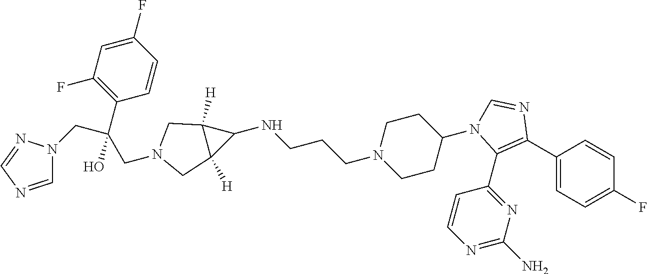 [SMILES: Nc1nccc(-c2c(-c3ccc(F)cc3)ncn2C2CCN(CCCNC3[C@H]4CN(C[C@@](O)(Cn5cncn5)c5ccc(F)cc5F)C[C@@H]34)CC2)n1] |
| I-82 | (2S)-1-((1R,5S)-6-((3-(4-(5-(2- aminopyrimidin-4-yl)-4-(4- fluorophenyl)-1H-imidazol-1- yl)piperidin-1-yl)propyl)amino)- 3-azabicyclo[3.1.0]hexan-3-yl)- 2-(2,4-difluorophenyl)-3-(1H- 1,2,4-triazol-1-yl)propan-2-ol | 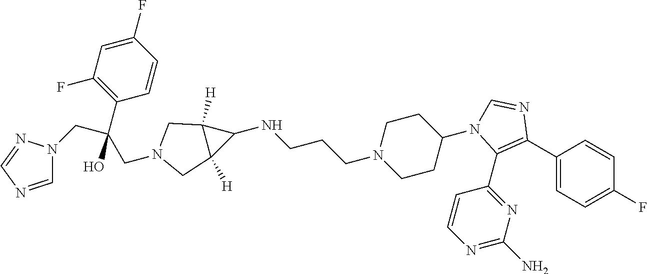 [SMILES: Nc1nccc(-c2c(-c3ccc(F)cc3)ncn2C2CCN(CCCNC3[C@H]4CN(C[C@](O)(Cn5cncn5)c5ccc(F)cc5F)C[C@@H]34)CC2)n1] |
| I-83 | 1-((1R,5S)-6-((3-(4-(5-(2- aminopyrimidin-4-yl)-4-(4- fluorophenyl)-1H-imidazol-1- yl)piperidin-1-yl)propyl)amino)- 3-azabicyclo[3.1.0]hexan-3-yl)- 2-(2,4-difluorophenyl)-3-(1H- 1,2,4-triazol-1-yl)propan-2-ol | 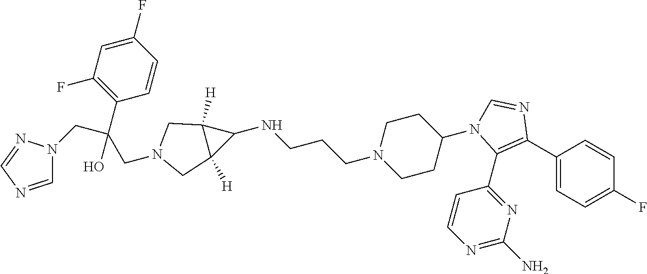 [SMILES: Nc1nccc(-c2c(-c3ccc(F)cc3)ncn2C2CCN(CCCNC3[C@H]4CN(CC(O)(Cn5cncn5)c5ccc(F)cc5F)C[C@@H]34)CC2)n1] |
| I-84 | 1-((1R,5S)-6-((2-(4-(5-(2- aminopyrimidin-4-yl)-4-(4- fluorophenyl)-1H-imidazol-1- yl)piperidin-1-yl)ethyl)amino)- 3-azabicyclo[3.1.0]hexan-3-yl)- 2-(2,4-difluorophenyl)-3-(1H- 1,2,4-triazol-1-yl)propan-2-ol | 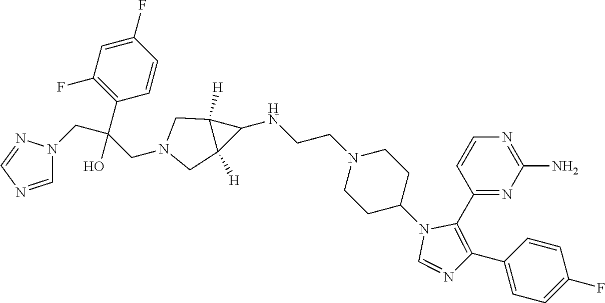 [SMILES: Nc1nccc(-c2c(-c3ccc(F)cc3)ncn2C2CCN(CCNC3[C@H]4CN(CC(O)(Cn5cncn5)c5ccc(F)cc5F)C[C@@H]34)CC2)n1] |
| I-85 | 2-(2-(3-(4-(5-(2- aminopyrimidin-4-yl)-4-(4- fluorophenyl)-1H-imidazol-1- yl)piperidin-1-yl)-3- oxopropoxy)ethoxy)-N-(2-(2,4- difluorophenyl)-2-hydroxy-3- (1H-1,2,4-triazol-1- yl)propyl)acetamide | 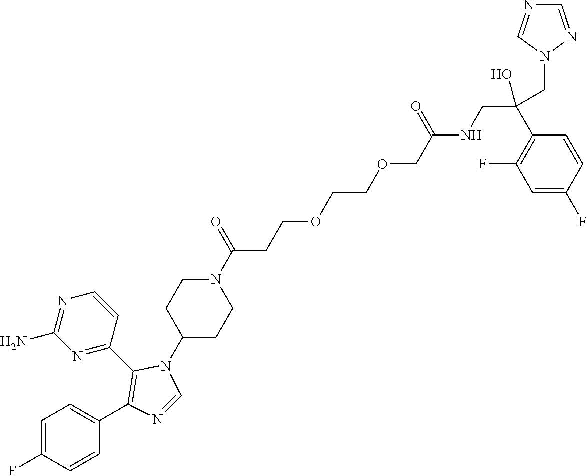 [SMILES: Nc1nccc(-c2c(-c3ccc(F)cc3)ncn2C2CCN(C(=O)CCOCCOCC(=O)NCC(O)(Cn3cncn3)c3ccc(F)cc3F)CC2)n1] |
| I-86 | 3-(2-(3-(4-(5-(2- aminopyrimidin-4-yl)-4-(4- fluorophenyl)-1H-imidazol-1- yl)piperidin-1-yl)-3- oxopropoxy)ethoxy)-N-(2-(2,4- difluorophenyl)-2-hydroxy-3- (1H-1,2,4-triazol-1- yl)propyl)propanamide | 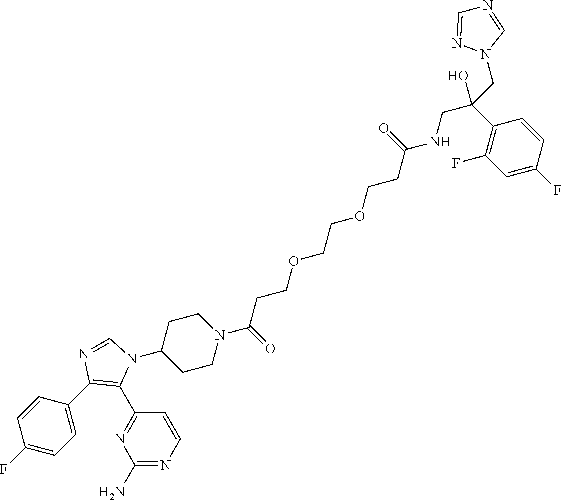 [SMILES: Nc1nccc(-c2c(-c3ccc(F)cc3)ncn2C2CCN(C(=O)CCOCCOCCC(=O)NCC(O)(Cn3cncn3)c3ccc(F)cc3F)CC2)n1] |
| I-87 | N-(2-(2,4-difluorophenyl)-2- hydroxy-3-(1H-1,2,4-triazol-1- yl)propyl)-3-(2-(3-((2-(4- fluorophenyl)-4-(pyridin-4- yl)pyrazolo[1,5-a]pyridin-7- yl)amino)-3- oxopropoxy)ethoxy) propanamide | 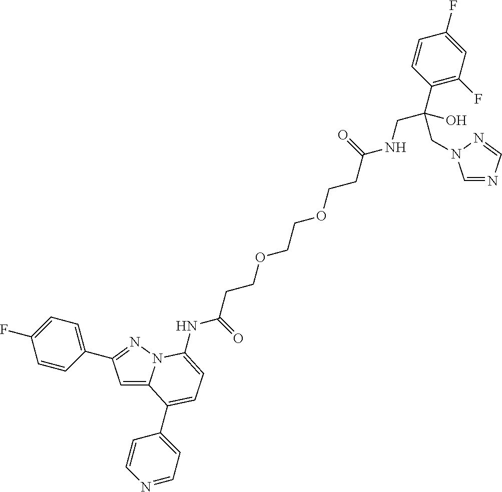 [SMILES: O=C(CCOCCOCCC(=O)Nc1ccc(-c2ccncc2)c2cc(-c3ccc(F)cc3)nn12)NCC(O)(Cn1cncn1)c1ccc(F)cc1F] |
| I-88 | 2-(4-fluorophenyl)-4-(pyridin-4- yl)pyrazolo[1,5-a]pyridin-7-yl 3- (2-(3-((2-(2,4-difluorophenyl)- 2-hydroxy-3-(1H-1,2,4-triazol- 1-yl)propyl)amino)-3- oxopropoxy)ethoxy)propanoate | 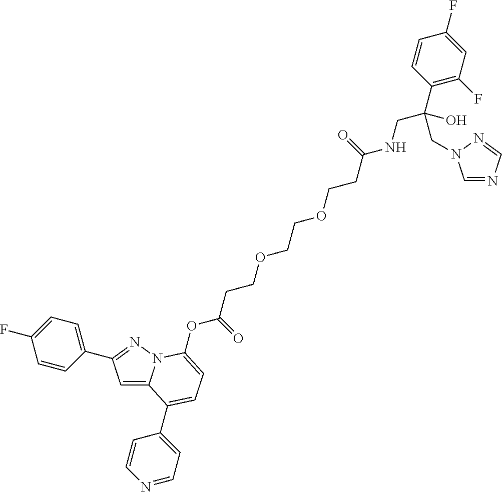 [SMILES: O=C(CCOCCOCCC(=O)Oc1ccc(-c2ccncc2)c2cc(-c3ccc(F)cc3)nn12)NCC(O)(Cn1cncn1)c1ccc(F)cc1F] |
| I-89 | 2-(2,4-difluorophenyl)- 1-(4-(2- (4-(3-(4-fluorophenyl)-1- methyl-1H-pyrazol-4-yl)pyridin- 3-yl)ethyl)piperidin-1-yl)-3-(1H- 1,2,4-triazol-1-yl)propan-2-ol | 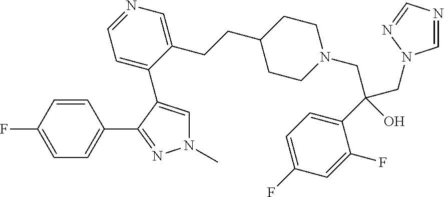 [SMILES: Cn1cc(-c2ccncc2CCC2CCN(CC(O)(Cn3cncn3)c3ccc(F)cc3F)CC2)c(-c2ccc(F)cc2)n1] |
| I-90 | 2-(2,4-difluorophenyl)- 1-(4-((4- (3-(4-fluorophenyl)-1-methyl- 1H-pyrazol-4-yl)pyridin-3- yl)methoxy)piperidin-1-yl)-3- (1H-1,2,4-triazol-1-yl)propan- 2-ol | 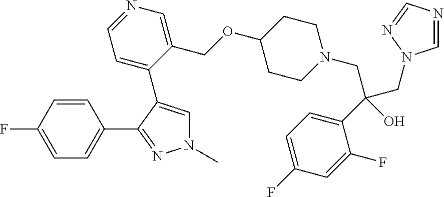 [SMILES: Cn1cc(-c2ccncc2COC2CCN(CC(O)(Cn3cncn3)c3ccc(F)cc3F)CC2)c(-c2ccc(F)cc2)n1] |
| I-91 | 1-(1-((2-(2-(4-(5-(2- aminopyrimidin-4-yl)-4-(4- fluorophenyl)-1H-imidazol-1- yl)piperidin-1- yl)ethoxy)ethoxy)methyl) piperidin-4-yl)-2-(2,4- difluorophenyl)-3-(1H-1,2,4- triazol-1-yl)propan-2-ol | 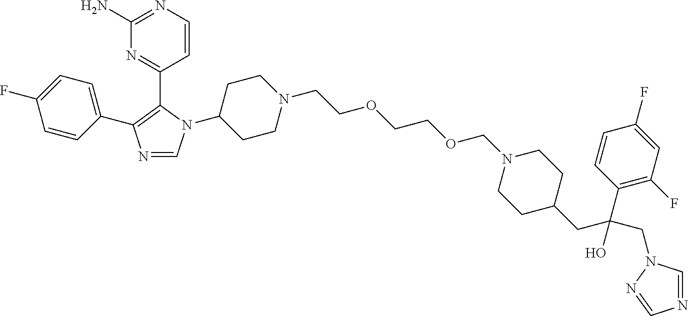 [SMILES: Nc1nccc(-c2c(-c3ccc(F)cc3)ncn2C2CCN(CCOCCOCN3CCC(CC(O)(Cn4cncn4)c4ccc(F)cc4F)CC3)CC2)n1] |
| I-92 | (S)-1-(1-((2-(2-(4-(5-(2- aminopyrimidin-4-yl)-4-(4- fluorophenyl)-1H-imidazol-1- yl)piperidin-1- yl)ethoxy)ethoxy)methyl) piperidin-4-yl)-2-(2,4- difluorophenyl)-3-(1H-1,2,4- triazol-1-yl)propan-2-ol | 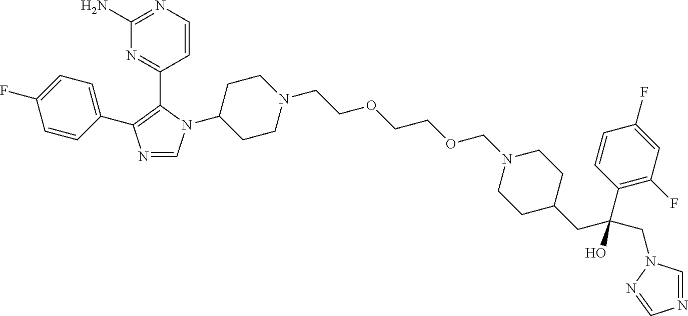 [SMILES: Nc1nccc(-c2c(-c3ccc(F)cc3)ncn2C2CCN(CCOCCOCN3CCC(C[C@@](O)(Cn4cncn4)c4ccc(F)cc4F)CC3)CC2)n1] |
| I-93 | (R)-1-(1-((2-(2-(4-(5-(2- aminopyrimidin-4-yl)-4-(4- fluorophenyl)-1H-imidazol-1- yl)piperidin-1- yl)ethoxy)ethoxy)methyl) piperidin-4-yl)-2-(2,4- difluorophenyl)-3-(1H-1,2,4- triazol-1-yl)propan-2-ol | 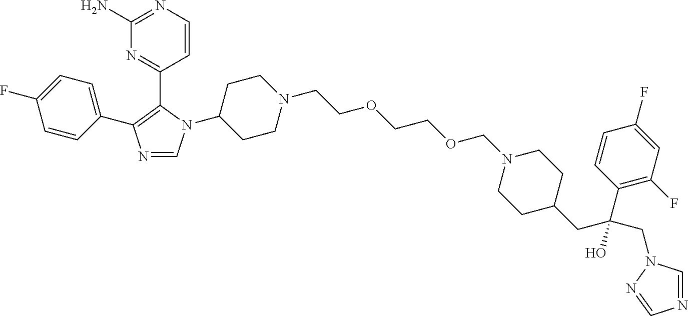 [SMILES: Nc1nccc(-c2c(-c3ccc(F)cc3)ncn2C2CCN(CCOCCOCN3CCC(C[C@](O)(Cn4cncn4)c4ccc(F)cc4F)CC3)CC2)n1] |
| I-94 | 1-(4-((2-(2-(2-(4-(5-(2- aminopyrimidin-4-yl)-4-(4- fluorophenyl)-1H-imidazol-1- yl)piperidin-1- yl)ethoxy)ethoxy)ethyl)amino) piperidin-1-yl)-2-(2,4- difluorophenyl)-3-(1H-1,2,4- triazol-1-yl)propan-2-ol | 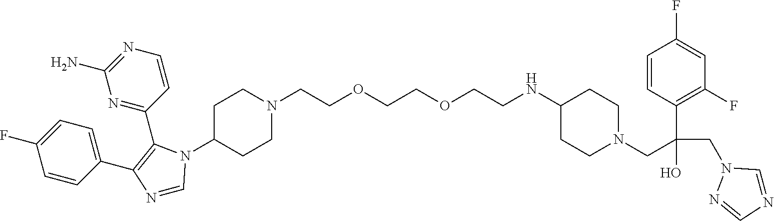 [SMILES: Nc1nccc(-c2c(-c3ccc(F)cc3)ncn2C2CCN(CCOCCOCCNC3CCN(CC(O)(Cn4cncn4)c4ccc(F)cc4F)CC3)CC2)n1] |
| I-95 | (R)-1-(4-((2-(2-(2-(4-(5-(2- aminopyrimidin-4-yl)-4-(4- fluorophenyl)-1H-imidazol-1- yl)piperidin-1- yl)ethoxy)ethoxy)ethyl)amino) piperidin-1-yl)-2-(2,4- difluorophenyl)-3-(1H-1,2,4- triazol-1-yl)propan-2-ol | 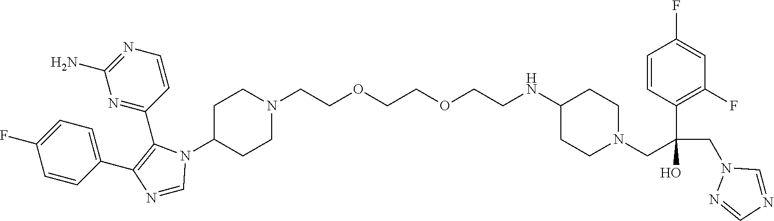 [SMILES: Nc1nccc(-c2c(-c3ccc(F)cc3)ncn2C2CCN(CCOCCOCCNC3CCN(C[C@@](O)(Cn4cncn4)c4ccc(F)cc4F)CC3)CC2)n1] |
| I-96 | (S)-1-(4-((2-(2-(2-(4-(5-(2- aminopyrimidin-4-yl)-4-(4- fluorophenyl)-1H-imidazol-1- yl)piperidin-1- yl)ethoxy)ethoxy)ethyl)amino) piperidin-1-yl)-2-(2,4- difluorophenyl)-3-(1H-1,2,4- triazol-1-yl)propan-2-ol | 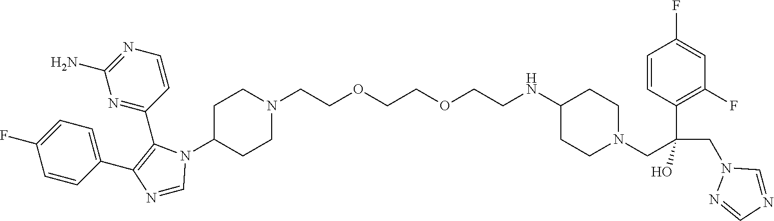 [SMILES: Nc1nccc(-c2c(-c3ccc(F)cc3)ncn2C2CCN(CCOCCOCCNC3CCN(C[C@](O)(Cn4cncn4)c4ccc(F)cc4F)CC3)CC2)n1] |
| I-97 | (S)-1-(4-((3-(4-(5-(2- aminopyrimidin-4-yl)-4-(4- fluorophenyl)-1H-imidazol-1- yl)piperidin-1- yl)propyl)amino)piperidin-1-yl)- 2-(2,4-difluorophenyl)-3-(1H- 1,2,4-triazol-1-yl)propan-2-ol | 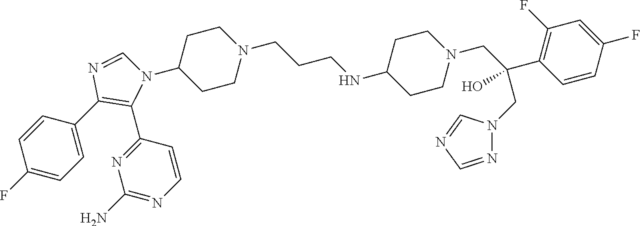 [SMILES: Nc1nccc(-c2c(-c3ccc(F)cc3)ncn2C2CCN(CCCNC3CCN(C[C@](O)(Cn4cncn4)c4ccc(F)cc4F)CC3)CC2)n1] |
| I-98 | 1-(4-((3-(4-(5-(2- aminopyrimidin-4-yl)-4-(4- fluorophenyl)-1H-imidazol-1- yl)piperidin-1- yl)propyl)amino)piperidin-1-yl)- 2-(2,4-difluorophenyl)-3-(1H- 1,2,4-triazol-1-yl)propan-2-ol | 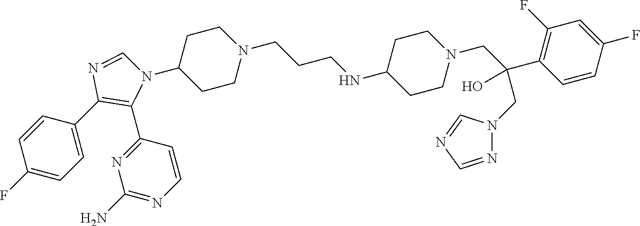 [SMILES: Nc1nccc(-c2c(-c3ccc(F)cc3)ncn2C2CCN(CCCNC3CCN(CC(O)(Cn4cncn4)c4ccc(F)cc4F)CC3)CC2)n1] |
| I-99 | (R)-1-(4-((3-(4-(5-(2- aminopyrimidin-4-yl)-4-(4- fluorophenyl)-1H-imidazol-1- yl)piperidin-1- yl)propyl)amino)piperidin-1-yl)- 2-(2,4-difluorophenyl)-3-(1H- 1,2,4-triazol-1-yl)propan-2-ol | 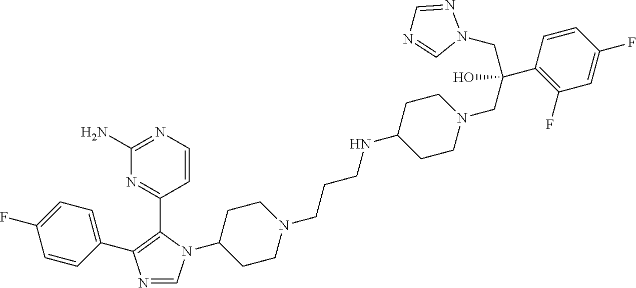 [SMILES: Nc1nccc(-c2c(-c3ccc(F)cc3)ncn2C2CCN(CCCNC3CCN(C[C@@](O)(Cn4cncn4)c4ccc(F)cc4F)CC3)CC2)n1] |
| I-100 | N-(1-(2-(2,4-difluorophenyl)-2- hydroxy-3-(1H-1,2,4-triazol-1- yl)propyl)piperidin-4-yl)-2-(4- fluorophenyl)-3-(pyridin-4- yl)pyrazolo[1,5-a]pyridine-6- carboxamide | 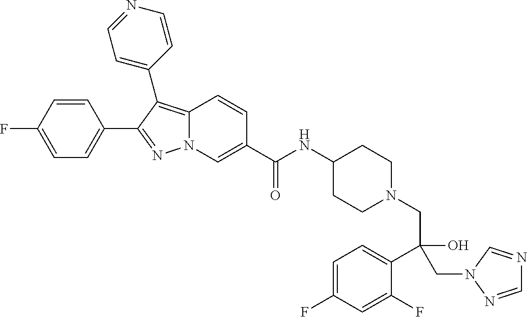 [SMILES: O=C(NC1CCN(CC(O)(Cn2cncn2)c2ccc(F)cc2F)CC1)c1ccc2c(-c3ccncc3)c(-c3ccc(F)cc3)nn2c1] |
| I-101 | 2-(2,4-difluorophenyl)- 1-(4- (((2-(4-fluorophenyl)-3-(pyridin- 4-yl)pyrazolo[1,5-a]pyridin-6- yl)methyl)amino)piperidin-1- yl)-3-(1H-1,2,4-triazol-1- yl)propan-2-ol | 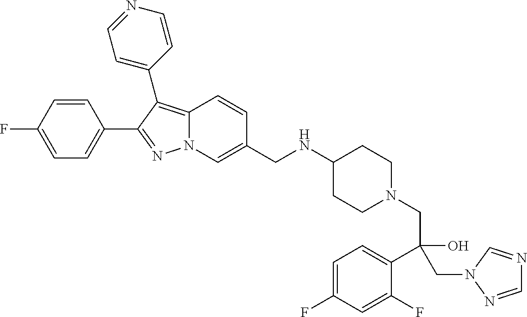 [SMILES: OC(CN1CCC(NCc2ccc3c(-c4ccncc4)c(-c4ccc(F)cc4)nn3c2)CC1)(Cn1cncn1)c1ccc(F)cc1F] |
| I-102 | (S)-N-(1-(2-(2,4- difluorophenyl)-2-hydroxy-3- (1H-1,2,4-triazol-1- yl)propyl)piperidin-4-yl)-2-(4- fluorophenyl)-3-(pyridin-4- yl)pyrazolo[1,5-a]pyridine-6- carboxamide | 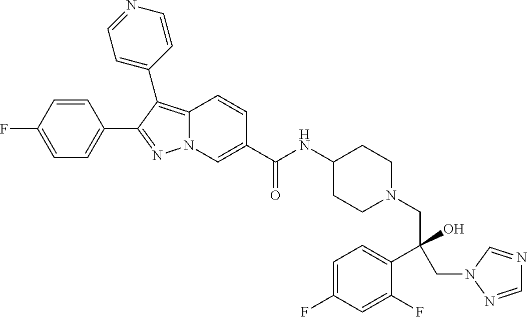 [SMILES: O=C(NC1CCN(C[C@](O)(Cn2cncn2)c2ccc(F)cc2F)CC1)c1ccc2c(-c3ccncc3)c(-c3ccc(F)cc3)nn2c1] |
| I-103 | (R)-N-(1-(2-(2,4- difluorophenyl)-2-hydroxy-3- (1H-1,2,4-triazol-1- yl)propyl)piperidin-4-yl)-2-(4- fluorophenyl)-3-(pyridin-4- yl)pyrazolo[1,5-a]pyridine-6- carboxamide | 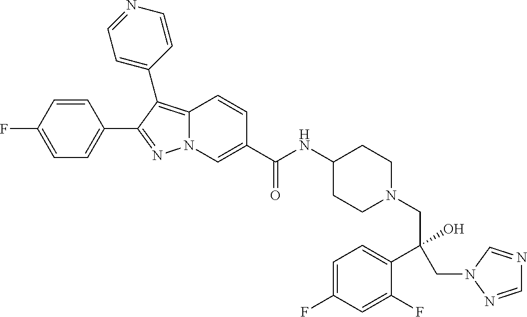 [SMILES: O=C(NC1CCN(C[C@@](O)(Cn2cncn2)c2ccc(F)cc2F)CC1)c1ccc2c(-c3ccncc3)c(-c3ccc(F)cc3)nn2c1] |
| I-104 | (R)-2-(2,4-difluorophenyl)-1-(4- (((2-(4-fluorophenyl)-3-(pyridin- 4-yl)pyrazolo[1,5-a]pyridin-6- yl)methyl)amino)piperidin-1- yl)-3-(1H-1,2,4-triazol-1- yl)propan-2-ol | 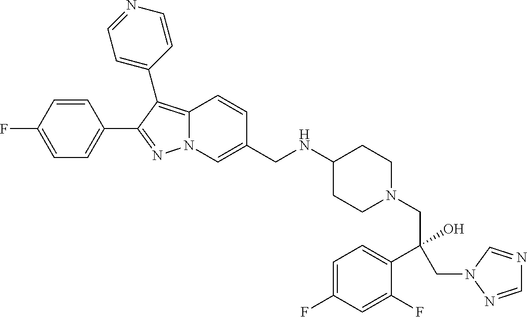 [SMILES: O[C@](CN1CCC(NCc2ccc3c(-c4ccncc4)c(-c4ccc(F)cc4)nn3c2)CC1)(Cn1cncn1)c1ccc(F)cc1F] |
| I-105 | (S)-2-(2,4-difluorophenyl)-1-(4- (((2-(4-fluorophenyl)-3-(pyridin- 4-yl)pyrazolo[1,5-a]pyridin-6- yl)methyl)amino)piperidin-1- yl)-3-(1H-1,2,4-triazol-1- yl)propan-2-ol | 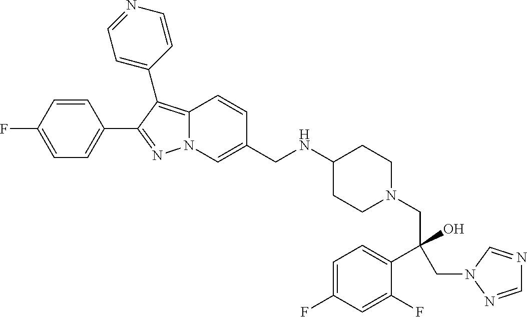 [SMILES: O[C@@](CN1CCC(NCc2ccc3c(-c4ccncc4)c(-c4ccc(F)cc4)nn3c2)CC1)(Cn1cncn1)c1ccc(F)cc1F] |
| I-106 | 3-(2,4-difluorophenyl)-N-(4-(6- fluoro-2-(4- fluorophenyl)pyrazolo[1,5- a]pyridin-3-yl)pyrimidin-2-yl)-3- hydroxy-4-(1H-1,2,4-triazol-1- yl)butanamide | 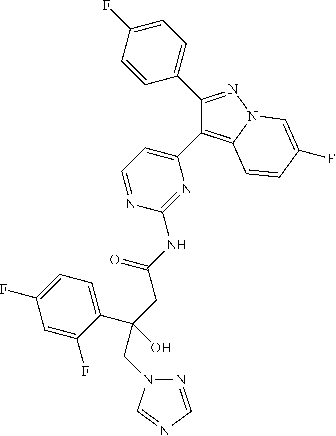 [SMILES: O=C(CC(O)(Cn1cncn1)c1ccc(F)cc1F)Nc1nccc(-c2c(-c3ccc(F)cc3)nn3cc(F)ccc23)n1] |
| I-107 | (R)-3-(2,4-difluorophenyl)-N- (4-(6-fluoro-2-(4- fluorophenyl)pyrazolo[1,5- a]pyridin-3-yl)pyrimidin-2-yl)-3- hydroxy-4-(1H-1,2,4-triazol-1- yl)butanamide | 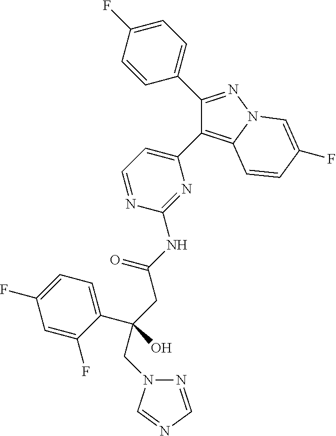 [SMILES: O=C(C[C@](O)(Cn1cncn1)c1ccc(F)cc1F)Nc1nccc(-c2c(-c3ccc(F)cc3)nn3cc(F)ccc23)n1] |
| I-108 | (S)-3-(2,4-difluorophenyl)-N- (4-(6-fluoro-2-(4- fluorophenyl)pyrazolo[1,5- a]pyridin-3-yl)pyrimidin-2-yl)-3- hydroxy-4-(1H-1,2,4-triazol-1- yl)butanamide | 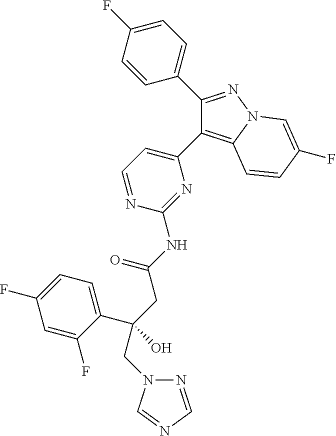 [SMILES: O=C(C[C@@](O)(Cn1cncn1)c1ccc(F)cc1F)Nc1nccc(-c2c(-c3ccc(F)cc3)nn3cc(F)ccc23)n1] |
| I-109 | 3-(2,4-difluorophenyl)-N-(4-(6- fluoro-2-(4- fluorophenyl)pyrazolo[1,5- a]pyridin-3-yl)pyridin-2-yl)-3- hydroxy-4-(1H-1,2,4-triazol-1- yl)butanamide | 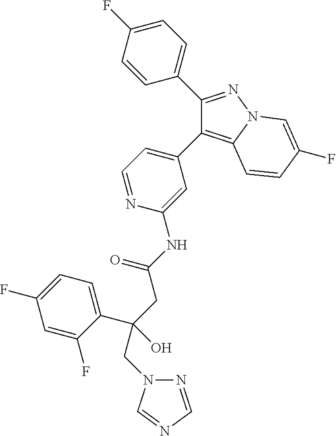 [SMILES: O=C(CC(O)(Cn1cncn1)c1ccc(F)cc1F)Nc1cc(-c2c(-c3ccc(F)cc3)nn3cc(F)ccc23)ccn1] |
| I-110 | (R)-3-(2,4-difluorophenyl)-N- (4-(6-fluoro-2-(4- fluorophenyl)pyrazolo[1,5- a]pyridin-3-yl)pyridin-2-yl)-3- hydroxy-4-(1H-1,2,4-triazol-1- yl)butanamide | 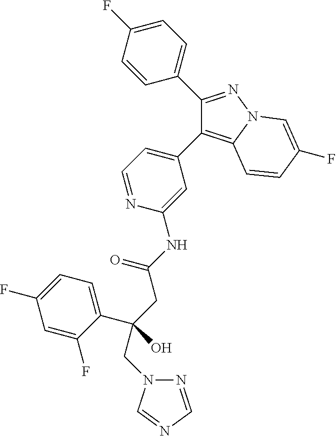 [SMILES: O=C(C[C@](O)(Cn1cncn1)c1ccc(F)cc1F)Nc1cc(-c2c(-c3ccc(F)cc3)nn3cc(F)ccc23)ccn1] |
| I-111 | (S)-3-(2,4-difluorophenyl)-N- (4-(6-fluoro-2-(4- fluorophenyl)pyrazolo[1,5- a]pyridin-3-yl)pyridin-2-yl)-3- hydroxy-4-(1H-1,2,4-triazol-1- yl)butanamide | 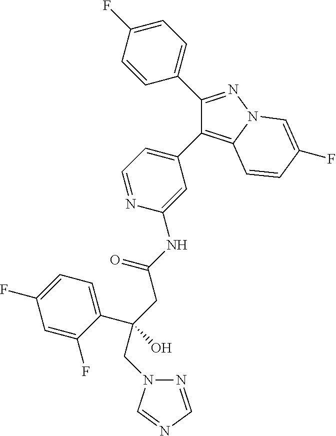 [SMILES: O=C(C[C@@](O)(Cn1cncn1)c1ccc(F)cc1F)Nc1cc(-c2c(-c3ccc(F)cc3)nn3cc(F)ccc23)ccn1] |
| I-112 | 2-(2,4-difluorophenyl)-4-((4-(6- fluoro-2-(4- fluorophenyl)pyrazolo[1,5- a]pyridin-3-yl)pyridin-2- yl)amino)-1-(1H-1,2,4-triazol-1- yl)butan-2-ol | 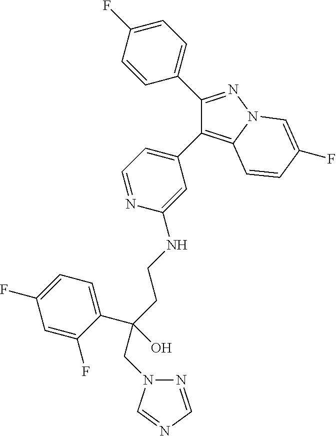 [SMILES: OC(CCNc1cc(-c2c(-c3ccc(F)cc3)nn3cc(F)ccc23)ccn1)(Cn1cncn1)c1ccc(F)cc1F] |
| I-113 | (R)-2-(2,4-difluorophenyl)-4- ((4-(6-fluoro-2-(4- fluorophenyl)pyrazolo[1,5- a]pyridin-3-yl)pyridin-2- yl)amino)-1-(1H-1,2,4-triazol-1- yl)butan-2-ol | 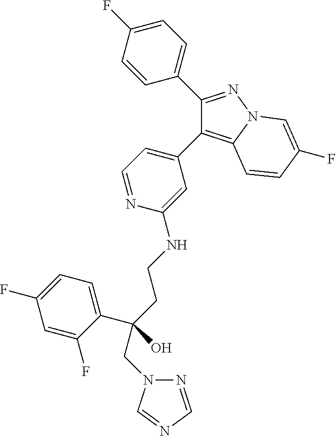 [SMILES: O[C@@](CCNc1cc(-c2c(-c3ccc(F)cc3)nn3cc(F)ccc23)ccn1)(Cn1cncn1)c1ccc(F)cc1F] |
| I-114 | (S)-2-(2,4-difluorophenyl)-4- ((4-(6-fluoro-2-(4- fluorophenyl)pyrazolo[1,5- a]pyridin-3-yl)pyridin-2- yl)amino)-1-(1H-1,2,4-triazol-1- yl)butan-2-ol | 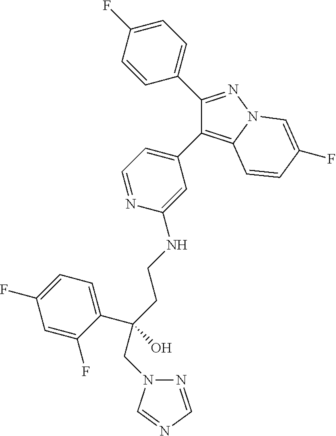 [SMILES: O[C@](CCNc1cc(-c2c(-c3ccc(F)cc3)nn3cc(F)ccc23)ccn1)(Cn1cncn1)c1ccc(F)cc1F] |
| I-115 | N-(2-(2,4-difluorophenyl)-2- hydroxy-3-(1H-1,2,4-triazol-1- yl)propyl)-2-(4-fluorophenyl)-3- (pyridin-4-yl)pyrazolo[1,5- a]pyridine-6-carboxamide | 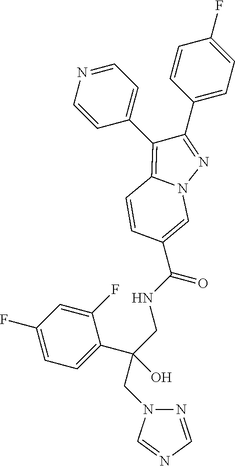 [SMILES: O=C(NCC(O)(Cn1cncn1)c1ccc(F)cc1F)c1ccc2c(-c3ccncc3)c(-c3ccc(F)cc3)nn2c1] |
| I-116 | (S)-N-(2-(2,4-difluorophenyl)- 2-hydroxy-3-(1H-1,2,4-triazol- 1-yl)propyl)-2-(4-fluorophenyl)- 3-(pyridin-4-yl)pyrazolo[1,5- a]pyridine-6-carboxamide | 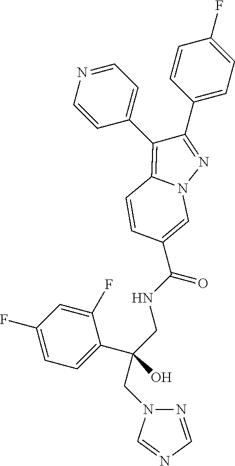 [SMILES: O=C(NC[C@](O)(Cn1cncn1)c1ccc(F)cc1F)c1ccc2c(-c3ccncc3)c(-c3ccc(F)cc3)nn2c1] |
| I-117 | (R)-N-(2-(2,4-difluorophenyl)- 2-hydroxy-3-(1H-1,2,4-triazol- 1-yl)propyl)-2-(4-fluorophenyl)- 3-(pyridin-4-yl)pyrazolo[1,5- a]pyridine-6-carboxamide | 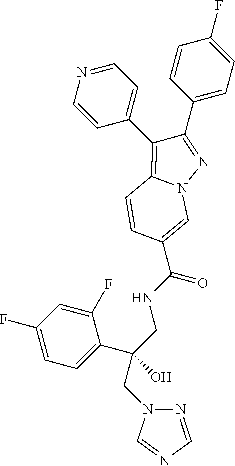 [SMILES: O=C(NC[C@@](O)(Cn1cncn1)c1ccc(F)cc1F)c1ccc2c(-c3ccncc3)c(-c3ccc(F)cc3)nn2c1] |
| I-118 | (S)-2-(2,4-difluorophenyl)-1- (((4-(6-fluoro-2-(4- fluorophenyl)pyrazolo[1,5- a]pyridin-3-yl)pyridin-2- yl)methyl)amino)-3-(1H-1,2,4- triazol-1-yl)propan-2-ol | 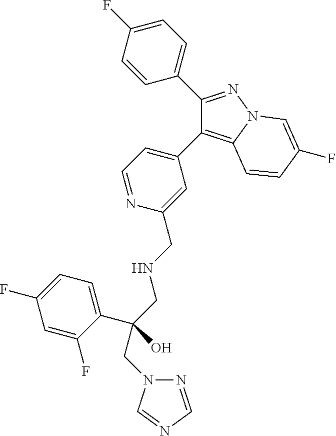 [SMILES: O[C@@](CNCc1cc(-c2c(-c3ccc(F)cc3)nn3cc(F)ccc23)ccn1)(Cn1cncn1)c1ccc(F)cc1F] |
| I-119 | (S)-2-(2,4-difluorophenyl)-1- (((4-(6-fluoro-2-(4- fluorophenyl)pyrazolo[1,5- a]pyridin-3-yl)pyrimidin-2- yl)methyl)amino)-3-(1H-1,2,4- triazol-1-yl)propan-2-ol | 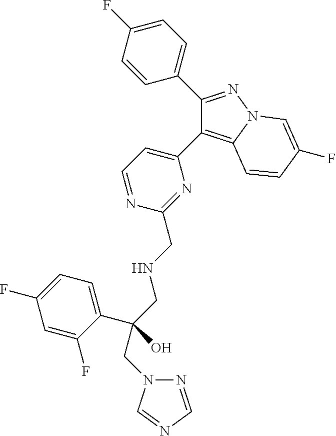 [SMILES: O[C@@](CNCc1nccc(-c2c(-c3ccc(F)cc3)nn3cc(F)ccc23)n1)(Cn1cncn1)c1ccc(F)cc1F] |
| I-120 | (S)-2-(2,4-difluorophenyl)-1- (((4-(6-fluoro-2-(4- fluorophenyl)pyrazolo[1,5- a]pyridin-3-yl)pyridin-3- yl)methyl)amino)-3-(1H-1,2,4- triazol-1-yl)propan-2-ol | 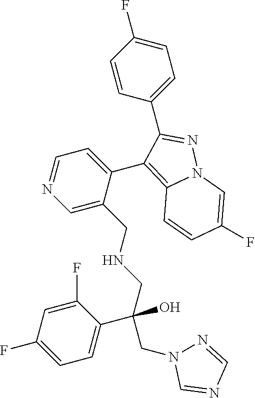 [SMILES: O[C@@](CNCc1cnccc1-c1c(-c2ccc(F)cc2)nn2cc(F)ccc12)(Cn1cncn1)c1ccc(F)cc1F] |
| I-121 | (R)-2-(2,4-difluorophenyl)-4- (((4-(6-fluoro-2-(4- fluorophenyl)pyrazolo[1,5- a]pyridin-3-yl)pyridin-3- yl)methyl)amino)-1-(1H-1,2,4- triazol-1-yl)butan-2-ol | 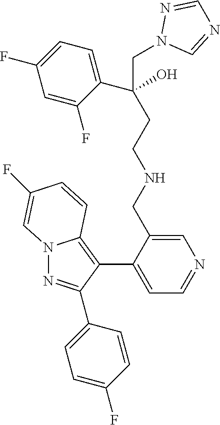 [SMILES: O[C@@](CCNCc1cnccc1-c1c(-c2ccc(F)cc2)nn2cc(F)ccc12)(Cn1cncn1)c1ccc(F)cc1F] |
| I-122 | (S)-2-(2,4-difluorophenyl)-1- ((2-(4-(6-fluoro-2-(4- fluorophenyl)pyrazolo[1,5- a]pyridin-3-yl)pyrimidin-2- yl)ethyl)amino)-3-(1H-1,2,4- triazol-1-yl)propan-2-ol | 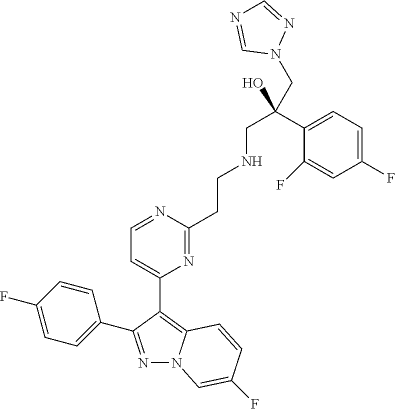 [SMILES: O[C@@](CNCCc1nccc(-c2c(-c3ccc(F)cc3)nn3cc(F)ccc23)n1)(Cn1cncn1)c1ccc(F)cc1F] |
| I-123 | (R)-2-(2,4-difluorophenyl)-1- (((4-(6-fluoro-2-(4- fluorophenyl)pyrazolo[1,5- a]pyridin-3-yl)pyrimidin-2- yl)methyl)amino)-3-(1H-1,2,4- triazol-1-yl)propan-2-ol | 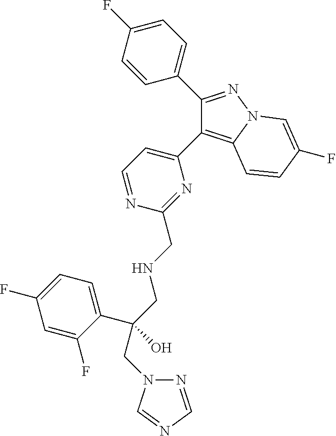 [SMILES: O[C@](CNCc1nccc(-c2c(-c3ccc(F)cc3)nn3cc(F)ccc23)n1)(Cn1cncn1)c1ccc(F)cc1F] |
| I-124 | (R)-2-(2,4-difluorophenyl)-1-(4- (4-(6-fluoro-2-(4- fluorophenyl)pyrazolo[1,5- a]pyridin-3-yl)pyrimidin-2- yl)piperazin-1-yl)-3-(1H-1,2,4- triazol-1-yl)propan-2-ol | 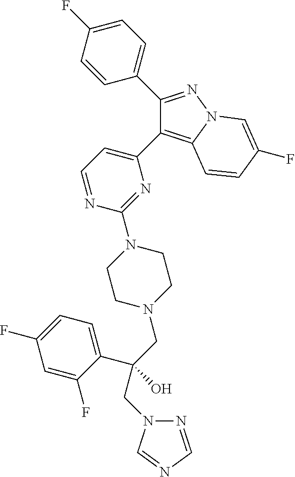 [SMILES: O[C@](CN1CCN(c2nccc(-c3c(-c4ccc(F)cc4)nn4cc(F)ccc34)n2)CC1)(Cn1cncn1)c1ccc(F)cc1F] |
| I-125 | (R)-2-(2,4-difluorophenyl)-1-(4- (4-(6-fluoro-2-(4- fluorophenyl)pyrazolo[1,5- a]pyridin-3-yl)pyrimidin-2- yl)piperidin-1-yl)-3-(1H-1,2,4- triazol-1-yl)propan-2-ol | 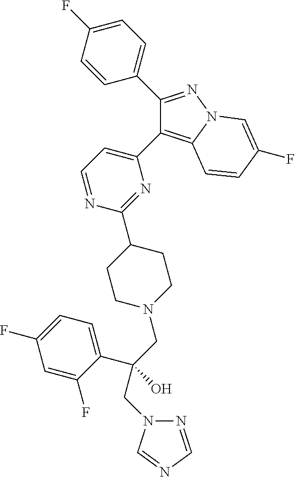 [SMILES: O[C@](CN1CCC(c2nccc(-c3c(-c4ccc(F)cc4)nn4cc(F)ccc34)n2)CC1)(Cn1cncn1)c1ccc(F)cc1F] |
| I-126 | (S)-2-(2,4-difluorophenyl)-1-(4- (4-(6-fluoro-2-(4- fluorophenyl)pyrazolo[1,5- a]pyridin-3-yl)pyrimidin-2- yl)piperidin-1-yl)-3-(1H-1,2,4- triazol-1-yl)propan-2-ol | 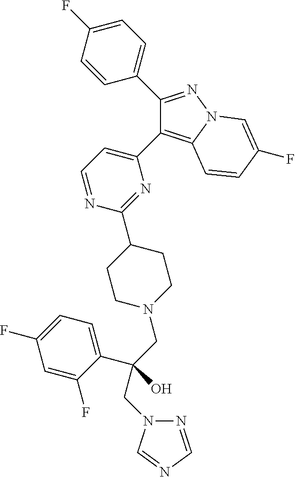 [SMILES: O[C@@](CN1CCC(c2nccc(-c3c(-c4ccc(F)cc4)nn4cc(F)ccc34)n2)CC1)(Cn1cncn1)c1ccc(F)cc1F] |
| I-127 | (S)-2-(2,4-difluorophenyl)-1-(4- (4-(6-fluoro-2-(4- fluorophenyl)pyrazolo[1,5- a]pyridin-3-yl)pyrimidin-2- yl)piperazin-1-yl)-3-(1H-1,2,4- triazol-1-yl)propan-2-ol | 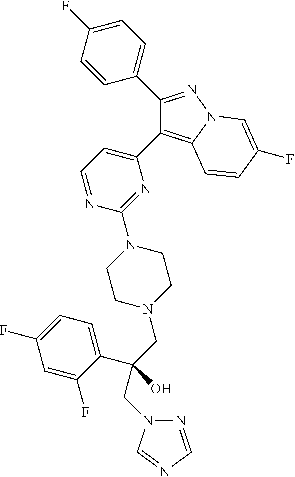 [SMILES: O[C@@](CN1CCN(c2nccc(-c3c(-c4ccc(F)cc4)nn4cc(F)ccc34)n2)CC1)(Cn1cncn1)c1ccc(F)cc1F] |
| I-128 | 1-((3-(2-aminopyridin-4-yl)-2- (4-fluorophenyl)pyrazolo[1,5- a]pyridin-7-yl)oxy)-2-(2,4- difluorophenyl)-3-(1H-1,2,4- triazol-1-yl)propan-2-ol | 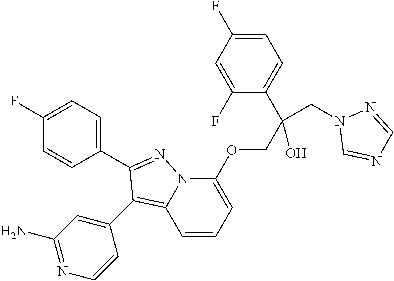 [SMILES: Nc1cc(-c2c(-c3ccc(F)cc3)nn3c(OCC(O)(Cn4cncn4)c4ccc(F)cc4F)cccc23)ccn1] |
| I-129 | (S)-1-((3-(2-aminopyridin-4-yl)- 2-(4-fluorophenyl)pyrazolo[1,5- a]pyridin-7-yl)oxy)-2-(2,4- difluorophenyl)-3-(1H-1,2,4- triazol-1-yl)propan-2-ol | 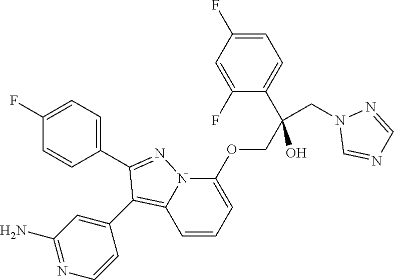 [SMILES: Nc1cc(-c2c(-c3ccc(F)cc3)nn3c(OC[C@@](O)(Cn4cncn4)c4ccc(F)cc4F)cccc23)ccn1] |
| I-130 | (R)-1-((3-(2-aminopyridin-4-yl)- 2-(4-fluorophenyl)pyrazolo[1,5- a]pyridin-7-yl)oxy)-2-(2,4- difluorophenyl)-3-(1H-1,2,4- triazol-1-yl)propan-2-ol | 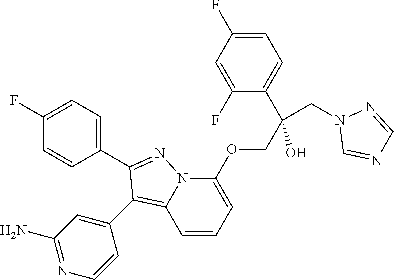 [SMILES: Nc1cc(-c2c(-c3ccc(F)cc3)nn3c(OC[C@](O)(Cn4cncn4)c4ccc(F)cc4F)cccc23)ccn1] |
| I-131 | (R)-2-(2,4-difluorophenyl)-1- ((2-(4-fluorophenyl)-3-(pyridin- 4-yl)pyrazolo[1,5-a]pyridin-7- yl)oxy)-3-(1H-1,2,4-triazol-1- yl)propan-2-ol | 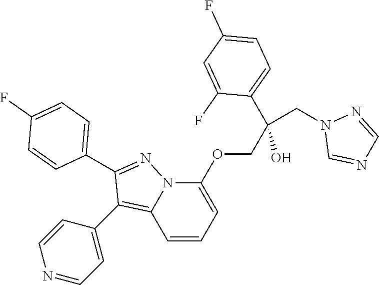 [SMILES: O[C@@](COc1cccc2c(-c3ccncc3)c(-c3ccc(F)cc3)nn12)(Cn1cncn1)c1ccc(F)cc1F] |
| I-132 | (S)-2-(2,4-difluorophenyl)-1- ((2-(4-fluorophenyl)-3-(pyridin- 4-yl)pyrazolo[1,5-a]pyridin-7- yl)oxy)-3-(1H-1,2,4-triazol-1- yl)propan-2-ol | 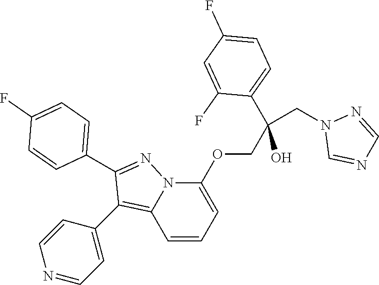 [SMILES: O[C@](COc1cccc2c(-c3ccncc3)c(-c3ccc(F)cc3)nn12)(Cn1cncn1)c1ccc(F)cc1F] |
| I-133 | (R)-2-(2,4-difluorophenyl)-1- ((2-(4-fluorophenyl)-3-(pyridin- 4-yl)pyrazolo[1,5-a]pyridin-7- yl)amino)-3-(1H-1,2,4-triazol-1- yl)propan-2-ol | 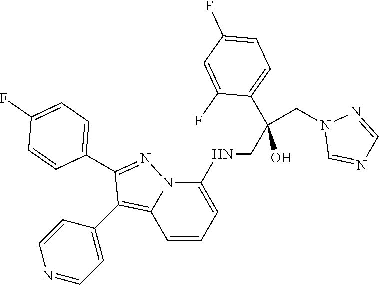 [SMILES: O[C@](CNc1cccc2c(-c3ccncc3)c(-c3ccc(F)cc3)nn12)(Cn1cncn1)c1ccc(F)cc1F] |
| I-134 | (S)-2-(2,4-difluorophenyl)-1- ((2-(4-fluorophenyl)-3-(pyridin- 4-yl)pyrazolo[1,5-a]pyridin-7- yl)amino)-3-(1H-1,2,4-triazol-1- yl)propan-2-ol | 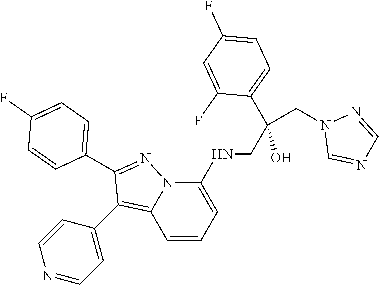 [SMILES: O[C@@](CNc1cccc2c(-c3ccncc3)c(-c3ccc(F)cc3)nn12)(Cn1cncn1)c1ccc(F)cc1F] |
| I-135 | 2-(2,4-difluorophenyl)-1-((2-(4- fluorophenyl)-3-(pyridin-4- yl)pyrazolo[1,5-a]pyridin-7- yl)amino)-3-(1H-1,2,4-triazol-1- yl)propan-2-ol | 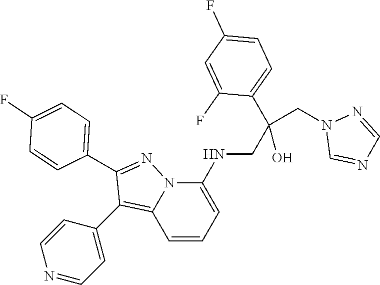 [SMILES: OC(CNc1cccc2c(-c3ccncc3)c(-c3ccc(F)cc3)nn12)(Cn1cncn1)c1ccc(F)cc1F] |
| I-136 | 1-((3-(2-aminopyridin-4-yl)-2- (4-fluorophenyl)pyrazolo[1,5- a]pyridin-7-yl)amino)-2-(2,4- difluorophenyl)-3-(1H-1,2,4- triazol-1-yl)propan-2-ol | 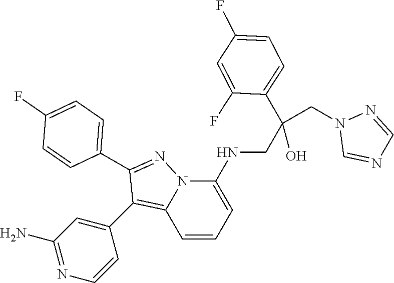 [SMILES: Nc1cc(-c2c(-c3ccc(F)cc3)nn3c(NCC(O)(Cn4cncn4)c4ccc(F)cc4F)cccc23)ccn1] |
| I-137 | (R)-1-((3-(2-aminopyridin-4-yl)- 2-(4-fluorophenyl)pyrazolo[1,5- a]pyridin-7-yl)amino)-2-(2,4- difluorophenyl)-3-(1H-1,2,4- triazol-1-yl)propan-2-ol | 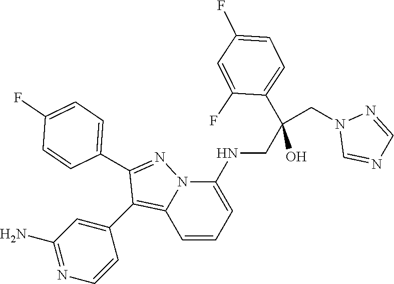 [SMILES: Nc1cc(-c2c(-c3ccc(F)cc3)nn3c(NC[C@@](O)(Cn4cncn4)c4ccc(F)cc4F)cccc23)ccn1] |
| I-138 | (S)-1-((3-(2-aminopyridin-4-yl)- 2-(4-fluorophenyl)pyrazolo[1,5- a]pyridin-7-yl)amino)-2-(2,4- difluorophenyl)-3-(1H-1,2,4- triazol-1-yl)propan-2-ol | 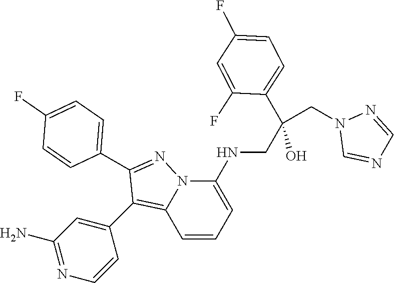 [SMILES: Nc1cc(-c2c(-c3ccc(F)cc3)nn3c(NC[C@](O)(Cn4cncn4)c4ccc(F)cc4F)cccc23)ccn1] |
| I-139 | 2-(2,4-difluorophenyl)- 1-((2-(4- fluorophenyl)-3-(pyridin-4- yl)pyrazolo[1,5-a]pyridin-7- yl)oxy)-3-(1H-1,2,4-triazol-1- yl)propan-2-ol | 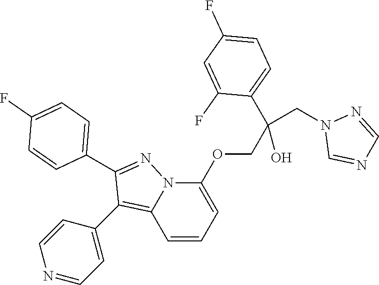 [SMILES: OC(COc1cccc2c(-c3ccncc3)c(-c3ccc(F)cc3)nn12)(Cn1cncn1)c1ccc(F)cc1F] |
| I-140 | 1-(4-(3-(2-aminopyridin-4-yl)-2- (4-fluorophenyl)pyrazolo[1,5- a]pyridin-7-yl)piperazin-1-yl)-2- (2,4-difluorophenyl)-3-(1H- 1,2,4-triazol-1-yl)propan-2-ol | 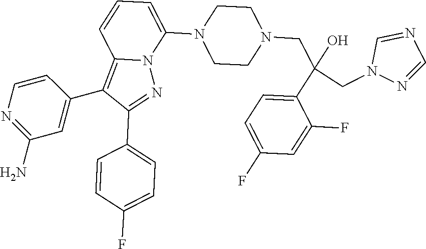 [SMILES: Nc1cc(-c2c(-c3ccc(F)cc3)nn3c(N4CCN(CC(O)(Cn5cncn5)c5ccc(F)cc5F)CC4)cccc23)ccn1] |
| I-141 | 1-(4-(3-(2-aminopyrimidin-4- yl)-2-(4- fluorophenyl)pyrazolo[1,5- a]pyridin-7-yl)piperazin-1-yl)-2- (2,4-difluorophenyl)-3-(1H- 1,2,4-triazol-1-yl)propan-2-ol | 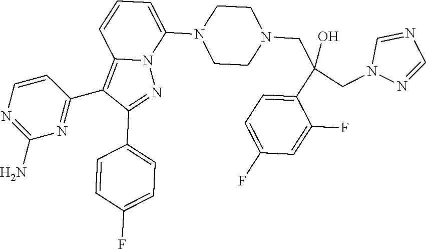 [SMILES: Nc1nccc(-c2c(-c3ccc(F)cc3)nn3c(N4CCN(CC(O)(Cn5cncn5)c5ccc(F)cc5F)CC4)cccc23)n1] |
| I-142 | 2-(2,4-difluorophenyl)- 1-(4-(2- (4-fluorophenyl)-3-(2-((5- methyl-1H-pyrazol-4- yl)amino)pyrimidin-4- yl)pyrazolo[1,5-a]pyridin-7- yl)piperazin-1-yl)-3-(1H-1,2,4- triazol-1-yl)propan-2-ol | 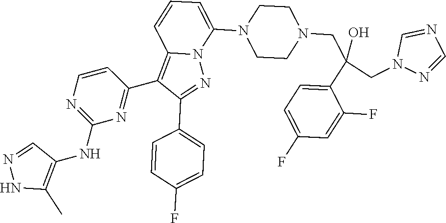 [SMILES: Cc1[nH]ncc1Nc1nccc(-c2c(-c3ccc(F)cc3)nn3c(N4CCN(CC(O)(Cn5cncn5)c5ccc(F)cc5F)CC4)cccc23)n1] |
| I-143 | 1-(4-(3-(2-((1-(2,2- difluorocyclopropyl)-5-methyl- 1H-pyrazol-4- yl)amino)pyrimidin-4-yl)-2-(4- fluorophenyl)pyrazolo[1,5- a]pyridin-7-yl)piperazin-1-yl)-2- (2,4-difluorophenyl)-3-(1H- 1,2,4-triazol-1-yl)propan-2-ol | 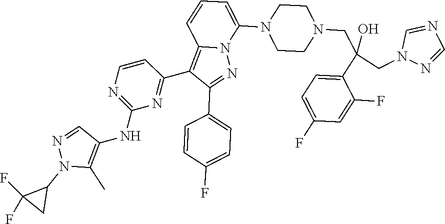 [SMILES: Cc1c(Nc2nccc(-c3c(-c4ccc(F)cc4)nn4c(N5CCN(CC(O)(Cn6cncn6)c6ccc(F)cc6F)CC5)cccc34)n2)cnn1C1CC1(F)F] |
| I-144 | 2-(2,4-difluorophenyl)- 1-((4-(6- fluoro-2-(4- fluorophenyl)pyrazolo[1,5- a]pyridin-3-yl)pyridin-2- yl)amino)-3-(1H-1,2,4-triazol-1- yl)propan-2-ol | 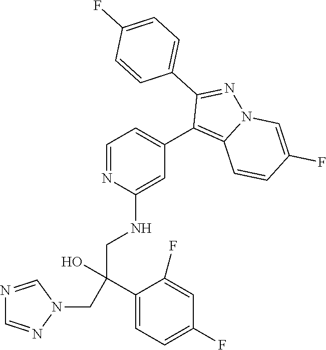 [SMILES: OC(CNc1cc(-c2c(-c3ccc(F)cc3)nn3cc(F)ccc23)ccn1)(Cn1cncn1)c1ccc(F)cc1F] |
| I-145 | N-(2-(2,4-difluorophenyl)-2- hydroxy-3-(1H-1,2,4-triazol-1- yl)propyl)-3-(2-(3-((2-(4- fluorophenyl)-4-(pyridin-4- yl)pyrazolo[1,5-a]pyridin-7- yl)amino)-3- oxopropoxy)ethoxy)propanami de | 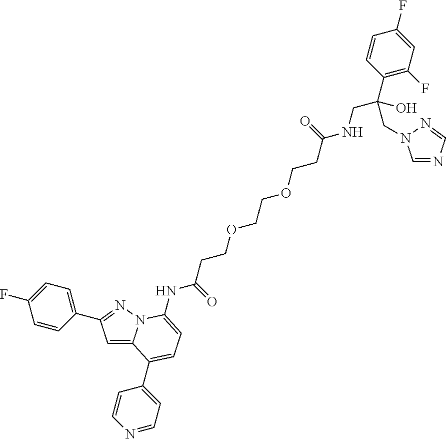 [SMILES: O=C(CCOCCOCCC(=O)Nc1ccc(-c2ccncc2)c2cc(-c3ccc(F)cc3)nn12)NCC(O)(Cn1cncn1)c1ccc(F)cc1F] |
| I-146 | 2-(4-fluorophenyl)-4-(pyridin-4- yl)pyrazolo[1,5-a]pyridin-7-yl 3- (2-(3-((2-(2,4-difluorophenyl)- 2-hydroxy-3-(1H-1,2,4-triazol- 1-yl)propyl)amino)-3- oxopropoxy)ethoxy)propanoate | 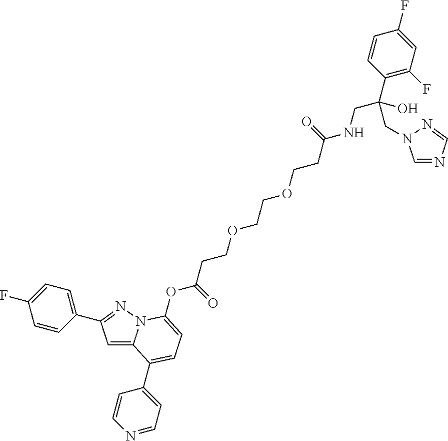 [SMILES: O=C(CCOCCOCCC(=O)Oc1ccc(-c2ccncc2)c2cc(-c3ccc(F)cc3)nn12)NCC(O)(Cn1cncn1)c1ccc(F)cc1F] |
| I-147 | N-((1-(2-(2,4-difluorophenyl)-2- hydroxy-3-(1H-1,2,4-triazol-1- yl)propyl)piperidin-4- yl)methyl)-2-(4-fluorophenyl)- 3-(pyridin-4-yl)pyrazolo[1,5- a]pyridine-6-carboxamide | 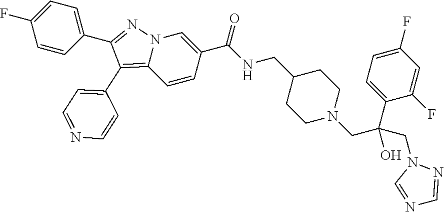 [SMILES: O=C(NCC1CCN(CC(O)(Cn2cncn2)c2ccc(F)cc2F)CC1)c1ccc2c(-c3ccncc3)c(-c3ccc(F)cc3)nn2c1] |
| I-148 | N-(3-(2,4-difluorophenyl)-3- hydroxy-4-(1H-1,2,4-triazol-1- yl)butyl)-2-(4-fluorophenyl)-3- (pyridin-4-yl)pyrazolo[1,5- a]pyridine-6-carboxamide | 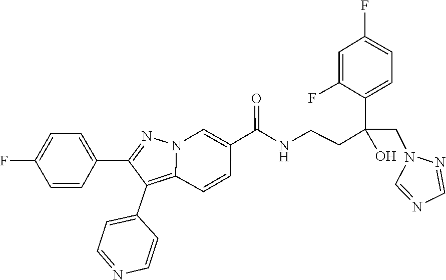 [SMILES: O=C(NCCC(O)(Cn1cncn1)c1ccc(F)cc1F)c1ccc2c(-c3ccncc3)c(-c3ccc(F)cc3)nn2c1] |
| I-149 | (6-chloro-1,2,3,4-tetrahydro- 9H-pyrido[3,4-b]indol-9-yl)(2- (4-fluorophenyl)-3-(pyridin-4- yl)pyrazolo[1,5-a]pyridin-6- yl)methanone | 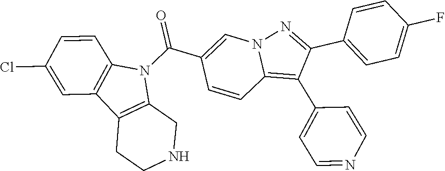 [SMILES: O=C(c1ccc2c(-c3ccncc3)c(-c3ccc(F)cc3)nn2c1)n1c2c(c3cc(Cl)ccc31)CCNC2] |
| I-150 | N-(3-(6-chloro-1,2,3,4- tetrahydro-9H-pyrido[3,4- b]indol-9-yl)propyl)-2-(4- fluorophenyl)-3-(pyridin-4- yl)pyrazolo[1,5-a]pyridine-6- carboxamide | 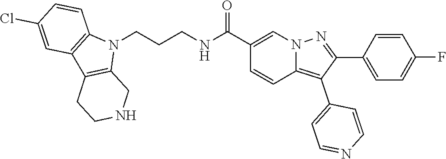 [SMILES: O=C(NCCCn1c2c(c3cc(Cl)ccc31)CCNC2)c1ccc2c(-c3ccncc3)c(-c3ccc(F)cc3)nn2c1] |
| I-151 | N-(2-(2,4-difluorophenyl)-2- hydroxy-3-(1H-1,2,4-triazol-1- yl)propyl)-2-(4-fluorophenyl)-3- (pyridin-4-yl)pyrazolo[1,5- a]pyridine-7-carboxamide | 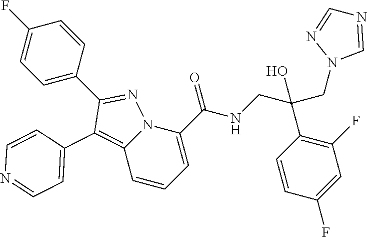 [SMILES: O=C(NCC(O)(Cn1cncn1)c1ccc(F)cc1F)c1cccc2c(-c3ccncc3)c(-c3ccc(F)cc3)nn12] |
| I-152 | 2-(2,4-difluorophenyl)- 1-(((2- (4-fluorophenyl)-3-(pyridin-4- yl)pyrazolo[1,5-a]pyridin-6- yl)methyl)amino)-3-(1H-1,2,4- triazol-1-yl)propan-2-ol | 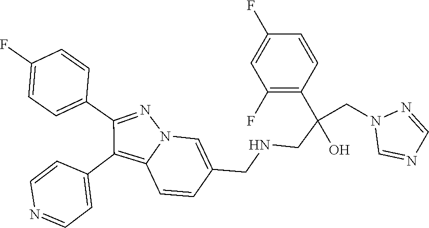 [SMILES: OC(CNCc1ccc2c(-c3ccncc3)c(-c3ccc(F)cc3)nn2c1)(Cn1cncn1)c1ccc(F)cc1F] |
| I-153 | 2-(2,4-difluorophenyl)- 1-((2- ((2-(4-fluorophenyl)-3-(pyridin- 4-yl)pyrazolo[1,5-a]pyridin-7- yl)oxy)ethyl)amino)-3-(1H- 1,2,4-triazol-1-yl)propan-2-ol | 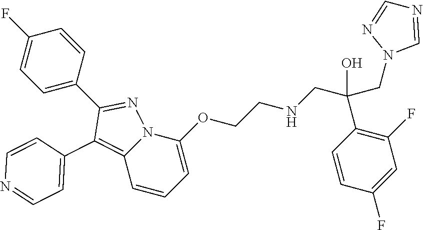 [SMILES: OC(CNCCOc1cccc2c(-c3ccncc3)c(-c3ccc(F)cc3)nn12)(Cn1cncn1)c1ccc(F)cc1F] |
| I-154 | 2-(2,4-difluorophenyl)- 1-(4-((2- (4-fluorophenyl)-3-(pyridin-4- yl)pyrazolo[1,5-a]pyridin-7- yl)oxy)piperidin-1-yl)-3-(1H- 1,2,4-triazol-1-yl)propan-2-ol | 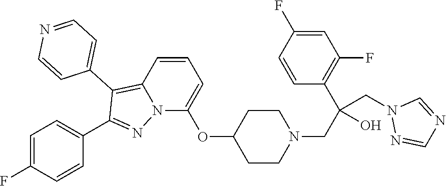 [SMILES: OC(CN1CCC(Oc2cccc3c(-c4ccncc4)c(-c4ccc(F)cc4)nn23)CC1)(Cn1cncn1)c1ccc(F)cc1F] |
| I-155 | N-(1-(2-(2,4-difluorophenyl)-2- hydroxy-3-(1H-1,2,4-triazol-1- yl)propyl)piperidin-4-yl)-2-(4- fluorophenyl)-3-(pyridin-4- yl)pyrazolo[1,5-a]pyridine-7- carboxamide | 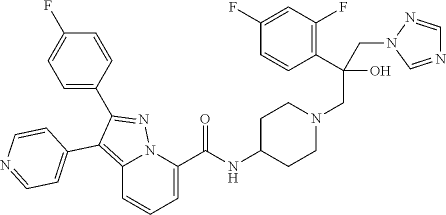 [SMILES: O=C(NC1CCN(CC(O)(Cn2cncn2)c2ccc(F)cc2F)CC1)c1cccc2c(-c3ccncc3)c(-c3ccc(F)cc3)nn12] |
| I-156 | N-(3-(6-chloro-1,2,3,4- tetrahydro-9H-pyrido[3,4- b]indol-9-yl)propyl)-2-(4- fluorophenyl)-3-(pyridin-4- yl)pyrazolo[1,5-a]pyridine-7- carboxamide | 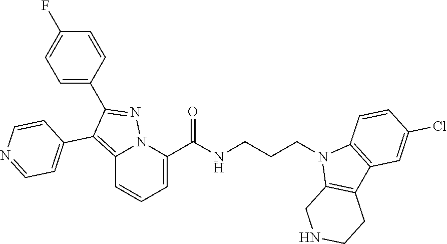 [SMILES: O=C(NCCCn1c2c(c3cc(Cl)ccc31)CCNC2)c1cccc2c(-c3ccncc3)c(-c3ccc(F)cc3)nn12] |
| I-157 | 6-chloro-9-(3-((2-(4- fluorophenyl)-3-(pyridin-4- yl)pyrazolo[1,5-a]pyridin-7- yl)oxy)propyl)-2,3,4,9- tetrahydro-1H-pyrido[3,4- b]indole | 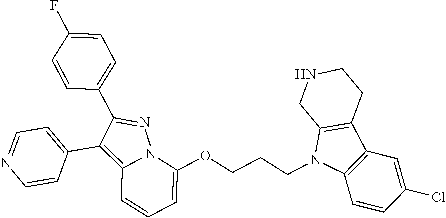 [SMILES: Fc1ccc(-c2nn3c(OCCCn4c5c(c6cc(Cl)ccc64)CCNC5)cccc3c2-c2ccncc2)cc1] |
| I-158 | N-((1-(2-((4- carbamimidoylphenyl)amino)et hyl)piperidin-4-yl)methyl)-2-(4- fluorophenyl)-3-(pyridin-4- yl)pyrazolo[1,5-a]pyridine-7- carboxamide | 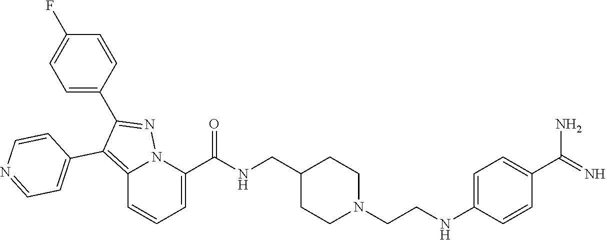 [SMILES: N=C(N)c1ccc(NCCN2CCC(CNC(=O)c3cccc4c(-c5ccncc5)c(-c5ccc(F)cc5)nn34)CC2)cc1] |
| I-159 | N-(4-carbamimidoylphenyl)-2- (4-fluorophenyl)-3-(pyridin-4- yl)pyrazolo[1,5-a]pyridine-7- carboxamide | 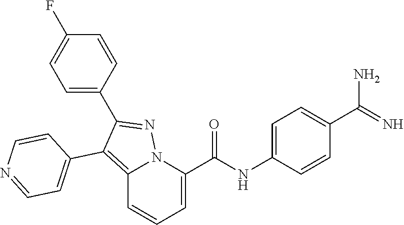 [SMILES: N=C(N)c1ccc(NC(=O)c2cccc3c(-c4ccncc4)c(-c4ccc(F)cc4)nn23)cc1] |
| I-160 | N-((1-(4-(1-acetyl-9H- pyrido[3,4-b]indol-9- yl)butyl)piperidin-4-yl)methyl)- 2-(4-fluorophenyl)-3-(pyridin-4- yl)pyrazolo[1,5-a]pyridine-7- carboxamide | 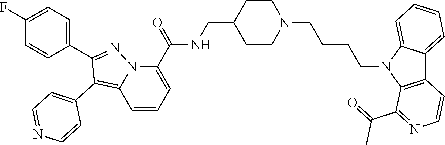 [SMILES: CC(=O)c1nccc2c3ccccc3n(CCCCN3CCC(CNC(=O)c4cccc5c(-c6ccncc6)c(-c6ccc(F)cc6)nn45)CC3)c12] |
| I-161 | N-((1-(9H-pyrido[3,4-b]indole- 1-carbonyl)piperidin-4- yl)methyl)-2-(4-fluorophenyl)- 3-(pyridin-4-yl)pyrazolo[1,5- a]pyridine-7-carboxamide | 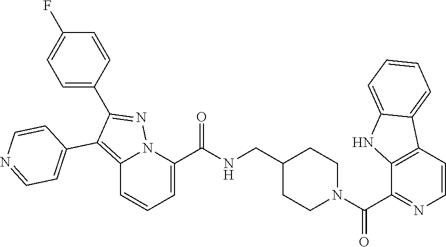 [SMILES: O=C(NCC1CCN(C(=O)c2nccc3c2[nH]c2ccccc23)CC1)c1cccc2c(-c3ccncc3)c(-c3ccc(F)cc3)nn12] |
| I-162 | N-(1-(9H-pyrido[3,4-b]indole-1- carbonyl)piperidin-4-yl)-2-(4- fluorophenyl)-3-(pyridin-4- yl)pyrazolo[1,5-a]pyridine-7- carboxamide | 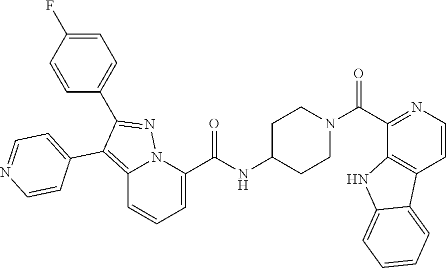 [SMILES: O=C(NC1CCN(C(=O)c2nccc3c2[nH]c2ccccc23)CC1)c1cccc2c(-c3ccncc3)c(-c3ccc(F)cc3)nn12] |
| I-163 | N-(3-(2-(4-fluorophenyl)-3- (pyridin-4-yl)pyrazolo[1,5- a]pyridine-7- carboxamido)propyl)-9H- pyrido[3,4-b]indole-1- carboxamide | 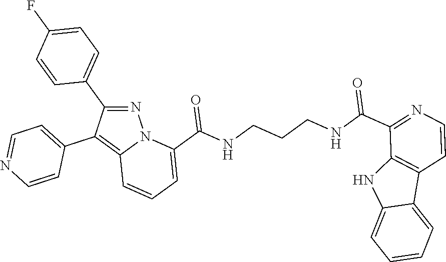 [SMILES: O=C(NCCCNC(=O)c1cccc2c(-c3ccncc3)c(-c3ccc(F)cc3)nn12)c1nccc2c1[nH]c1ccccc12] |
| I-164 | 3-(2-(4-fluorophenyl)-3- (pyridin-4-yl)pyrazolo[1,5- a]pyridine-7- carboxamido)propyl 9H- pyrido[3,4-b]indole-1- carboxylate | 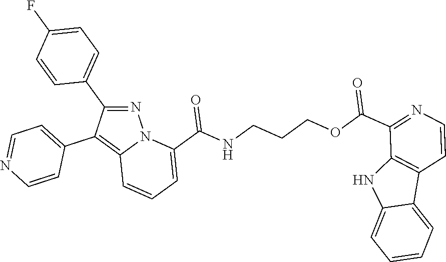 [SMILES: O=C(OCCCNC(=O)c1cccc2c(-c3ccncc3)c(-c3ccc(F)cc3)nn12)c1nccc2c1[nH]c1ccccc12] |
| I-165 | 3-(2-(4-fluorophenyl)-3- (pyridin-4-yl)pyrazolo[1,5- a]pyridine-6- carboxamido)propyl 9H- pyrido[3,4-b]indole-1- carboxylate | 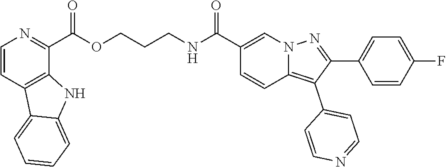 [SMILES: O=C(NCCCOC(=O)c1nccc2c1[nH]c1ccccc12)c1ccc2c(-c3ccncc3)c(-c3ccc(F)cc3)nn2c1] |
| I-166 | N-(1-(9H-pyrido[3,4-b]indole-1- carbonyl)piperidin-4-yl)-2-(4- fluorophenyl)-3-(pyridin-4- yl)pyrazolo[1,5-a]pyridine-6- carboxamide | 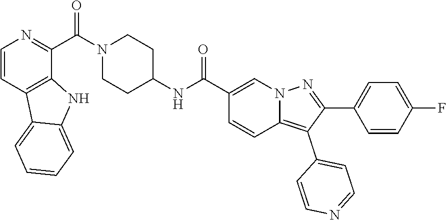 [SMILES: O=C(NC1CCN(C(=O)c2nccc3c2[nH]c2ccccc23)CC1)c1ccc2c(-c3ccncc3)c(-c3ccc(F)cc3)nn2c1] |
| I-167 | N-(3-(2-(4-fluorophenyl)-3- (pyridin-4-yl)pyrazolo[1,5- a]pyridine-6- carboxamido)propyl)-9H- pyrido[3,4-b]indole-1- carboxamide | 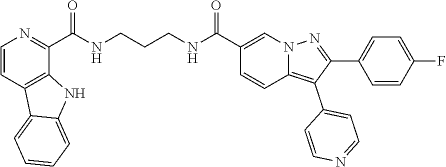 [SMILES: O=C(NCCCNC(=O)c1nccc2c1[nH]c1ccccc12)c1ccc2c(-c3ccncc3)c(-c3ccc(F)cc3)nn2c1] |
| I-168 | N-(1-((9H-pyrido[3,4-b]indol-1- yl)methyl)piperidin-4-yl)-2-(4- fluorophenyl)-3-(pyridin-4- yl)pyrazolo[1,5-a]pyridine-7- carboxamide | 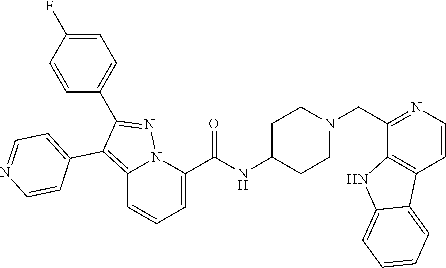 [SMILES: O=C(NC1CCN(Cc2nccc3c2[nH]c2ccccc23)CC1)c1cccc2c(-c3ccncc3)c(-c3ccc(F)cc3)nn12] |
| I-169 | N-(3-(((9H-pyrido[3,4-b]indol- 1-yl)methyl)amino)propyl)-2- (4-fluorophenyl)-3-(pyridin-4- yl)pyrazolo[1,5-a]pyridine-7- carboxamide | 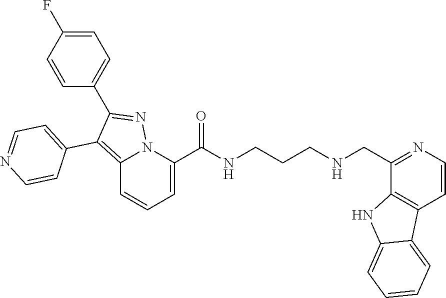 [SMILES: O=C(NCCCNCc1nccc2c1[nH]c1ccccc12)c1cccc2c(-c3ccncc3)c(-c3ccc(F)cc3)nn12] |
| I-170 | N-(1-(9H-pyrido[3,4-b]indole-1- carbonyl)piperidin-4-yl)-2-(4- fluorophenyl)-3-(pyridin-4- yl)pyrazolo[1,5-a]pyridine-6- carboximidamide | 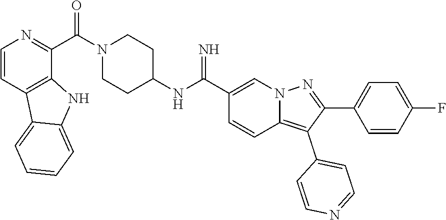 [SMILES: N=C(NC1CCN(C(=O)c2nccc3c2[nH]c2ccccc23)CC1)c1ccc2c(-c3ccncc3)c(-c3ccc(F)cc3)nn2c1] |
| I-171 | N-(3-(2-(4-fluorophenyl)-3- (pyridin-4-yl)pyrazolo[1,5- a]pyridine-6- carboximidamido)propyl)-9H- pyrido[3,4-b]indole-1- carboxamide | 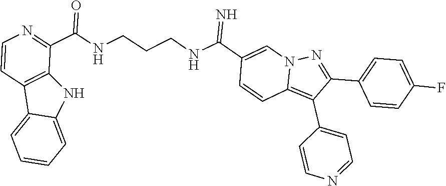 [SMILES: N=C(NCCCNC(=O)c1nccc2c1[nH]c1ccccc12)c1ccc2c(-c3ccncc3)c(-c3ccc(F)cc3)nn2c1] |
| I-172 | N-(3-(1-acetyl-9H-pyrido[3,4- b]indol-9-yl)propyl)-2-(4- fluorophenyl)-3-(pyridin-4- yl)pyrazolo[1,5-a]pyridine-7- carboxamide | 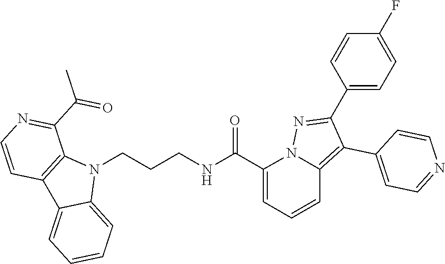 [SMILES: CC(=O)c1nccc2c3ccccc3n(CCCNC(=O)c3cccc4c(-c5ccncc5)c(-c5ccc(F)cc5)nn34)c12] |
| I-173 | N-(1-(1-acetyl-9H-pyrido[3,4- b]indole-9-carbonyl)piperidin- 4-yl)-2-(4-fluorophenyl)-3- (pyridin-4-yl)pyrazolo[1,5- a]pyridine-7-carboxamide | 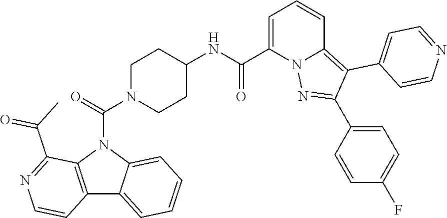 [SMILES: CC(=O)c1nccc2c3ccccc3n(C(=O)N3CCC(NC(=O)c4cccc5c(-c6ccncc6)c(-c6ccc(F)cc6)nn45)CC3)c12] |
| I-174 | 9-(3-(2-(4-fluorophenyl)-3- (pyridin-4-yl)pyrazolo[1,5- a]pyridine-7- carboxamido)propyl)-9H- pyrido[3,4-b]indole-1- carboxylic acid | 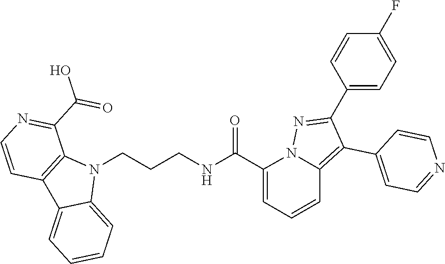 [SMILES: O=C(O)c1nccc2c3ccccc3n(CCCNC(=O)c3cccc4c(-c5ccncc5)c(-c5ccc(F)cc5)nn34)c12] |
| I-175 | 9-(4-(2-(4-fluorophenyl)-3- (pyridin-4-yl)pyrazolo[1,5- a]pyridine-7- carboxamido)piperidine-1- carbonyl)-9H-pyrido[3,4- b]indole-1-carboxylic acid | 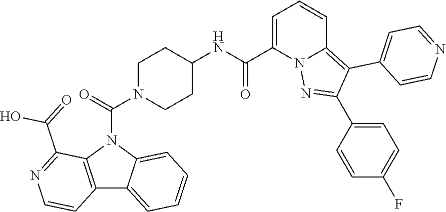 [SMILES: O=C(O)c1nccc2c3ccccc3n(C(=O)N3CCC(NC(=O)c4cccc5c(-c6ccncc6)c(-c6ccc(F)cc6)nn45)CC3)c12] |
| I-176 | 9-(3-(2-(4-fluorophenyl)-3- (pyridin-4-yl)pyrazolo[1,5- a]pyridine-7- carboxamido)propyl)-9H- pyrido[3,4-b]indole-1- carboxamide | 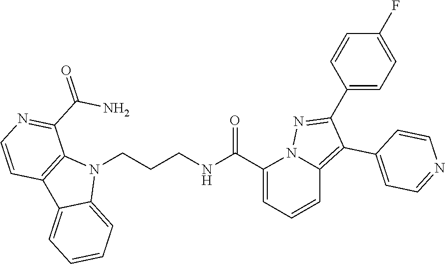 [SMILES: NC(=O)c1nccc2c3ccccc3n(CCCNC(=O)c3cccc4c(-c5ccncc5)c(-c5ccc(F)cc5)nn34)c12] |
| I-177 | 2-(2-(4-fluorophenyl)-3- (pyridin-4-yl)pyrazolo[1,5- a]pyridine-7-carboxamido) ethyl 1-carbamoyl-9H-pyrido[3,4- b]indole-8-carboxylate | 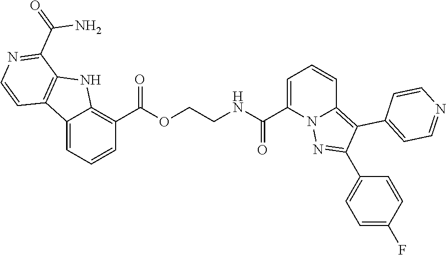 [SMILES: NC(=O)c1nccc2c1[nH]c1c(C(=O)OCCNC(=O)c3cccc4c(-c5ccncc5)c(-c5ccc(F)cc5)nn34)cccc12] |
| I-178 | 8-((2-(2-(4-fluorophenyl)-3- (pyridin-4-yl)pyrazolo[1,5- a]pyridine-7- carboxamido)ethoxy)carbonyl) -9H-pyrido[3,4-b]indole-1- carboxylic acid | 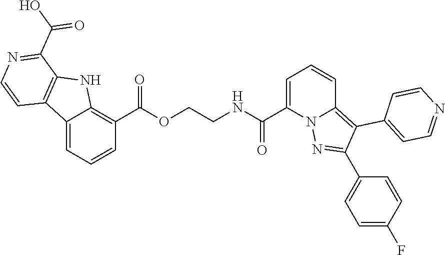 [SMILES: O=C(OCCNC(=O)c1cccc2c(-c3ccncc3)c(-c3ccc(F)cc3)nn12)c1cccc2c1[nH]c1c(C(=O)O)nccc12] |
| I-179 | 8-((2-(2-(4-fluorophenyl)-3- (pyridin-4-yl)pyrazolo[1,5- a]pyridine-7- carboxamido)ethyl)carbamoyl)- 9H-pyrido[3,4-b]indole-1- carboxylic acid | 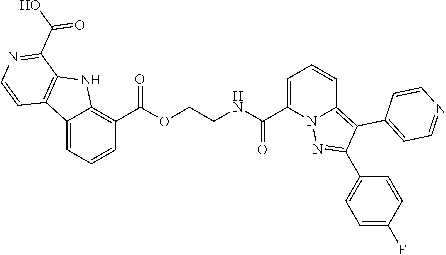 [SMILES: O=C(OCCNC(=O)c1cccc2c(-c3ccncc3)c(-c3ccc(F)cc3)nn12)c1cccc2c1[nH]c1c(C(=O)O)nccc12] |
| I-180 | N⁸-(2-(2-(4-fluorophenyl)-3- (pyridin-4-yl)pyrazolo[1,5- a]pyridine-7- carboxamido)ethyl)-9H- pyrido[3,4-b]indole-1,8- dicarboxamide | 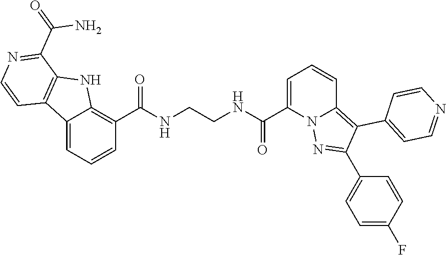 [SMILES: NC(=O)c1nccc2c1[nH]c1c(C(=O)NCCNC(=O)c3cccc4c(-c5ccncc5)c(-c5ccc(F)cc5)nn34)cccc12] |
| I-181 | N⁸-(2-(2-(4-fluorophenyl)-3- (pyridin-4-yl)pyrazolo[1,5- a]pyridine-7- carboxamido)ethyl)-9H- pyrido[3,4-b]indole-1,9- dicarboxamide | 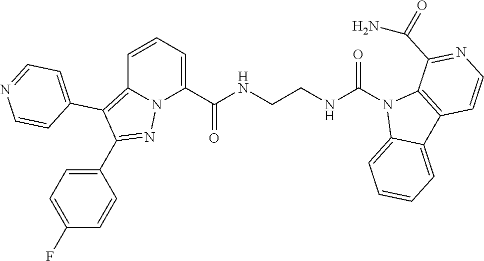 [SMILES: NC(=O)c1nccc2c3ccccc3n(C(=O)NCCNC(=O)c3cccc4c(-c5ccncc5)c(-c5ccc(F)cc5)nn34)c12] |
| I-182 | 9-((2-(2-(4-fluorophenyl)-3- (pyridin-4-yl)pyrazolo[1,5- a]pyridine-7- carboxamido)ethyl)carbamoyl)- 9H-pyrido[3,4-b]indole-1- carboxylic acid | 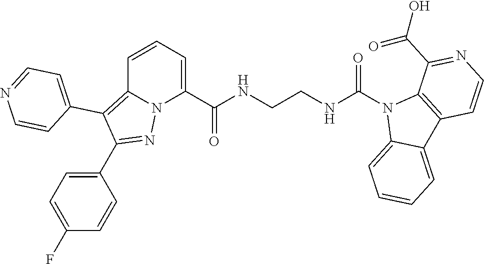 [SMILES: O=C(O)c1nccc2c3ccccc3n(C(=O)NCCNC(=O)c3cccc4c(-c5ccncc5)c(-c5ccc(F)cc5)nn34)c12] |
| I-183 | 1-acetyl-N-(2-(2-(4- fluorophenyl)-3-(pyridin-4- yl)pyrazolo[1,5-a]pyridine-7- carboxamido)ethyl)-9H- pyrido[3,4-b]indole-9- carboxamide | 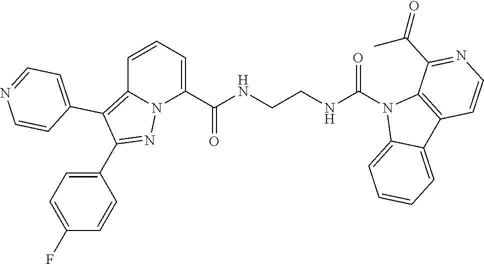 [SMILES: CC(=O)c1nccc2c3ccccc3n(C(=O)NCCNC(=O)c3cccc4c(-c5ccncc5)c(-c5ccc(F)cc5)nn34)c12] |
| I-184 | N-((1r,4r)-4-(2-(4- fluorophenyl)-3-(pyridin-4- yl)pyrazolo[1,5-a]pyridine-7- carboxamido)cyclohexyl)-9H- pyrido[3,4-b]indole-1- carboxamide | 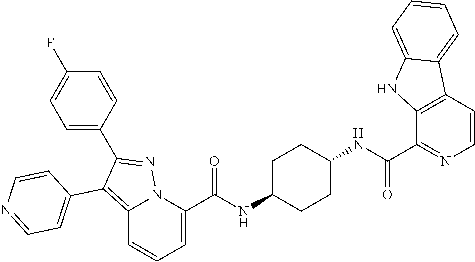 [SMILES: O=C(N[C@H]1CC[C@H](NC(=O)c2cccc3c(-c4ccncc4)c(-c4ccc(F)cc4)nn23)CC1)c1nccc2c1[nH]c1ccccc12] |
| I-185 | 9-((1r,4r)-4-(2-(4-fluorophenyl)- 3-(pyridin-4-yl)pyrazolo[1,5- a]pyridine-7- carboxamido)cyclohexyl)-9H- pyrido[3,4-b]indole-1- carboxamide | 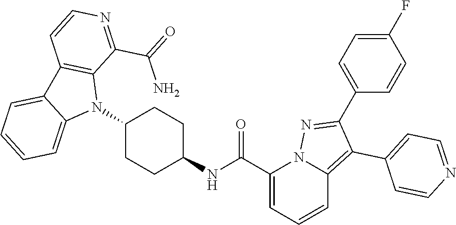 [SMILES: NC(=O)c1nccc2c3ccccc3n([C@H]3CC[C@H](NC(=O)c4cccc5c(-c6ccncc6)c(-c6ccc(F)cc6)nn45)CC3)c12] |
| I-186 | 9-((1r,4r)-4-(2-(4-fluorophenyl)- 3-(pyridin-4-yl)pyrazolo[1,5- a]pyridine-7- carboxamido)cyclohexyl)-9H- pyrido[3,4-b]indole-1- carboxylic acid | 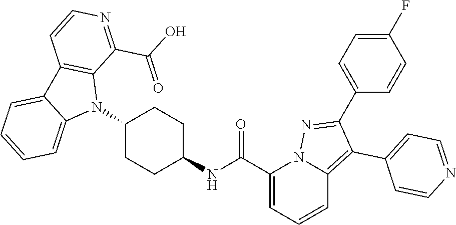 [SMILES: O=C(O)c1nccc2c3ccccc3n([C@H]3CC[C@H](NC(=O)c4cccc5c(-c6ccncc6)c(-c6ccc(F)cc6)nn45)CC3)c12] |
| I-187 | N-((1r,4r)-4-(1-acetyl-9H- pyrido[3,4-b]indol-9- yl)cyclohexyl)-2-(4- fluorophenyl)-3-(pyridin-4- yl)pyrazolo[1,5-a]pyridine-7- carboxamide | 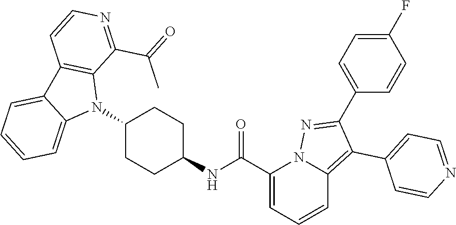 [SMILES: CC(=O)c1nccc2c3ccccc3n([C@H]3CC[C@H](NC(=O)c4cccc5c(-c6ccncc6)c(-c6ccc(F)cc6)nn45)CC3)c12] |
| I-188 | 6-((1-(2-(2,4-difluorophenyl)-2- hydroxy-3-(1H-imidazol-1- yl)propyl)piperidin-4- yl)methyl)-4-(3-(4- fluorophenyl)-1-methyl-1H- pyrazol-4-yl)-6,7-dihydro-5H- pyrrolo[3,4-b]pyridin-5-one | 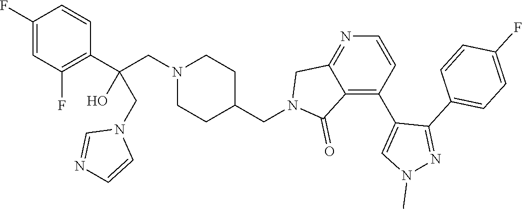 [SMILES: Cn1cc(-c2ccnc3c2C(=O)N(CC2CCN(CC(O)(Cn4ccnc4)c4ccc(F)cc4F)CC2)C3)c(-c2ccc(F)cc2)n1] |
| I-189 | 3-((4-(3-(4-fluorophenyl)-1- methyl-1H-pyrazol-4-yl)pyridin- 3-yl)methoxy)-1-(9H- pyrido[3,4-b]indol-1-yl)propan- 1-one | 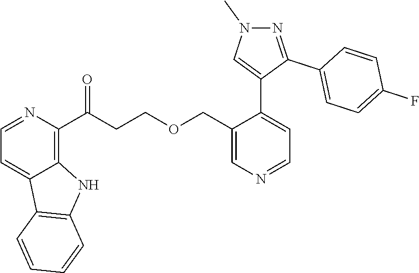 [SMILES: Cn1cc(-c2ccncc2COCCC(=O)c2nccc3c2[nH]c2ccccc23)c(-c2ccc(F)cc2)n1] |
| I-190 | 3-(((4-(3-(4-fluorophenyl)-1- methyl-1H-pyrazol-4-yl)pyridin- 3-yl)methyl)amino)-1-(9H- pyrido[3,4-b]indol-1-yl)propan- 1-one | 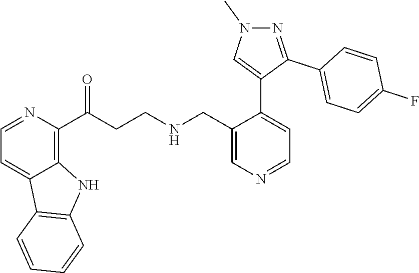 [SMILES: Cn1cc(-c2ccncc2CNCCC(=O)c2nccc3c2[nH]c2ccccc23)c(-c2ccc(F)cc2)n1] |
| I-191 | 4-(3-(4-fluorophenyl)-1-methyl- 1H-pyrazol-4-yl)-6-(2-(1- ((2E,6E)-3,7,11- trimethyldodeca-2,6, 10-trien-1- yl)piperidin-4-yl)ethyl)-6,7- dihydro-5H-pyrrolo[3,4- b]pyridin-5-one | 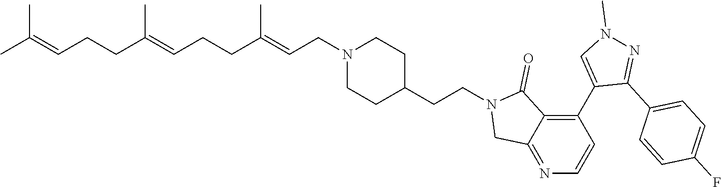 [SMILES: CC(C)=CCC/C(C)=C/CC/C(C)=C/CN1CCC(CCN2Cc3nccc(-c4cn(C)nc4-c4ccc(F)cc4)c3C2=O)CC1] |
| I-192 | 4-(3-(4-fluorophenyl)-1-methyl- 1H-pyrazol-4-yl)-6-((2E,6E)- 3,7,11-trimethyldodeca-2,6,10- trien-1-yl)-6,7-dihydro-5H- pyrrolo[3,4-b]pyridin-5-one | 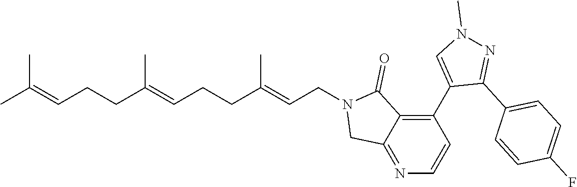 [SMILES: CC(C)=CCC/C(C)=C/CC/C(C)=C/CN1Cc2nccc(-c3cn(C)nc3-c3ccc(F)cc3)c2C1=O] |
| I-193 | (9Z,12Z,15Z)-N-(3-(4-(2-(4-(3- (4-fluorophenyl)-1-methyl-1H- pyrazol-4-yl)-5-oxo-5,7- dihydro-6H-pyrrolo[3,4- b]pyridin-6-yl)ethyl)piperidin-1- yl)propyl)octadeca-9,12,15- trienamide | 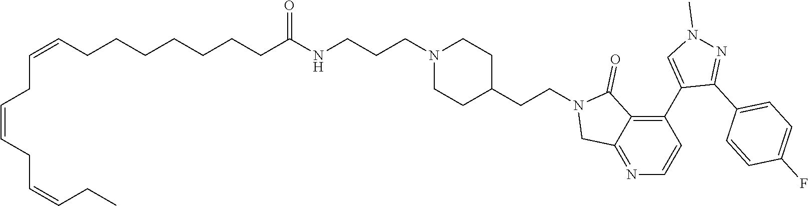 [SMILES: CC/C=C\C/C=C\C/C=C\CCCCCCCC(=O)NCCCN1CCC(CCN2Cc3nccc(-c4cn(C)nc4-c4ccc(F)cc4)c3C2=O)CC1] |
| I-194 | 6-(2-(1-(3-((3-((3- aminopropyl)amino)propyl) amino)propyl)piperidin-4-yl) ethyl)-4-(3-(4-fluorophenyl)- 1-methyl-1H-pyrazol-4-yl)- 6,7-dihydro-5H-pyrrolo [3,4-b]pyridin-5-one | 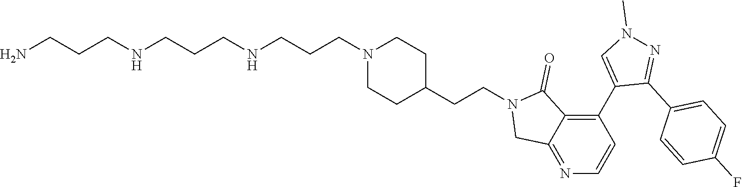 [SMILES: Cn1cc(-c2ccnc3c2C(=O)N(CCC2CCN(CCCNCCCNCCCN)CC2)C3)c(-c2ccc(F)cc2)n1] |
| I-195 | 4-((2-(4-(2-(4-(3-(4- fluorophenyl)-1-methyl-1H- pyrazol-4-yl)-5-oxo-5,7- dihydro-6H-pyrrolo[3,4- b]pyridin-6-yl)ethyl)piperidin-1- yl)ethyl)amino)benzimidamide | 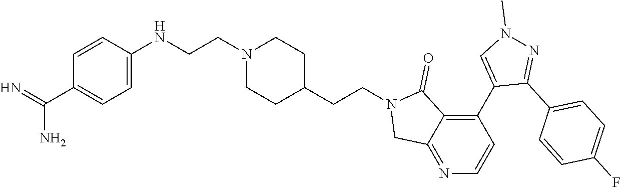 [SMILES: Cn1cc(-c2ccnc3c2C(=O)N(CCC2CCN(CCNc4ccc(C(=N)N)cc4)CC2)C3)c(-c2ccc(F)cc2)n1] |
| I-196 | 4-((4-(4-(3-(4-fluorophenyl)-1- methyl-1H-pyrazol-4-yl)-5-oxo- 5,7-dihydro-6H-pyrrolo[3,4- b]pyridin-6- yl)butyl)amino)benzimidamide | 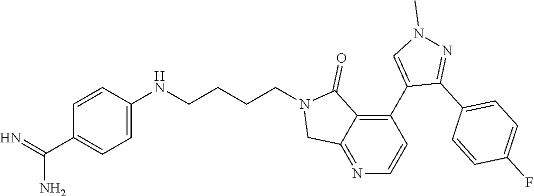 [SMILES: Cn1cc(-c2ccnc3c2C(=O)N(CCCCNc2ccc(C(=N)N)cc2)C3)c(-c2ccc(F)cc2)n1] |
| I-197 | 4-((3-(4-(3-(4-fluorophenyl)-1- methyl-1H-pyrazol-4-yl)-5-oxo- 5,7-dihydro-6H-pyrrolo[3,4- b]pyridin-6- yl)propyl)amino)benzimidamide | 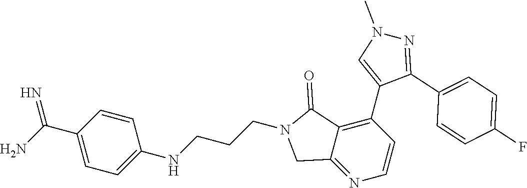 [SMILES: Cn1cc(-c2ccnc3c2C(=O)N(CCCNc2ccc(C(=N)N)cc2)C3)c(-c2ccc(F)cc2)n1] |
| I-198 | 1-(3-((4-(4-(3-(4-fluorophenyl)- 1-methyl-1H-pyrazol-4-yl)-5- oxo-5,7-dihydro-6H- pyrrolo[3,4-b]pyridin-6- yl)butyl)amino)propyl)guanidine | 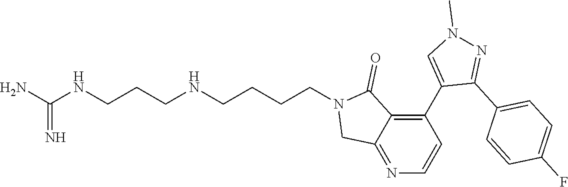 [SMILES: Cn1cc(-c2ccnc3c2C(=O)N(CCCCNCCCNC(=N)N)C3)c(-c2ccc(F)cc2)n1] |
| I-200 | 6-((1-(9H-pyrido[3,4-b]indole- 1-carbonyl)piperidin-4- yl)methyl)-4-(3-(4- fluorophenyl)-1-methyl-1H- pyrazol-4-yl)-6,7-dihydro-5H- pyrrolo[3,4-b]pyridin-5-one | 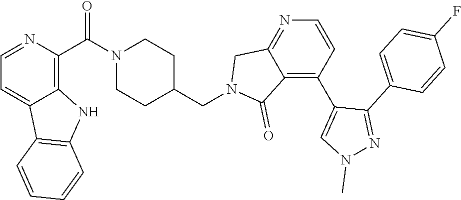 [SMILES: Cn1cc(-c2ccnc3c2C(=O)N(CC2CCN(C(=O)c4nccc5c4[nH]c4ccccc45)CC2)C3)c(-c2ccc(F)cc2)n1] |
| I-201 | 6-((1-(3-(1-acetyl-9H- pyrido[3,4-b]indol-9- yl)propyl)piperidin-4- yl)methyl)-4-(3-(4- fluorophenyl)-1-methyl-1H- pyrazol-4-yl)-6,7-dihydro-5H- pyrrolo[3,4-b]pyridin-5-one | 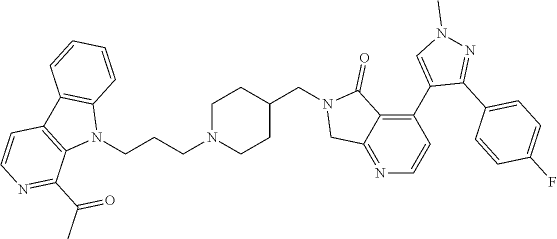 [SMILES: CC(=O)c1nccc2c3ccccc3n(CCCN3CCC(CN4Cc5nccc(-c6cn(C)nc6-c6ccc(F)cc6)c5C4=O)CC3)c12] |
| I-202 | N-(2-(4-((4-(3-(4-fluorophenyl)- 1-methyl-1H-pyrazol-4-yl)-5- oxo-5,7-dihydro-6H- pyrrolo[3,4-b]pyridin-6- yl)methyl)piperidin-1-yl)ethyl)- 9H-pyrido[3,4-b]indole-1- carboxamide | 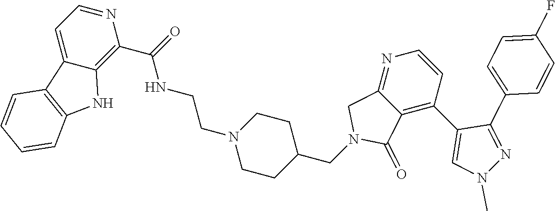 [SMILES: Cn1cc(-c2ccnc3c2C(=O)N(CC2CCN(CCNC(=O)c4nccc5c4[nH]c4ccccc45)CC2)C3)c(-c2ccc(F)cc2)n1] |
| I-203 | 6-((1-(4-(1-acetyl-9H- pyrido[3,4-b]indol-9-yl)-4- oxobutyl)piperidin-4-yl)methyl)- 4-(3-(4-fluorophenyl)-1-methyl- 1H-pyrazol-4-yl)-6,7-dihydro- 5H-pyrrolo[3,4-b]pyridin-5-one | 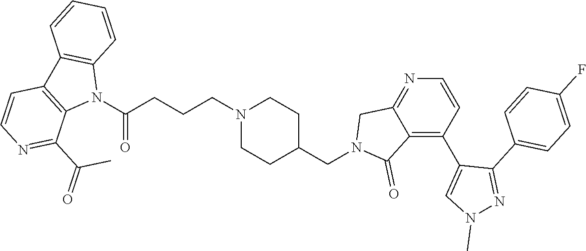 [SMILES: CC(=O)c1nccc2c3ccccc3n(C(=O)CCCN3CCC(CN4Cc5nccc(-c6cn(C)nc6-c6ccc(F)cc6)c5C4=O)CC3)c12] |
| I-204 | N-(2-(4-(4-(3-(4-fluorophenyl)- 1-methyl-1H-pyrazol-4-yl)-5- oxo-5,7-dihydro-6H- pyrrolo[3,4-b]pyridin-6- yl)piperidin-1-yl)ethyl)-9H- pyrido[3,4-b]indole-1- carboxamide | 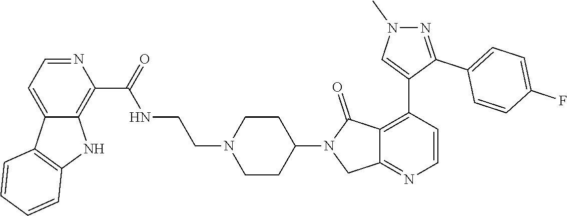 [SMILES: Cn1cc(-c2ccnc3c2C(=O)N(C2CCN(CCNC(=O)c4nccc5c4[nH]c4ccccc45)CC2)C3)c(-c2ccc(F)cc2)n1] |
| I-205 | 6-(1-(4-(1-acetyl-9H- pyrido[3,4-b]indol-9-yl)-4- oxobutyl)piperidin-4-yl)-4-(3- (4-fluorophenyl)-1-methyl-1H- pyrazol-4-yl)-6,7-dihydro-5H- pyrrolo[3,4-b]pyridin-5-one | 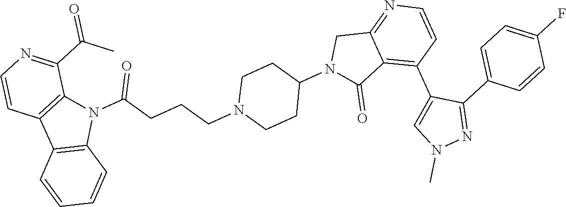 [SMILES: CC(=O)c1nccc2c3ccccc3n(C(=O)CCCN3CCC(N4Cc5nccc(-c6cn(C)nc6-c6ccc(F)cc6)c5C4=O)CC3)c12] |
| I-206 | 6-(1-(4-(1-acetyl-9H- pyrido[3,4-b]indol-9- yl)butyl)piperidin-4-yl)-4-(3-(4- fluorophenyl)-1-methyl-1H- pyrazol-4-yl)-6,7-dihydro-5H- pyrrolo[3,4-b]pyridin-5-one | 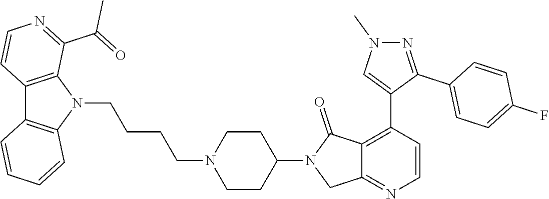 [SMILES: CC(=O)c1nccc2c3ccccc3n(CCCCN3CCC(N4Cc5nccc(-c6cn(C)nc6-c6ccc(F)cc6)c5C4=O)CC3)c12] |
| I-207 | 6-(1-(3-(1-acetyl-9H- pyrido[3,4-b]indol-9- yl)propyl)piperidin-4-yl)-4-(3- (4-fluorophenyl)-1-methyl-1H- pyrazol-4-yl)-6,7-dihydro-5H- pyrrolo[3,4-b]pyridin-5-one | 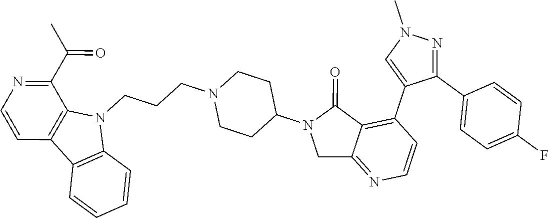 [SMILES: CC(=O)c1nccc2c3ccccc3n(CCCN3CCC(N4Cc5nccc(-c6cn(C)nc6-c6ccc(F)cc6)c5C4=O)CC3)c12] |
| I-208 | 1-6-((1-(3-(9H-pyrido[3,4- b]indol-1-yl)acryloyl)piperidin- 4-yl)methyl)-4-(3-(4- fluorophenyl)-1-methyl-1H- pyrazol-4-yl)-6,7-dihydro-5H- pyrrolo[3,4-b]pyridin-5-one | 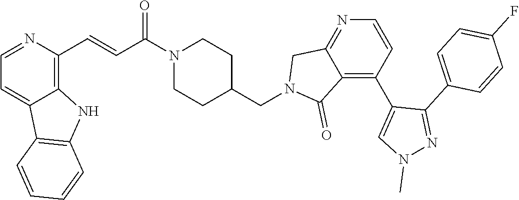 [SMILES: Cn1cc(-c2ccnc3c2C(=O)N(CC2CCN(C(=O)/C=C/c4nccc5c4[nH]c4ccccc45)CC2)C3)c(-c2ccc(F)cc2)n1] |
| I-209 | (E)-6-((1-(3-(9H-pyrido[3,4- b]indol-1-yl)but-2- enoyl)piperidin-4-yl)methyl)-4- (3-(4-fluorophenyl)-1-methyl- 1H-pyrazol-4-yl)-6,7-dihydro- 5H-pyrrolo[3,4-b]pyridin-5-one | 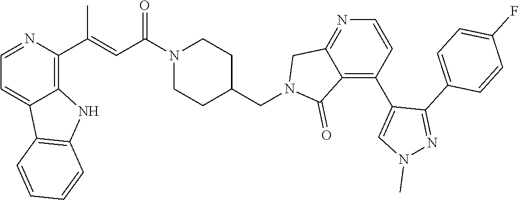 [SMILES: C/C(=C\C(=O)N1CCC(CN2Cc3nccc(-c4cn(C)nc4-c4ccc(F)cc4)c3C2=O)CC1)c1nccc2c1[nH]c1ccccc12] |
| I-210 | (S)-5-(4-(5-(2-aminopyrimidin- 4-yl)-4-(4-fluorophenyl)-1H- imidazol-1-yl)piperidin-1-yl)-2- (2,4-difluorophenyl)-1-(1H- 1,2,4-triazol-1-yl)pent-3-yn-2-ol | 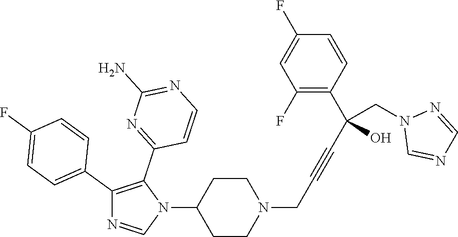 [SMILES: Nc1nccc(-c2c(-c3ccc(F)cc3)ncn2C2CCN(CC#C[C@@](O)(Cn3cncn3)c3ccc(F)cc3F)CC2)n1] |
| I-211 | (R)-5-(4-(5-(2-aminopyrimidin- 4-yl)-4-(4-fluorophenyl)-1H- imidazol-1-yl)piperidin-1-yl)-2- (2,4-difluorophenyl)-1-(1H- 1,2,4-triazol-1-yl)pent-3-yn-2-ol | 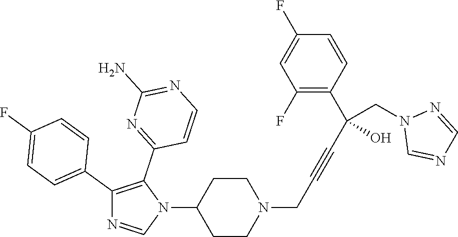 [SMILES: Nc1nccc(-c2c(-c3ccc(F)cc3)ncn2C2CCN(CC#C[C@](O)(Cn3cncn3)c3ccc(F)cc3F)CC2)n1] |
| I-212 | 4-(1-(1-(3-(8-chloro-2-methyl- 1,2,3,4-tetrahydro-9H- pyrido[3,4-b]indol-9- yl)propyl)piperidin-4-yl)-4-(4- fluorophenyl)-1H-imidazol-5- yl)pyrimidin-2-amine | 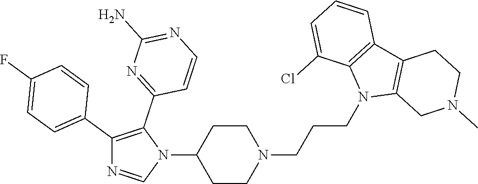 [SMILES: CN1CCc2c(n(CCCN3CCC(n4cnc(-c5ccc(F)cc5)c4-c4ccnc(N)n4)CC3)c3c(Cl)cccc23)C1] |
| I-213 | 4-(1-(1-(2-((2-(8-chloro-2- methyl-1,2,3,4-tetrahydro-9H- pyrido[3,4-b]indol-9- yl)ethyl)amino)ethyl)piperidin- 4-yl)-4-(4-fluorophenyl)-1H- imidazol-5-yl)pyrimidin-2- amine | 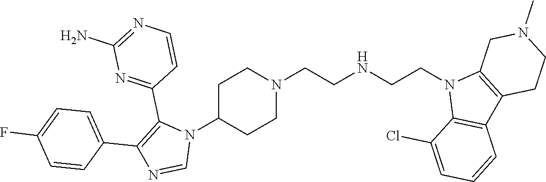 [SMILES: CN1CCc2c(n(CCNCCN3CCC(n4cnc(-c5ccc(F)cc5)c4-c4ccnc(N)n4)CC3)c3c(Cl)cccc23)C1] |
| I-214 | 4-(1-(1-(2-((2-(8-chloro- 1,2,3,4-tetrahydro-9H- pyrido[3,4-b]indol-9- yl)ethyl)amino)ethyl)piperidin- 4-yl)-4-(4-fluorophenyl)-1H- imidazol-5-yl)pyrimidin-2- amine | 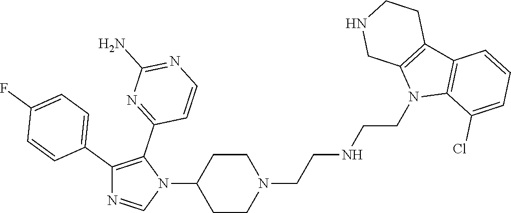 [SMILES: Nc1nccc(-c2c(-c3ccc(F)cc3)ncn2C2CCN(CCNCCn3c4c(c5cccc(Cl)c53)CCNC4)CC2)n1] |
| I-215 | 4-(4-(4-fluorophenyl)-1-(1- ((2E,6E)-3,7,11- trimethyldodeca-2,6, 10-trien-1- yl)piperidin-4-yl)-1H-imidazol- 5-yl)pyrimidin-2-amine | 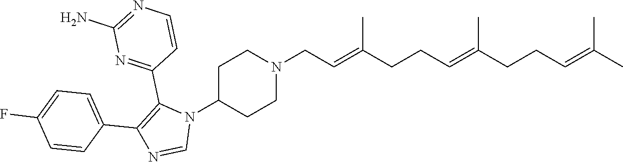 [SMILES: CC(C)=CCC/C(C)=C/CC/C(C)=C/CN1CCC(n2cnc(-c3ccc(F)cc3)c2-c2ccnc(N)n2)CC1] |
| I-216 | (9Z, 12Z, 15Z)-N-(3-(4-(5-(2- aminopyrimidin-4-yl)-4-(4- fluorophenyl)-1H-imidazol-1- yl)piperidin-1- yl)propyl)octadeca-9,12,15- trienamide | 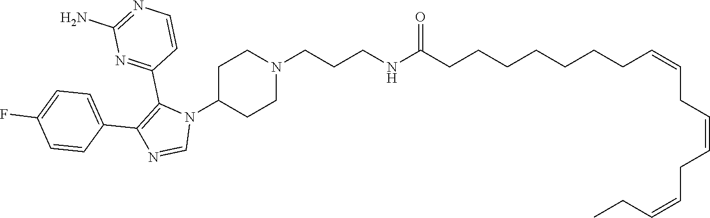 [SMILES: CC/C=C\C/C=C\C/C=C\CCCCCCCC(=O)NCCCN1CCC(n2cnc(-c3ccc(F)cc3)c2-c2ccnc(N)n2)CC1] |
| I-217 | (9Z,12E,15Z)-1-(4-(5-(2- aminopyrimidin-4-yl)-4-(4- fluorophenyl)-1H-imidazol-1- yl)piperidin-1-yl)-13- methyloctadeca-9, 12, 15-trien- 1-one | 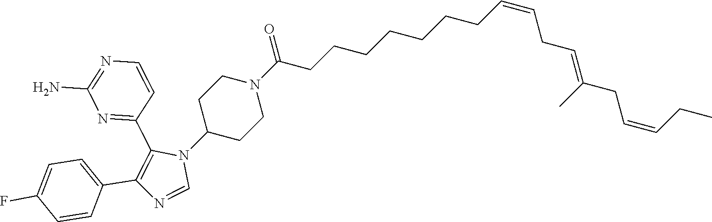 [SMILES: CC/C=C\C/C(C)=C/C/C=C\CCCCCCCC(=O)N1CCC(n2cnc(-c3ccc(F)cc3)c2-c2ccnc(N)n2)CC1] |
| I-218 | (9Z,12E,15Z)-1-(4-(5-(2- aminopyrimidin-4-yl)-4-(4- fluorophenyl)-1H-imidazol-1- yl)piperidin-1-yl)-13- methyloctadeca-9, 12, 15-trien- 1-one | 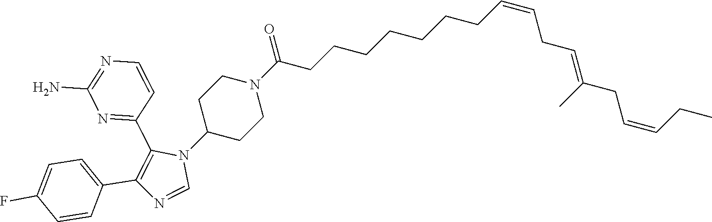 [SMILES: CC/C=C\C/C(C)=C/C/C=C\CCCCCCCC(=O)N1CCC(n2cnc(-c3ccc(F)cc3)c2-c2ccnc(N)n2)CC1] |
| I-219 | N1-(3-aminopropyl)-N⁸-(3-(4- (5-(2-aminopyrimidin-4-yl)-4- (4-fluorophenyl)-1H-imidazol- 1-yl)piperidin-1- yl)propyl)propane-1,3-diamine | 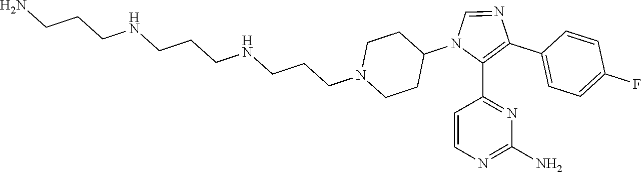 [SMILES: NCCCNCCCNCCCN1CCC(n2cnc(-c3ccc(F)cc3)c2-c2ccnc(N)n2)CC1] |
| I-220 | 4-(4-(4-fluorophenyl)- 1-(1-(3- (triphenyl-λ⁵- phosphaneyl)propyl)piperidin- 4-yl)-1H-imidazol-5- yl)pyrimidin-2-amine | 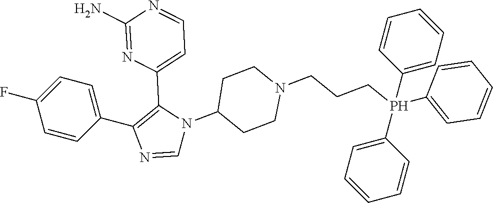 |
| I-221 | 4-((2-(4-(5-(2-aminopyrimidin- 4-yl)-4-(4-fluorophenyl)-1H- imidazol-1-yl)piperidin-1- yl)ethyl)amino)benzimidamide | 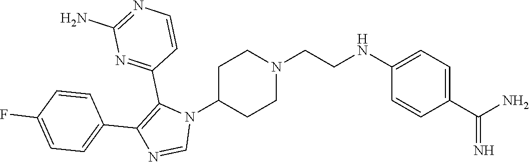 [SMILES: N=C(N)c1ccc(NCCN2CCC(n3cnc(-c4ccc(F)cc4)c3-c3ccnc(N)n3)CC2)cc1] |
| I-222 | 4-(4-(4-fluorophenyl)- 1-(1-(3- (1,2,3,4-tetrahydro-9H- pyrido[3,4-b]indol-9- yl)propyl)piperidin-4-yl)-1H- imidazol-5-yl)pyrimidin-2- amine | 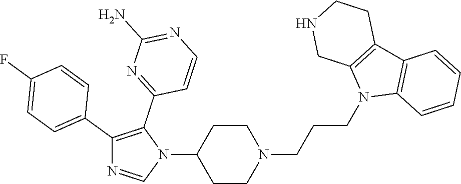 [SMILES: Nc1nccc(-c2c(-c3ccc(F)cc3)ncn2C2CCN(CCCn3c4c(c5ccccc53)CCNC4)CC2)n1] |
| I-223 | 4-(4-(4-fluorophenyl)-1-(1-(3- (5,6,7,8-tetrahydro-9H- pyrrolo[2,3-b:5,′′-c′]dipyridin-9- yl)propyl)piperidin-4-yl)-1H- imidazol-5-yl)pyrimidin-2- amine | 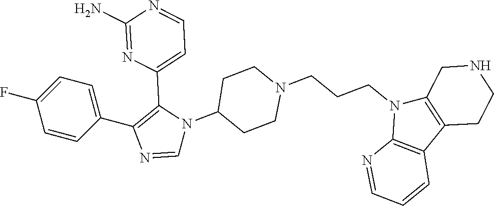 [SMILES: Nc1nccc(-c2c(-c3ccc(F)cc3)ncn2C2CCN(CCCn3c4c(c5cccnc53)CCNC4)CC2)n1] |
| I-224 | 1-(4-(5-(2-aminopyrimidin-4- yl)-4-(4-fluorophenyl)-1H- imidazol-1-yl)-[′,4′-bipiperidin′- 1′-yl)-2-(2,4-difluorophenyl)-3- (1H-1,2,4-triazol-1-yl)propan- 2-ol | 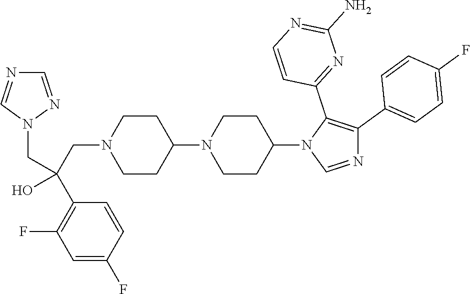 [SMILES: Nc1nccc(-c2c(-c3ccc(F)cc3)ncn2C2CCN(C3CCN(CC(O)(Cn4cncn4)c4ccc(F)cc4F)CC3)CC2)n1] |
| I-225 | 1-(4-((4-(5-(2-aminopyrimidin- 4-yl)-4-(4-fluorophenyl)-1H- imidazol-1-yl)piperidin-1- yl)methyl)piperidin-1-yl)-2-(2,4- difluorophenyl)-3-(1H-1,2,4- triazol-1-yl)propan-2-ol | 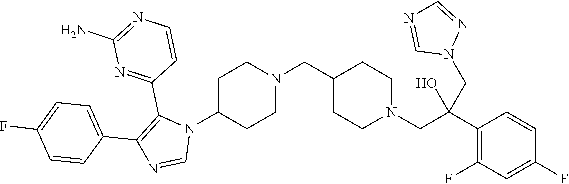 [SMILES: Nc1nccc(-c2c(-c3ccc(F)cc3)ncn2C2CCN(CC3CCN(CC(O)(Cn4cncn4)c4ccc(F)cc4F)CC3)CC2)n1] |
| I-226 | (4-(5-(2-aminopyrimidin-4-yl)- 4-(4-fluorophenyl)-1H- imidazol-1-yl)piperidin-1-yl)(1- (2-(2,4-difluorophenyl)-2- hydroxy-3-(1H-1,2,4-triazol-1- yl)propyl)piperidin-4- yl)methanone | 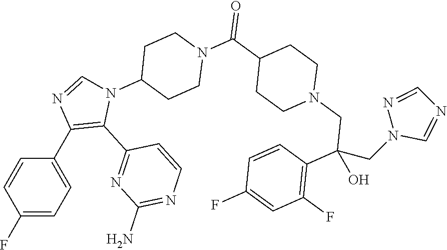 [SMILES: Nc1nccc(-c2c(-c3ccc(F)cc3)ncn2C2CCN(C(=O)C3CCN(CC(O)(Cn4cncn4)c4ccc(F)cc4F)CC3)CC2)n1] |
| I-227 | 4-(4-(5-(2-aminopyrimidin-4- yl)-4-(4-fluorophenyl)-1H- imidazol-1-yl)piperidin-1-yl)-1- (9H-pyrido[3,4-b]indol-1- yl)butan-1-one | 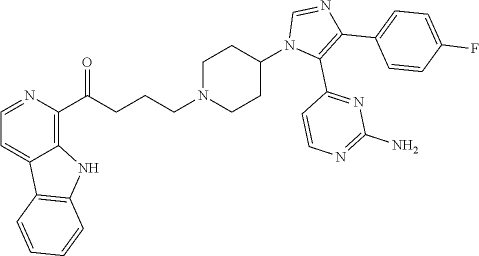 [SMILES: Nc1nccc(-c2c(-c3ccc(F)cc3)ncn2C2CCN(CCCC(=O)c3nccc4c3[nH]c3ccccc34)CC2)n1] |
| I-228 | 5-(4-(5-(2-aminopyrimidin-4- yl)-4-(4-fluorophenyl)-1H- imidazol-1-yl)piperidin-1-yl)-2- (2,4-difluorophenyl)-1-(1H- 1,2,4-triazol-1-yl)pent-3-yn-2-ol | 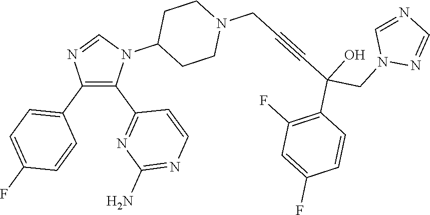 [SMILES: Nc1nccc(-c2c(-c3ccc(F)cc3)ncn2C2CCN(CC#CC(O)(Cn3cncn3)c3ccc(F)cc3F)CC2)n1] |
| I-229 | tert-butyl 9-(3-(4-(5-(2- aminopyrimidin-4-yl)-4-(4- fluorophenyl)-1H-imidazol-1- yl)piperidin-1-yl)propyl)-6- chloro-1,3,4,9-tetrahydro-2H- pyrido[3,4-b]indole-2- carboxylate | 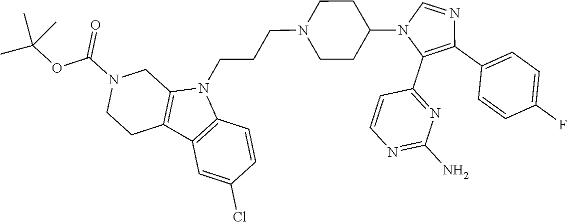 [SMILES: CC(C)(C)OC(=O)N1CCc2c(n(CCCN3CCC(n4cnc(-c5ccc(F)cc5)c4-c4ccnc(N)n4)CC3)c3ccc(Cl)cc23)C1] |
| I-230 | 6-((1-(2-(2,4-difluorophenyl)-2- hydroxy-3-(1H-1,2,4-triazol-1- yl)propyl)piperidin-4- yl)methyl)-4-(3-(4- fluorophenyl)-1-methyl-1H- pyrazol-4-yl)-6,7-dihydro-5H- pyrrolo[3,4-b]pyridin-5-one | 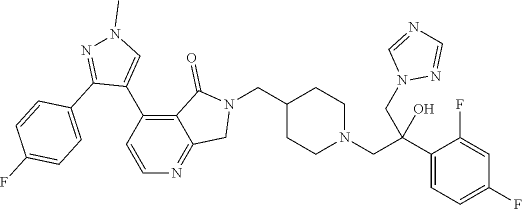 [SMILES: Cn1cc(-c2ccnc3c2C(=O)N(CC2CCN(CC(O)(Cn4cncn4)c4ccc(F)cc4F)CC2)C3)c(-c2ccc(F)cc2)n1] |
| I-231 | 4-(1-(1-(3-(8-chloro-1,2,3,4- tetrahydro-9H-pyrido[3,4- b]indol-9-yl)propyl)piperidin-4- yl)-4-(4-fluorophenyl)-1H- imidazol-5-yl)pyrimidin-2- amine | 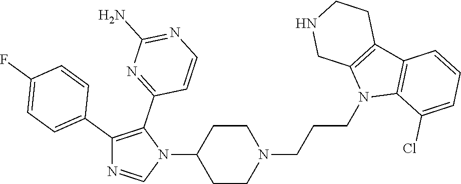 [SMILES: Nc1nccc(-c2c(-c3ccc(F)cc3)ncn2C2CCN(CCCn3c4c(c5cccc(Cl)c53)CCNC4)CC2)n1] |
| I-232 | 4-(1-(1-(3-(6-chloro-1,2,3,4- tetrahydro-9H-pyrido[3,4- b]indol-9-yl)propyl)piperidin-4- yl)-4-(4-fluorophenyl)-1H- imidazol-5-yl)pyrimidin-2- amine | 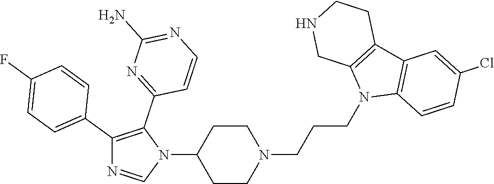 [SMILES: Nc1nccc(-c2c(-c3ccc(F)cc3)ncn2C2CCN(CCCn3c4c(c5cc(Cl)ccc53)CCNC4)CC2)n1] |
| I-233 | 2-(2,4-difluorophenyl)-4-((4-(3- (4-fluorophenyl)-1-methyl-1H- pyrazol-4-yl)pyridin-3- yl)methoxy)-1-(1H-1,2,4- triazol-1-yl)butan-2-ol | 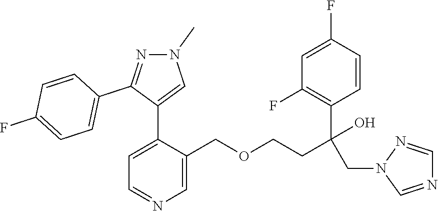 [SMILES: Cn1cc(-c2ccncc2COCCC(O)(Cn2cncn2)c2ccc(F)cc2F)c(-c2ccc(F)cc2)n1] |
| I-234 | 3-(4-(5-(2-aminopyrimidin-4- yl)-4-(4-fluorophenyl)-1H- imidazol-1-yl)piperidin-1-yl)-N- (1-(2-(2,4-difluorophenyl)-2- hydroxy-3-(1H-1,2,4-triazol-1- yl)propyl)piperidin-4- yl)propanamide | 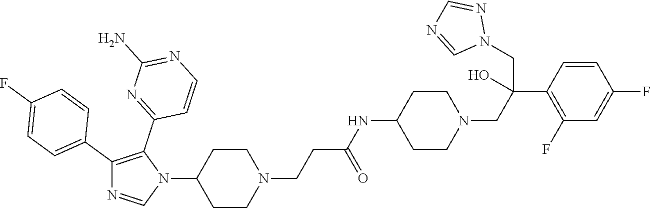 [SMILES: Nc1nccc(-c2c(-c3ccc(F)cc3)ncn2C2CCN(CCC(=O)NC3CCN(CC(O)(Cn4cncn4)c4ccc(F)cc4F)CC3)CC2)n1] |
| I-235 | (R)-2-(2,4-difluorophenyl)-4- ((4-(3-(4-fluorophenyl)-1- methyl-1H-pyrazol-4-yl)pyridin- 3-yl)methoxy)-1-(1H-1,2,4- triazol-1-yl)butan-2-ol | 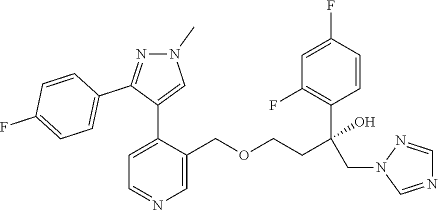 [SMILES: Cn1cc(-c2ccncc2COCC[C@](O)(Cn2cncn2)c2ccc(F)cc2F)c(-c2ccc(F)cc2)n1] |
| I-236 | (S)-2-(2,4-difluorophenyl)-4- ((4-(3-(4-fluorophenyl)-1- methyl-1H-pyrazol-4-yl)pyridin- 3-yl)methoxy)-1-(1H-1,2,4- triazol-1-yl)butan-2-ol | 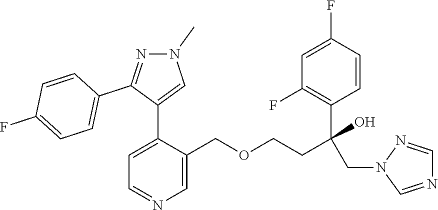 [SMILES: Cn1cc(-c2ccncc2COCC[C@@](O)(Cn2cncn2)c2ccc(F)cc2F)c(-c2ccc(F)cc2)n1] |
| I-237 | (R)-6-((1-(2-(2,4- difluorophenyl)-2-hydroxy-3- (1H-1,2,4-triazol-1- yl)propyl)piperidin-4- yl)methyl)-4-(3-(4- fluorophenyl)-1-methyl-1H- pyrazol-4-yl)-6,7-dihydro-5H- pyrrolo[3,4-b]pyridin-5-one | 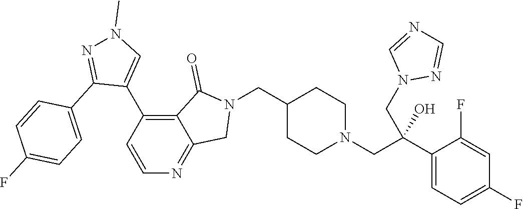 [SMILES: Cn1cc(-c2ccnc3c2C(=O)N(CC2CCN(C[C@@](O)(Cn4cncn4)c4ccc(F)cc4F)CC2)C3)c(-c2ccc(F)cc2)n1] |
| I-238 | (S)-6-((1-(2-(2,4- difluorophenyl)-2-hydroxy-3- (1H-1,2,4-triazol-1- yl)propyl)piperidin-4- yl)methyl)-4-(3-(4- fluorophenyl)-1-methyl-1H- pyrazol-4-yl)-6,7-dihydro-5H- pyrrolo[3,4-b]pyridin-5-one | 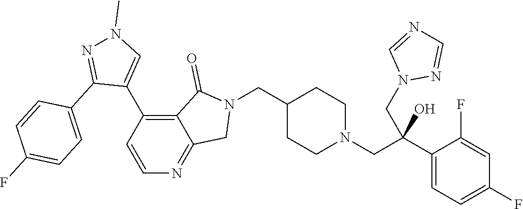 [SMILES: Cn1cc(-c2ccnc3c2C(=O)N(CC2CCN(C[C@](O)(Cn4cncn4)c4ccc(F)cc4F)CC2)C3)c(-c2ccc(F)cc2)n1] |
| I-239 | (4-(3-(4-fluorophenyl)-1- methyl-1H-pyrazol-4-yl)pyridin- 3-yl)methyl 1-(2-(2,4- difluorophenyl)-2-hydroxy-3- (1H-1,2,4-triazol-1- yl)propyl)piperidine-4- carboxylate | 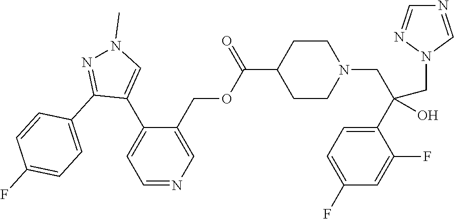 [SMILES: Cn1cc(-c2ccncc2COC(=O)C2CCN(CC(O)(Cn3cncn3)c3ccc(F)cc3F)CC2)c(-c2ccc(F)cc2)n1] |
| I-240 | 6-(2-(1-(2-(2,4-difluorophenyl)- 2-hydroxy-3-(1H-1,2,4-triazol- 1-yl)propyl)piperidin-4- yl)ethyl)-4-(3-(4-fluorophenyl)- 1-methyl-1H-pyrazol-4-yl)-6,7- dihydro-5H-pyrrolo[3,4- b]pyridin-5-one | 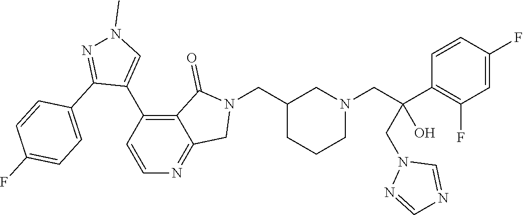 [SMILES: Cn1cc(-c2ccnc3c2C(=O)N(CC2CCCN(CC(O)(Cn4cncn4)c4ccc(F)cc4F)C2)C3)c(-c2ccc(F)cc2)n1] |
| I-241 | 6-(2-(2,4-difluorophenyl)-2- hydroxy-3-(1H-1,2,4-triazol-1- yl)propyl)-4-(3-(4- fluorophenyl)-1-methyl-1H- pyrazol-4-yl)-6,7-dihydro-5H- pyrrolo[3,4-b]pyridin-5-one | 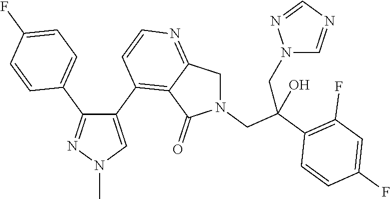 [SMILES: Cn1cc(-c2ccnc3c2C(=O)N(CC(O)(Cn2cncn2)c2ccc(F)cc2F)C3)c(-c2ccc(F)cc2)n1] |
| I-242 | 6-(1-(2-(2,4-difluorophenyl)-2- hydroxy-3-(1H-1,2,4-triazol-1- yl)propyl)piperidin-4-yl)-4-(3- (4-fluorophenyl)-1-methyl-1H- pyrazol-4-yl)-6,7-dihydro-5H- pyrrolo[3,4-b]pyridin-5-one | 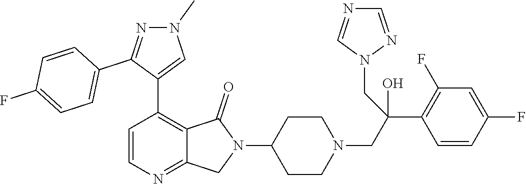 [SMILES: Cn1cc(-c2ccnc3c2C(=O)N(C2CCN(CC(O)(Cn4cncn4)c4ccc(F)cc4F)CC2)C3)c(-c2ccc(F)cc2)n1] |
| I-243 | 2-(2,4-difluorophenyl)- 1-((2-(4- fluorophenyl)-3-(pyridin-4- yl)pyrazolo[1,5-a]pyridin-7- yl)oxy)-3-(1H-1,2,4-triazol-1- yl)propan-2-ol | 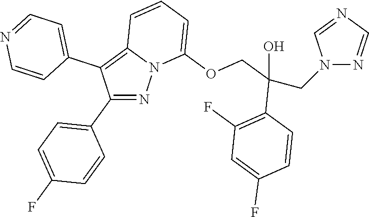 [SMILES: OC(COc1cccc2c(-c3ccncc3)c(-c3ccc(F)cc3)nn12)(Cn1cncn1)c1ccc(F)cc1F] |
| I-244 | (4-(3-(4-fluorophenyl)-1- methyl-1H-pyrazol-4-yl)pyridin- 3-yl)methyl (1-(2-(2,4- difluorophenyl)-2-hydroxy-3- (1H-1,2,4-triazol-1- yl)propyl)piperidin-4- yl)carbamate | 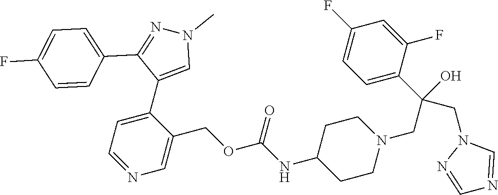 [SMILES: Cn1cc(-c2ccncc2COC(=O)NC2CCN(CC(O)(Cn3cncn3)c3ccc(F)cc3F)CC2)c(-c2ccc(F)cc2)n1] |
| I-245 | 1-(2-(2,4-difluorophenyl)-2- hydroxy-3-(1H-1,2,4-triazol-1- yl)propyl)piperidin-4-yl ((4-(3- (4-fluorophenyl)-1-methyl-1H- pyrazol-4-yl)pyridin-3- yl)methyl)carbamate | 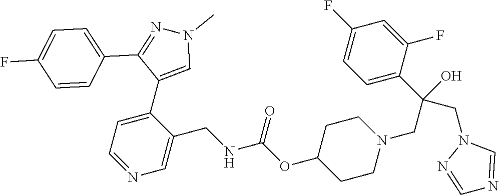 [SMILES: Cn1cc(-c2ccncc2CNC(=O)OC2CCN(CC(O)(Cn3cncn3)c3ccc(F)cc3F)CC2)c(-c2ccc(F)cc2)n1] |
| I-246 | 2-(2,4-difluorophenyl)- 1-(4- (((4-(3-(4-fluorophenyl)-1- methyl-1H-pyrazol-4-yl)pyridin- 3-yl)methoxy)methyl)piperidin- 1-yl)-3-(1H-1,2,4-triazol-1- yl)propan-2-ol | 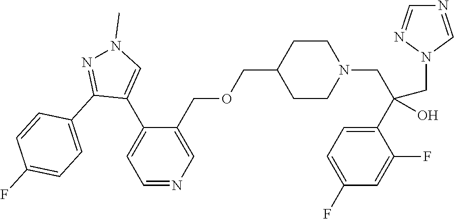 [SMILES: Cn1cc(-c2ccncc2COCC2CCN(CC(O)(Cn3cncn3)c3ccc(F)cc3F)CC2)c(-c2ccc(F)cc2)n1] |
| I-247 | (R)-6-(2-(1-(2-(2,4- difluorophenyl)-2-hydroxy-3-(1H- 1,2,4-triazol-1-yl)propyl)piperidin- 4-yl)ethyl)-4-(3-(4-fluorophenyl)-1- methyl-1H-pyrazol-4-yl)-6,7- dihydro-5H-pyrrolo[3,4-b]pyridin- 5-one | 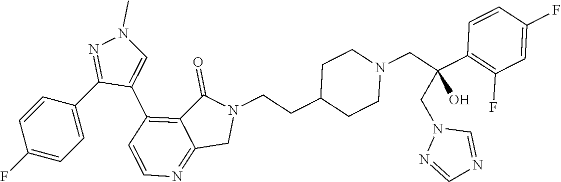 [SMILES: Cn1cc(-c2ccnc3c2C(=O)N(CCC2CCN(C[C@@](O)(Cn4cncn4)c4ccc(F)cc4F)CC2)C3)c(-c2ccc(F)cc2)n1] |
| I-248 | (S)-6-(2-(1-(2-(2,4- difluorophenyl)-2-hydroxy-3-(1H- 1,2,4-triazol-1-yl)propyl)piperidin- 4-yl)ethyl)-4-(3-(4-fluorophenyl)-1- methyl-1H-pyrazol-4-yl)-6,7- dihydro-5H-pyrrolo[3,4-b]pyridin- 5-one | 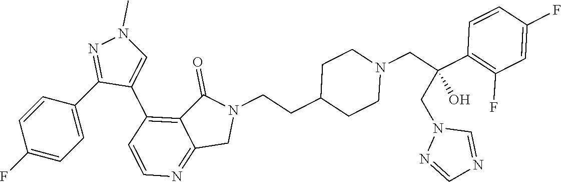 [SMILES: Cn1cc(-c2ccnc3c2C(=O)N(CCC2CCN(C[C@](O)(Cn4cncn4)c4ccc(F)cc4F)CC2)C3)c(-c2ccc(F)cc2)n1] |
| I-249 | 2-(2,4-difluorophenyl)-1,3-di(1H- 1,2,4-triazol-1-yl)propan-2-yl 2-(4- ((4-(3-(4-fluorophenyl)-1-methyl- 1H-pyrazol-4-yl)-5-oxo-5,7- dihydro-6H-pyrrolo[3,4-b]pyridin- 6-yl)methyl)piperidin-1-yl)acetate | 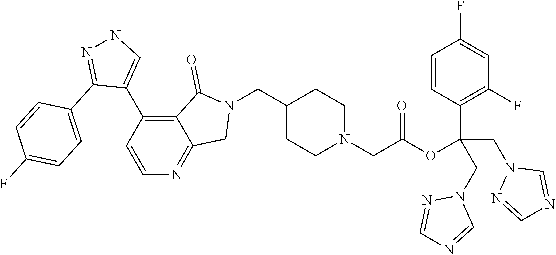 [SMILES: O=C(CN1CCC(CN2Cc3nccc(-c4c[nH]nc4-c4ccc(F)cc4)c3C2=O)CC1)OC(Cn1cncn1)(Cn1cncn1)c1ccc(F)cc1F] |
| I-250 | 6-(2-(1-(2-(2,4-difluorophenyl)-2- hydroxy-3-(1H-1,2,3-triazol-1- yl)propyl)piperidin-4-yl)ethyl)-4-(3- (4-fluorophenyl)-1-methyl-1H- pyrazol-4-yl)-6,7-dihydro-5H- pyrrolo[3,4-b]pyridin-5-one | 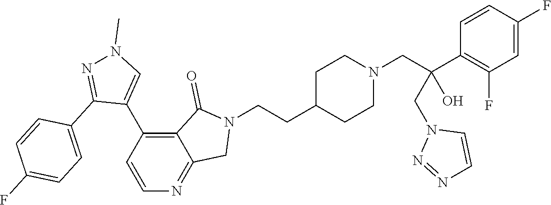 [SMILES: Cn1cc(-c2ccnc3c2C(=O)N(CCC2CCN(CC(O)(Cn4ccnn4)c4ccc(F)cc4F)CC2)C3)c(-c2ccc(F)cc2)n1] |
| I-251 | 2-(2,4-difluorophenyl)-1-(((2-(4- fluorophenyl)-3-(pyridin-4- yl)pyrazolo[1,5-a]pyridin-6- yl)methyl)amino)-3-(1H-1,2,3- triazol-1-yl)propan-2-ol | 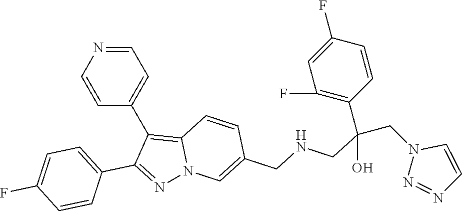 [SMILES: OC(CNCc1ccc2c(-c3ccncc3)c(-c3ccc(F)cc3)nn2c1)(Cn1ccnn1)c1ccc(F)cc1F] |
| I-252 | (R)-2-(2,4-difluorophenyl)-1-(((2- (4-fluorophenyl)-3-(pyridin-4- yl)pyrazolo[1,5-a]pyridin-6- yl)methyl)amino)-3-(1H-1,2,4- triazol-1-yl)propan-2-ol | 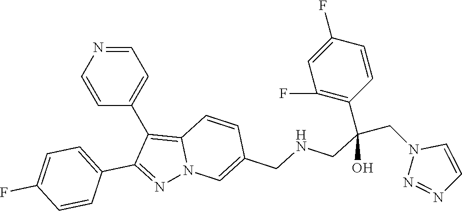 [SMILES: O[C@](CNCc1ccc2c(-c3ccncc3)c(-c3ccc(F)cc3)nn2c1)(Cn1ccnn1)c1ccc(F)cc1F] |
| I-253 | (S)-2-(2,4-difluorophenyl)-1-(((2- (4-fluorophenyl)-3-(pyridin-4- yl)pyrazolo[1,5-a]pyridin-6- yl)methyl)amino)-3-(1H-1,2,4- triazol-1-yl)propan-2-ol | 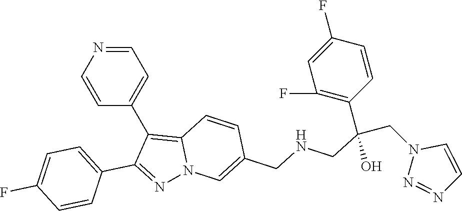 [SMILES: O[C@@](CNCc1ccc2c(-c3ccncc3)c(-c3ccc(F)cc3)nn2c1)(Cn1ccnn1)c1ccc(F)cc1F] |
| I-254 | 2-(2,4-difluorophenyl)-1-(4-(((2-(4- fluorophenyl)-3-(pyridin-4- yl)pyrazolo[1,5-a]pyridin-6- yl)methyl)amino)piperidin-1-yl)-3- (1H-1,2,4-triazol-1-yl)propan-2-ol | 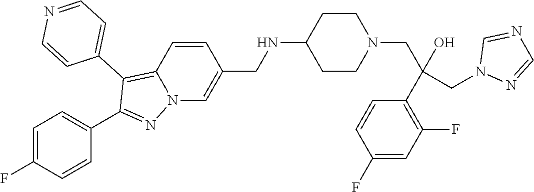 [SMILES: OC(CN1CCC(NCc2ccc3c(-c4ccncc4)c(-c4ccc(F)cc4)nn3c2)CC1)(Cn1cncn1)c1ccc(F)cc1F] |
| I-255 | 3-(2,4-difluorophenyl)-N-((2-(4- fluorophenyl)-3-(pyridin-4- yl)pyrazolo[1,5-a]pyridin-6- yl)methyl)-3-hydroxy-4-(1H-1,2,4- triazol-1-yl)butanamide | 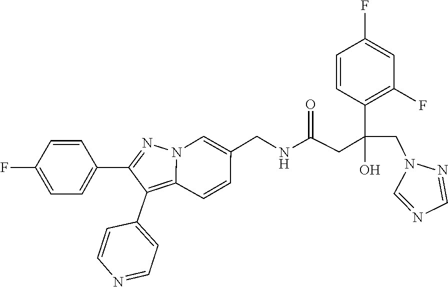 [SMILES: O=C(CC(O)(Cn1cncn1)c1ccc(F)cc1F)NCc1ccc2c(-c3ccncc3)c(-c3ccc(F)cc3)nn2c1] |
| I-256 | 1-((3S,5S)-5-((1H-1,2,4-triazol-1- yl)methyl)-5-(2,4- difluorophenyl)tetrahydrofuran-3- yl)-N-((2-(4-fluorophenyl)-3- (pyridin-4-yl)pyrazolo[1,5- a]pyridin-6- yl)methyl)methanamine | 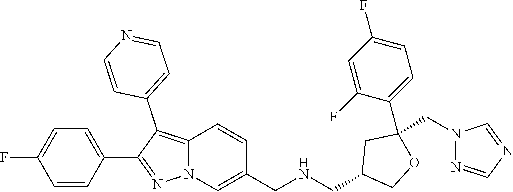 [SMILES: Fc1ccc(-c2nn3cc(CNC[C@H]4CO[C@](Cn5cncn5)(c5ccc(F)cc5F)C4)ccc3c2-c2ccncc2)cc1] |
| I-257 | 1-((3S,5R)-5-((1H-1,2,4-triazol-1- yl)methyl)-5-(2,4- difluorophenyl)tetrahydrofuran-3- yl)-N-((2-(4-fluorophenyl)-3- (pyridin-4-yl)pyrazolo[1,5- a]pyridin-6- yl)methyl)methanamine | 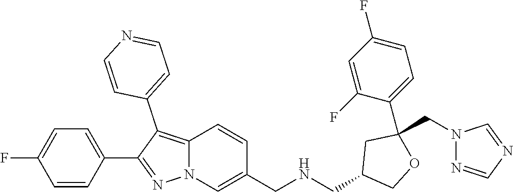 [SMILES: Fc1ccc(-c2nn3cc(CNC[C@H]4CO[C@@](Cn5cncn5)(c5ccc(F)cc5F)C4)ccc3c2-c2ccncc2)cc1] |
| I-258 | 2-(2,4-difluorophenyl)-4-(((2-(4- fluorophenyl)-3-(pyridin-4- yl)pyrazolo[1,5-a]pyridin-6- yl)methyl)amino)-1-(1H-1,2,4- triazol-1-yl)butan-2-ol | F 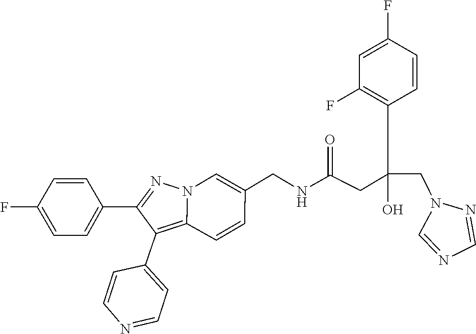 [SMILES: O=C(CC(O)(Cn1cncn1)c1ccc(F)cc1F)NCc1ccc2c(-c3ccncc3)c(-c3ccc(F)cc3)nn2c1] |
| I-259 | 2-(2,4-difluorophenyl)-1-(((2-(4- fluorophenyl)-3-(pyridin-4- yl)pyrazolo[1,5-a]pyridin-6- yl)methyl)(methyl)amino)-3-(1H- 1,2,4-triazol-1-yl)propan-2-ol | 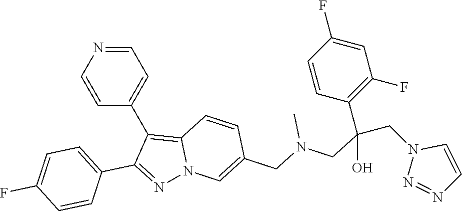 [SMILES: CN(Cc1ccc2c(-c3ccncc3)c(-c3ccc(F)cc3)nn2c1)CC(O)(Cn1ccnn1)c1ccc(F)cc1F] |
| I-260 | 2-(2,4-difluorophenyl)-1-(((2-(4- fluorophenyl)-3-(pyridin-4- yl)pyrazolo[1,5-a]pyridin-6- yl)methyl)amino)-3-(1H-tetrazol-1- yl)propan-2-ol | 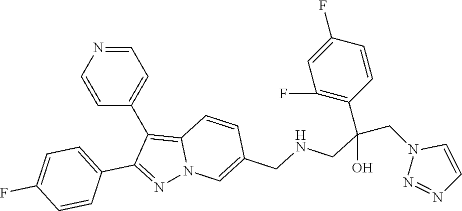 [SMILES: OC(CNCc1ccc2c(-c3ccncc3)c(-c3ccc(F)cc3)nn2c1)(Cn1ccnn1)c1ccc(F)cc1F] |
| I-261 | 2-(2,4-difluorophenyl)-1-(((2-(4- fluorophenyl)-3-(pyridin-4- yl)pyrazolo[1,5-a]pyridin-6- yl)methyl-d2)amino)-3-(1H-1,2,4- triazol-1-yl)propan-2-ol | 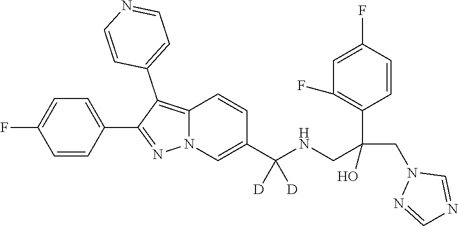 [SMILES: [2H]C([2H])(NCC(O)(Cn1cncn1)c1ccc(F)cc1F)c1ccc2c(-c3ccncc3)c(-c3ccc(F)cc3)nn2c1] |
| I-262 | 2-(2,4-difluorophenyl)-1, 1- difluoro-1-(5-(4-((((2-(4- fluorophenyl)-3-(pyridin-4- yl)pyrazolo[1,5-a]pyridin-6- yl)methyl)amino)methyl)phenyl)py ridin-2-yl)-3-(1H-1,2,4-triazol-1- yl)propan-2-ol | 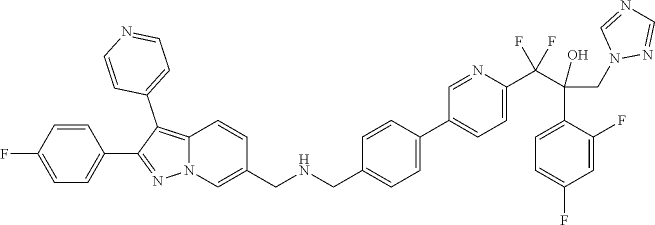 [SMILES: OC(Cn1cncn1)(c1ccc(F)cc1F)C(F)(F)c1ccc(-c2ccc(CNCc3ccc4c(-c5ccncc5)c(-c5ccc(F)cc5)nn4c3)cc2)cn1] |
| I-263 | (R)-2-(2,4-difluorophenyl)-1-(((S)- 1-(2-(4-fluorophenyl)-3-(pyridin-4- yl)pyrazolo[1,5-a]pyridin-6- yl)ethyl)amino)-3-(1H-1,2,4- triazol-1-yl)propan-2-ol | 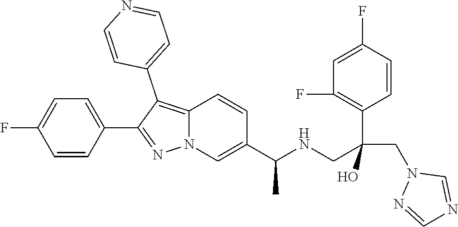 [SMILES: C[C@H](NC[C@@](O)(Cn1cncn1)c1ccc(F)cc1F)c1ccc2c(-c3ccncc3)c(-c3ccc(F)cc3)nn2c1] |
| I-264 | (S)-2-(2,4-difluorophenyl)-1-(((S)- 1-(2-(4-fluorophenyl)-3-(pyridin-4- yl)pyrazolo[1,5-a]pyridin-6- yl)ethyl)amino)-3-(1H-1,2,4- triazol-1-yl)propan-2-ol | 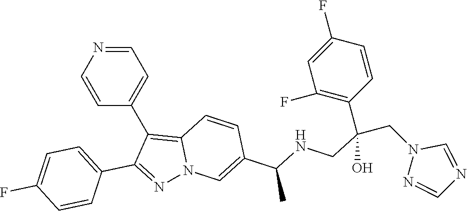 [SMILES: C[C@H](NC[C@](O)(Cn1cncn1)c1ccc(F)cc1F)c1ccc2c(-c3ccncc3)c(-c3ccc(F)cc3)nn2c1] |
| I-265 | 2-(2,4-difluorophenyl)-1,1- difluoro-1-(5-(4-(((2-(4- fluorophenyl)-3-(pyridin-4- yl)pyrazolo[1,5-a]pyridin-6- yl)methyl)amino)phenyl)pyridin-2- yl)-3-(1H-1,2,4-triazol-1- yl)propan-2-ol | 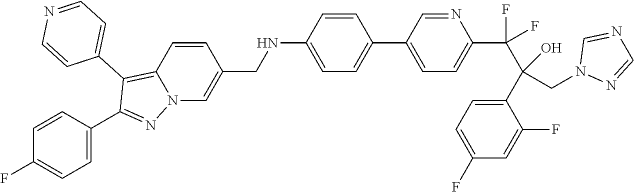 [SMILES: OC(Cn1cncn1)(c1ccc(F)cc1F)C(F)(F)c1ccc(-c2ccc(NCc3ccc4c(-c5ccncc5)c(-c5ccc(F)cc5)nn4c3)cc2)cn1] |
| I-266 | (2R,3R)-2-(2,4-difluorophenyl)-3- (((2-(4-fluorophenyl)-3-(pyridin-4- yl)pyrazolo[1,5-a]pyridin-6- yl)methyl)amino)-1-(1H-1,2,4- triazol-1-yl)butan-2-ol | 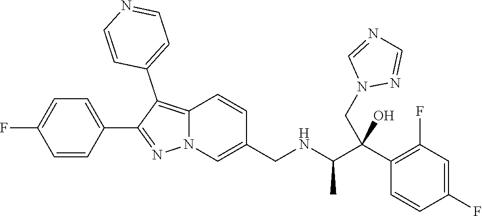 [SMILES: C[C@@H](NCc1ccc2c(-c3ccncc3)c(-c3ccc(F)cc3)nn2c1)[C@](O)(Cn1cncn1)c1ccc(F)cc1F] |
| I-267 | 2-(2,4-difluorophenyl)-1, 1- difluoro-1-(5-((4-((((2-(4- fluorophenyl)-3-(pyridin-4- yl)pyrazolo[1,5-a]pyridin-6- yl)methyl)amino)methyl)phenyl) ethynyl)pyridin-2-yl)-3-(1H- 1,2,4-triazol-1-yl)propan-2-ol | 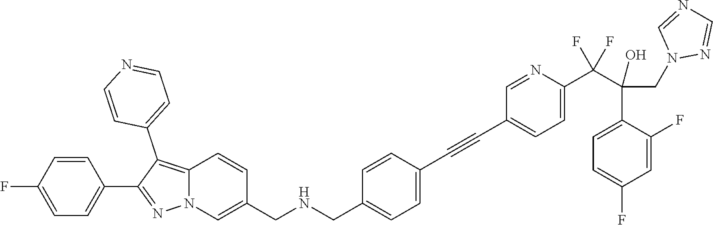 [SMILES: OC(Cn1cncn1)(c1ccc(F)cc1F)C(F)(F)c1ccc(C#Cc2ccc(CNCc3ccc4c(-c5ccncc5)c(-c5ccc(F)cc5)nn4c3)cc2)cn1] |
| I-268 | 2-(2,4-difluorophenyl)-1, 1- difluoro-1-(5-((4-(((2-(4- fluorophenyl)-3-(pyridin-4- yl)pyrazolo[1,5-a]pyridin-6- yl)methyl)amino)phenyl)ethynyl) pyridin-2-yl)-3-(1H-1,2,4- triazol-1-yl)propan-2-ol | 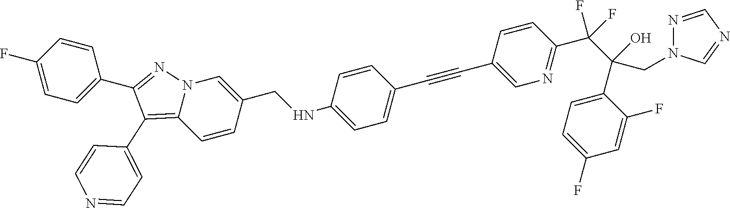 [SMILES: OC(Cn1cncn1)(c1ccc(F)cc1F)C(F)(F)c1ccc(C#Cc2ccc(NCc3ccc4c(-c5ccncc5)c(-c5ccc(F)cc5)nn4c3)cc2)cn1] |
| I-269 | 2-(2,4-difluorophenyl)-1, 1- difluoro-1-(5-(4-((((2-(4- fluorophenyl)-3-(pyridin-4- yl)pyrazolo[1,5-a]pyridin-6- yl)methyl)amino)methyl)phenyl) pyridin-2-yl)-3-(1H-tetrazol-1- yl)propan-2-ol | 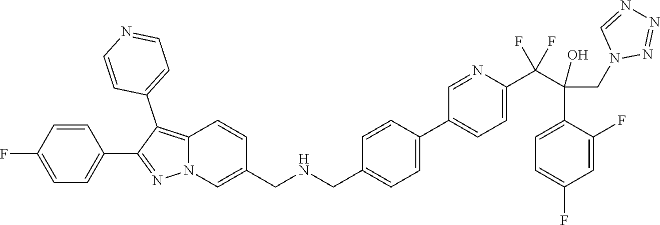 [SMILES: OC(Cn1cnnn1)(c1ccc(F)cc1F)C(F)(F)c1ccc(-c2ccc(CNCc3ccc4c(-c5ccncc5)c(-c5ccc(F)cc5)nn4c3)cc2)cn1] |
| I-270 | 2-(2,4-difluorophenyl)-1, 1- difluoro-1-(5-((4-(2-(((2-(4- fluorophenyl)-3-(pyridin-4- yl)pyrazolo[1,5-a]pyridin-6- yl)methyl)amino)ethoxy)phenyl) ethynyl)pyridin-2-yl)-3-(1H- 1,2,4-triazol-1-yl)propan-2-ol | 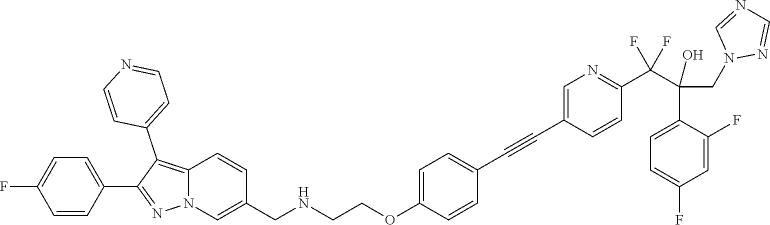 [SMILES: OC(Cn1cncn1)(c1ccc(F)cc1F)C(F)(F)c1ccc(C#Cc2ccc(OCCNCc3ccc4c(-c5ccncc5)c(-c5ccc(F)cc5)nn4c3)cc2)cn1] |
| I-271 | 4-(3-(((2-(4-fluorophenyl)-3- (pyridin-4-yl)pyrazolo[1,5- a]pyridin-6- yl)methyl)amino)propoxy) benzimidamide | 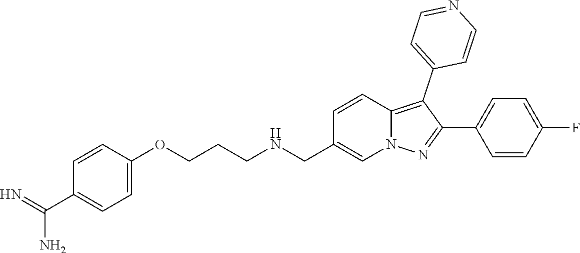 [SMILES: N=C(N)c1ccc(OCCCNCc2ccc3c(-c4ccncc4)c(-c4ccc(F)cc4)nn3c2)cc1] |
| I-272 | 4-(3-(((2-(4-fluorophenyl)-3- (pyridin-4-yl)pyrazolo[1,5- a]pyridin-6- yl)methyl)(methyl)amino) propoxy)benzimidamide | 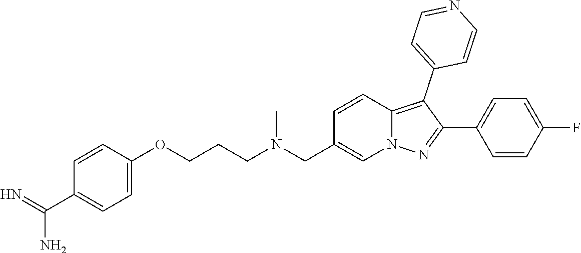 [SMILES: CN(CCCOc1ccc(C(=N)N)cc1)Cc1ccc2c(-c3ccncc3)c(-c3ccc(F)cc3)nn2c1] |
| I-273 | (R)-4-(3-((1-(2-(4-fluorophenyl)-3- (pyridin-4-yl)pyrazolo[1,5- a]pyridin-6- yl)ethyl)amino)propoxy) benzimidamide | 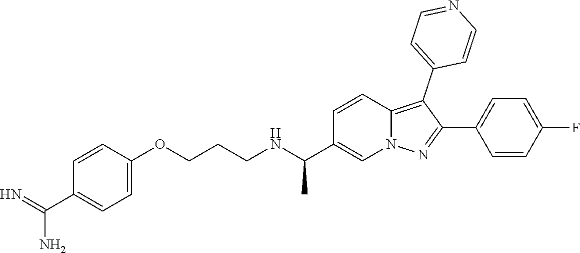 [SMILES: C[C@@H](NCCCOc1ccc(C(=N)N)cc1)c1ccc2c(-c3ccncc3)c(-c3ccc(F)cc3)nn2c1] |
| I-274 | (S)-4-(3-((1-(2-(4-fluorophenyl)-3- (pyridin-4-yl)pyrazolo[1,5- a]pyridin-6- yl)ethyl)amino)propoxy) benzimidamide | 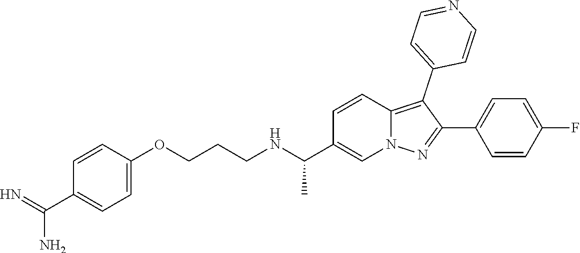 [SMILES: C[C@H](NCCCOc1ccc(C(=N)N)cc1)c1ccc2c(-c3ccncc3)c(-c3ccc(F)cc3)nn2c1] |
| I-275 | 4-(4-(((2-(4-fluorophenyl)-3- (pyridin-4-yl)pyrazolo[1,5- a]pyridin-6- yl)methyl)amino)butoxy) benzimidamide | 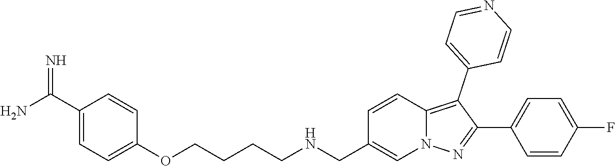 [SMILES: N=C(N)c1ccc(OCCCCNCc2ccc3c(-c4ccncc4)c(-c4ccc(F)cc4)nn3c2)cc1] |
| I-276 | 4-(3-(4-(((2-(4-fluorophenyl)-3- (pyridin-4-yl)pyrazolo[1,5- a]pyridin-6- yl)methyl)amino)piperidin-1- yl)propoxy)benzimidamide | 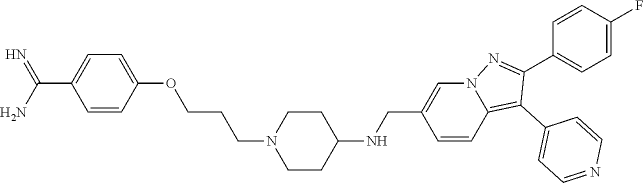 [SMILES: N=C(N)c1ccc(OCCCN2CCC(NCc3ccc4c(-c5ccncc5)c(-c5ccc(F)cc5)nn4c3)CC2)cc1] |
| I-277 | 4-(4-(4-(3-(4-fluorophenyl)-1- methyl-1H-pyrazol-4-yl)-5-oxo- 5,7-dihydro-6H-pyrrolo[3,4- b]pyridin-6- yl)butoxy)benzimidamide | 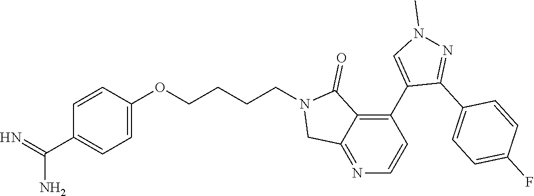 [SMILES: Cn1cc(-c2ccnc3c2C(=O)N(CCCCOc2ccc(C(=N)N)cc2)C3)c(-c2ccc(F)cc2)n1] |
| I-278 | 4-(3-(4-(3-(4-fluorophenyl)-1- methyl-1H-pyrazol-4-yl)-5-oxo- 5,7-dihydro-6H-pyrrolo[3,4- b]pyridin-6- yl)propoxy)benzimidamide | 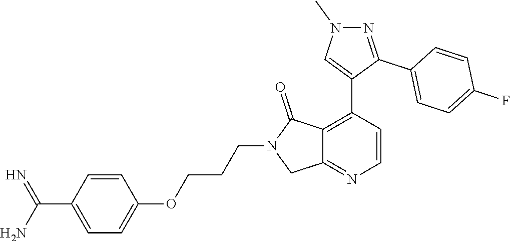 [SMILES: Cn1cc(-c2ccnc3c2C(=O)N(CCCOc2ccc(C(=N)N)cc2)C3)c(-c2ccc(F)cc2)n1] |
| I-279 | 2-(2,4-difluorophenyl)-5-(((2-(4- fluorophenyl)-3-(pyridin-4- yl)pyrazolo[1,5-a]pyridin-6- yl)methyl)amino)-1-(1H-1,2,4- triazol-1-yl)pentan-2-ol | 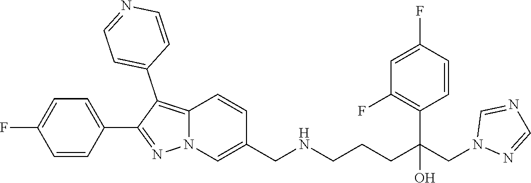 [SMILES: OC(CCCNCc1ccc2c(-c3ccncc3)c(-c3ccc(F)cc3)nn2c1)(Cn1cncn1)c1ccc(F)cc1F] |
| I-280 | 2-(2,4-difluorophenyl)-5-(((2-(4- fluorophenyl)-3-(pyridin-4- yl)pyrazolo[1,5-a]pyridin-6- yl)methyl)amino)-1-(1H-1,2,4- triazol-1-yl)pent-3-yn-2-ol | 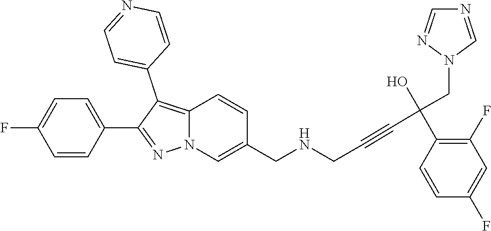 [SMILES: OC(C#CCNCc1ccc2c(-c3ccncc3)c(-c3ccc(F)cc3)nn2c1)(Cn1cncn1)c1ccc(F)cc1F] |
| 1-281 | 4-(3-(((2-(4-fluorophenyl)-3- (pyridin-4-yl)pyrazolo[1,5- a]pyridin-6- yl)methyl)amino)propoxy) benzimidamide HCI salt | 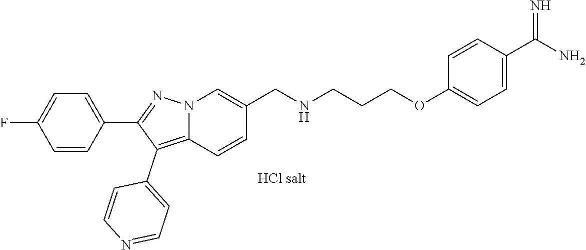 [SMILES: N=C(N)c1ccc(OCCCNCc2ccc3c(-c4ccncc4)c(-c4ccc(F)cc4)nn3c2)cc1] |

or, an enantiomer thereof (where relevant), or a pharmaceutically acceptable salt, solvate and/or prodrug thereof, or an isotopic variant thereof, for example wherein the isotopic variant is deuterium and the compound of Formula I comprises one or more deuterium atoms.

The present application further includes a pharmaceutical composition comprising one or more conjugate compounds of the present application, or a pharmaceutically acceptable salt, solvate and/or prodrug thereof, and a pharmaceutically acceptable carrier and/or diluent. In some embodiments, the pharmaceutical composition further comprising an additional therapeutic agent.

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

The compounds of the present application may also exist in different tautomeric forms and it is intended that any tautomeric forms which the compounds form, as well as mixtures thereof, are included within the scope of the present application.

The compounds of the present application may further exist in varying polymorphic forms and it is contemplated that any polymorphs, or mixtures thereof, which form are included within the scope of the present application.

In the compounds of the present application and pharmaceutically acceptable salts, solvates and/or prodrugs thereof, the atoms may exhibit their natural isotopic abundances, or one or more of the atoms may be artificially enriched in a particular isotope having the same atomic number, but an atomic mass or mass number different from the atomic mass or mass number predominantly found in nature. The present application is meant to include all suitable isotopic variations of the compounds of application. For example, different isotopic forms of hydrogen (H) include protium (¹H), deuterium (²H) and tritium (³H). Protium is the predominant hydrogen isotope found in nature. In some embodiments, the compounds of the application comprise one of more deuterium atoms.

The compounds of the present application may further be radiolabeled and accordingly all radiolabeled versions of the compounds of the application are included within the scope of the present application. Therefore, the compounds of the application also include those in which one or more radioactive atoms are incorporated within their structure.

In an embodiment the pharmaceutically acceptable salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Solvates of compounds of the application include, for example, those made with solvents that are pharmaceutically acceptable. Examples of such solvents include water (resulting solvate is called a hydrate) and ethanol and the like.

Prodrugs of the compounds of the present application may be, for example, conventional esters formed with available hydroxy, thiol, amino or carboxyl groups. Some common esters which have been utilized as prodrugs are phenyl esters, aliphatic (C₁-C₂₄) esters, acyloxymethyl esters, carbamates and amino acid esters.

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier.

A compound of the application including salts and/or solvates thereof is suitably used on their own but will generally be administered in the form of a composition in which the one or more compounds of the application (the active ingredient) is in association with an acceptable carrier. Depending on the mode of administration, the composition will comprise from about 0.05 wt % to about 99 wt % or about 0.10 wt % to about 70 wt %, of the active ingredient, and from about 1 wt % to about 99.95 wt % or about 30 wt % to about 99.90 wt % of an acceptable carrier, all percentages by weight being based on the total composition.

The compounds of the application may be administered to a subject in a variety of forms depending on the selected route of administration, as will be understood by those skilled in the art. A compound of the application may be administered, for example, by oral, parenteral, buccal, sublingual, nasal, rectal, patch, pump or transdermal administration and the pharmaceutical compositions formulated accordingly. Administration can be by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists.

A compound of the application may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound may be incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. Liposomes can be formed from a variety of phospholipids, such as cholesterol, stearylamine or phosphatidylcholines. For oral administration in a capsule form, useful carriers or diluents include lactose and dried corn starch.

Liquid preparations for oral administration may take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents may be added. Such liquid preparations for oral administration may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

A compound of the application may also be administered parenterally. Solutions of a compound of the application can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

The compounds of the application may be formulated for parenteral administration by injection, including using conventional catheterization techniques or infusion. Formulations for injection may be presented in unit dosage form, e.g., in ampoules or in multi-dose containers, with an added preservative. The compositions may take such forms as sterile suspensions, solutions or emulsions in oily or aqueous vehicles, and may contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

Compositions for nasal administration may conveniently be formulated as aerosols, drops, gels and powders.

For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, PA, 1980, pp. 1530-1533 for further discussion of suppository dosage forms.

Compounds of the application may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, compounds of the application may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

In an embodiment, compounds of the application may be coupled with viral, non-viral or other vectors. Viral vectors may include retrovirus, lentivirus, adenovirus, herpesvirus, poxvirus, alphavirus, vaccinia virus or adeno-associated viruses. Non-viral vectors may include nanoparticles, cationic lipids, cationic polymers, metallic nanoparticles, nanorods, liposomes, micelles, microbubbles, cell-penetrating peptides, or lipospheres. Nanoparticles may include silica, lipid, carbohydrate, or other pharmaceutically acceptable polymers.

In some embodiments, depending on the mode of administration, the pharmaceutical composition will comprise from about 0.05 wt % to about 99 wt % or about 0.10 wt % to about 70 wt %, of the active ingredient (one or more compounds of the application), and from about 1 wt % to about 99.95 wt % or about 30 wt % to about 99.90 wt % of one or more pharmaceutically acceptable carriers, all percentages by weight being based on the total composition.

In an embodiment, a compound of the present application is administered with another therapeutic agent simultaneously or sequentially in separate unit dosage forms or together in a single unit dosage form. Accordingly, the present application provides a single unit dosage form comprising one or more compounds of the application (e.g. a compound of Formula (I)), an additional therapeutic agent, and a pharmaceutically acceptable carrier.

To be clear, in the above, the term “a compound” also includes embodiments wherein one or more compounds are referenced.

### III. Methods and Uses of the Application

The compounds of the application have been shown to be capable of inhibiting fungal activity.

Accordingly, the present application further includes a method of treating or preventing a fungal-related disease, disorder or condition comprising administering a therapeutically effective amount of one or more conjugate compounds of the present application to a subject in need thereof.

The present application also includes a method of inhibiting or preventing fungal growth comprising administering a therapeutically effective amount of one or more conjugate compounds of the present application to a subject in need thereof.

Also provided is a method of inhibiting fungal CK1 activity comprising administering a therapeutically effective amount of one or more conjugate compounds of the present application to a subject in need thereof. In some embodiments, the method comprises selectively inhibiting fungal CK1 activity.

The present application further provides a method of treating a fungal-related disease, disorder or condition that is treatable by inhibiting fungal CK1 comprising administering a therapeutically effective amount of one or more conjugate compounds of the present application in combination with another known agent useful for treatment of a fungal-related disease, disorder or condition that is treatable by inhibiting fungal CK1 to a subject in need thereof.

The application also includes a use of one or more conjugate compounds of the application for treating or preventing a fungal-related disease, disorder or condition as well as a use of one or more conjugates compounds of the application for the preparation of a medicament for treating or preventing a fungal-related disease, disorder or condition. The application further includes one or more conjugate compounds of the application for use in treating or preventing a fungal-related disease, disorder or condition.

As the conjugate compounds of the application have been shown to be capable of inhibiting fungal activity, the conjugate compounds of the application are useful for treating or preventing a fungal-related disease, disorder or condition by inhibiting fungal activity. Therefore the conjugate compounds of the present application are useful as medicaments. Accordingly, the present application includes a conjugate compound of the application for use as a medicament.

In some embodiments, the fungal-related disease, disorder or condition comprises mycosis including superficial, subcutaneous and systemic mycosis. In some embodiements, the fungal-related disease, disorder or condition includes dermatomycosis, candidiasis, pneumocytosis, Pityriasis versicolor, aspergillosis, mucormycosis, talaromycosis, basidiobolomycosis, blastomycosis, chromoblastomycosis cryptococcosis, coccidioidomycosis, conidiobolomycosis, eumycetoma, histoplasmosis, lobomycosis, paracoccidioidomycosis, phaeohyphomycosis, scedosporisis, sporotrichosis and emmonsiosis.

Accordingly, the present application thus provides a method of treating or preventing a fungal infection or mycosis comprising administering a therapeutically effective amount of one or more conjugate compounds of the present application to a subject in need thereof. In some embodiments, the mycosis is candidiasis,

In some embodiments, fungi that cause infections include the yeasts, molds, and/or dimorphic fungi responsible for the infections specified herein.

When used in combination with other agents or therapies useful in treating fungal-related diseases, disorders or conditions, it is an embodiment that the conjugate compounds of the application are administered contemporaneously with those agents or therapies. As used herein, “contemporaneous administration” of two substances or therapies to a subject means providing each of the two substances or therapies so that they are both biologically active in the individual at the same time. The exact details of the administration will depend on the pharmacokinetics of the two substances or therapies in the presence of each other, and can include administering the two substances or therapies within a few hours of each other, or even administering one substance or therapy within 24 hours of administration of the other, if the pharmacokinetics are suitable. Design of suitable dosing regimens is routine for one skilled in the art. In particular embodiments, the substances or therapies will be administered substantially simultaneously, i.e., within minutes of each other, or in a single composition in the case of administration of two substances. It is a further embodiment of the present application that a combination of agents or therapies is administered to a subject in a non-contemporaneous fashion.

In some embodiments, the subject is a mammal. In some embodiments, the subject is a livestock, such as cattle, sheep, goat, poultry, etc. In some embodiments, the subject is human. In some embodiments, mycoses in human affect skin, mucosa such as mouth and vagina, nails, organs such as lungs and brain, eyes, nose, sinuses, bones, joints, etc.

In some embodiments, the subject is a crop, such as rice, wheat, barley, oat, rye, sugarcane and other sugar crops, maize (corn), potatoes, palm, canola, flax, safflower cassava, legume pulses such as beans, soybeans, peas, chickpeas, peanuts, lentils, lupins, mesquite, carob, tamarind, alfalfa, and clover, sunflower, rape, mustard, sorghum, millet, hemp, sugar beet, groundnuts, sweet potatoes, bananas, cotton, yams, various nuts or other vegetables or fruits.

In the context of treating a fungal-related disease, disorder or condition, an effective amount is an amount that, for example, inhibits fungal activity, compared to the inhibition without administration of the one or more conjugate compounds. Effective amounts may vary according to factors such as the disease state, age, sex and/or weight of the subject, type of fungi. The amount of a given compound that will correspond to such an amount will vary depending upon various factors, such as the given drug or compound, the pharmaceutical formulation, the route of administration, the type of condition, disease or disorder, the identity of the subject being treated, and the like, but can nevertheless be routinely determined by one skilled in the art. The effective amount is one that following treatment therewith manifests as an improvement in or reduction of any disease symptom.

The dosage of compounds of the application can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. Compounds of the application may be administered initially in a suitable dosage that may be adjusted as required, depending on the clinical response. Dosages will generally be selected to maintain a serum level of compounds of the application from about 0.01 μg/cc to about 1000 μg/cc, or about 0.1 μg/cc to about 100 μg/cc. As a representative example, oral dosages of one or more compounds of the application will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg. In an embodiment of the application, compositions are formulated for oral administration and the compounds are suitably in the form of tablets containing 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient per tablet. Compounds of the application may be administered in a single daily, weekly or monthly dose or the total daily dose may be divided into two, three or four daily doses.

In some embodiments, the compounds of the application are administered at least once a week. However, in another embodiment, the compounds are administered to the subject from about one time per two weeks, three weeks or one month. In another embodiment, the compounds are administered about one time per week to about once daily. In another embodiment, the compounds are administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds are administered to the subject in an amount and for duration sufficient to treat the subject.

### IV. Methods of Preparing the Compounds of the Application

Compounds of the present application can be prepared by various synthetic processes. The choice of particular structural features and/or substituents may influence the selection of one process over another. The selection of a particular process to prepare a given conjugate compound of Formula (I) is within the purview of the person of skill in the art. Some starting materials for preparing compounds of the present application are available from commercial chemical sources. Other starting materials, are readily prepared from available precursors using straightforward transformations that are well known in the art.

In some embodiments, the compounds of Formula I are assembled by attaching the linker group to one of A or B following by attachment of the other of A or B. Standard chemistries known in the art can be used to assemble the compounds of Formula I, including but not limited to, nucleophilic displacements, cross-couplings, Michael reactions and/or activating group strategies. As many of the A and B groups are known, or are based on known compounds, compounds that can be used to react with the linker group are readily available either from commercial sources or using synthetic methods known in the art. For example, many such compounds have at least one functional group in their structure capable of forming a covalent bond with the linker group. Examples of such include an amine, thiol, halo, hydroxyl, alkoxy, carboxyl, ester, amide and/or oxo group.

In some embodiments, the compounds of Formula (I) have the structure of Formula (I-A):

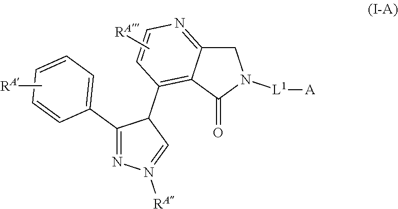

In some embodiments, compounds of Formula (I-A) are synthesized by reacting intermediate 1a with amine Ib in a solvent, according to known reactions, to provide intermediate Ic, which is then reduced under known reduction conditions to provide compounds of Formula (I-A), as shown in Scheme 1. In some embodiments, R^{A′}, R^{A″} and R^{A″′} are independently selected from H, halo, C₁₋₆alkyl and C₁₋₆haloalkyl. In some embodiments, R^{A′}, R^{A″} and R^{A″′} are independently selected from H, F, CH₃, CH₂CH₃, CF₂H, CF₃, CFH₂, CH₂CF₂H, and CH₂CF₃. In some embodiments, R^{A′}, R^{A″} and R^{A″′} are independently selected from H, F and CH₃. In some embodiments, R^{A′}, R^{A″}, R^{A′″} and A have the meaning for the corresponding groups in compounds I-1, I-6 to I-13, I-25, I-188, I-191 to I-209, I-230, I-237, I-238, and I-240 to I-242 shown in Table 2.

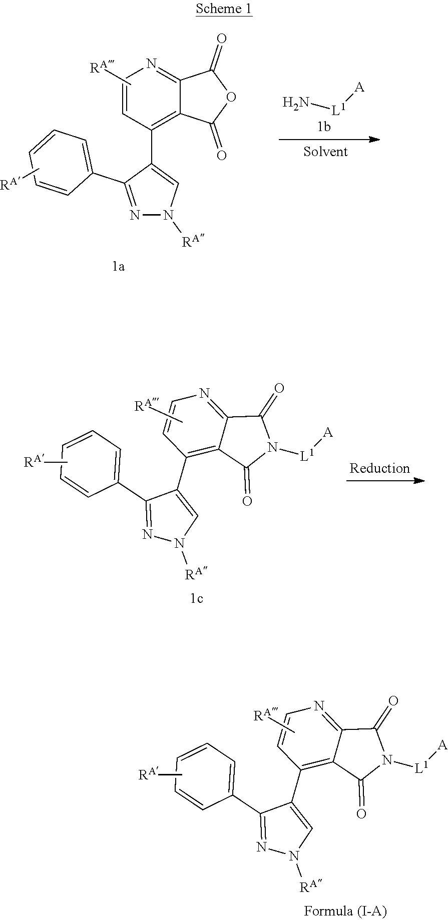

In some embodiments, the compounds of Formula (I) have the structure of Formula (I-B):

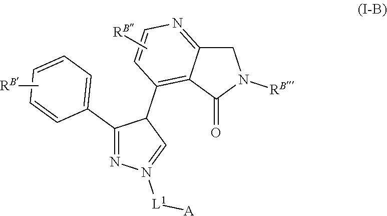

In some embodiments, the compounds (I) of Formula (I-B) are synthesized, by reacting intermediate 2a bearing protecting group PG, with amine 2b in a solvent according to known reactions, to provide intermediate 2c. Intermediate 2c may then be reduced to provide intermediate 2d. Protecting group PG may be deprotected using known deprotection methods to provide intermediate 2e. 2e is reacted with compound 2f, bearing leaving group LG, under known coupling conditions to obtain Formula (I-B), as shown in Scheme 2. In some embodiments, R^{B′}, R^{B″} and R^{B″′} are independently selected from H, halo, C₁₋₆alkyl and C₁₋₆haloalkyl. In some embodiments, R^{B′}, R^{B″} and R^{B″′} are independently selected from H, F, CH₃, CH₂CH₃, CF₂H, CF₃, CFH₂, CH₂CF₂H, and CH₂CF₃. In some embodiments, R^{B′}, R^{B″} and R^{B″′} are independently selected from H, F and CH₃. In some embodiments, R^{B′}, R^{B″}, R^{B″′}, L¹ and A have the meaning for the corresponding groups in compounds I-2, I-18 to I-22, and I-26 to I-31 shown in Table 2

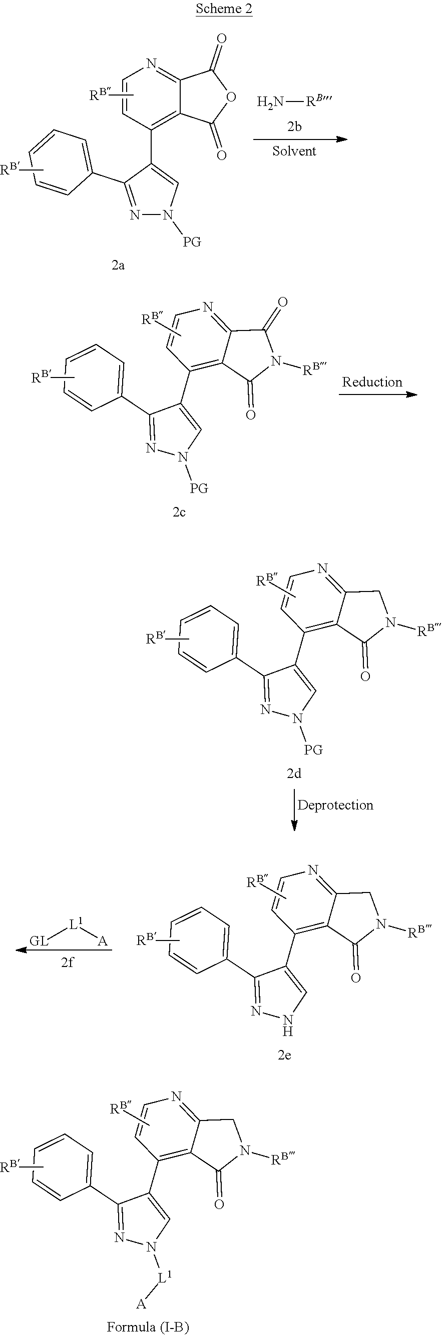

In some embodiments, the compounds of Formula (I) have the structure of Formula (I-C):

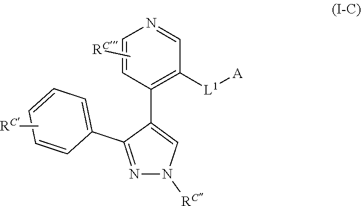

In some embodiments, the compounds of Formula (I-C) are synthesized by reacting intermediate 3a with amine 3b in a solvent according to known reactions, to provide intermediate 3c. Intermediate 3c may then be reacted with halide 3d through alkylation conditions to provide intermediate 3e. Intermediate 3e may then be halogenated to provide 3f and boronated to provide 3g according to known conditions, before being submitted to Suzuki coupling conditions in the presence of compound 3 h to provide intermediate 3i. Functional Group (FG) on intermediate 3i may then be substituted according to various known methods to provide intermediate 3j and finally compounds of Formula (I-C), as shown in Scheme 3. In some embodiments, R^{C′}, R^{C″} and R^{C″′} are independently selected from H, halo, C₁₋₆alkyl and C₁₋₆haloalkyl. In some embodiments, R^{C′}, R^{C″} and R^{C″′} are independently selected from H, F, CH₃, CH₂CH₃, CF₂H, CF₃, CFH₂, CH₂CF₂H, and CH₂CF₃. In some embodiments, R^{C′}, R^{C″} and R^{C″′} are independently selected from H, F and CH₃. In some embodiments, linker L¹ and A are as defined in Formula (I). In some embodiments, R^{C′}, R^{C″}, R^{C″′}, L¹ and A are as defined for the corresponding groups in compounds I-3 to I-5, I-14 to I-17, I-23, I-24, I-89, I-90, I-189, I-190, I-233, I-235, I-236, I-239 and I-244 to I-246 in Table 2.

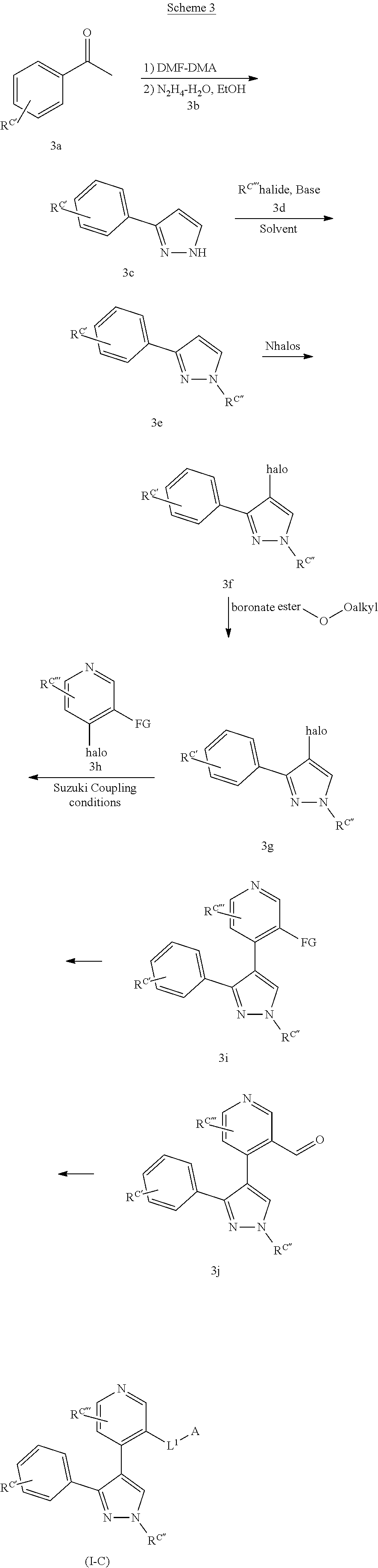

In some embodiments, the compounds of Formula (I) have the structure of Formula (I-D):

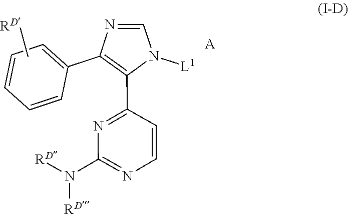

In some embodiments, the compounds of Formula (I-D) are synthesized by reacting intermediate 4a with amine 4b or a protected version thereof, in the presence of a base in a solvent according to known reactions, to provide intermediate 4c. Intermediate 4c may then be coupled with compound 4d to provide intermediate 4e. If needed any protecting group in intermediate 4e may be deprotected using known deprotection methods and then submitted to known condensation condition to afford compounds of Formula (I-D), as shown in Scheme 4. In some embodiments, R^{D′}, R^{D″} and R^{D′″} are independently selected from H, halo, C₁₋₆alkyl, C₁₋₆haloalkyl and C₃₋₁₀heteroaryl optionally substituted with C₁₋₆alkyl or C₃₋₆cycloalkyl. In some embodiments, R^{D′}, R^{D″} and R^{D″′} are independently selected from H, F, CH₃, CH₂CH₃, CF₂H, CF₃, CFH₂, CH₂CF₂H, CH₂CF₃ and pyrazole. In some embodiments, R^{D′}, R^{D″} and R^{D′″} are independently selected from H, F, CH₃ and

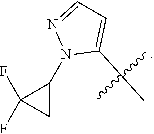 [SMILES: *c1ccnn1C1CC1(F)F]

In some embodiments, L¹ and A are as defined in Formula (I). In some embodiments, R^{D′}, R^{D″}, R^{D″′}, L¹ and A are as defined for the corresponding groups in compounds I-32 to I-86, I-91 to I-99, I-210 to I-229, I-231, I-232 and I-234 shown in Table 2.

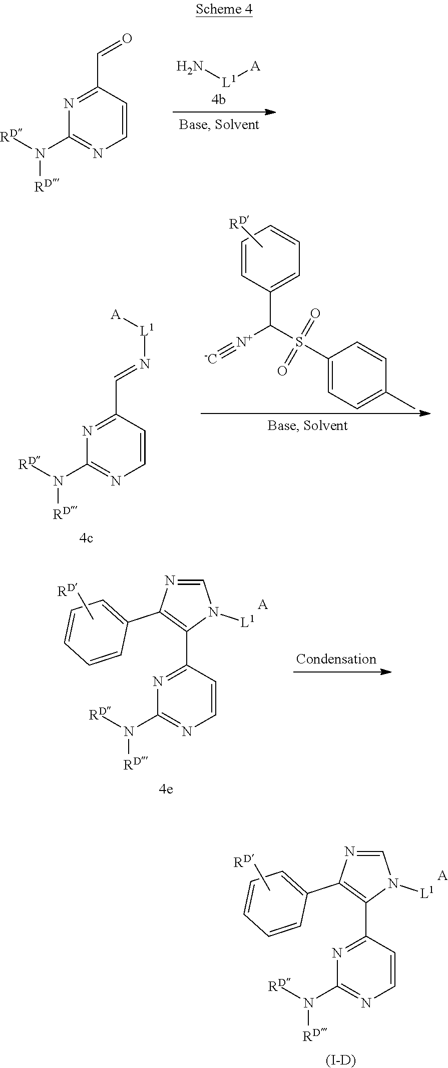

In some embodiments, the compounds of Formula (I) have the structure of Formula (I-E), wherein one of R^{E′} and R^{E″} is L¹-A:

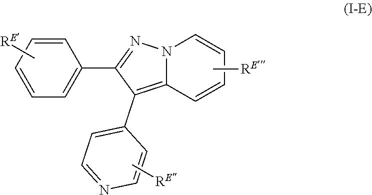

In some embodiments, compounds of Formula (I-E) are synthesized by reacting intermediate 5a bearing a suitable protecting group (PG) with a base 5b to provide intermediate 5c. Intermediate 5c may then be reacted with compound 5d to provide intermediate 5e, which may be subjected to known hydrolysis conditions to provide intermediate 5f. Intermediate 5f may then be coupled with borate compound 5g under known Suzuki coupling conditions to obtain compounds of Formula (I-E), as shown in Scheme 5.

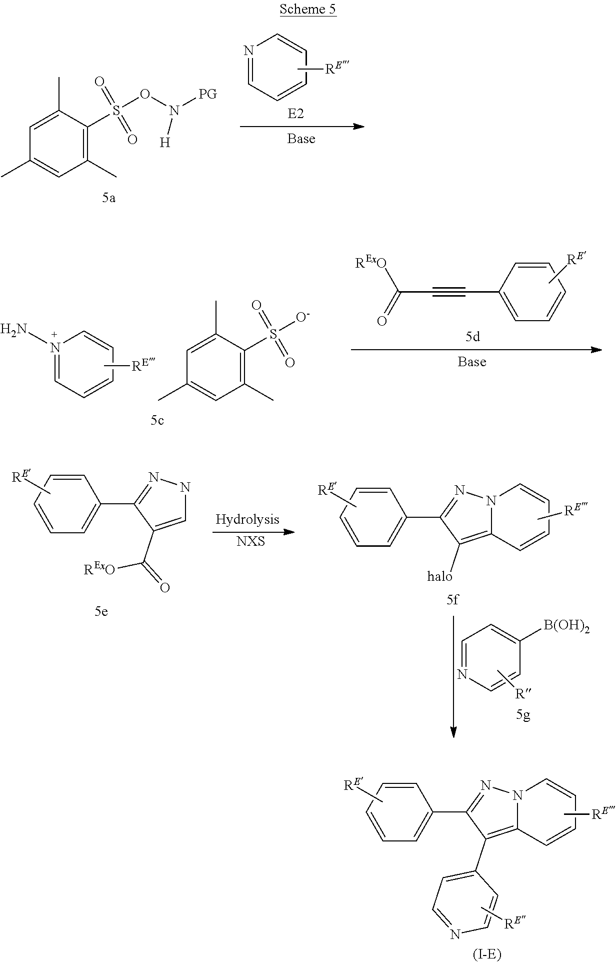

Similar processes may be conducted for compounds where the pyridine group is a pyrimidine as shown in Formula (I-F)

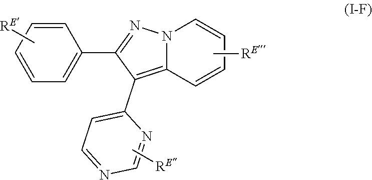

or where the pyridine is attached at the 4-position rather than the 3-position as shown in Formula (I-G)

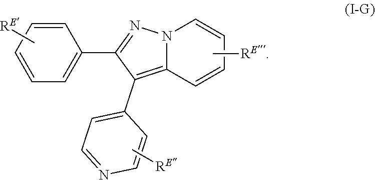

In some embodiments, R^{E′} is selected from H, halo, C₁₋₆alkyl and C₁₋₆haloalkyl. In some embodiments, one of R^{E″} and R^{E″′} is selected from H, halo, C₁₋₆alkyl, C₁₋₆haloalkyl and C₃₋₁₀heteroaryl optionally substituted with C₁₋₆alkyl or C₃₋₆cycloalkyl, and the other is L¹-A. In some embodiments, R^{E′}, and one of R^{E″} and R^{E″′} are independently selected from H, F, CH₃, CH₂CH₃, CF₂H, CF₃, CFH₂, CH₂CF₂H CH₂CF₃ and pyrazole. In some embodiments, R^{E′}, one of R^{E″} and R^{E″′} are independently selected from H, F, CH₃ and

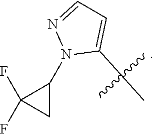 [SMILES: *c1ccnn1C1CC1(F)F]

In some embodiments, R^{E′}, R^{E″}, R^{E″′}, L¹ and A are as defined for the corresponding groups in compounds I-87, I-88, I-100 to I-187 and I-243 shown in Table 2.

Throughout the processes it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994).

Preparation of isotopic variants of the compounds of the application can be done using methods known in the art, for example, using reagents that are isotopically enriched with an isotopic variant, including for example deuterated Al-based reducing agents such as lithium aluminum deuteride, deuterated alkylating reagents and the like.

Techniques for purification of intermediates and final products include, for example, straight and reversed phase chromatography on column or rotating plate, recrystallisation, distillation and liquid-liquid or solid-liquid extraction, which will be readily understood by one skilled in the art.

Salts of the compounds of the application are generally formed by dissolving the neutral compound in an inert organic solvent and adding either the desired acid or base and isolating the resulting salt by either filtration or other known means.

The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”.

Prodrugs of the compounds of the present application may be, for example, conventional esters formed with available hydroxy, thiol, amino or carboxyl groups. For example, available hydroxy or amino groups may be acylated using an activated acid in the presence of a base, and optionally, in inert solvent (e.g. an acid chloride in pyridine). Some common esters which have been utilized as prodrugs are phenyl esters, aliphatic (C₁-C₂₄) esters, acyloxymethyl esters, carbamates and amino acid esters.

The following non-limiting examples are illustrative of the present application.

### EXAMPLES

### General Methods

All starting materials used herein were commercially available or earlier described in the literature. The ¹H and ¹³C NMR spectra were recorded either on Bruker 300, Bruker DPX400 or Varian+400 spectrometers operating at 300, 400 and 400 MHz for ¹H NMR respectively, using TMS or the residual solvent signal as an internal reference, in deuterated chloroform as solvent unless otherwise indicated. All reported chemical shifts are in ppm on the delta-scale, and the fine splitting of the signals as appearing in the recordings is generally indicated, for example as s: singlet, br s: broad singlet, d: doublet, t: triplet, q: quartet, m: multiplet. Unless otherwise indicated, in the tables below, ¹H NMR data was obtained at 400 MHz, using CDCl₃ as the solvent.

Purification of products was carried out using Chem Elut Extraction Columns (Varian, cat #1219-8002), Mega BE-SI (Bond Elut Silica) SPE Columns (Varian, cat #12256018; 12256026; 12256034) or by flash chromatography in silica-filled glass columns.

### Preparation of Intermediates and Specific Examples

### Example 1—Synthesis of Compound 6-(2-(2,4-difluorophenyl)-2-hydroxy-3-(1H-imidazol-1-yl)propyl)-4-(3-(4-fluorophenyl)-1-methyl-1H-pyrazol-4-yl)-6,7-dihydro-5H-pyrrolo[3,4-b]pyridin-5-one (I-1)

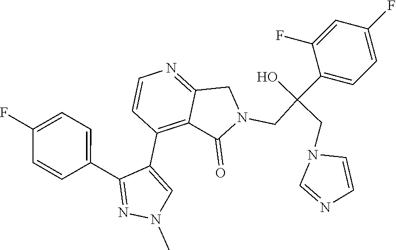 [SMILES: Cn1cc(-c2ccnc3c2C(=O)N(CC(O)(Cn2ccnc2)c2ccc(F)cc2F)C3)c(-c2ccc(F)cc2)n1]

Procedures for the synthesis of 6-(2-(2,4-difluorophenyl)-2-hydroxy-3-(1H-imidazol-1-yl)propyl)-4-(3-(4-fluorophenyl)-1-methyl-1H-pyrazol-4-yl)-6,7-dihydro-5H-pyrrolo[3,4-b]pyridin-5-one are shown in Scheme 6:

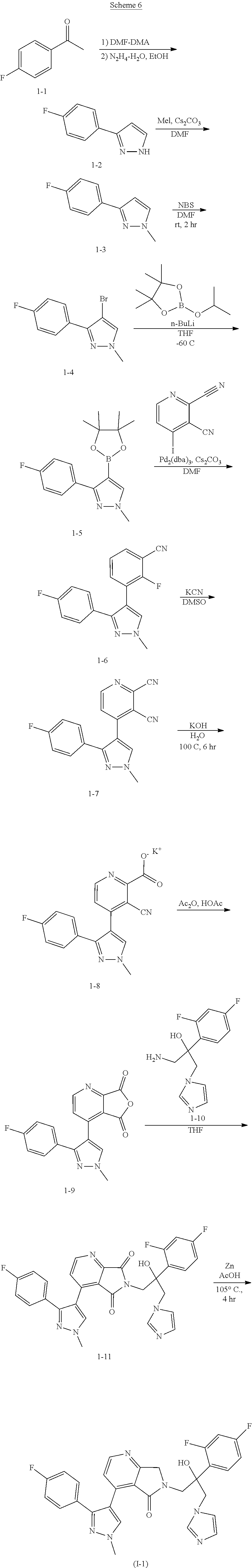

### 3-(4-fluorophenyl)-1H-pyrazole (1-2)

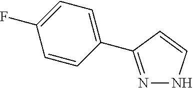 [SMILES: Fc1ccc(-c2cc[nH]n2)cc1]

To 1-(4-fluorophenyl)ethan-1-one 1-1 (100 g, 725 mmol) was added DMF-DMA (100 g, 847 mmol). The reaction was stirred at 100° C. for 16 hours with nitrogen protection. Then the reaction was concentrated and evaporated into dryness to give a reddish brown solid. The product was redissolved in 500 mL ethanol, and 40 mL hydrazine monohydrate was added thereto and heated at 70° C. for 4 hours. The reaction solution was poured into water, extracted with ethyl acetate and dried over sodium sulfate, and the solvent was evaporated. The product was recrystallized from ethyl acetate and hexane to give 110 g pale yellow crystals of intermediate 1-2. ¹H-NMR (CDCl₃) δ: 6.57 (d, J=2.4 Hz, 1H), 7.05-7.12 (m, 2H), 7.60 (d, J=2.4 Hz, 1H), 7.70-7.76 (m, 2H).

### 3-(4-fluorophenyl)-1-methyl-1H-pyrazole (1-3)

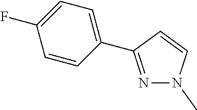

To a solution of 3-(4-fluorophenyl)pyrazole 1-2 (110 g, 679 mmol) in dry DMF (1000 mL) was added Cs₂CO₃ (453 g, 1358 mmol) at 0° C. for 15 min. iodomethane (50 mL, 815 mmol) was added and slowly warmed to room temperature. The reaction was allowed to stir at room temperature for 2 hours. The reaction mixture was diluted with water (1000 mL), extracted with EtOAc (3×1000 mL), dried (Na₂SO₄), concentrated and purified with flash column (dichloromethane:methanol 50:1) to give intermediate 1-3 as a white solid (50 g, 41.9% yield). MS (ESi+): 177 (M+H)+.

### 4-bromo-3-(4-fluorophenyl)-1-methyl-1H-pyrazole (1-4)

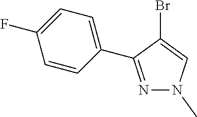

To a solution of 3-(4-fluorophenyl)-1-methyl-1H-pyrazole (50 g, 284 mmol) 1-3 in DMF (500 ml) was added NBS (50 g, 284 mmol). The reaction mixture was stirred at room temperature for 2 hours, quenched with water (500 ml), extracted with EtOAc (500 ml*3). The organic layer was washed with brine, dried over anhydrous sodium sulfate, and concentrated under reduced pressure to give 42 g of intermediate 1-4 as a white solid with 58.4% yield.

### 3-(4-fluorophenyl)-1-methyl-4-(4,4,5,5-tetramethyl-1,3,2-dioxaborolan-2-yl)-1H-pyrazole (1-5)

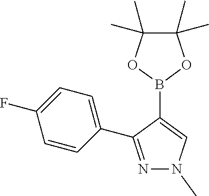 [SMILES: Cn1cc(B2OC(C)(C)C(C)(C)O2)c(-c2ccc(F)cc2)n1]

To a solution of 4-bromo-3-(4-fluorophenyl)-1-methyl-1H-pyrazole 1-4 (20 g, 79 mmol) in THF (200 mL) under nitrogen at −78° C. was added n-BuLi (hexane, 2.5 M, 35 mL, 87 mmol.) over 10 min. After 45 min, 2-isopropoxy-4,4,5,5-tetramethyl-1,3,2-dioxaborolane (17.8 mL, 87 mmol) was added. After 1 h 45 min, saturated aqueous NH₄Cl and EtOAc were added. The cold bath was removed, and the reaction mixture was stirred to room temperature. The layers were separated, and the organic layer was washed with brine, dried over Na₂SO₄, filtered, and concentrated in vacuo. Silica gel chromatography using hexanes:EtOAc (3:1) as eluent afforded 3-(2-fluorophenyl)-1-methyl-4-(4,4,5,5-tetramethyl-1,3,2-dioxaborolan-2-yl)-1H-pyrazole 1-5 as a white solid (8 g, 33.5% yield).

### 3-fluoro-4-(3-(4-fluorophenyl)-1-methyl-1H-pyrazol-4-yl)picolinonitrile (1-6)

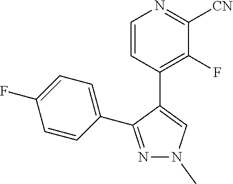 [SMILES: Cn1cc(-c2ccnc(C#N)c2F)c(-c2ccc(F)cc2)n1]

3-(4-fluorophenyl)-1-methyl-4-(4,4,5,5-tetramethyl-1,3,2-dioxaborolan-2-yl)-1H-pyrazole 1-5 (8 g, 26.5 mmol) and 3-fluoro-4-iodopicolinonitrile (5.5 g, 21 mmol) were combined in 100 mL DMF, and treated with Cs₂CO₃ (13.2 g, 40.8 mmol). The resulting suspension was sparged with nitrogen for 20 min and treated with tris(dibenzylideneacetone)dipalladium(0) (1 g, 1.1 mmol) in a single portion. The nitrogen sparging was continued for an additional 20 min, and stirring of the dark suspension was continued for 30 min at room temperature. The reaction was warmed to 50° C. for 6 hours and was allowed to cool to room temperature. The thick slurry was added to 150 mL EtOAc, filtered through Celite™, and the organic layer was washed with 3×30 mL brine. The organic layer was dried over anhydrous Na₂SO₄ and was concentrated in vacuo to give 12 g of a crude solid. The solid was purified by flash column with a 5-80% EtOAc/heptane gradient to afford 5.8 g of intermediate 1-6 as a white solid with 74% yield. MS m/z 297.0 (M+1); ¹H NMR (400 MHz, CDCl₃) δ 8.28 (d, J=5.1, 1H), 7.84 (d, J=2.9, 1H), 7.40 (dd, J=5.3, 2.2, 2H), 7.25 (dd, J=5.8, 4.9, 1H), 7.09 (dd, J=8.6, 8.6, 2H), 4.02 (s, 3H).

### 4-(3-(4-fluorophenyl)-1-methyl-1H-pyrazol-4-yl)pyridine-2,3-dicarbonitrile (1-7)

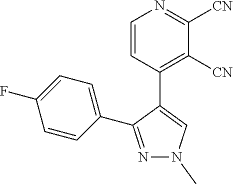 [SMILES: Cn1cc(-c2ccnc(C#N)c2C#N)c(-c2ccc(F)cc2)n1]

To 3-fluoro-4-(3-(4-fluorophenyl)-1-methyl-1H-pyrazol-4-yl)picolinonitrile 1-6 (5.8 g, 19.6 mmol) in DMSO (60 mL) with gentle heating was added potassium cyanide (1.36 g, 20.3 mmol) and heated to 50° C. and stirred. After 2 hours, the mixture was cooled in an ice bath and 0.1N aqueous NaOH (50 mL) was added. The resulting slurry was stirred for 5 min, solids were collected, rinsed with water and air dried to afford 4 g of compound 1-7 as a light cream colored solid with 67% yield. ¹H NMR (400 MHz, CDCl₃) δ 8.56 (d, J=5.2, 1H), 8.02 (s, 1H), 7.33 (dd, J=8.9, 5.2, 2H), 7.24 (d, J=5.5, 1H), 7.06 (dd, J=8.6, 8.6, 2H), 4.02 (s, 3H).

### 4-(3-(4-fluorophenyl)-1-methyl-1H-pyrazol-4-yl)pyridine-2,3-dicarboxylic acid dipotassium salt (1-8)

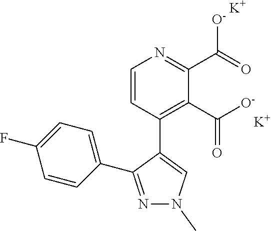 [SMILES: [K+] ; Cn1cc(-c2ccnc(C(=O)[O-])c2C(=O)[O-])c(-c2ccc(F)cc2)n1 ; [K+]]

4-(3-(4-fluorophenyl)-1-methyl-1H-pyrazol-4-yl)pyridine-2,3-dicarbonitrile 1-7 (4 g, 13.2 mmol) and aqueous KOH (14.7 g, 262 mmol) were dissolved in 100 mL of water and heated at 100° C. for 24 hours. The mixture was cooled in ice to precipitate a thick white solid. The material was warmed back to room temperature, filtered and air dried to yield 5 g (91%) of compound 1-8 as a cream colored solid: LCMS m/z 342.1 (M+1).

### 4-(3-(4-fluorophenyl)-1-methyl-1H-pyrazol-4-yl)furo[3,4-b]pyridine-5,7-dione (1-9)

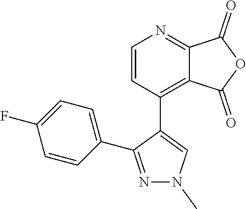 [SMILES: Cn1cc(-c2ccnc3c2C(=O)OC3=O)c(-c2ccc(F)cc2)n1]

4-(3-(4-fluorophenyl)-1-methyl-1H-pyrazol-4-yl)pyridine-2,3-dicarboxylic acid dipotassium salt (1.00 g, 2.4 mmol) was slurred with acetic acid (7 mL) and acetic anhydride (2.5 mL) and heated to 110° C. for 4 hours. The resulting homogeneous mixture of compound 1-9 was cooled and directly used for the next step.

### 6-(2-(2,4-difluorophenyl)-2-hydroxy-3-(1H-imidazol-1-yl)propyl)-4-(3-(4-fluorophenyl)-1-methyl-1H-pyrazol-4-yl)-5H-pyrrolo[3,4-b]pyridine-5,7(6H)-dione (1-11)

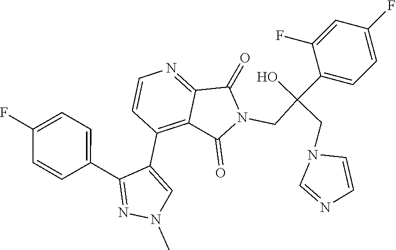 [SMILES: Cn1cc(-c2ccnc3c2C(=O)N(CC(O)(Cn2ccnc2)c2ccc(F)cc2F)C3=O)c(-c2ccc(F)cc2)n1]

To the reaction mixture from the previous step comprising compound 1-9 was added 1-amino-2-(2,4-difluorophenyl)-3-(1H-imidazol-1-yl)propan-2-ol 1-10 (0.167 mL, 1.53 mmol) in THF (4.7 mL). The mixture was heated at reflux for 16 hours, cooled and then concentrated to a brown solid. This material was purified with reverse-phase column with H₂O/MeOH 5%-95% to afford 110 mg product 1-11 as a yellow colored solid with 8.2% yield for 2 steps.: LCMS m/z 559 (M+1).

### 6-(2-(2,4-difluorophenyl)-2-hydroxy-3-(1H-imidazol-1-yl)propyl)-4-(3-(4-fluorophenyl)-1-methyl-1H-pyrazol-4-yl)-6,7-dihydro-5H-pyrrolo[3,4-b]pyridin-5-one (I-1)

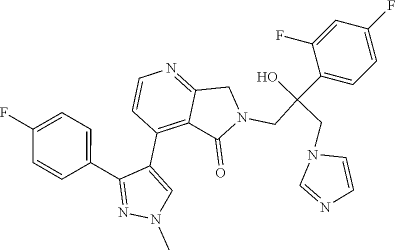 [SMILES: Cn1cc(-c2ccnc3c2C(=O)N(CC(O)(Cn2ccnc2)c2ccc(F)cc2F)C3)c(-c2ccc(F)cc2)n1]

To a flask equipped with overhead stirring was added 6-(2-(2,4-difluorophenyl)-2-hydroxy-3-(1H-imidazol-1-yl)propyl)-4-(3-(4-fluorophenyl)-1-methyl-1H-pyrazol-4-yl)-5H-pyrrolo[3,4-b]pyridine-5,7(6H)-dione 1-11 (110 mg, 0.2 mmol) and zinc dust (<10 micron) (111 mg, 2 mmol) followed by acetic acid (3 mL). The mixture was heated to 105° C. and stirred for 4.5 hours, then cooled to room temperature. Celite was added to the reaction mixture, and this was filtered to remove the zinc. The filter pad was rinsed with EtOAc and the filtrate was concentrated and purified with reverse-phase column with H₂O/MeOH 5%-95% to afford 10 mg of compound 1-1 as a white solid with 9.2% yield. LCMS m/z 545 (M+1); ¹H NMR (400 MHz, Methanol-d₄) δ 8.46 (d, J=5.3 Hz, 1H), 8.03 (s, 1H), 7.45-7.37 (m, 2H), 7.36-7.32 (m, 2H), 7.09 (d, J=5.3 Hz, 1H), 7.03 (dd, J=9.9, 7.7 Hz, 3H), 6.87 (s, 1H), 6.84 (d, J=2.6 Hz, 1H), 6.74 (s, 1H), 4.55 (q, J=8.2, 6.8 Hz, 4H), 4.28 (dd, J=14.5, 7.1 Hz, 2H), 4.00 (s, 3H), 3.91 (d, J=14.6 Hz, 1H).

### Example 2—Synthesis of Compound 4-(1-(3-(2,4-difluorophenyl)-3-hydroxy-4-(1H-1,2,4-triazol-1-yl)butyl)-3-(4-fluorophenyl)-1H-pyrazol-4-yl)-6-methyl-6,7-dihydro-5H-pyrrolo[3,4-b]pyridin-5-one (I-2)

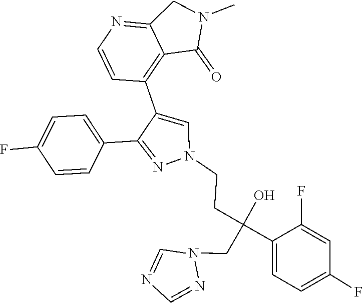 [SMILES: CN1Cc2nccc(-c3cn(CCC(O)(Cn4cncn4)c4ccc(F)cc4F)nc3-c3ccc(F)cc3)c2C1=O]

Procedures for the synthesis of compound 4-(1-(3-(2,4-difluorophenyl)-3-hydroxy-4-(1H-1,2,4-triazol-1-yl)butyl)-3-(4-fluorophenyl)-1H-pyrazol-4-yl)-6-methyl-6,7-dihydro-5H-pyrrolo[3,4-b]pyridin-5-one (I-2) are shown below in Scheme 7:

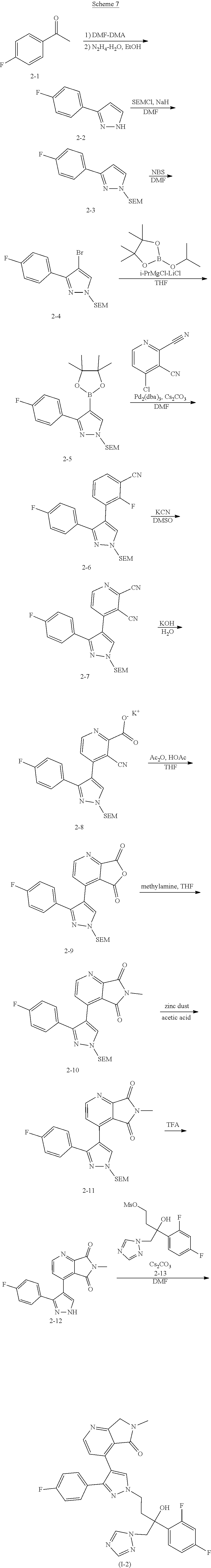

### 3-(4-fluorophenyl)-1H-pyrazole (2-2)

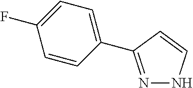 [SMILES: Fc1ccc(-c2cc[nH]n2)cc1]

To 1-(4-fluorophenyl)ethan-1-one 2-1 (100 g, 725 mmol) was added DMF-DMA (100 g, 847 mmol). The reaction was stirred at 100° C. for 16 hours with nitrogen protection. Then the reaction was concentrated and evaporated into dryness to give a reddish brown solid. The product was redissolved in 500 mL ethanol, and 40 mL hydrazine monohydrate was added thereto and heated at 70° C. for 4 hours. The reaction solution was poured into water, extracted with ethyl acetate and dried over sodium sulfate, and the solvent was evaporated. The product was recrystallized from ethyl acetate and hexane to give 110 g pale yellow crystals of compound 2-2. ¹H-NMR (CDCl₃) δ: 6.57 (d, J=2.4 Hz, 1H), 7.05-7.12 (m, 2H), 7.60 (d, J=2.4 Hz, 1H), 7.70-7.76 (m, 2H).

### 3-(4-fluorophenyl)-1-((2-(trimethylsilyl)ethoxy)methyl)-1H-pyrazole (2-3)

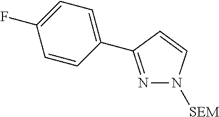 [SMILES: C[Si](C)(C)CCOCn1ccc(-c2ccc(F)cc2)n1]

To a solution of 3-(4-fluorophenyl)-1H-pyrazole (10 g, 61.7 mmol) in anhydrous N,N-dimethylformamide (100 ml) was slowly added NaH (3.2 g, 67.9 mmol, 60% in mineral oil) at room temperature. The suspension was stirred for 15 minutes and 2-(Trimethylsilyl)ethoxymethyl chloride (12.8 ml, 61.7 mmol) was added. The mixture was stirred at room temperature for 3 hours. The reaction mixture was diluted with water (250 mL) and extracted with ethyl acetate (3×50 mL). The combined organic layer was washed with water (50 mL), brine, dried over MgSO₄, filtered and concentrated in vacuo to yield product 2-3 with its isomer as a yellow waxy solid (15.3 g) with 85% yield.

### 4-bromo-3-(4-fluorophenyl-1-((2-(trimethylsilyl)ethoxy)methyl)-1H-pyrazole (2-4)

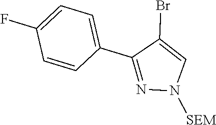 [SMILES: C[Si](C)(C)CCOCn1cc(Br)c(-c2ccc(F)cc2)n1]

To a solution of 3-(4-fluorophenyl)-1-((2-(trimethylsilyl)ethoxy)methyl)-1H-pyrazole 2-3 (15.3 g, 52.4 mmol) in DMF (100 ml) was added NBS (9.2 g, 52.4 mmol). The reaction mixture was stirred at room temperature for 2 hours, quenched with water (100 mL), extracted with EtOAc (100 mL*3). The organic layer was washed with brine, dried over anhydrous sodium sulfate, and concentrated under reduced pressure to give 18 g of compound 2-4 as a white solid with 92.8% yield.

### 3-(4-fluorophenyl)-4-(4,4,5,5-tetramethyl-1,3,2-dioxaborolan-2-yl)-1-((2-(trimethylsilyl)ethoxy)methyl)-1H-pyrazole (2-5)

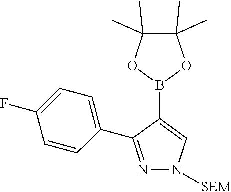 [SMILES: CC1(C)OB(c2cn(COCC[Si](C)(C)C)nc2-c2ccc(F)cc2)OC1(C)C]

To a solution of 4-bromo-3-(4-fluorophenyl)-1-((2-(trimethylsilyl)ethoxy)methyl)-1H-pyrazole 2-4 (18 g, 48.6 mmol) in THF (200 mL) under nitrogen at −78° C. was added isopropylmagnesium chloride lithium chloride complex (1.3 M, 49 mL, 63.2 mmol) over 10 min. After 45 min, 2-isopropoxy-4,4,5,5-tetramethyl-1,3,2-dioxaborolane (12 mL, 58.3 mmol) was added. After 1 h 45 min, saturated aqueous NH₄Cl and EtOAc were added. The cold bath was removed, and the reaction mixture was stirred to room temperature. The layers were separated, and the organic layer was washed with brine, dried over Na₂SO₄, filtered, and concentrated in vacuo. Silica gel chromatography using hexanes:EtOAc (3:1) as eluent afforded compound 2-5 as a white solid (10 g, 49.2% yield).

### 3-fluoro-4-(3-(4-fluorophenyl)-1-((2-(trimethylsilyl)ethoxy)methyl)-1H-pyrazol-4-yl)picolinonitrile (2-6)

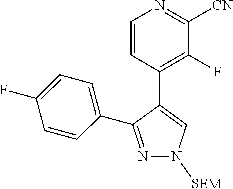 [SMILES: C[Si](C)(C)CCOCn1cc(-c2ccnc(C#N)c2F)c(-c2ccc(F)cc2)n1]

3-(4-fluorophenyl)-4-(4,4,5,5-tetramethyl-1,3,2-dioxaborolan-2-yl)-1-((2-(trimethylsilyl)ethoxy)methyl)-1H-pyrazole 2-5 (10 g, 23.9 mmol) and 3-fluoro-4-iodopicolinonitrile (5.0 g, 19 mmol) were combined in 100 mL DMF and treated with Cs₂CO₃ (12.0 g, 37.1 mmol). The resulting suspension was sparged with nitrogen for 20 min and treated with tris(dibenzylideneacetone)dipalladium(0) (0.91 g, 1.0 mmol) in a single portion. The nitrogen sparging was continued for an additional 20 min, and stirring of the dark suspension was continued for 30 min at room temperature. The reaction was warmed to 50° C. for 6 hours and was allowed to cool to room temperature. The thick slurry was added to 150 mL EtOAc, filtered through Celite, and the organic layer was washed with 3×30 mL brine. The organic layer was dried over anhydrous Na₂SO₄ and was concentrated in vacuo to give 12 g of a crude solid. The solid was purified by flash column with a 5-80% EtOAc/heptane gradient to afford 9.0 g of compound 2-6 as a white solid with 91% yield. MS m/z 413.0 (M+1).

### 4-(3-(4-fluorophenyl)-1-((2-(trimethylsilyl)ethoxy)methyl)-1H-pyrazol-4-yl)pyridine-2,3-dicarbonitrile (2-7)

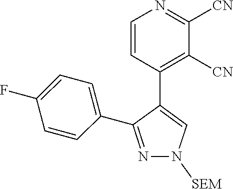 [SMILES: C[Si](C)(C)CCOCn1cc(-c2ccnc(C#N)c2C#N)c(-c2ccc(F)cc2)n1]

To a solution of 3-fluoro-4-(3-(4-fluorophenyl)-1-((2-(trimethylsilyl)ethoxy)methyl)-1H-pyrazol-4-yl)picolinonitrile 2-6 (9 g, 21.8 mmol) in DMSO (50 mL) with gentle heating was added potassium cyanide (1.52 g, 22.6 mmol) and the mixture was heated to 50° C. and stirred. After 2 hours, the mixture was cooled in an ice bath and 0.1N aqueous NaOH (50 mL) was added. The resulting slurry was stirred for 5 min, solids were collected, rinsed with water and air dried to afford 7.3 g of compound 2-7 as a light cream colored solid with 80% yield.

### 4-(3-(4-fluorophenyl)-1-((2-(trimethylsilyl)ethoxy)methyl)-1H-pyrazol-4-yl)pyridine-2,3-dicarboxylate acid dipotassium salt (2-8)

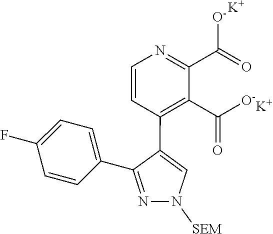 [SMILES: [K+] ; C[Si](C)(C)CCOCn1cc(-c2ccnc(C(=O)[O-])c2C(=O)[O-])c(-c2ccc(F)cc2)n1 ; [K+]]

4-(3-(4-fluorophenyl)-1-((2-(trimethylsilyl)ethoxy)methyl)-1H-pyrazol-4-yl)pyridine-2,3-dicarbonitrile 2-7 (7 g, 17.4 mmol) and aqueous KOH (9.8 g, 174 mmol, were dissolved in 100 mL water and heated at 100° C. for 24 hours. The mixture was cooled in ice to precipitate a thick white solid. The material was warmed back to room temperature, filtered and air dried to yield 4.2 g (45.3%) of product 2-8 as a cream colored solid: LCMS m/z 456 (M−1).

### 4-(3-(4-fluorophenyl)-1-((2-(trimethylsilyl)ethoxy)methyl)-1H-pyrazol-4-yl)furo[3,4-b]pyridine-5,7-dione (2-9)

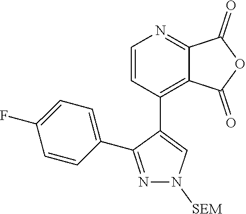 [SMILES: C[Si](C)(C)CCOCn1cc(-c2ccnc3c2C(=O)OC3=O)c(-c2ccc(F)cc2)n1]

4-(3-(4-fluorophenyl)-1-((2-(trimethylsilyl)ethoxy)methyl)-1H-pyrazol-4-yl)pyridine-2,3-dicarboxylate acid dipotassium salt 2-8 (4.2 g, 7.9 mmol) was slurried with acetic acid (7 mL) and acetic anhydride (2.5 mL) and heated to 110° C. for 4 hours. The resulting homogeneous mixture containing intermediate 2-9 was cooled and directly used for the next step.

### 4-(3-(4-fluorophenyl)-1-((2-(trimethylsilyl)ethoxy)methyl)-1H-pyrazol-4-yl)-6-methyl-5H-pyrrolo[3,4-b]pyridine-5,7(6H)-dione (2-10)

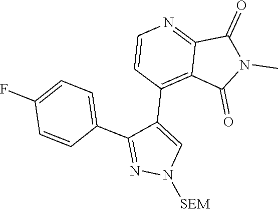 [SMILES: CN1C(=O)c2nccc(-c3cn(COCC[Si](C)(C)C)nc3-c3ccc(F)cc3)c2C1=O]

To the reaction mixture from the previous step comprising compound 2-9 was added methylamine in THF (4 mL, 2.0M 7.9 mmol) and the mixture was heated at reflux for 16 hours, cooled and then concentrated to a brown solid. This material was purified with reverse-phase column with H₂O/MeOH 5%-95% to afford 1.2 g of compound 2-10 as a yellow colored solid with 33.6% yield for 2 steps.: LCMS m/z 453 (M+1).

### 4-(3-(4-fluorophenyl)-1-((2-(trimethylsilyl)ethoxy)methyl)-1H-pyrazol-4-yl)-6-methyl-6,7-dihydro-5H-pyrrolo[3,4-b]pyridin-5-one (2-11)

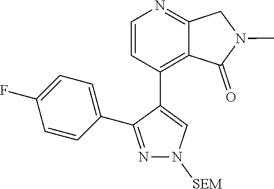 [SMILES: CN1Cc2nccc(-c3cn(COCC[Si](C)(C)C)nc3-c3ccc(F)cc3)c2C1=O]

To a flask equipped with overhead stirring was added 4-(3-(4-fluorophenyl)-1-((2-(trimethylsilyl)ethoxy)methyl)-1H-pyrazol-4-yl)-6-methyl-5H-pyrrolo[3,4-b]pyridine-5,7(6H)-dione 2-10 (1.2 g, 2.7 mmol) and zinc dust (<10 micron) (877 mg, 13.5 mmol) followed by acetic acid (20 mL). The mixture was heated to 105° C. and stirred for 4 hours, then cooled to room temperature. Celite was added to the reaction mixture, and this was filtered to remove the zinc. The filter pad was rinsed with EtOAc and the filtrate was concentrated and purified by reverse-phase column with H₂O/MeOH 5%-95% to afford 300 mg of compound 2-11 as a white solid with 25% yield. LCMS m/z 439 (M+1).

### 4-(3-(4-fluorophenyl)-1H-pyrazol-4-yl)-6-methyl-6,7-dihydro-5H-pyrrolo[3,4-b]pyridin-5-one (2-12)

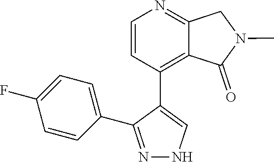 [SMILES: CN1Cc2nccc(-c3c[nH]nc3-c3ccc(F)cc3)c2C1=O]

A stirred solution of 4-(3-(4-fluorophenyl)-1-((2-(trimethylsilyl)ethoxy)methyl)-1H-pyrazol-4-yl)-6-methyl-6,7-dihydro-5H-pyrrolo[3,4-b]pyridin-5-one 2-11 (300 mg, 0.68 mmol) in DCM (2.0 ml) at ambient temperature was treated with TFA (2.0 ml) and the resulting mixture was stirred for 4 hours. The mixture was concentrated in vacuo and the residue purified with reverse-phase column with H₂O/MeOH 5%-95% to afford 200 mg of compound 2-12 as a white solid with 95% yield.

### 4-(1-(3-(2,4-difluorophenyl)-3-hydroxy-4-(1H-1,2,4-triazol-1-yl)butyl)-3-(4-fluorophenyl)-1H-pyrazol-4-yl)-6-methyl-6,7-dihydro-5H-pyrrolo[3,4-b]pyridin-5-one (I-2)

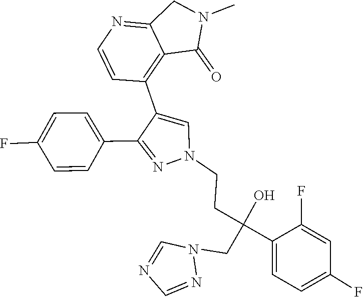 [SMILES: CN1Cc2nccc(-c3cn(CCC(O)(Cn4cncn4)c4ccc(F)cc4F)nc3-c3ccc(F)cc3)c2C1=O]

A solution of 4-(3-(4-fluorophenyl)-1H-pyrazol-4-yl)-6-methyl-6,7-dihydro-5H-pyrrolo[3,4-b]pyridin-5-one 2-12 (100 mg, 0.32 mmol) in DMF (3 ml) was treated with 3-(2,4-difluorophenyl)-3-hydroxy-4-(1H-1,2,4-triazol-1-yl)butyl methanesulfonate 2-13 (174 mg, 0.5 mmol) and cesium carbonate (325 mg, 1 mmol). The mixture was stirred at room temperature for 16 hours to completion. The reaction was directly purified by reverse-phase column with H₂O/MeOH 5%-95% to afford 54 mg of compound (I-2) as a white solid with 30.2% yield. ¹H NMR (400 MHz, Methanol-d₄) δ 8.32 (d, J=5.4 Hz, 1H), 8.27 (s, 1H), 8.03 (s, 1H), 7.76 (s, 1H), 7.36 (td, J=9.1, 6.7 Hz, 1H), 7.23-7.18 (m, 2H), 7.12 (t, J=8.7 Hz, 2H), 6.87-6.77 (m, 3H), 4.63-4.49 (m, 2H), 4.45 (s, 2H), 4.15 (ddd, J=13.9, 10.4, 5.7 Hz, 1H), 3.81-3.73 (m, 1H), 3.16 (s, 3H), 2.53 (td, J=12.7, 11.1, 5.0 Hz, 1H), 2.35-2.27 (m, 1H).

### Example 3—Preparation of (R)-6-(2-(1-(2-(2,4-difluorophenyl)-2-hydroxy-3-(1H-1,2,4-triazol-1-yl)propyl)piperidin-4-yl)ethyl)-4-(3-(4-fluorophenyl)-1-methyl-1H-pyrazol-4-yl)-6,7-dihydro-5H-pyrrolo[3,4-b]pyridin-5-one (I-247) and (S)-6-(2-(1-(2-(2,4-difluorophenyl)-2-hydroxy-3-(1H-1,2,4-triazol-1-yl)propyl)piperidin-4-yl)ethyl)-4-(3-(4-fluorophenyl)-1-methyl-1H-pyrazol-4-yl)-6,7-dihydro-5H-pyrrolo[3,4-b]pyridin-5-one (I-248)

The preparation of compounds 1-247 and 1-248 is made by chiral separation as shown below in Scheme 8:

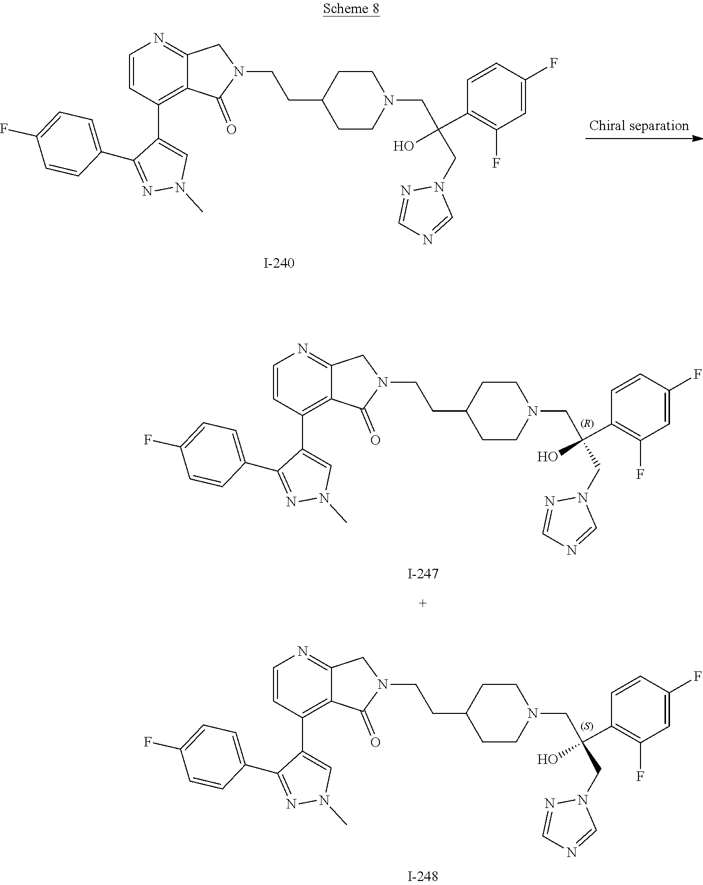

### (R)-6-(2-(1-(2-(2,4-difluorophenyl)-2-hydroxy-3-(1H-1,2,4-triazol-1-yl)propyl)piperidin-4-yl)ethyl)-4-(3-(4-fluorophenyl)-1-methyl-1H-pyrazol-4-yl)-6,7-dihydro-5H-pyrrolo[3,4-b]pyridin-5-one (I-247) 4 and (S)-6-(2-(1-(2-(2,4-difluorophenyl)-2-hydroxy-3-(1H-1,2,4-triazol-1-yl)propyl)piperidin-4-yl)ethyl)-4-(3-(4-fluorophenyl)-1-methyl-1H-pyrazol-4-yl)-6,7-dihydro-5H-pyrrolo[3,4-b]pyridin-5-one (I-248)

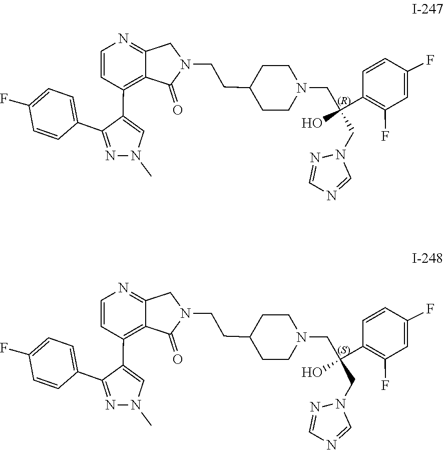

The chiral separation of 6-(2-(1-(2-(2,4-difluorophenyl)-2-hydroxy-3-(1H-1,2,4-triazol-1-yl)propyl)piperidin-4-yl)ethyl)-4-(3-(4-fluorophenyl)-1-methyl-1H-pyrazol-4-yl)-6,7-dihydro-5H-pyrrolo[3,4-b]pyridin-5-one (I-240) was carried out by instrument: SFC-150 mgm (waters). Column: Daicel AD-3 (25*250 mm, 10 um); Mobile phase: CO2/MeOH [0.2% NH₃ (7M in MeOH)]=50/50; Flow rate: 100 ml/min; Back pressure: 100 bar; Detection wavelength: 214 nm; Cycle time: 15 min; Sample solution: 350 mg dissolved in 9 ml MeOH; Injection volume: 4 ml

The peak 1 (1st peak) was compound (R)-6-(2-(1-(2-(2,4-difluorophenyl)-2-hydroxy-3-(1H-1,2,4-triazol-1-yl)propyl)piperidin-4-yl)ethyl)-4-(3-(4-fluorophenyl)-1-methyl-1H-pyrazol-4-yl)-6,7-dihydro-5H-pyrrolo[3,4-b]pyridin-5-one (I-247)(1.04 min, 100% ee).

LCMS: Calculated Exact Mass=656.3, Found [M+H]+ (ESI+)=657.2. ¹H NMR (400 MHz, DMSO) δ 8.50 (d, J=5.2 Hz, 1H), 8.30 (s, 1H), 8.15 (s, 1H), 7.74 (s, 1H), 7.40 (dd, J=15.9, 8.9 Hz, 1H), 7.34 (dd, J=8.7, 5.6 Hz, 2H), 7.20-7.10 (m, 3H), 7.01-6.92 (m, 2H), 5.59 (s, 1H), 4.60-4.49 (m, 2H), 4.45 (s, 2H), 3.94 (s, 3H), 3.47 (t, J=7.4 Hz, 2H), 2.82 (d, J=14.2 Hz, 1H), 2.65 (d, J=13.7 Hz, 2H), 2.56 (d, J=11.5 Hz, 1H), 2.13-1.96 (m, 2H), 1.63-1.39 (m, 4H), 1.05 (dd, J=20.8, 8.9 Hz, 3H).

The peak 2 (2nd peak) was compound (S)-6-(2-(1-(2-(2,4-difluorophenyl)-2-hydroxy-3-(1H-1,2,4-triazol-1-yl)propyl)piperidin-4-yl)ethyl)-4-(3-(4-fluorophenyl)-1-methyl-1H-pyrazol-4-yl)-6,7-dihydro-5H-pyrrolo[3,4-b]pyridin-5-one (I-248) (2.73 min, 100% ee).

LCMS: Calculated Exact Mass=656.3, Found [M+H]+ (ESI+)=657.2. ¹H NMR (400 MHz, DMSO) δ 8.50 (d, J=5.2 Hz, 1H), 8.30 (s, 1H), 8.15 (s, 1H), 7.74 (s, 1H), 7.40 (dd, J=15.9, 8.9 Hz, 1H), 7.34 (dd, J=8.7, 5.6 Hz, 2H), 7.20-7.10 (m, 3H), 7.01-6.92 (m, 2H), 5.59 (s, 1H), 4.60-4.49 (m, 2H), 4.45 (s, 2H), 3.94 (s, 3H), 3.47 (t, J=7.4 Hz, 2H), 2.82 (d, J=14.2 Hz, 1H), 2.65 (d, J=13.7 Hz, 2H), 2.56 (d, J=11.5 Hz, 1H), 2.13-1.96 (m, 2H), 1.63-1.39 (m, 4H), 1.05 (dd, J=20.8, 8.9 Hz, 3H).

### Example 4—Synthesis of 2-(2,4-difluorophenyl)-1,3-di(1H-1,2,4-triazol-1-yl)propan-2-yl 2-(4-((4-(3-(4-fluorophenyl)-1-methyl-1H-pyrazol-4-yl)-5-oxo-5,7-dihydro-6H-pyrrolo[3,4-b]pyridin-6-yl)methyl)piperidin-1-yl)acetate (I-250)

Procedures for the synthesis of compound 2-(2,4-difluorophenyl)-1,3-di(1H-1,2,4-triazol-1-yl)propan-2-yl 2-(4-((4-(3-(4-fluorophenyl)-1-methyl-1H-pyrazol-4-yl)-5-oxo-5,7-dihydro-6H-pyrrolo[3,4-b]pyridin-6-yl)methyl)piperidin-1-yl)acetate (I-250) are shown below in Scheme 9:

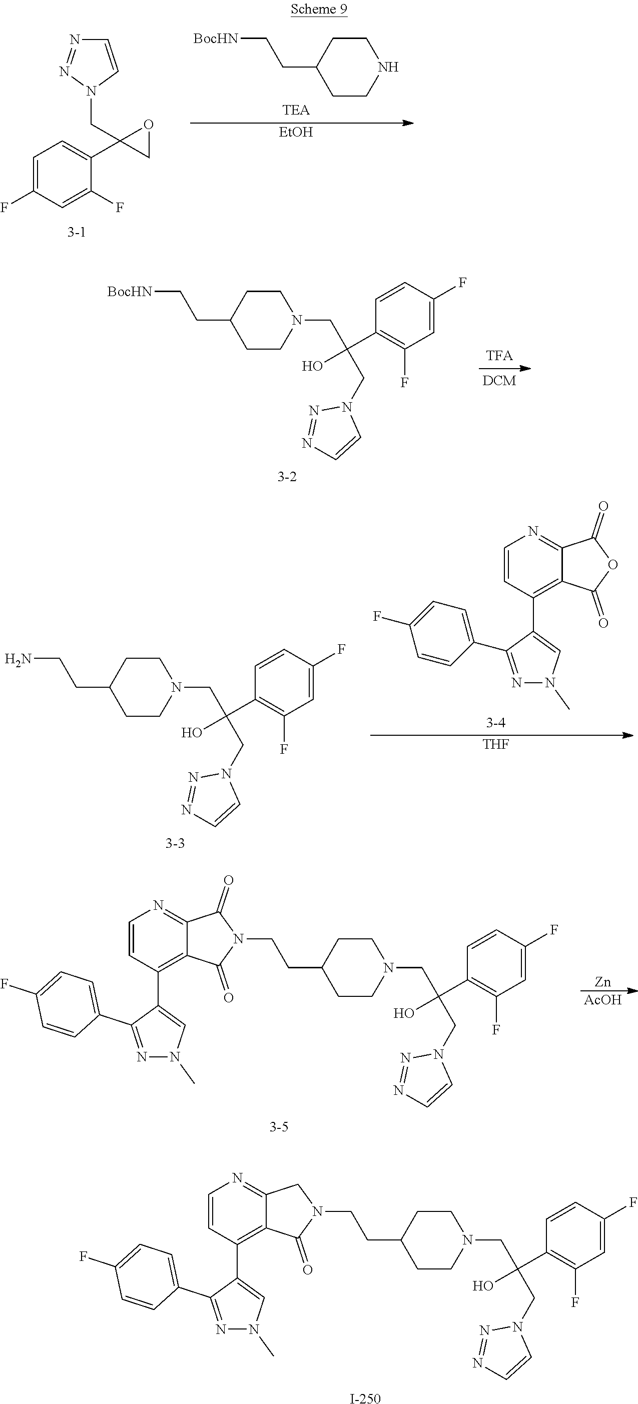

### tert-butyl (2-(1-(2-(2,4-difluorophenyl)-2-hydroxy-3-(1H-1,2,3-triazol-1-yl)propyl)piperidin-4-yl)ethyl)carbamate (3-2)

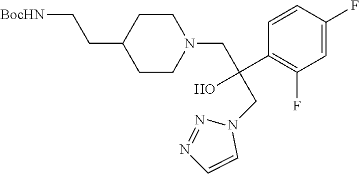 [SMILES: CC(C)(C)OC(=O)NCCC1CCN(CC(O)(Cn2ccnn2)c2ccc(F)cc2F)CC1]

1-((2-(2,4-difluorophenyl)oxiran-2-yl)methyl)-1H-1,2,3-triazole 3-1 (500 mg, 2.1 mmol) was dissolved in 5 mL EtOH with NEt₃ (638 mg, 6.3 mmol) and tert-butyl (2-(piperidin-4-yl)ethyl)carbamate (718 mg, 3.2 mmol). The reaction was stirred at 80° C. for 16 hours. Then the reaction was concentrated, diluted with 30 mL EtOAc, washed with 20 mL water twice and 30 mL brine. The organic layer was dried with Na₂SO₄, concentrated and purified by flash (DCM/MeOH=10/1) to afford tert-butyl (2-(1-(2-(2,4-difluorophenyl)-2-hydroxy-3-(1H-1,2,3-triazol-1-yl)propyl)piperidin-4-yl)ethyl)carbamate 3-2, 900 mg as a white solid with 91% yield. LCMS: Calculated Exact Mass=465.2, Found [M+H]+ (ESI+)=466.1.

### 1-(4-(2-aminoethyl)piperidin-1-yl)-2-(2,4-difluorophenyl)-3-(1H-1,2,3-triazol-1-yl)propan-2-ol (3-3)

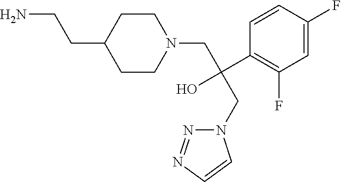 [SMILES: NCCC1CCN(CC(O)(Cn2ccnn2)c2ccc(F)cc2F)CC1]

tert-butyl (2-(1-(2-(2,4-difluorophenyl)-2-hydroxy-3-(1H-1,2,3-triazol-1-yl)propyl)piperidin-4-yl)ethyl)carbamate 3-2 (900 mg, 1.93 mmol) was dissolved in 10 mL DCM and added 2 mL TFA at 0° C. The mixture was stirred for 2 hours at room temperature. When the reaction was complete, the solvent was evaporated to afford crude product 3-3. LCMS: Calculated Exact Mass=365.2, Found [M+H]+ (ESI+)=366.1.

### 6-(2-(1-(2-(2,4-difluorophenyl)-2-hydroxy-3-(1H-1,2,3-triazol-1-yl)propyl)piperidin-4-yl)ethyl)-4-(3-(4-fluorophenyl)-1-methyl-1H-pyrazol-4-yl)-5H-pyrrolo[3,4-b]pyridine-5,7(6H)-dione (3-5)

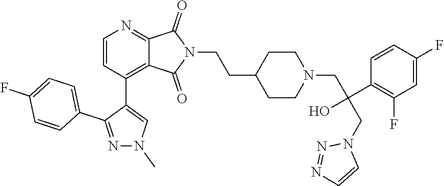 [SMILES: Cn1cc(-c2ccnc3c2C(=O)N(CCC2CCN(CC(O)(Cn4ccnn4)c4ccc(F)cc4F)CC2)C3=O)c(-c2ccc(F)cc2)n1]

To a solution of 4-(3-(4-fluorophenyl)-1-methyl-1H-pyrazol-4-yl)furo[3,4-b]pyridine-5 (150 mg, 0.46 mmol) 3-4 and 1-(4-(2-aminoethyl)piperidin-1-yl)-2-(2,4-difluorophenyl)-3-(1H-1,2,3-triazol-1-yl)propan-2-ol 3-3 (252 mg, 0.69 mmol) in THF (10 mL) were heated at reflux for 16 hours, cooled and then concentrated to a brown solid. This material was purified with reverse-phase column with H₂O/MeOH 5%-95% to afford 123 mg product 3-5 as a yellow solid with 40% yield. LCMS: Calculated Exact Mass=670.3, Found [M+H]+ (ESI+)=671.1.

### 2-(2,4-difluorophenyl)-1,3-di(1H-1,2,4-triazol-1-yl)propan-2-yl 2-(4-((4-(3-(4-fluorophenyl)-1-methyl-1H-pyrazol-4-yl)-5-oxo-5,7-dihydro-6H-pyrrolo[3,4-b]pyridin-6-yl)methyl)piperidin-1-yl)acetate (I-250)

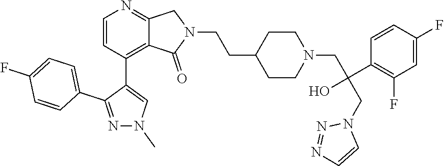 [SMILES: Cn1cc(-c2ccnc3c2C(=O)N(CCC2CCN(CC(O)(Cn4ccnn4)c4ccc(F)cc4F)CC2)C3)c(-c2ccc(F)cc2)n1]

A solution of 6-(2-(1-(2-(2,4-difluorophenyl)-2-hydroxy-3-(1H-1,2,3-triazol-1-yl)propyl)piperidin-4-yl)ethyl)-4-(3-(4-fluorophenyl)-1-methyl-1H-pyrazol-4-yl)-5H-pyrrolo[3,4-b]pyridine-5,7(6H)-dione 3-5 (123 mg, 0.18 mmol) and zinc dust (<10 micron) (119 mg, 1.8 mmol) in acetic acid (3 mL) was heated to 105° C. and stirred for 4 hours, then cooled to room temperature. The reaction mixture was filtered to remove the zinc and the filter pad was rinsed with EtOAc (50 mL) and the filtrate was concentrated and purified with reverse-phase column with H₂O/MeOH 5%-95% to afford 2-(2,4-difluorophenyl)-1,3-di(1H-1,2,4-triazol-1-yl)propan-2-yl 2-(4-((4-(3-(4-fluorophenyl)-1-methyl-1H-pyrazol-4-yl)-5-oxo-5,7-dihydro-6H-pyrrolo[3,4-b]pyridin-6-yl)methyl)piperidin-1-yl)acetate 1-250 (39 mg) as a white solid with 33% yield.

LCMS: Calculated Exact Mass=656.3, Found [M+H]+ (ESI+)=657.2. ¹H NMR (400 MHz, DMSO) δ 8.49 (d, J=5.2 Hz, 1H), 8.14 (s, 1H), 7.86 (d, J=0.8 Hz, 1H), 7.56 (s, 1H), 7.35 (ddd, J=10.7, 8.8, 5.5 Hz, 3H), 7.20-7.09 (m, 3H), 6.98 (d, J=5.2 Hz, 1H), 6.93 (td, J=8.5, 2.4 Hz, 1H), 5.67 (s, 1H), 4.74 (dd, J=51.0, 14.1 Hz, 2H), 4.44 (s, 2H), 3.94 (s, 3H), 3.46 (t, J=7.2 Hz, 2H), 2.83 (d, J=13.8 Hz, 1H), 2.66 (t, J=14.4 Hz, 2H), 2.56 (s, 1H), 2.06 (dt, J=27.3, 10.5 Hz, 2H), 1.60-1.41 (m, 4H), 1.18-0.97 (m, 3H).

### Example 5—Synthesis of 2-(2,4-difluorophenyl)-1,3-di(1H-1,2,4-triazol-1-yl)propan-2-yl 2-(4-((4-(3-(4-fluorophenyl)-1-methyl-1H-pyrazol-4-yl)-5-oxo-5,7-dihydro-6H-pyrrolo[3,4-b]pyridin-6-yl)methyl)piperidin-1-yl)acetate (I-249)

Procedures for the synthesis of compound 2-(2,4-difluorophenyl)-1,3-di(1H-1,2,4-triazol-1-yl)propan-2-yl 2-(4-((4-(3-(4-fluorophenyl)-1-methyl-1H-pyrazol-4-yl)-5-oxo-5,7-dihydro-6H-pyrrolo[3,4-b]pyridin-6-yl)methyl)piperidin-1-yl)acetate (I-249) are shown below in Scheme 10:

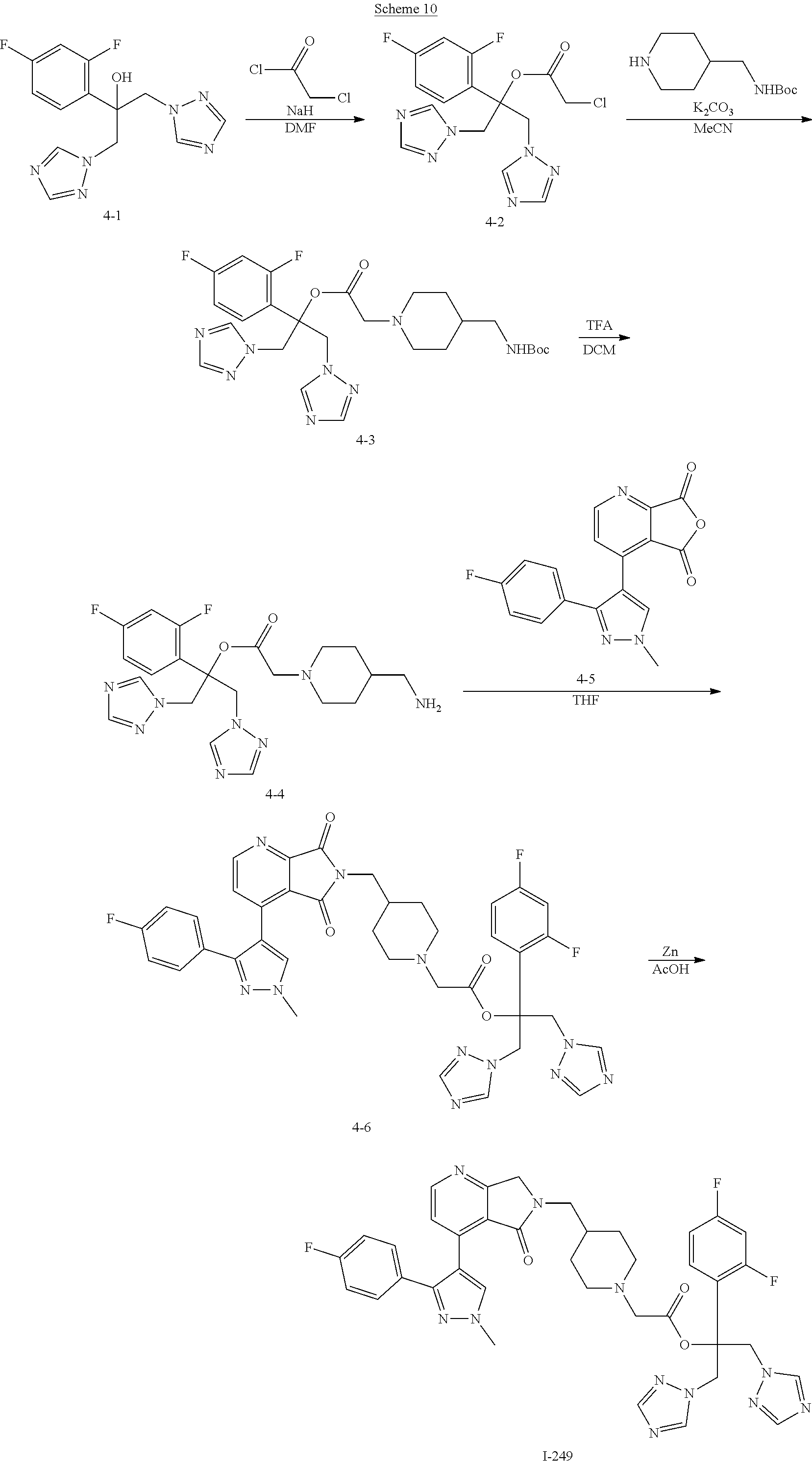

### 2-(2,4-difluorophenyl)-1,3-di(1H-1,2,4-triazol-1-yl)propan-2-yl 2-chloroacetate (4-2)

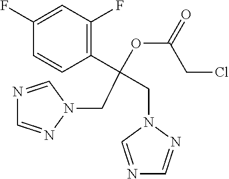 [SMILES: O=C(CCl)OC(Cn1cncn1)(Cn1cncn1)c1ccc(F)cc1F]

2-(2,4-difluorophenyl)-1,3-di(1H-1,2,4-triazol-1-yl)propan-2-ol 4-1 (9.2 g, 30 mmol) was dissolved in 100 mL THF, followed by adding NaH (60%, 3.6 g, 90 mmol) with nitrogen protection at 0° C. Then the reaction was stirred at 70° C. for 1 hour. Then 2-chloroacetyl chloride (10 g, 90 mmol) was added at room temperature. The reaction mixture was stirred at room temperature for 16 hours. The reaction was slowly poured into 300 mL saturated NH₄Cl aqueous solution, extracted with 100 mL EtOAc 3 times. The combined organic layer was washed with 200 mL brine, dried with Na₂SO₄, concentrated in vacuum. The residue was purified with flash column to obtained 2-(2,4-difluorophenyl)-1,3-di(1H-1,2,4-triazol-1-yl)propan-2-yl 2-chloroacetate 4-2 (2.8 g) as a yellow solid with 24% yield. LCMS: Calculated Exact Mass=382.1, Found [M+H]+ (ESI+)=383.2.

### 2-(2,4-difluorophenyl)-1,3-di(1H-1,2,4-triazol-1-yl)propan-2-yl 2-(4-(((tert-butoxycarbonyl)amino)methyl)piperidin-1-yl)acetate (4-3)

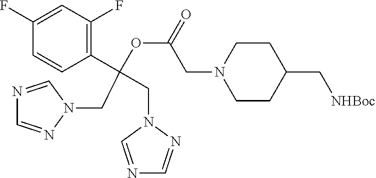 [SMILES: CC(C)(C)OC(=O)NCC1CCN(CC(=O)OC(Cn2cncn2)(Cn2cncn2)c2ccc(F)cc2F)CC1]

2-(2,4-difluorophenyl)-1,3-di(1H-1,2,4-triazol-1-yl)propan-2-yl 2-chloroacetate 4-2 (2.6 g, 6.8 mmol) was dissolved in 30 mL MeCN with tert-butyl (piperidin-4-ylmethyl)carbamate (1.6 g, 7.5 mmol), followed by adding K₂CO₃ (1.9 g, 13.6 mmol) at room temperature. The reaction was stirred at the same temperature for 1 hour. Then the reaction was concentrated in vacuum, diluted in 100 mL EtOAc, washed with 50 mL H₂O twice, and 100 mL brine. The organic layer was dried with Na₂SO₄, concentrated in vacuum. The residue was purified by flash (DCM/MeOH=10:1) to obtained 2-(2,4-difluorophenyl)-1,3-di(1H-1,2,4-triazol-1-yl)propan-2-yl 2-(4-(((tert-butoxycarbonyl)amino)methyl)piperidin-1-yl)acetate 4-3 (800 mg) as a white solid with 21% yield. LCMS: Calculated Exact Mass=560.3, Found [M+H]+ (ESI+)=561.2.

### 2-(2,4-difluorophenyl)-1,3-di(1H-1,2,4-triazol-1-yl)propan-2-yl 2-(4-(aminomethyl)piperidin-1-yl)acetate (4-4)

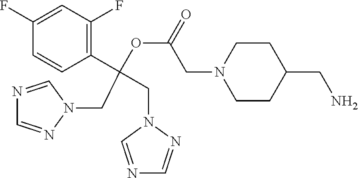 [SMILES: NCC1CCN(CC(=O)OC(Cn2cncn2)(Cn2cncn2)c2ccc(F)cc2F)CC1]

2-(2,4-difluorophenyl)-1,3-di(1H-1,2,4-triazol-1-yl)propan-2-yl 2-(4-(((tert-butoxycarbonyl)amino)methyl)piperidin-1-yl)acetate 4-3 (800 mg, 1.43 mmol) was dissolved in 5 mL DCM and 1 mL TFA was added at 0° C. The reaction was stirred at 0° C. for 1 hour. Then the mixture was concentrated in vacuum and purified by flash (DCM/MeOH=10/1) to obtain 2-(2,4-difluorophenyl)-1,3-di(1H-1,2,4-triazol-1-yl)propan-2-yl 2-(4-(aminomethyl)piperidin-1-yl)acetate 4-4 (200 mg) as a white solid with 30% yield. LCMS: Calculated Exact Mass=460.2, Found [M+H]+ (ESI+)=461.2.

### 2-(2,4-difluorophenyl)-1,3-di(1H-1,2,4-triazol-1-yl)propan-2-yl 2-(4-((4-(3-(4-fluorophenyl)-1-methyl-1H-pyrazol-4-yl)-5,7-dioxo-5,7-dihydro-6H-pyrrolo[3,4-b]pyridin-6-yl)methyl)piperidin-1-yl)acetate (4-6)

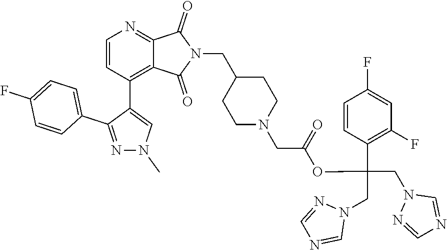 [SMILES: Cn1cc(-c2ccnc3c2C(=O)N(CC2CCN(CC(=O)OC(Cn4cncn4)(Cn4cncn4)c4ccc(F)cc4F)CC2)C3=O)c(-c2ccc(F)cc2)n1]

To a solution of 4-(3-(4-fluorophenyl)-1-methyl-1H-pyrazol-4-yl)furo[3,4-b]pyridine-5 (150 mg, 0.46 mmol) 4-5 and 2-(2,4-difluorophenyl)-1,3-di(1H-1,2,4-triazol-1-yl)propan-2-yl 2-(4-(aminomethyl)piperidin-1-yl)acetate 4-4 (318 mg, 0.69 mmol) in THF (10 mL) were heated at reflux for 16 hour, cooled and then concentrated to a brown solid. This material was purified with reverse-phase column with H₂O/MeOH 5%-95% to afford 2-(2,4-difluorophenyl)-1,3-di(1H-1,2,4-triazol-1-yl)propan-2-yl 2-(4-((4-(3-(4-fluorophenyl)-1-methyl-1H-pyrazol-4-yl)-5,7-dioxo-5,7-dihydro-6H-pyrrolo[3,4-b]pyridin-6-yl)methyl)piperidin-1-yl)acetate 4-6 (100 mg) as a yellow solid with 28% yield. LCMS: Calculated Exact Mass=765.3, Found [M+H]+ (ESI+)=766.2.

### 2-(2,4-difluorophenyl)-1,3-di(1H-1,2,4-triazol-1-yl)propan-2-yl 2-(4-((4-(3-(4-fluorophenyl)-1-methyl-1H-pyrazol-4-yl)-5-oxo-5,7-dihydro-6H-pyrrolo[3,4-b]pyridin-6-yl)methyl)piperidin-1-yl)acetate (I-249)

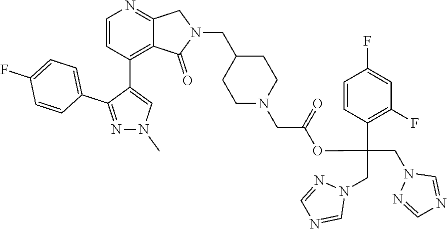 [SMILES: Cn1cc(-c2ccnc3c2C(=O)N(CC2CCN(CC(=O)OC(Cn4cncn4)(Cn4cncn4)c4ccc(F)cc4F)CC2)C3)c(-c2ccc(F)cc2)n1]

To a flask equipped with overhead stirring was added 2-(2,4-difluorophenyl)-1,3-di(1H-1,2,4-triazol-1-yl)propan-2-yl 2-(4-((4-(3-(4-fluorophenyl)-1-methyl-1H-pyrazol-4-yl)-5,7-dioxo-5,7-dihydro-6H-pyrrolo[3,4-b]pyridin-6-yl)methyl)piperidin-1-yl)acetate 4-6 (100 mg, 0.13 mmol) and zinc dust (<10 micron) (130 mg, 2 mmol) followed by acetic acid (3 mL). The mixture was heated to 105° C. and stirred for 4 hours. The reaction mixture was filtered to remove the zinc and the filter pad was rinsed with EtOAc (50 mL) and the filtrate was concentrated and purified with reverse-phase column with H₂O/MeOH 5%-95% to afford 2-(2,4-difluorophenyl)-1,3-di(1H-1,2,4-triazol-1-yl)propan-2-yl 2-(4-((4-(3-(4-fluorophenyl)-1-methyl-1H-pyrazol-4-yl)-5-oxo-5,7-dihydro-6H-pyrrolo[3,4-b]pyridin-6-yl)methyl)piperidin-1-yl)acetate 1-249 (7 mg) as a white solid with 7% yield.

LCMS: Calculated Exact Mass=751.3, Found [M+H]+ (ESI+)=752.2. ¹H NMR (400 MHz, DMSO) δ 8.51 (d, J=5.2 Hz, 1H), 8.31 (s, 2H), 8.17 (s, 1H), 7.93 (s, 2H), 7.32 (dd, J=8.7, 5.6 Hz, 2H), 7.29-7.21 (m, 1H), 7.13 (t, J=8.9 Hz, 2H), 7.07 (d, J=6.9 Hz, 1H), 7.02-6.94 (m, 2H), 5.17 (d, J=14.2 Hz, 2H), 5.02 (d, J=14.9 Hz, 2H), 4.48 (s, 2H), 3.94 (s, 3H), 3.37 (s, 2H), 3.21 (s, 2H), 2.80 (d, J=10.8 Hz, 2H), 2.12 (t, J=11.5 Hz, 2H), 1.68 (s, 1H), 1.52 (d, J=10.2 Hz, 2H), 1.15 (d, J=12.4 Hz, 2H).

### Example 6—Synthesis of 2-(2,4-difluorophenyl)-4-(((4-(3-(4-fluorophenyl)-1-methyl-1H-pyrazol-4-yl)pyridin-3-yl)methyl)amino)-1-(1H-1,2,4-triazol-1-yl)butan-2-ol (I-3) and analogs (I-4) and (I-5)

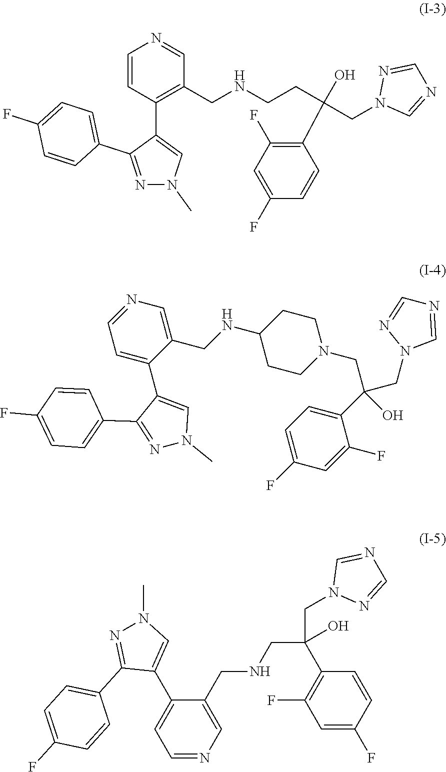 [SMILES: Cn1cc(-c2ccncc2CNCCC(O)(Cn2cncn2)c2ccc(F)cc2F)c(-c2ccc(F)cc2)n1 ; Cn1cc(-c2ccncc2CNC2CCN(CC(O)(Cn3cncn3)c3ccc(F)cc3F)CC2)c(-c2ccc(F)cc2)n1 ; Cn1cc(-c2ccncc2CNCC(O)(Cn2cncn2)c2ccc(F)cc2F)c(-c2ccc(F)cc2)n1]

Procedures for the synthesis of common intermediate 4-(3-(4-fluorophenyl)-1-methyl-1H-pyrazol-4-yl)nicotinaldehyde (5-9) are shown in Scheme 11:

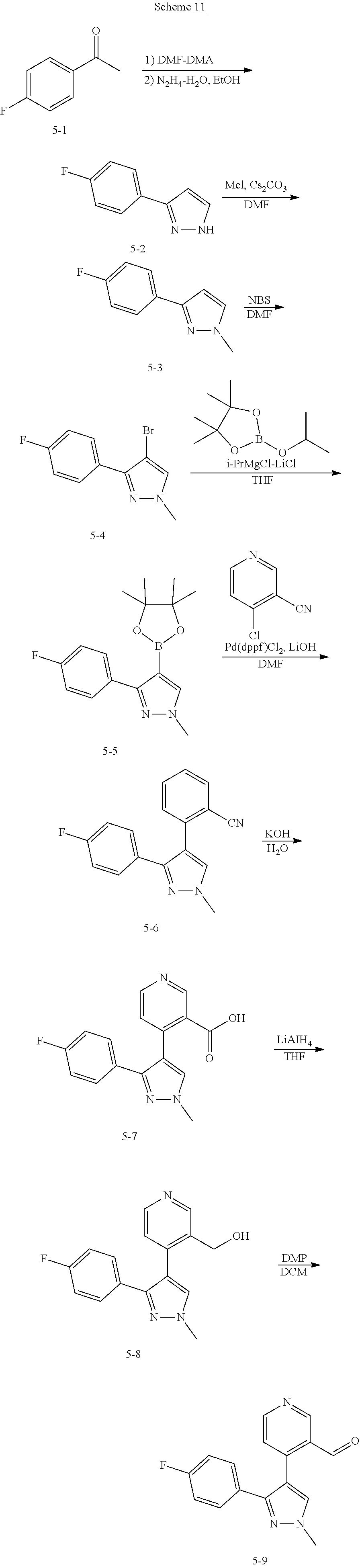

Intermediates 5-2, 5-3, 5-4 and 5-5 were obtained according to procedures as described in Example 1 for intermediates 1-2, 1-3, 1-4 and 1-5.

### 4-(3-(4-fluorophenyl)-1-methyl-1H-pyrazol-4-yl)nicotinonitrile (5-6)

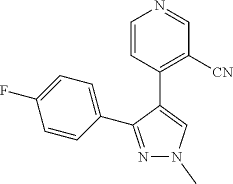 [SMILES: Cn1cc(-c2ccncc2C#N)c(-c2ccc(F)cc2)n1]

3-(4-fluorophenyl)-1-methyl-4-(4,4,5,5-tetramethyl-1,3,2-dioxaborolan-2-yl)-1H-pyrazole 5-5 (25 g, 67 mmol) and 4-chloronicotinonitrile (18.5 g, 134 mmol) were combined in 400 mL DMF and treated with LiOH (4.8 g, 201 mmol). The resulting suspension was sparged with nitrogen for 20 min and treated with Pd(dppf)Cl₂ (4.9 g, 6.7 mmol) in a single portion. The nitrogen sparging was continued for an additional 20 min, and stirring of the dark suspension was continued for 30 min at room temperature. The reaction was warmed to 80° C. for 16 hours and was allowed to cool to room temperature. The thick slurry was added to 250 mL EtOAc, filtered through Celite. The organic layer was washed with 3×300 mL brine. The solid was purified by flash column with a 5-80% EtOAc/heptane gradient to afford 10 g of compound 5-6 as a white solid with 53.7% yield. MS m/z 279.0 (M+1).

### 4-(3-(4-fluorophenyl)-1-methyl-1H-pyrazol-4-yl)nicotinic acid (5-7)

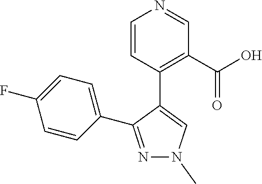 [SMILES: Cn1cc(-c2ccncc2C(=O)O)c(-c2ccc(F)cc2)n1]

To a solution of 4-(3-(4-fluorophenyl)-1-methyl-1H-pyrazol-4-yl)nicotinonitrile 5-6 (10 g, 36 mmol) in EtOH (120 mL) with gentle heating was added 10N NaOH (0.5 mL) and stirred at 90° C. for 2 hours. The reaction mixture was then concentrated, diluted with 200 mLEtOAc, extracted with 200 mL H₂O. The aqueous layer was acidified with 1M HCl to pH=3.10 g of intermediate 5-7 was collected by suction filtration as a solid with 93.5% yield.

### (4-(3-(4-fluorophenyl)-1-methyl-1H-pyrazol-4-yl)pyridin-3-yl)methanol (5-8)

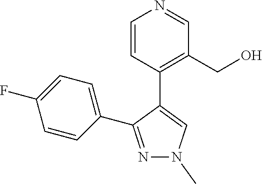 [SMILES: Cn1cc(-c2ccncc2CO)c(-c2ccc(F)cc2)n1]

To a solution of 4-(3-(4-fluorophenyl)-1-methyl-1H-pyrazol-4-yl)nicotinic acid 5-7 (5.0 g, 16.8 mmol) in THF (40 mL) at 0° C. was added LiAlH₄ (1272 mg, 33.6 mmol). The resulting mixture was stirred at 0° C. for 5 min and then warmed to room temperature for 1 hour, quenched with 0.5 mL water, 0.5 mL 15% NaOH solution and then with 2 mL water sequentially. After being stirred at room temperature for 15 min, sodium sulfate was added to the mixture and stirred for another 15 min and then filtered. The resulting solution was concentrated to afford the 1.6 g crude product 5-8 which was used for next step directly without further purification. LCMS (ESI) m/z=284 (M+1).

### 4-(3-(4-fluorophenyl)-1-methyl-1H-pyrazol-4-yl)nicotinaldehyde (5-9)

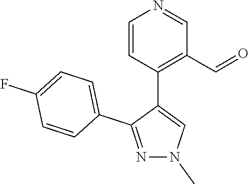 [SMILES: Cn1cc(-c2ccncc2C=O)c(-c2ccc(F)cc2)n1]

To a solution of (4-(3-(4-fluorophenyl)-1-methyl-1H-pyrazol-4-yl)pyridin-3-yl)methanol (400 g, 1.4 mmol) 5-8 in DCM (5 mL), DIB (700 mg, 8.6 mmol) and TEMPO (45 mg, 0.28 mmol) were added. The reaction mixture was stirred at room temperature overnight, and then the solvent was concentrated. The residue was purified by column chromatography to give with flash column to afford 30 mg desired product 5-9 as a white solid with 7.5% yield. LCMS (ESI) m/z=282.2 (M+1) 1H NMR (400 MHz, DMSO) δ 9.88 (s, 1H), 8.89 (s, 1H), 8.73 (d, J=5.2 Hz, 1H), 8.16 (s, 1H), 7.32 (td, J=5.2, 2.3 Hz, 3H), 7.18-7.13 (m, 2H), 3.97 (s, 3H).

Procedure for the synthesis of compound 2-(2,4-difluorophenyl)-4-(((4-(3-(4-fluorophenyl)-1-methyl-1H-pyrazol-4-yl)pyridin-3-yl)methyl)amino)-1-(1H-1,2,4-triazol-1-yl)butan-2-ol (I-3) is shown in Scheme 12:

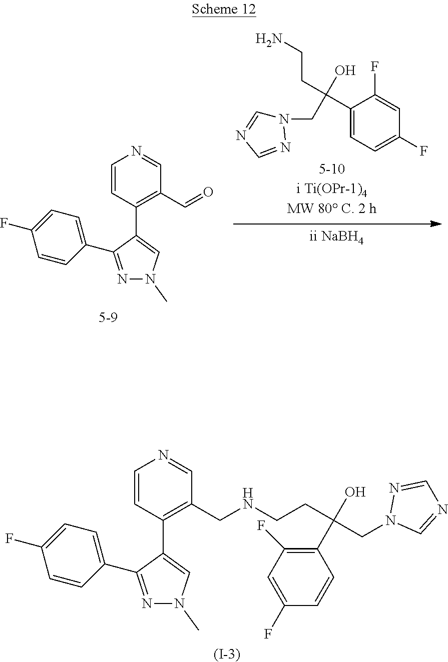

### 2-(2,4-difluorophenyl)-4-(((4-(3-(4-fluorophenyl)-1-methyl-1H-pyrazol-4-yl)pyridin-3-yl)methyl)amino)-1-(1H-1,2,4-triazol-1-yl)butan-2-ol (I-3)

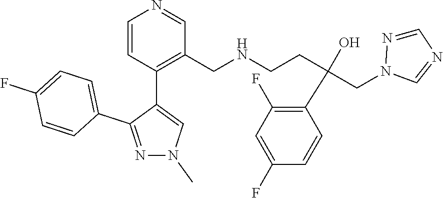 [SMILES: Cn1cc(-c2ccncc2CNCCC(O)(Cn2cncn2)c2ccc(F)cc2F)c(-c2ccc(F)cc2)n1]

380 mg 4-(3-(4-fluorophenyl)-1-methyl-1H-pyrazol-4-yl)nicotinaldehyde 5-9 (1.5 mmol) was mixed with 4-amino-2-(2,4-difluorophenyl)-1-(1H-1,2,4-triazol-1-yl)butan-2-ol 5-10 (2 mmol, 536 mg), and catalytic amount Ti(OiPr)₄ in 4 mL EtOH. The reaction was stirred at 80° C. microwave for 2 hours. Then NaBH₄ (170 mg, 4.5 mmol) was added at room temperature, and the reaction was allowed to stir at room temperature for another 2 hours. The reaction mixture was concentrated, purified by reverse-phase column with H₂O/MeOH 5%-95% to afford desired product (I-3) as a white solid 80 mg with 10% yield. MS (ESi+): 534 (M+H)+. ¹H NMR (400 MHz, DMSO-d₆) δ 8.52 (s, 1H), 8.40 (d, J=5.0 Hz, 1H), 8.25 (s, 1H), 7.88 (s, 1H), 7.74 (s, 1H), 7.26 (dd, J=8.8, 6.0 Hz, 3H), 7.15 (d, J=2.8 Hz, 1H), 7.13-7.05 (m, 4H), 6.91 (d, J=2.6 Hz, 1H), 4.41 (s, 2H), 3.93 (s, 3H), 3.38 (d, J=13.3 Hz, 1H), 3.27 (s, 1H), 2.36-2.31 (m, 1H), 2.16 (s, 1H), 1.99 (d, J=14.0 Hz, 1H), 1.75-1.68 (m, 1H).

Procedure for the synthesis of compound 2-(2,4-difluorophenyl)-1-(4-(((4-(3-(4-fluorophenyl)-1-methyl-1H-pyrazol-4-yl)pyridin-3-yl)methyl)amino)piperidin-1-yl)-3-(1H-1,2,4-triazol-1-yl)propan-2-ol (I-4) is shown in Scheme 13:

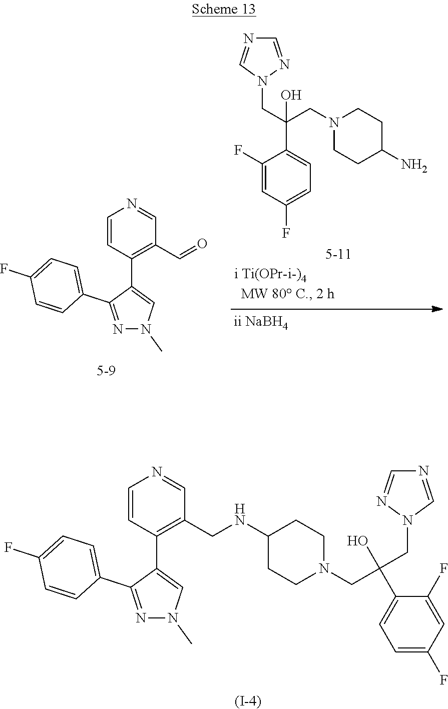 [SMILES: Cn1cc(-c2ccncc2C=O)c(-c2ccc(F)cc2)n1 ; NC1CCN(CC(O)(Cn2cncn2)c2ccc(F)cc2F)CC1 ; Cn1cc(-c2ccncc2CNC2CCN(CC(O)(Cn3cncn3)c3ccc(F)cc3F)CC2)c(-c2ccc(F)cc2)n1 ; "(I-4)"]

### 2-(2,4-difluorophenyl)-1-(4-(((4-(3-(4-fluorophenyl)-1-methyl-1H-pyrazol-4-yl)pyridin-3-yl)methyl)amino)piperidin-1-yl)-3-(1H-1,2,4-triazol-1-yl)propan-2-ol (I-4)

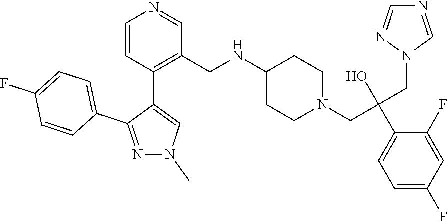 [SMILES: Cn1cc(-c2ccncc2CNC2CCN(CC(O)(Cn3cncn3)c3ccc(F)cc3F)CC2)c(-c2ccc(F)cc2)n1]

480 mg 4-(3-(4-fluorophenyl)-1-methyl-1H-pyrazol-4-yl)nicotinaldehyde 5-9 (1.7 mmol) was mixed with 1-(4-aminopiperidin-1-yl)-2-(2,4-difluorophenyl)-3-(1H-1,2,4-triazol-1-yl)propan-2-ol 5-11 (2.6 mmol, 864 mg), and a catalytic amount of Ti(OiPr)₄ in 4 mL EtOH. The reaction was stirred at 80° C. microwave for 2 hours. Then NaBH₄ (193 mg, 5.1 mmol) was added at room temperature, and the reaction was allowed to stir at room temperature for another 2 hours. The reaction mixture was concentrated, purified by reverse-phase column with H₂O/MeOH 5%-95% to afford desired product (I-4) as a white solid 75 mg with 7.3% yield. MS (ESi+): 603 (M+H)+. ¹H NMR (400 MHz, DMSO-d₆) δ 8.61 (s, 1H), 8.37 (d, J=5.0 Hz, 1H), 8.27 (s, 1H), 7.95 (s, 1H), 7.73 (s, 1H), 7.37 (td, J=9.0, 6.8 Hz, 1H), 7.28 (ddd, J=8.7, 5.5, 2.6 Hz, 2H), 7.17-7.07 (m, 3H), 7.04 (d, J=5.0 Hz, 1H), 6.94 (td, J=8.5, 2.6 Hz, 1H), 5.55 (s, 1H), 4.51 (d, J=4.0 Hz, 2H), 3.91 (s, 3H), 3.44 (s, 2H), 2.77 (dd, J=13.7, 1.8 Hz, 1H), 2.59 (d, J=13.9 Hz, 2H), 2.08 (d, J=4.3 Hz, 1H), 2.00-1.93 (m, 2H), 1.39 (s, 2H), 0.99 (q, J=11.1 Hz, 2H).

Procedure for the synthesis of compound 2-(2,4-difluorophenyl)-1-(((4-(3-(4-fluorophenyl)-1-methyl-1H-pyrazol-4-yl)pyridin-3-yl)methyl)amino)-3-(1H-1,2,4-triazol-1-yl)propan-2-ol (I-5) is shown in Scheme 14:

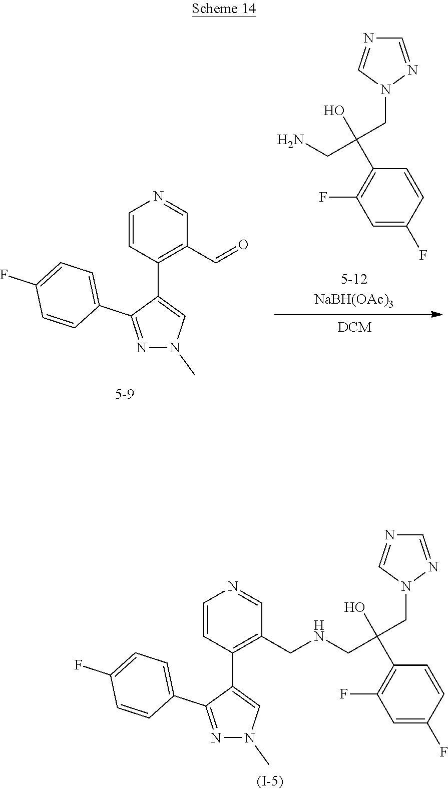

### 2-(2,4-difluorophenyl)-1-(((4-(3-(4-fluorophenyl)-1-methyl-1H-pyrazol-4-yl)pyridin-3-yl)methyl)amino)-3-(1H-1,2,4-triazol-1-yl)propan-2-ol (I-5)

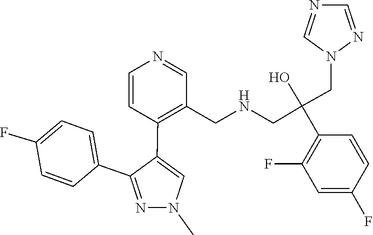 [SMILES: Cn1cc(-c2ccncc2CNCC(O)(Cn2cncn2)c2ccc(F)cc2F)c(-c2ccc(F)cc2)n1]

120 mg 4-(3-(4-fluorophenyl)-1-methyl-1H-pyrazol-4-yl)nicotinaldehyde 5-9 (0.67 mmol) was mixed with 1-amino-2-(2,4-difluorophenyl)-3-(1H-1,2,4-triazol-1-yl)propan-2-ol 5-12 (1.0 mmol, 254 mg), in 4 mL DCM, followed by adding NaBH(OAc)₃ (282 mg, 1.33 mmol). The reaction was allowed to stir at room temperature for 2 hours. The reaction mixture was concentrated, purified by reverse-phase column with H₂O/MeOH 5%-95% to afford desired product (I-5) as a white solid 56.4 mg with 16.2% yield. MS (ESi+): 520 (M+H)+. ¹H NMR (400 MHz, DMSO-d₆) δ 8.55 (s, 1H), 8.36 (d, J=5.0 Hz, 1H), 8.22 (s, 1H), 7.85 (s, 1H), 7.71 (s, 1H), 7.34-7.28 (m, 1H), 7.27-7.23 (m, 2H), 7.13-7.06 (m, 3H), 7.01 (d, J=5.0 Hz, 1H), 6.93-6.86 (m, 1H), 5.67 (s, 1H), 4.46 (d, J=3.0 Hz, 2H), 3.91 (s, 3H), 3.46 (s, 2H), 2.82 (d, J=12.3 Hz, 1H), 2.71 (d, J=12.3 Hz, 1H).

### Example 7—Synthesis of 1-(4-(5-(2-aminopyrimidin-4-yl)-4-(4-fluorophenyl)-1H-imidazol-1-yl)piperidin-1-yl)-2-(2,4-difluorophenyl)-3-(1H-1,2,4-triazol-1-yl)propan-2-ol (I-32), its enantiomers (I-33) and (I-34) and analogs (I-35) and (I-54)

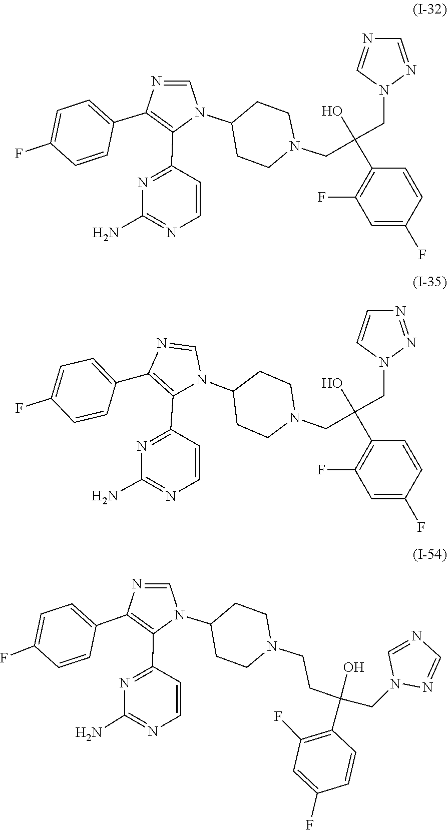 [SMILES: Nc1nccc(-c2c(-c3ccc(F)cc3)ncn2C2CCN(CC(O)(Cn3cncn3)c3ccc(F)cc3F)CC2)n1 ; Nc1nccc(-c2c(-c3ccc(F)cc3)ncn2C2CCN(CC(O)(Cn3ccnn3)c3ccc(F)cc3F)CC2)n1 ; Nc1nccc(-c2c(-c3ccc(F)cc3)ncn2C2CCN(CCC(O)(Cn3cncn3)c3ccc(F)cc3F)CC2)n1]

Procedures for the synthesis of common intermediate 4-(4-(4-fluorophenyl)-1-(piperidin-4-yl)-1H-imidazol-5-yl)pyrimidin-2-amine 6-9 are shown in Scheme 15:

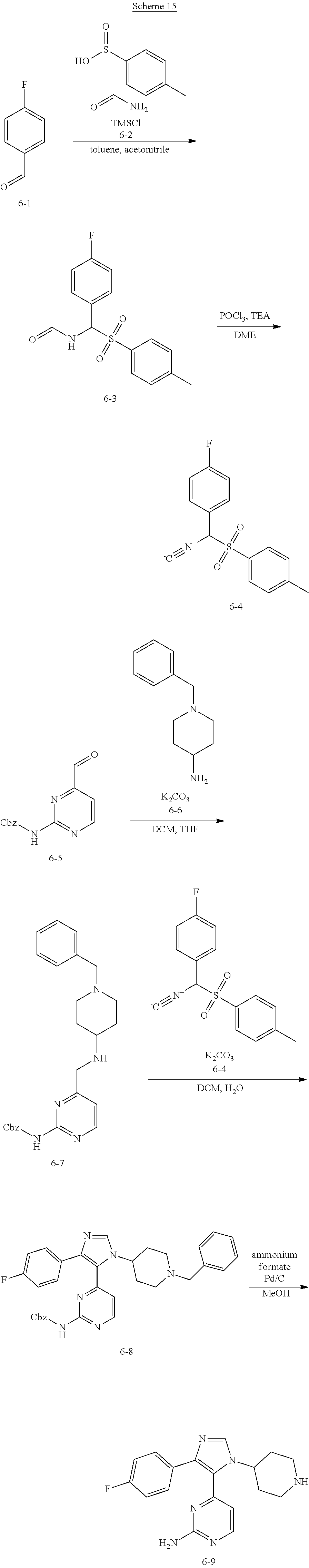

### N-((4-fluorophenyl)(tosyl)methyl)formamide (6-3)

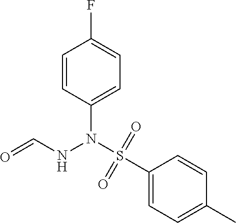 [SMILES: Cc1ccc(S(=O)(=O)N(NC=O)c2ccc(F)cc2)cc1]

20 g (161.3 mmol) of 4-fluorobenzaldehyde 6-1, 16 mL (2.5 eq 403 mmol) of formamide and 22.5 mL (177.4 mmol) of chlorotrimethylsilane were dissolved in 55 mL of acetonitrile and 55 mL of toluene. The reaction was stirred at 50° C. for 4 hours, 37.7 g (241.7 mmol) of p-toluenesulfinic acid 6-2 was added and heating was continued for an additional 4 hours. The solution was cooled to room temperature and 55 mL of TBME was added, followed by adding 250 mL water. The resulting mixture was cooled to 0° C., held there for 1 hour and the precipitated white solid was collected by filtration. The reaction flask was washed with 35 mL of TBME and the rinse was poured over the filter cake. The filter cake was dried to afford 30 g (56%) of desired product 6-3 without further purification.

### 1-fluoro-4-(isocyano(tosyl)methyl)benzene (6-4)

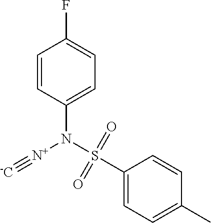

N-((4-fluorophenyl)(tosyl)methyl)formamide 6-3 (20 g, 65.1 mmol) in DME (200 mL) was cooled to −10° C. POCl₃ (18.2 mL, 195.3 mmol) was added followed by the dropwise addition of triethylamine (55.2 mL, 387.6 mmol) in DME (300 mL), keeping the internal temperature below −5° C. The mixture was gradually warmed to ambient temperature over 1 hour, poured into H₂O and extracted with EtOAc. The organic phase was washed with saturated aq. NaHCO₃, dried (Na₂SO₄), and concentrated. The resulting residue was triturated with petroleum ether and filtered to afford the compound 6-4 (10 g, 53.2% yield): ¹H NMR (CDCl₃) δ 7.63 (d, 2H), 7.33 (m, 4H), 7.10 (t, 2H), 5.60 (s, 1H), 2.50 (s, 3H).

### Benzyl (4-(((1-benzylpiperidin-4-yl)imino)methyl)pyrimidin-2-yl)carbamate (6-7)

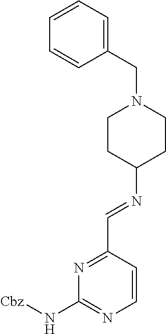 [SMILES: O=C(Nc1nccc(/C=N/C2CCN(Cc3ccccc3)CC2)n1)OCc1ccccc1]

45% aq. KOH (8.56 g, 152.5 mmol) was added to an ice-cold solution of benzyl (4-formylpyrimidin-2-yl)carbamate 6-5 (5.5 g, 21.4 mmol) in aq. HCl (38.2 ml, 145.2 mmol), while the temperature was maintained below 15° C. To the neutralized solution, CH₂Cl₂ (100 mL) and K₂CO₃ (3.0 g, 21.4 mmol) were added followed by 4-amino-1-benzylpiperidine 6-6 (2. 69 g, 16 mmol). The reaction mixture was gradually warmed to room temperature and stirring was continued for 2 hours until no more starting material remained. The reaction mixture was diluted with CH₂Cl₂, washed with water, dried (Na₂SO₄), filtered and concentrated. The crude material 6-7 was used in the next step without purification.

### Benzyl (4-(1-(1-benzylpiperidin-4-yl)-4-(4-fluorophenyl)-1H-imidazol-5-yl)pyrimidin-2-yl)carbamate (6-8)

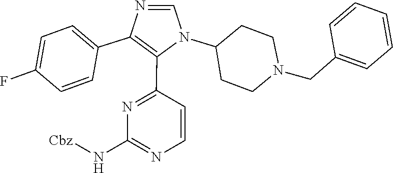 [SMILES: O=C(Nc1nccc(-c2c(-c3ccc(F)cc3)ncn2C2CCN(Cc3ccccc3)CC2)n1)OCc1ccccc1]

1-fluoro-4-(isocyano(tosyl)methyl)benzene 6-4 (6.8 g, 23.5 mmol) and 20% aq. K₂CO₃ (6.5 g, 47 mmol) were added to a solution of crude benzyl (4-(((1-benzylpiperidin-4-yl)imino)methyl)pyrimidin-2-yl)carbamate 6-7 (21.4 mmol) in CH₂Cl₂ directly from the previous step at 20° C. and stirred for 16 hours. Additional imine (2.14 mmol) and K₂CO₃ (2.27 g, 15.6 mmol) were added and the reaction mixture was stirred at 30° C. overnight. The reaction mixture was diluted with CH₂Cl₂, and washed with water, dried (MgSO₄), filtered and concentrated. Flash chromatography with (DCM/MeOH/EtOAc 20:1) afforded desired product 6-8 as a light yellow solid (7.5 g, 56.8%). ¹H NMR (DMSO-d₆, 400 MHz) δ 1.91-1.94 (4H, m), 2.01-2.08 (2H, m), 2.82 (2H, br d, J=10.8 Hz), 3.45 (2H, s), 4.84\hat{ }1.88 (1H, m), 5.20 (2H, s), 6.86 (1H, d, J=5.2 Hz), 7.13-7.17 (2H, m), 7.22-7.45 (2H, m), 8.14 (1H, s), 8.51 (1H, d, J=5.2 Hz), 10.73 (1H, s).

### 4-(4-(4-fluorophenyl)-1-(piperidin-4-yl)-1H-imidazol-5-yl)pyrimidin-2-amine (6-9)

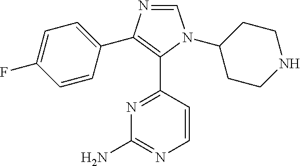 [SMILES: Nc1nccc(-c2c(-c3ccc(F)cc3)ncn2C2CCNCC2)n1]

10% Pd/C (0.75 g) and ammonium formate (8.36 g, 133 mmol) were added to a solution of benzyl (4-(1-(1-benzylpiperidin-4-yl)-4-(4-fluorophenyl)-1H-imidazol-5-yl)pyrimidin-2-yl)carbamate 6-8 (7.5 g, 13.3 mmol) in methanol (100 mL). The resulting reaction mixture was heated to 50° C. and stirred overnight. The reaction mixture was filtered through Celite and concentrated under reduced pressure to afford a residue of 6-9 without purification (4 g, 89%). ¹H NMR (MeOH-d₄, 400 MHz) δ 2.08-2.17 (2H, m), 2.37-2.40 (2H, m), 3.02-3.09 (2H, m), 3.41-3.47 (2H, m), 4.85-4.94 (1H, m), 6.40 (1H, d, J=5.2 Hz), 7.06-7.11 (2H, m), 7.39-7.43 (2H, m), 8.03 (1H, s), 8.12 (1H, d, J=5.2 Hz), 8.53 (1H, s, NH).

Procedure for the synthesis of compound 1-(4-(5-(2-aminopyrimidin-4-yl)-4-(4-fluorophenyl)-1H-imidazol-1-yl)piperidin-1-yl)-2-(2,4-difluorophenyl)-3-(1H-1,2,4-triazol-1-yl)propan-2-ol (I-32) is shown in Scheme 16:

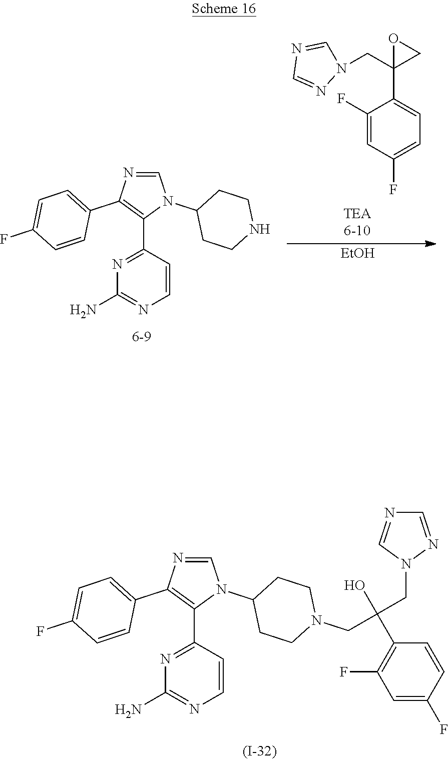 [SMILES: "EtOH" ; Nc1nccc(-c2c(-c3ccc(F)cc3)ncn2C2CCNCC2)n1 ; Fc1ccc(C2(Cn3cncn3)CO2)c(F)c1 ; Nc1nccc(-c2c(-c3ccc(F)cc3)ncn2C2CCN(CC(O)(Cn3cncn3)c3ccc(F)cc3F)CC2)n1 ; "(I-32)"]

### 1-(4-(5-(2-aminopyrimidin-4-yl)-4-(4-fluorophenyl)-1H-imidazol-1-yl)piperidin-1-yl)-2-(2,4-difluorophenyl)-3-(1H-1,2,4-triazol-1-yl)propan-2-ol (I-32)

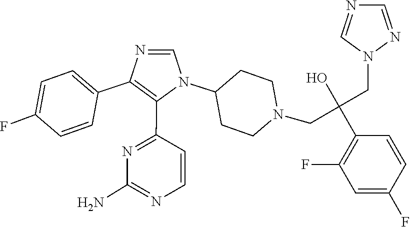 [SMILES: Nc1nccc(-c2c(-c3ccc(F)cc3)ncn2C2CCN(CC(O)(Cn3cncn3)c3ccc(F)cc3F)CC2)n1]

4-(4-(4-fluorophenyl)-1-(piperidin-4-yl)-1H-imidazol-5-yl)pyrimidin-2-amine 6-9 (250 mg, 0.74 mmol) and 1-((2-(2,4-difluorophenyl)oxiran-2-yl)methyl)-1H-1,2,4-triazole 6-10 (526 mg, 2.2 mmol) were dissolved in 4 mL EtOH, followed by adding Et₃N (0.3 mL, 2.2 mmol). The reaction was stirred at 80° C. in a microwave for 4 hours. Then the reaction mixture was concentrated, purified by reverse-phase column with H₂O/MeOH 5%-95% to afford 101 mg of compound (I-32) as a white solid with 23.7% yield. ¹H NMR (400 MHz, DMSO-d₆) δ 8.29 (s, 1H), 8.19 (d, J=5.0 Hz, 1H), 7.95 (s, 1H), 7.74 (s, 1H), 7.42 (ddd, J=8.8, 5.6, 2.7 Hz, 3H), 7.20-7.09 (m, 3H), 6.99-6.94 (m, 1H), 6.79 (s, 2H), 6.39 (d, J=5.0 Hz, 1H), 5.66 (s, 1H), 4.57 (s, 2H), 4.18 (d, J=6.7 Hz, 1H), 2.88 (d, J=13.6 Hz, 1H), 2.82 (d, J=11.6 Hz, 1H), 2.72 (d, J=14.4 Hz, 2H), 2.26-2.16 (m, 2H), 1.82 (m, 4H). LCMS: Calculated Exact Mass=576.2, Found [M+H]+ (ESI+)=576.3.

Procedure for the separation of compound (S)-1-(4-(5-(2-aminopyrimidin-4-yl)-4-(4-fluorophenyl)-1H-imidazol-1-yl)piperidin-1-yl)-2-(2,4-difluorophenyl)-3-(1H-1,2,4-triazol-1-yl)propan-2-ol (I-33) and (R)-1-(4-(5-(2-aminopyrimidin-4-yl)-4-(4-fluorophenyl)-1H-imidazol-1-yl)piperidin-1-yl)-2-(2,4-difluorophenyl)-3-(1H-1,2,4-triazol-1-yl)propan-2-ol (I-34) is shown in Scheme 17:

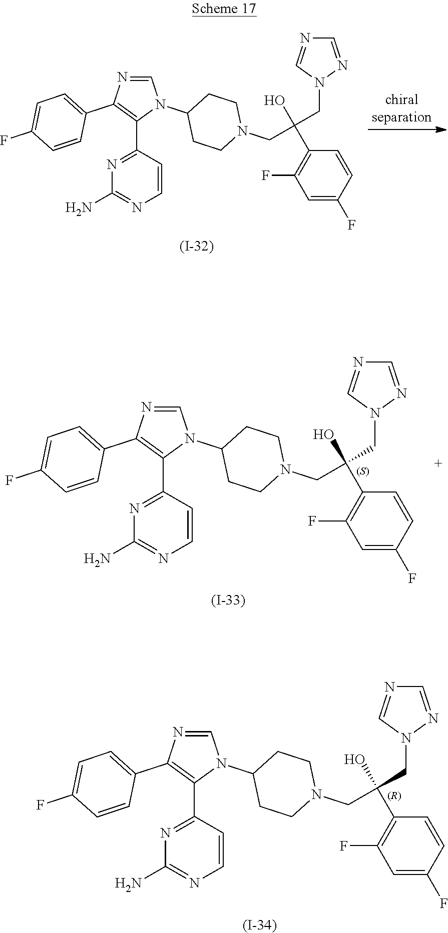

### (S)-1-(4-(5-(2-aminopyrimidin-4-yl)-4-(4-fluorophenyl)-1H-imidazol-1-yl)piperidin-1-yl)-2-(2,4-difluorophenyl)-3-(1H-1,2,4-triazol-1-yl)propan-2-ol (I-33) and (R)-1-(4-(5-(2-aminopyrimidin-4-yl)-4-(4-fluorophenyl)-1H-imidazol-1-yl)piperidin-1-yl)-2-(2,4-difluorophenyl)-3-(1H-1,2,4-triazol-yl)propan-2-ol (I-34)

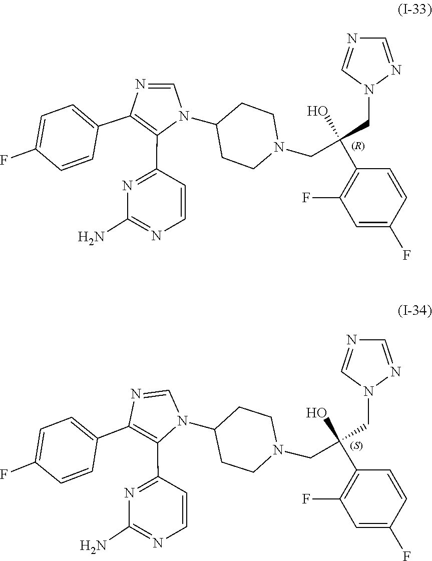

The chiral separation of 1-(4-(5-(2-aminopyrimidin-4-yl)-4-(4-fluorophenyl)-1H-imidazol-1-yl)piperidin-1-yl)-2-(2,4-difluorophenyl)-3-(1H-1,2,4-triazol-1-yl)propan-2-ol (I-32) was carried out by chiral HPLC. The two isomers were obtained. Chiral AD-H column (0.46 cm ID×15 cm), CO₂:IPA (0.05% DEA)=70:30; flow rate at 2.5 mL/min, monitored at 254 nm.

The peak 1 (1st peak) was compound (R)-1-(4-(5-(2-aminopyrimidin-4-yl)-4-(4-fluorophenyl)-1H-imidazol-1-yl)piperidin-1-yl)-2-(2,4-difluorophenyl)-3-(1H-1,2,4-triazol-1-yl)propan-2-ol (I-34) (5.79 min, 100% ee).

LCMS: Calculated Exact Mass=576.2, Found [M+H]⁺ (ESI+)=576.2. ¹H NMR (400 MHz, DMSO-d₆) δ 8.29 (s, 1H), 8.19 (d, J=5.0 Hz, 1H), 7.95 (s, 1H), 7.74 (s, 1H), 7.42 (ddd, J=8.7, 5.5, 2.6 Hz, 3H), 7.21-7.08 (m, 3H), 6.97 (td, J=8.5, 2.6 Hz, 1H), 6.79 (s, 2H), 6.39 (d, J=5.0 Hz, 1H), 5.66 (s, 1H), 4.58 (s, 2H), 4.25-4.14 (m, 1H), 2.85 (dd, J=25.1, 12.7 Hz, 2H), 2.77-2.69 (m, 2H), 2.27-2.13 (m, 2H), 1.83 (q, J=8.6, 6.5 Hz, 4H).

The peak 2 (2nd peak) was compound (S)-1-(4-(5-(2-aminopyrimidin-4-yl)-4-(4-fluorophenyl)-1H-imidazol-1-yl)piperidin-1-yl)-2-(2,4-difluorophenyl)-3-(1H-1,2,4-triazol-1-yl)propan-2-ol (I-33) (6.25 min, 99.8% ee).

LCMS: Calculated Exact Mass=576.2, Found [M+H]⁺ (ESI+)=576.2. ¹H NMR (400 MHz, DMSO-d₆) δ 8.29 (s, 1H), 8.19 (d, J=5.0 Hz, 1H), 7.95 (s, 1H), 7.74 (s, 1H), 7.42 (ddd, J=8.7, 5.6, 2.6 Hz, 3H), 7.20-7.09 (m, 3H), 6.97 (td, J=8.5, 2.6 Hz, 1H), 6.79 (s, 2H), 6.39 (d, J=5.0 Hz, 1H), 5.66 (s, 1H), 4.58 (s, 2H), 4.19 (dt, J=15.6, 9.3 Hz, 1H), 2.88 (d, J=14.0 Hz, 1H), 2.82 (d, J=11.5 Hz, 1H), 2.72 (d, J=14.1 Hz, 2H), 2.28-2.16 (m, 2H), 1.88-1.75 (m, 4H).

Procedures for the synthesis of compound 1-(4-(5-(2-aminopyrimidin-4-yl)-4-(4-fluorophenyl)-1H-imidazol-1-yl)piperidin-1-yl)-2-(2,4-difluorophenyl)-3-(1H-1,2,3-triazol-1-yl)propan-2-ol (I-35) are shown in Scheme 18:

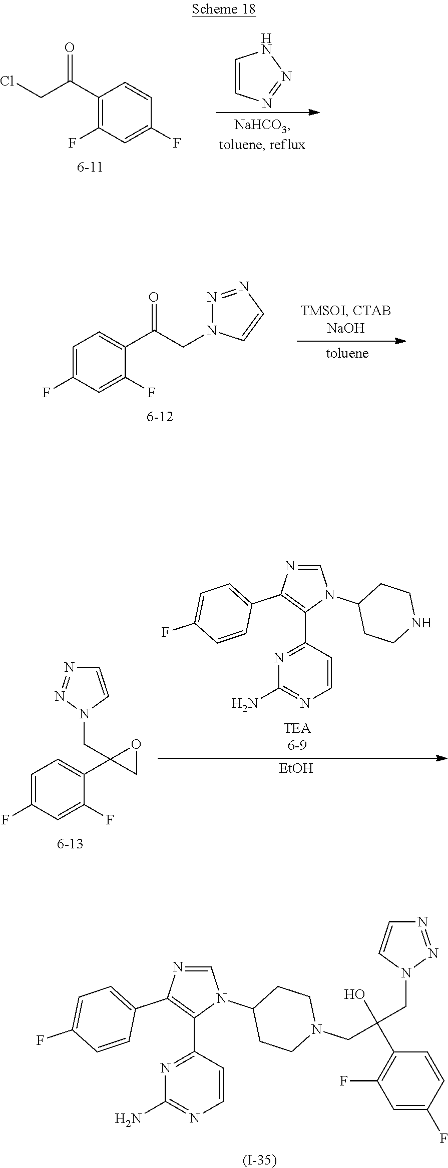

### 1-(2,4-difluorophenyl)-2-(1H-1,2,3-triazol-1-yl)ethan-1-one (6-12)

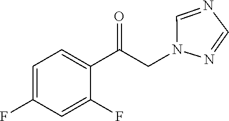 [SMILES: O=C(Cn1cncn1)c1ccc(F)cc1F]

A mixture of 2-chloro-2′ 4′-difluoroacetophenone 6-11 (5 g, 26 mmol), 1,2,3-triazole (1.85 mL, 32 mmol), and sodium bicarbonate (2.68 g, 32 mmol) in toluene (25 mL) was refluxed for 4 hours. The reaction mixture was poured into crushed ice and extracted with EtOAc (2×25 mL). The combined organic layer was washed with H₂O (2×10 mL) and brine (20 mL), dried over Na₂SO₄. The product was then purified by flash column chromatography using petroleum ether/ethyl acetate (50:50) to afford desired product 6-12 (3.3 g, 61%). ¹H NMR (400 MHz, CDCl₃) δ 8.06 (m, 1H), 7.81 (d, J=0.7 Hz, 1H), 7.71 (d, J=0.9 Hz, 1H), 7.07 (m, 1H), 6.99 (m, 1H), 5.81 (d, J=3.5 Hz, 2H).

### 1-((2-(2,4-difluorophenyl)oxiran-2-yl)methyl)-1H-1,2,3-triazole (6-13)

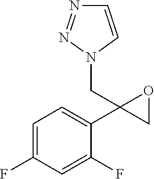 [SMILES: Fc1ccc(C2(Cn3ccnn3)CO2)c(F)c1]

To a solution of 1-(2,4-difluorophenyl)-2-(1H-1,2,3-triazol-1-yl)ethan-1-one 6-12 (1 g, 4.4 mmol) in toluene (20 mL) was added trimethylsulfoxonium iodide (1.18 g, 5.2 mmol) followed by the addition of 20% sodium hydroxide solution (1 mL). The reaction mixture was then heated at 60° C. for 4 hours. The mixture was diluted with EtOAc (10 mL) and poured into ice-water mixture. The organic layer was washed with H₂O (2×10 mL) and brine (20 mL), dried over Na₂SO₄. The product was then purified by flash column chromatography using petroleum ether/ethyl acetate (40:60) to afford compound 6-13 (810 mg, 78%). ¹H NMR (400 MHz CDCl₃) δ 7.62 (d, J=1.7 Hz, 2H), 7.11 (m, 1H), 6.78 (m, 2H), 5.05 (d, J=14.7 Hz, 1H), 4.65 (d, J=14.7 Hz, 1H), 2.88 (dd, J=12.6, 4.7 Hz, 2H).

### 1-(4-(5-(2-aminopyrimidin-4-yl)-4-(4-fluorophenyl)-1H-imidazol-1-yl)piperidin-1-yl)-2-(2,4-difluorophenyl)-3-(1H-1,2,3-triazol-1-yl)propan-2-ol (I-35)

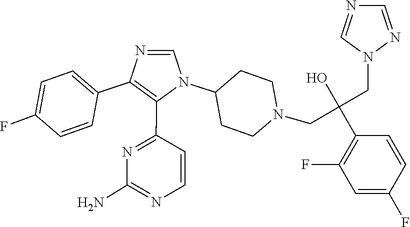 [SMILES: Nc1nccc(-c2c(-c3ccc(F)cc3)ncn2C2CCN(CC(O)(Cn3cncn3)c3ccc(F)cc3F)CC2)n1]

4-(4-(4-fluorophenyl)-1-(piperidin-4-yl)-1H-imidazol-5-yl)pyrimidin-2-amine 6-9 (100 mg, 0.3 mmol) and 1-((2-(2,4-difluorophenyl)oxiran-2-yl)methyl)-1H-1,2,3-triazole 6-13 (210 mg, 0.88 mmol) were dissolved in 4 mL EtOH, followed by adding Et₃N (0.12 mL, 0.88 mmol). The reaction was stirred at 80° C. in a microwave for 4 hours. Then the reaction mixture was concentrated, purified by reverse-phase column with H₂O/MeOH 5%-95% to afford 39.4 mg of compound (I-35) as a white solid with 22.8% yield. ¹H NMR (400 MHz, DMSO-d₆) δ 8.20 (d, J=5.0 Hz, 1H), 7.95 (s, 1H), 7.86 (d, J=1.0 Hz, 1H), 7.57 (d, J=1.0 Hz, 1H), 7.45-7.40 (m, 2H), 7.40-7.35 (m, 1H), 7.18 (ddd, J=11.8, 9.1, 2.5 Hz, 1H), 7.15-7.09 (m, 2H), 6.95 (td, J=8.4, 2.6 Hz, 1H), 6.79 (s, 2H), 6.39 (d, J=5.0 Hz, 1H), 5.75 (s, 1H), 4.85-4.68 (m, 2H), 4.19 (t, J=5.2 Hz, 1H), 2.92-2.87 (m, 1H), 2.83 (d, J=11.6 Hz, 1H), 2.73 (d, J=13.7 Hz, 2H), 2.28-2.14 (m, 2H), 1.93-1.72 (m, 4H). LCMS: Calculated Exact Mass=576.2, Found [M+H]+ (ESI+)=576.3.

Procedures for the synthesis of compound 1-(4-(5-(2-aminopyrimidin-4-yl)-4-(4-fluorophenyl)-1H-imidazol-1-yl)piperidin-1-yl)-2-(2,4-difluorophenyl)-3-(1H-1,2,3-triazol-1-yl)propan-2-ol (I-54) are shown below in Scheme 19:

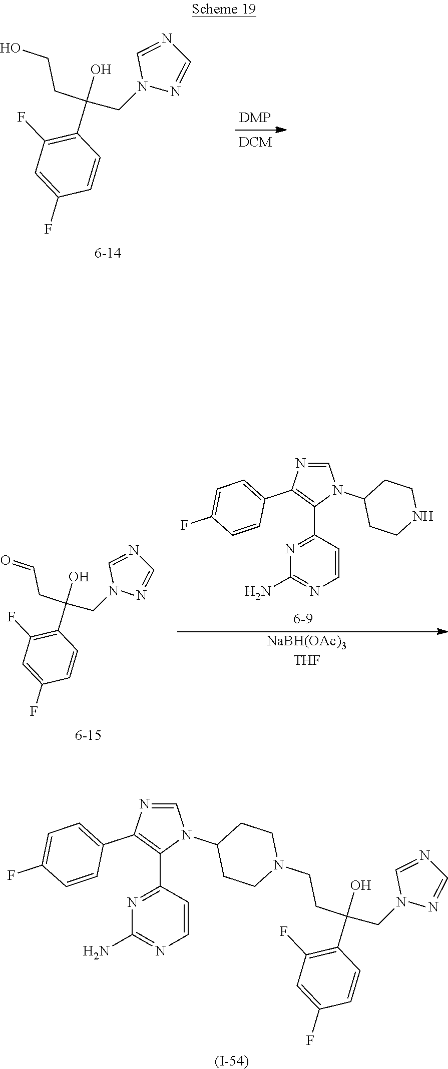 [SMILES: OCCC(O)(Cn1cncn1)c1ccc(F)cc1F ; "NaBH(OAc)3THF" ; O=CCC(O)(Cn1cncn1)c1ccc(F)cc1F ; Nc1nccc(-c2c(-c3ccc(F)cc3)ncn2C2CCNCC2)n1 ; Nc1nccc(-c2c(-c3ccc(F)cc3)ncn2C2CCN(CCC(O)(Cn3cncn3)c3ccc(F)cc3F)CC2)n1 ; "(I-54)"]

### 3-(2,4-difluorophenyl)-3-hydroxy-4-(1H-1,2,4-triazol-1-yl)butanal (6-15)

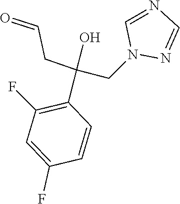 [SMILES: O=CCC(O)(Cn1cncn1)c1ccc(F)cc1F]

To a solution of 3-(2,4-difluorophenyl)-4-(1H-1,2,4-triazol-1-yl)butane-1,3-diol 6-14 (200 mg, 0.74 mmol) in CH₂Cl₂ (5 mL) was added Dess-Martin periodinane (0.62 g, 1.47 mmol) in few portions (over −10 mins), maintaining the temperature below 5° C. with water/ice bath cooling. After the reaction was complete, the mixture was poured into a saturated aqueous solution of sodium hydrogen carbonate (5 mL). The layers were separated and the aqueous layer extracted with DCM (10 mL). The combined organic extracts were washed with brine (10 mL), dried over sodium sulfate, and concentrated and purified with flash column to afford 160 mg of intermediate 6-15 as a white solid with 81% yield.

### 1-(4-(5-(2-aminopyrimidin-4-yl)-4-(4-fluorophenyl)-1H-imidazol-1-yl)piperidin-1-yl)-2-(2,4-difluorophenyl)-3-(1H-1,2,3-triazol-1-yl)propan-2-ol (I-54)

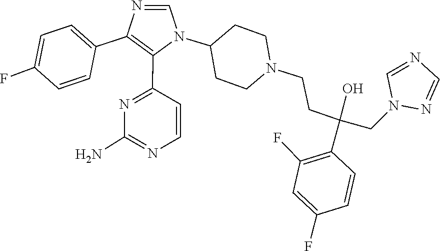 [SMILES: Nc1nccc(-c2c(-c3ccc(F)cc3)ncn2C2CCN(CCC(O)(Cn3cncn3)c3ccc(F)cc3F)CC2)n1]

4-(4-(4-fluorophenyl)-1-(piperidin-4-yl)-1H-imidazol-5-yl)pyrimidin-2-amine 6-9 (135 mg, 0.4 mmol) was mixed with 3-(2,4-difluorophenyl)-3-hydroxy-4-(1H-1,2,4-triazol-1-yl)butanal (160 mg, 0.6 mmol) 6-15, in 4 mL THF, followed by adding NaBH(OAc)₃ (127 mg, 0.6 mmol). The reaction was allowed to stir at room temperature for another 2 hours. The reaction mixture was concentrated, purified by reverse-phase column with H₂O/MeOH 5%-95% to afford desired compound (I-54) as a white solid (139 mg, 59% yield). MS (ESi+): 590 (M+H)+. ¹H NMR (400 MHz, DMSO-d₆) δ 8.29 (s, 1H), 8.19 (d, J=5.0 Hz, 1H), 8.04 (s, 1H), 7.76 (s, 1H), 7.47-7.41 (m, 3H), 7.21 (ddd, J=11.8, 9.1, 2.6 Hz, 1H), 7.15-7.09 (m, 2H), 7.00 (td, J=8.5, 2.6 Hz, 1H), 6.87 (s, 1H), 6.78 (s, 2H), 6.40 (d, J=5.0 Hz, 1H), 4.51 (s, 2H), 4.24 (s, 1H), 2.92 (d, J=7.4 Hz, 1H), 2.64 (d, J=7.6 Hz, 1H), 2.30-2.24 (m, 1H), 2.18-2.07 (m, 2H), 1.93 (dd, J=12.8, 6.4 Hz, 5H), 1.84 (d, J=8.3 Hz, 2H).

### Example 8—Synthesis of N-(2-(2,4-difluorophenyl)-2-hydroxy-3-(1H-1,2,4-triazol-1-yl)propyl)-2-(4-fluorophenyl)-3-(pyridin-4-yl)pyrazolo[1,5-a]pyridine-6-carboxamide (I-115) and N-(1-(2-(2,4-difluorophenyl)-2-hydroxy-3-(1H-1,2,4-triazol-1-yl)propyl)piperidin-4-yl)-2-(4-fluorophenyl)-3-(pyridin-4-yl)pyrazolo[1,5-a]pyridine-6-carboxamide (I-100)

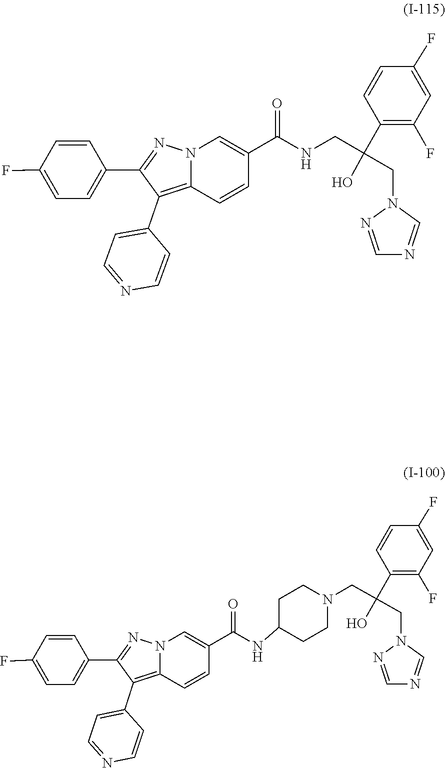 [SMILES: O=C(NCC(O)(Cn1cncn1)c1ccc(F)cc1F)c1ccc2c(-c3ccncc3)c(-c3ccc(F)cc3)nn2c1 ; O=C(NC1CCN(CC(O)(Cn2cncn2)c2ccc(F)cc2F)CC1)c1ccc2c(-c3ccncc3)c(-c3ccc(F)cc3)nn2c1]

Procedure of the synthesis of common intermediate 2-(4-fluorophenyl)-3-(pyridin-4-yl)pyrazolo[1,5-a]pyridine-6-carboxylic acid 7-10 is shown in Scheme 20:

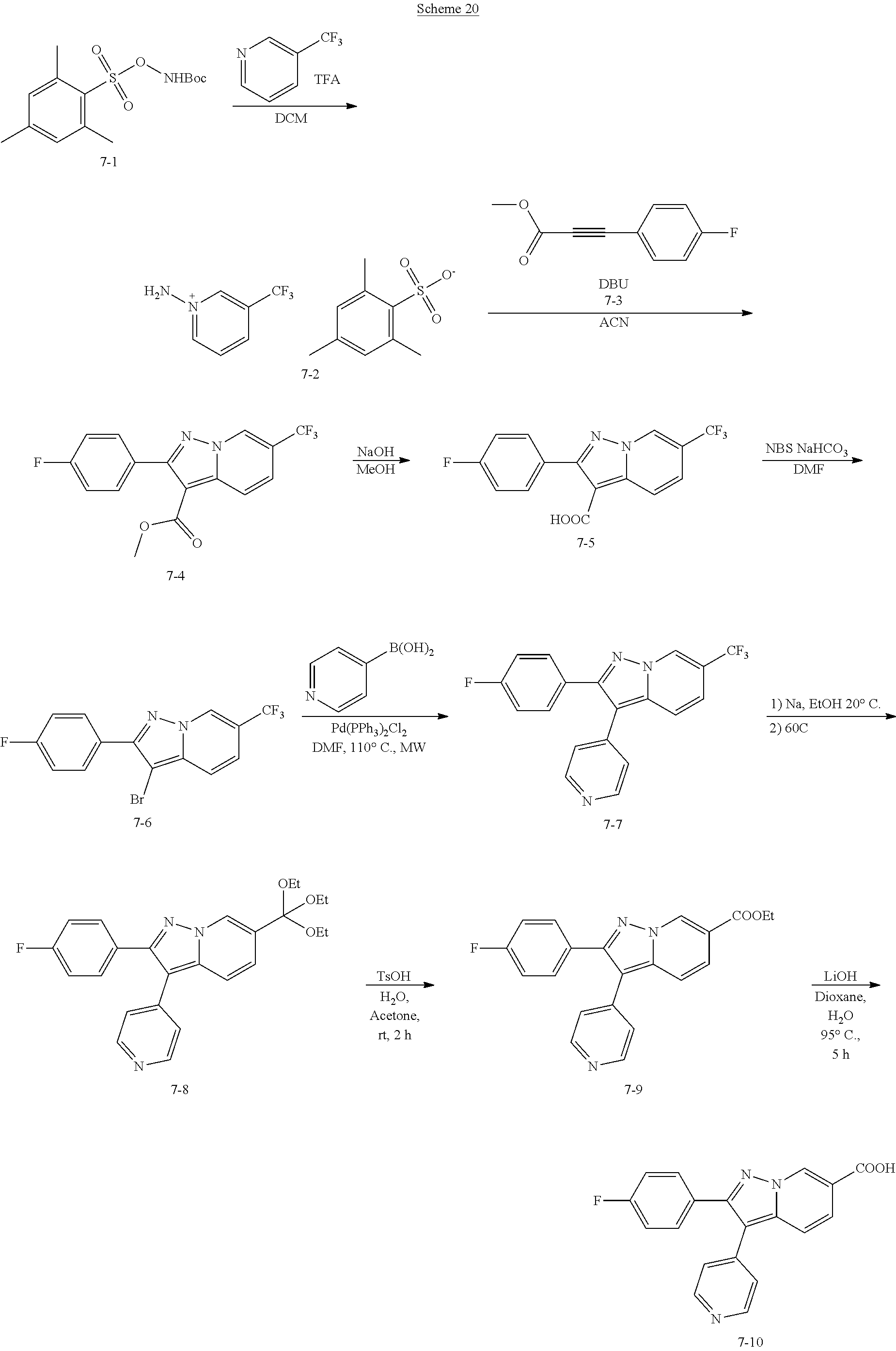

### 1-amino-3-(trifluoromethyl)pyridin-1-ium 2,4,6-trimethyl-benzylsulfonate (7-2)

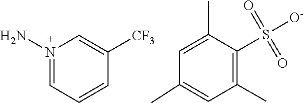

The TFA (20 mL) was added to N-tert-butoxycarbonyl-O-(mesitylsulfonyl)hydroxylamine 7-1 (10 g, 31.7 mmol) in portions at 0° C. over 15 min. The solution was stirred for 15 minutes at room temperature. The solution was poured into the ice water mixture and the resulting precipitate was collected by filtration. The solid was redissolved in DCM and dried by Na₂SO₄. The Na₂SO₄ was removed by filtration and the filtrate was added to a solution of 3-(trifluoromethyl)pyridine (4.6 g 31.7 mmol) in DCM. The mixture was stirred for 45 minutes and then filtered. To the filtrate was added MTBE and the product allowed to precipitate. The solid 7-2 was collected by filtration, washed with METB and direct used for the next step.

### Methyl 2-(4-fluorophenyl)-6-(trifluoromethyl)pyrazolo[1,5-a]pyridine-3-carboxylate (7-4)

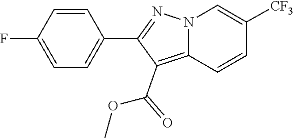 [SMILES: COC(=O)c1c(-c2ccc(F)cc2)nn2cc(C(F)(F)F)ccc12]

To a stirred solution of methyl 3-(4-fluorophenyl)propiolate 7-3 (1.78 g, 10 mmol) and 1-amino-3-(trifluoromethyl)pyridin-1-ium 2,4,6-trimethyl-benzylsulfonate 7-2 (31.7 mmol from the previous step) in dry acetonitrile (20 mL) was added, dropwise over 10 minutes, a acetonitrile solution (5 mL) of DBU (4.7 mL, 31.7 mmol). The mixture was allowed to stir at room temperature for 16 hours. The solvent was evaporated under vacuum and the residue dissolved in EtOAc, washed with water. The organic layer was dried, concentrated and flash column with PE:EA 10:1 to afford 700 mg of intermediate 7-4 as a white solid with 20.7% yield.

### 2-(4-fluorophenyl)-6-(trifluoromethyl)pyrazolo[1,5-a]pyridine-3-carboxylic acid (7-5)

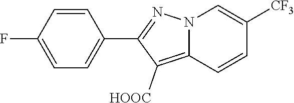 [SMILES: O=C(O)c1c(-c2ccc(F)cc2)nn2cc(C(F)(F)F)ccc12]

A solution of methyl 2-(4-fluorophenyl)-6-(trifluoromethyl)pyrazolo[1,5-a]pyridine-3-carboxylate 7-4 (700 mg, 2 mmol) in 2N aqueous NaOH solution (10 mL) and MeOH (5 mL) was heated at reflux for 3 hours. The mixture was filtered and the filtrate was washed with PE (10 mL) and then concentrated under vacuum to about half of volume. Conc. HCl was added to adjust the pH to 2 and the precipitate was collected by filtration and washed with water, dried to afford 470 mg of intermediate 7-5 as a white solid with 70% yield.

### 3-bromo-2-(4-fluorophenyl)-6-(trifluoromethyl)pyrazolo[1,5-a]pyridine (7-6)

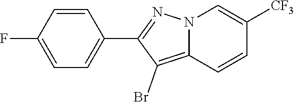 [SMILES: Fc1ccc(-c2nn3cc(C(F)(F)F)ccc3c2Br)cc1]

To a solution of 2-(4-fluorophenyl)-6-(trifluoromethyl)pyrazolo[1,5-a]pyridine-3-carboxylic acid 7-5 (470 mg, 1.45 mmol) in dry DMF (10 mL) was added sodium bicarbonate (365 mg, 4.35 mmol) followed by NBS (258 mg, 1.45 mmol) and the mixture was stirred at room temperature under nitrogen protection for 2 hours. The mixture was diluted with EtOAc (20 mL), washed with water (10 mL*2), dried, and concentrated. Flash column chromatography with DCM:MeOH 20:1 afforded the desired product 7-6 as a tan solid (470 mg, 90% yield).

### 2-(4-fluorophenyl)-3-(pyridin-4-yl)-6-(trifluoromethyl)pyrazolo[1,5-a]pyridine (7-7)

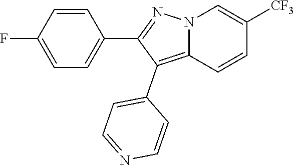 [SMILES: Fc1ccc(-c2nn3cc(C(F)(F)F)ccc3c2-c2ccncc2)cc1]

A solution of 3-bromo-2-(4-fluorophenyl)-6-(trifluoromethyl)pyrazolo[1,5-a]pyridine 7-6 (479 mg, 1.31 mmol), 2-fluoro-4-pyridinylboronic acid (177 mg, 1.44 mmol), sodium carbonate (208 mg, 1.97 mmol) and dichlorobis(triphenylphosphine)palladium (455 mg, 0.66 mmol) in DMF (5 mL) was stirred at 110° C. in a microwave for 2 hours. Then the reaction was cooled to room temperature and filtered through a pad of Celite and rinsed with EtOAc (50 mL). The filtrate was washed with water (20 mL*2), brine (20 mL), dried and concentrated. Reverse phase column with H₂O/MeOH 5%-95% afforded 300 mg of product 7-7 as a white solid with 64% yield.

### 2-(4-fluorophenyl)-3-(pyridin-4-yl)-6-(triethoxymethyl)pyrazolo[1,5-a]pyridine (7-8)

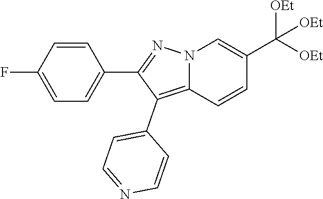 [SMILES: CCOC(OCC)(OCC)c1ccc2c(-c3ccncc3)c(-c3ccc(F)cc3)nn2c1]

To a sodium ethoxide (575 mg, 8.4 mmol) solution in ethanol, 2-(4-fluorophenyl)-3-(pyridin-4-yl)-6-(trifluoromethyl)pyrazolo[1,5-a]pyridine 7-7 (300 mg, 0.84 mmol) was added and the reaction mixture was stirred at 80° C. for 12 hours. The reaction mixture was cooled and concentrated in vacuo to approximately one-fourth of the original volume. The resulting mixture was diluted with water and extracted with ethyl acetate. The organic layer was washed with water and brine, and then dried over sodium sulfate. Filtration and concentration provided 300 mg of intermediate 7-8 as an off-white solid product with 82% yield.

### Ethyl 2-(4-fluorophenyl)-3-(pyridin-4-yl)pyrazolo[1,5-a]pyridine-6-carboxylate (7-9)

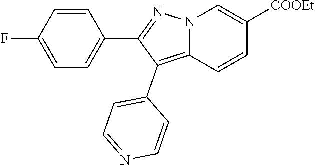 [SMILES: CCOC(=O)c1ccc2c(-c3ccncc3)c(-c3ccc(F)cc3)nn2c1]

To a solution of 2-(4-fluorophenyl)-3-(pyridin-4-yl)-6-(triethoxymethyl)pyrazolo[1,5-a]pyridine 7-8 (300 mg, 0.69 mmol) in acetone (4 mL) and water (1 mL) was added p-toluenesulfonic acid monohydrate (328 mg, 1.73 mmol). The reaction mixture was stirred at 40° C. for 6 hours. The pH of the reaction mixture was adjusted to slightly basic using saturated aqueous sodium bicarbonate solution. The reaction mixture was concentrated and purified by reverse phase column with H₂O/MeOH 5%-95% to afford desired product 7-9 (200 mg, 80% yield).

### 2-(4-fluorophenyl)-3-(pyridin-4-yl)pyrazolo[1,5-a]pyridine-6-carboxylic acid (7-10)

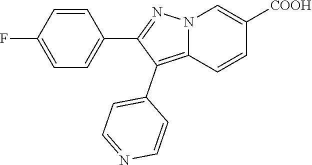 [SMILES: O=C(O)c1ccc2c(-c3ccncc3)c(-c3ccc(F)cc3)nn2c1]

To a solution of ethyl 2-(4-fluorophenyl)-3-(pyridin-4-yl)pyrazolo[1,5-a]pyridine-6-carboxylate 7-9 (200 mg, 0.55 mmol) in dioxane (4 mL) and water (1 mL) was added lithium hydroxide monohydrate (120 mg, 5 mmol). The reaction mixture was heated at 95° C. for 5 hours. The reaction mixture was concentrated in vacuo and purified by reverse phase column with H₂O/MeOH 5%-95% to afford a white solid product 7-10 (38.4 mg, 21% yield).

Procedure for the synthesis of N-(2-(2,4-difluorophenyl)-2-hydroxy-3-(1H-1,2,4-triazol-1-yl)propyl)-2-(4-fluorophenyl)-3-(pyridin-4-yl)pyrazolo[1,5-a]pyridine-6-carboxamide (I-115) is shown below, in Scheme 21:

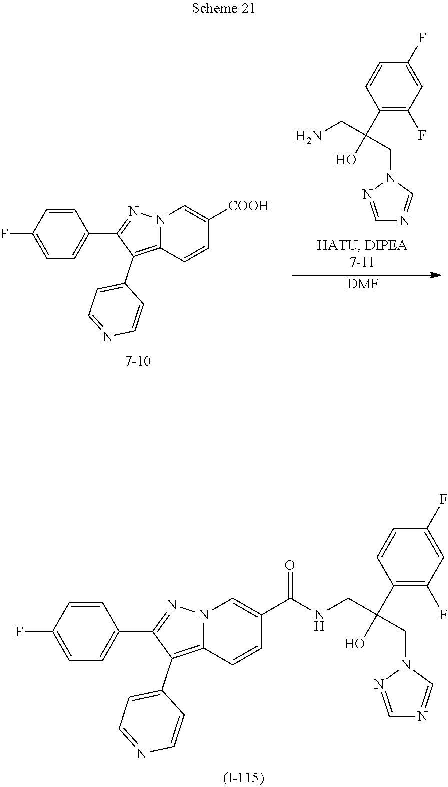

### N-(2-(2,4-difluorophenyl)-2-hydroxy-3-(1H-1,2,4-triazol-1-yl)propyl)-2-(4-fluorophenyl)-3-(pyridin-4-yl)pyrazolo[1,5-a]pyridine-6-carboxamide (I-115)

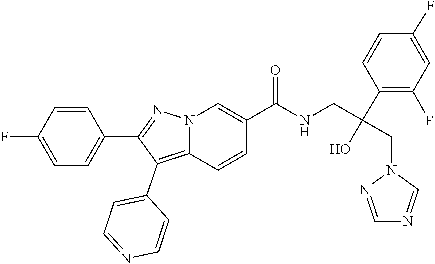 [SMILES: O=C(NCC(O)(Cn1cncn1)c1ccc(F)cc1F)c1ccc2c(-c3ccncc3)c(-c3ccc(F)cc3)nn2c1]

2-(4-fluorophenyl)-3-(pyridin-4-yl)pyrazolo[1,5-a]pyridine-6-carboxylic acid 7-10 (20 mg, 0.06 mmol), 1-amino-2-(2,4-difluorophenyl)-3-(1H-1,2,4-triazol-1-yl)propan-2-ol 7-11 (18.3 mg, 0.072 mmol), HATU (34.2 mg, 0.09 mmol), and DIPEA (31.5 μL, 0.18 mmol) were dissolved in 2 mL DMF. The solution was stirred at room temperature for 2 hours. Then the mixture was purified by reverse phase column with H₂O/MeOH 5%-95% to afford a white solid product (I-115) (15.5 mg, 45.4% yield). LCMS: Calculated Exact Mass=570.2, Found [M+H]+ (ESI+)=570.4. ¹H NMR (400 MHz, DMSO-d₆) δ 9.28 (t, J=1.3 Hz, 1H), 8.75 (t, J=6.1 Hz, 1H), 8.62-8.55 (m, 2H), 8.34 (s, 1H), 7.78 (dd, J=9.4, 0.9 Hz, 1H), 7.76 (s, 1H), 7.68 (dd, J=9.4, 1.6 Hz, 1H), 7.58-7.53 (m, 2H), 7.43 (td, J=9.0, 6.8 Hz, 1H), 7.35-7.26 (m, 4H), 7.20 (ddd, J=11.9, 9.2, 2.6 Hz, 1H), 6.94 (td, J=8.5, 2.6 Hz, 1H), 6.20 (s, 1H), 4.79-4.59 (m, 2H), 3.83 (t, J=5.5 Hz, 2H).

Procedure for the synthesis of N-(1-(2-(2,4-difluorophenyl)-2-hydroxy-3-(1H-1,2,4-triazol-1-yl)propyl)piperidin-4-yl)-2-(4-fluorophenyl)-3-(pyridin-4-yl)pyrazolo[1,5-a]pyridine-6-carboxamide (I-100) is shown in Scheme 22:

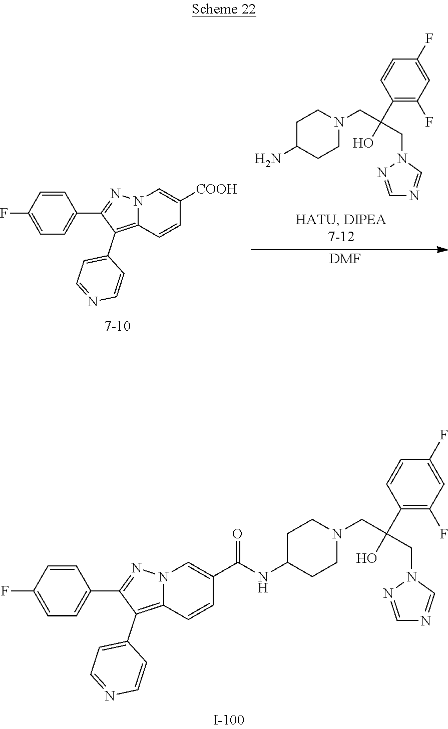

### N-(1-(2-(2,4-difluorophenyl)-2-hydroxy-3-(1H-1,2,4-triazol-1-yl)propyl)piperidin-4-yl)-2-(4-fluorophenyl)-3-(pyridin-4-yl)pyrazolo[1,5-a]pyridine-6-carboxamide (I-100)

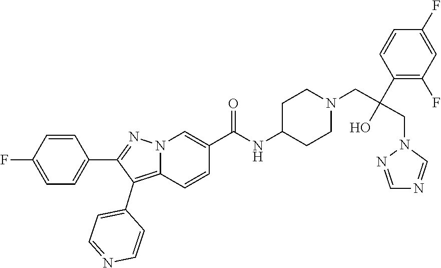 [SMILES: O=C(NC1CCN(CC(O)(Cn2cncn2)c2ccc(F)cc2F)CC1)c1ccc2c(-c3ccncc3)c(-c3ccc(F)cc3)nn2c1]

2-(4-fluorophenyl)-3-(pyridin-4-yl)pyrazolo[1,5-a]pyridine-6-carboxylic acid 7-10 (20 mg, 0.06 mmol), 1-(4-aminopiperidin-1-yl)-2-(2,4-difluorophenyl)-3-(1H-1,2,4-triazol-1-yl)propan-2-ol 7-12 (24.3 mg, 0.072 mmol), HATU (34.2 mg, 0.09 mmol) and DIPEA (31.5 μL, 0.18 mmol) were dissolved in 2 mL DMF. The solution was stirred at room temperature for 2 hours. Then the mixture was purified by reverse phase column with H₂O/MeOH 5%-95% to afford 10 mg of compound (I-100) as a white solid product with 25.6% yield. LCMS: Calculated Exact Mass=653.3, Found [M+H]+ (ESI+)=653.4. ¹H NMR (400 MHz, DMSO-d₆) δ 9.30 (d, J=1.4 Hz, 1H), 8.59 (d, J=5.0 Hz, 2H), 8.40 (d, J=7.6 Hz, 1H), 8.31 (s, 1H), 7.77 (s, 3H), 7.60-7.51 (m, 2H), 7.47-7.40 (m, 1H), 7.35-7.25 (m, 4H), 7.19-7.12 (m, 1H), 6.98 (td, J=8.5, 2.6 Hz, 1H), 5.70 (s, 1H), 4.57 (s, 2H), 3.71 (q, J=4.7 Hz, 1H), 2.93 (d, J=13.8 Hz, 1H), 2.81 (d, J=11.4 Hz, 1H), 2.69 (t, J=14.7 Hz, 2H), 2.21 (td, J=13.1, 10.3 Hz, 2H), 1.68 (d, J=12.1 Hz, 2H), 1.46 (q, J=12.8, 12.2 Hz, 2H).

### Example 9—Synthesis of 2-(2,4-difluorophenyl)-1-(((2-(4-fluorophenyl)-3-(pyridin-4-yl)pyrazolo[1,5-a]pyridin-6-yl)methyl)amino)-3-(1H-1,2,3-triazol-1-yl)propan-2-ol (I-251)

Procedures for the synthesis of compound 2-(2,4-difluorophenyl)-1-(((2-(4-fluorophenyl)-3-(pyridin-4-yl)pyrazolo[1,5-a]pyridin-6-yl)methyl)amino)-3-(1H-1,2,3-triazol-1-yl)propan-2-ol (I-251) is shown below in Scheme 23.

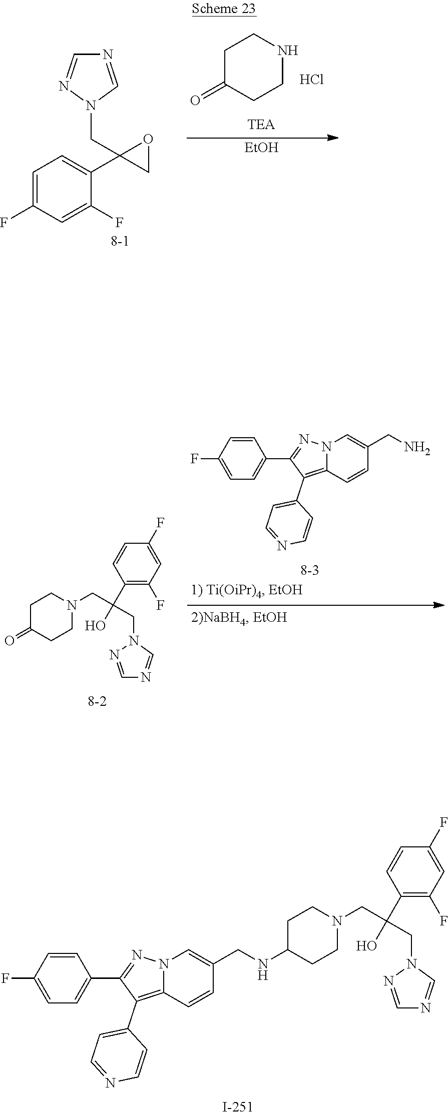

### 1-(2-(2,4-difluorophenyl)-2-hydroxy-3-(1H-1,2,4-triazol-1-yl)propyl)piperidin-4-one (8-2)

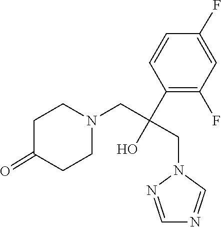 [SMILES: O=C1CCN(CC(O)(Cn2cncn2)c2ccc(F)cc2F)CC1]

1-((2-(2,4-difluorophenyl)oxiran-2-yl)methyl)-1H-1,2,3-triazole 8-1 (500 mg, 2.1 mmol) was dissolved in 5 mL EtOH with NEt₃ (638 mg, 6.3 mmol) and piperidin-4-one hydrochloride (432 mg, 3.2 mmol). The reaction was stirred at 90° C. in microwave for 16 hours. Then the reaction was concentrated, diluted with 30 mL EtOAc, washed with 20 mL water twice and 30 mL brine. The organic layer was dried with Na₂SO₄, concentrated and purified by flash (DCM/MeOH=10/1) to afford 1-(2-(2,4-difluorophenyl)-2-hydroxy-3-(1H-1,2,4-triazol-1-yl)propyl)piperidin-4-one 8-2 (500 mg) as a white solid with 70% yield. LCMS: Calculated Exact Mass=336.1, Found [M+H]+ (ESI+)=337.3.

### 2-(2,4-difluorophenyl)-1-(((2-(4-fluorophenyl)-3-(pyridin-4-yl)pyrazolo[1,5-a]pyridin-6-yl)methyl)amino)-3-(1H-1,2,3-triazol-1-yl)propan-2-ol (I-251)

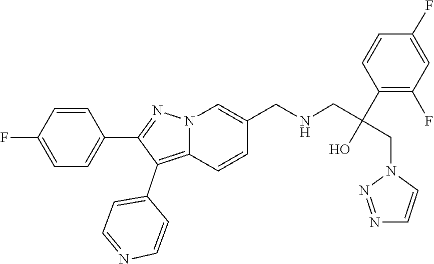 [SMILES: OC(CNCc1ccc2c(-c3ccncc3)c(-c3ccc(F)cc3)nn2c1)(Cn1ccnn1)c1ccc(F)cc1F]

(2-(4-fluorophenyl)-3-(pyridin-4-yl)pyrazolo[1,5-a]pyridin-6-yl)methanamine 8-3 (100 mg, 0.314 mmol) and 1-((2-(2,4-difluorophenyl)oxiran-2-yl)methyl)-1H-1,2,3-triazole 8-2 (149 mg, 0.628 mmol) were dissolved in 15 mL EtOH, followed by adding Et₃N (0.09 mL, 0.628 mmol). The reaction was stirred at 90° C. by microwave for 6 hours. The mixture was concentrated in vacuum, flash column with DCM/MeOH 0-5% to afford desired product 120 mg (0.216 mmol) as a yellow solid with 68% yield. It was purified by reverse-phase column with H₂O/MeOH 5%-95% to afford 2-(2,4-difluorophenyl)-1-(((2-(4-fluorophenyl)-3-(pyridin-4-yl)pyrazolo[1,5-a]pyridin-6-yl)methyl)amino)-3-(1H-1,2,3-triazol-1-yl)propan-2-ol 1-251 (52 mg, 0.093 mmol) as a white solid with 29.7% yield.

LCMS: Calculated Exact Mass=555.2, Found [M+H]+ (ESI+)=556.2. ¹H NMR (400 MHz, DMSO) δ 8.64 (s, 1H), 8.56 (dd, J=4.5, 1.6 Hz, 2H), 7.84 (d, J=0.9 Hz, 1H), 7.69 (d, J=9.4 Hz, 1H), 7.57-7.51 (m, 3H), 7.39-7.25 (m, 6H), 7.12 (ddd, J=11.9, 9.2, 2.5 Hz, 1H), 6.92 (td, J=8.5, 2.5 Hz, 1H), 5.87 (s, 1H), 4.78 (dd, J=29.8, 14.2 Hz, 2H), 3.75 (s, 2H), 3.29 (s, 1H), 2.96 (dd, J=31.3, 12.5 Hz, 2H).

### Example 10—Preparation of (R)-2-(2,4-difluorophenyl)-1-(((2-(4-fluorophenyl)-3-(pyridin-4-yl)pyrazolo[1,5-a]pyridin-6-yl)methyl)amino)-3-(1H-1,2,4-triazol-1-yl)propan-2-ol (I-252) and (S)-2-(2,4-difluorophenyl)-1-(((2-(4-fluorophenyl)-3-(pyridin-4-yl)pyrazolo[1,5-a]pyridin-6-yl)methyl)amino)-3-(1H-1,2,4-triazol-1-yl)propan-2-ol (I-253)

The preparation of compounds 1-252 and 1-253 is made by chiral separation as shown below in Scheme 24:

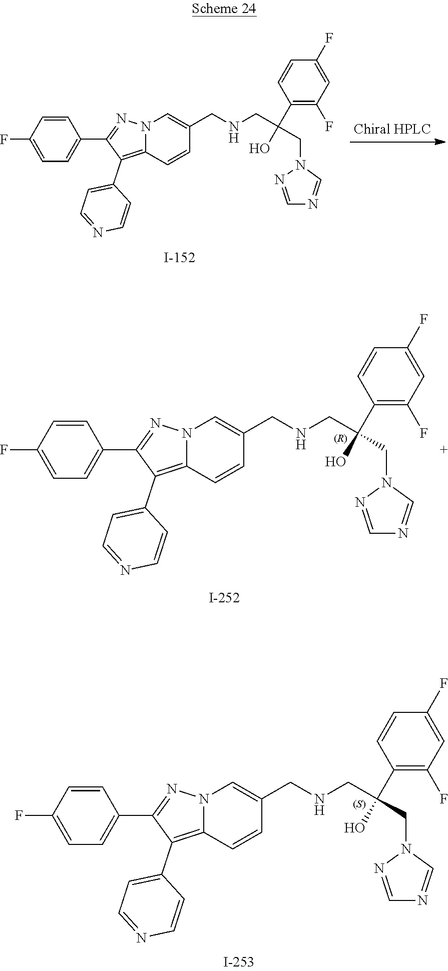

The chiral separation of 2-(2,4-difluorophenyl)-1-(((2-(4-fluorophenyl)-3-(pyridin-4-yl)pyrazolo[1,5-a]pyridin-6-yl)methyl)amino)-3-(1H-1,2,4-triazol-1-yl)propan-2-ol (I-152) was carried out by chiral HPLC. The two isomers (I-252 and 1-253) were obtained. Chiral IG column (4.6*250 mm 5 um); Mobile phase: n-Hexane (0.1% DEA):EtOH (0.1% DEA)=50:50; Flowrate: 1 mL/min; Temperature: 40° C.; Wavelength: 254 nm.

The peak 1 (1st peak) was compound (R)-2-(2,4-difluorophenyl)-1-(((2-(4-fluorophenyl)-3-(pyridin-4-yl)pyrazolo[1,5-a]pyridin-6-yl)methyl)amino)-3-(1H-1,2,4-triazol-1-yl)propan-2-ol (I-252) (12.29 min, 99% ee). LCMS: Calculated Exact Mass=555.6, Found [M+H]+ (ESI+)=556.2. ¹H NMR (400 MHz, DMSO) δ 8.63 (s, 1H), 8.59-8.53 (m, 2H), 8.28 (d, J=6.3 Hz, 1H), 7.72 (s, 1H), 7.69 (d, J=9.2 Hz, 1H), 7.54 (dd, J=8.7, 5.5 Hz, 2H), 7.38 (dd, J=16.0, 9.0 Hz, 1H), 7.31 (dd, J=4.9, 3.4 Hz, 3H), 7.27 (t, J=8.9 Hz, 2H), 7.11 (ddd, J=11.8, 9.2, 2.4 Hz, 1H), 6.94 (td, J=8.4, 2.4 Hz, 1H), 5.79 (s, 1H), 4.65-4.51 (m, 2H), 3.74 (s, 2H), 2.94 (dd, J=27.8, 12.6 Hz, 2H), 2.38 (s, 1H).

The peak 2 (2nd peak) was compound (s)-2-(2,4-difluorophenyl)-1-(((2-(4-fluorophenyl)-3-(pyridin-4-yl)pyrazolo[1,5-a]pyridin-6-yl)methyl)amino)-3-(1H-1,2,4-triazol-1-yl)propan-2-ol (I-253) (14.10 min, 99% ee). LCMS: Calculated Exact Mass=555.6, Found [M+H]+ (ESI+)=556.2. ¹H NMR (400 MHz, DMSO) δ 8.63 (s, 1H), 8.59-8.53 (m, 2H), 8.28 (d, J=6.3 Hz, 1H), 7.72 (s, 1H), 7.69 (d, J=9.2 Hz, 1H), 7.54 (dd, J=8.7, 5.5 Hz, 2H), 7.38 (dd, J=16.0, 9.0 Hz, 1H), 7.31 (dd, J=4.9, 3.4 Hz, 3H), 7.27 (t, J=8.9 Hz, 2H), 7.11 (ddd, J=11.8, 9.2, 2.4 Hz, 1H), 6.94 (td, J=8.4, 2.4 Hz, 1H), 5.79 (s, 1H), 4.65-4.51 (m, 2H), 3.74 (s, 2H), 2.94 (dd, J=27.8, 12.6 Hz, 2H), 2.38 (s, 1H).

### Example 11—Synthesis of Compound 2-(2,4-difluorophenyl)-1-(4-(((2-(4-fluorophenyl)-3-(pyridin-4-yl)pyrazolo[1,5-a]pyridin-6-yl)methyl)amino)piperidin-1-yl)-3-(1H-1,2,4-triazol-1-yl)propan-2-ol (I-254)

Procedures for the synthesis of compound 2-(2,4-difluorophenyl)-1-(4-(((2-(4-fluorophenyl)-3-(pyridin-4-yl)pyrazolo[1,5-a]pyridin-6-yl)methyl)amino)piperidin-1-yl)-3-(1H-1,2,4-triazol-1-yl)propan-2-ol (I-254) is shown below in Scheme 25:

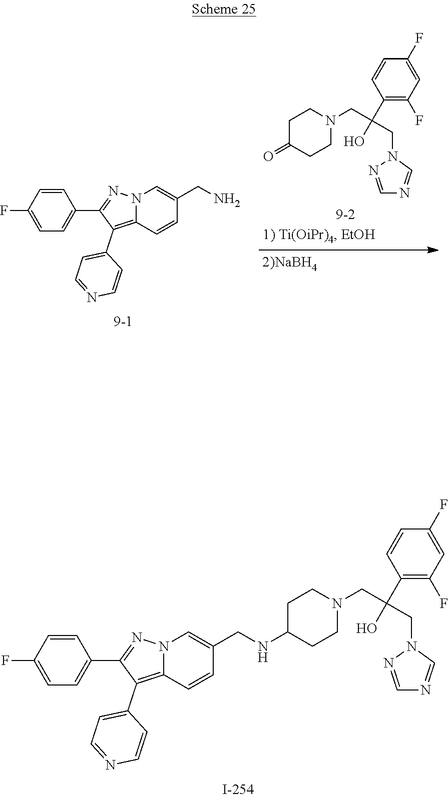 [SMILES: NCc1ccc2c(-c3ccncc3)c(-c3ccc(F)cc3)nn2c1 ; O=C1CCN(CC(O)(Cn2cncn2)c2ccc(F)cc2F)CC1 ; OC(CN1CCC(NCc2ccc3c(-c4ccncc4)c(-c4ccc(F)cc4)nn3c2)CC1)(Cn1cncn1)c1ccc(F)cc1F]

### 2-(2,4-difluorophenyl)-1-(4-(((2-(4-fluorophenyl)-3-(pyridin-4-yl)pyrazolo[1,5-a]pyridin-6-yl)methyl)amino)piperidin-1-yl)-3-(1H-1,2,4-triazol-1-yl)propan-2-ol (I-254)

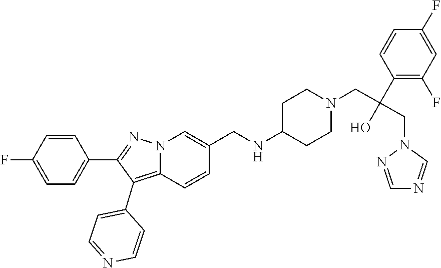 [SMILES: OC(CN1CCC(NCc2ccc3c(-c4ccncc4)c(-c4ccc(F)cc4)nn3c2)CC1)(Cn1cncn1)c1ccc(F)cc1F]

(2-(4-fluorophenyl)-3-(pyridin-4-yl)pyrazolo[1,5-a]pyridin-6-yl)methanamine (100 mg, 0.314 mmol) 9-1 and 1-(2-(2,4-difluorophenyl)-2-hydroxy-3-(1H-1,2,4-triazol-1-yl)propyl)piperidin-4-one (158 mg, 0.471 mmol) 9-2 were dissolved in 10 mL EtOH, followed by adding titanium tetraisopropanolate (two drops). The reaction was stirred at 90° C. by microwave for 2 hours. Then NaBH₄ (60 mg, 1.57 mmol) was added and stirred at room temperature for 15 minutes. The reaction solution was quenched with water (30 mL) and extracted with AcOEt (10 mL*3). The organic layer was washed with brine (20 mL), dried over Na₂SO₄, filtered, concentrated under reduced pressure and purified by reverse-phase column with H₂O/MeOH 5%-95% to afford 2-(2,4-difluorophenyl)-1-(4-(((2-(4-fluorophenyl)-3-(pyridin-4-yl)pyrazolo[1,5-a]pyridin-6-yl)methyl)amino)piperidin-1-yl)-3-(1H-1,2,4-triazol-1-yl)propan-2-ol 1-254 (17.5 mg, 0.027 mmol) as a white solid with 8.7% yield.

LCMS: Calculated Exact Mass=638.2, Found [M+H]+ (ESI+)=639.2. ¹H NMR (400 MHz, DMSO) δ 8.66 (s, 1H), 8.55 (d, J=6.0 Hz, 2H), 8.29 (s, 1H), 7.73 (s, 1H), 7.69 (d, J=9.1 Hz, 1H), 7.53 (dd, J=8.7, 5.5 Hz, 2H), 7.38 (dd, J=9.2, 4.1 Hz, 2H), 7.31-7.24 (m, 4H), 7.18-7.12 (m, 1H), 6.97-6.92 (m, 1H), 5.60 (s, 1H), 4.58-4.50 (m, 2H), 3.73 (s, 2H), 3.29 (s, 1H), 2.82 (d, J=14.8 Hz, 1H), 2.66 (t, J=14.4 Hz, 3H), 2.30 (s, 1H), 2.09 (dd, J=20.3, 9.0 Hz, 2H), 1.67 (s, 2H), 1.23-1.13 (m, 2H).

### Example 12—Synthesis of Compound 3-(2,4-difluorophenyl)-N-((2-(4-fluorophenyl)-3-(pyridin-4-yl)pyrazolo[1,5-a]pyridin-6-yl)methyl)-3-hydroxy-4-(1H-1,2,4-triazol-1-yl)butanamide (I-255)

Procedures for the synthesis of compound 3-(2,4-difluorophenyl)-N-((2-(4-fluorophenyl)-3-(pyridin-4-yl)pyrazolo[1,5-a]pyridin-6-yl)methyl)-3-hydroxy-4-(1H-1,2,4-triazol-1-yl)butanamide (I-255) is shown below in Scheme 26:

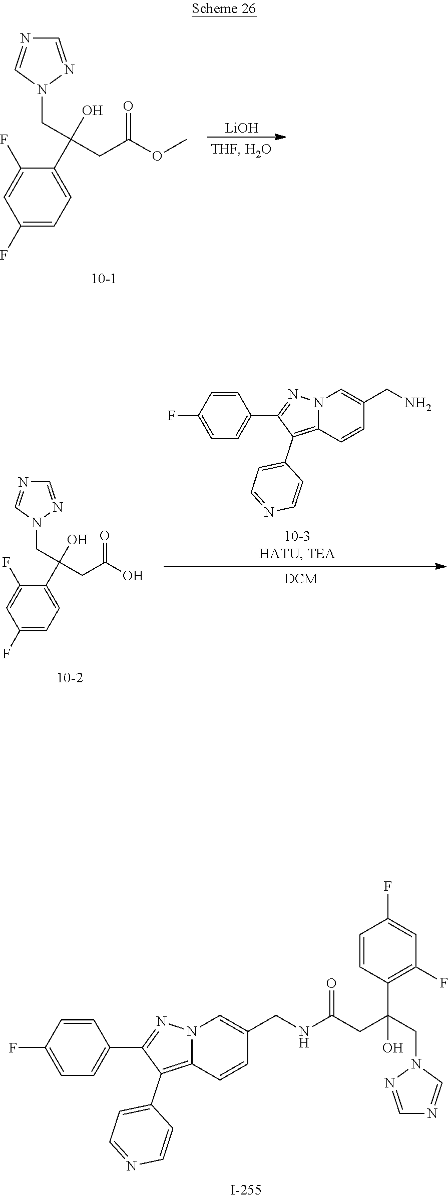

### 3-(2,4-difluorophenyl)-3-hydroxy-4-(1H-1,2,4-triazol-1-yl)butanoic acid (10-2)

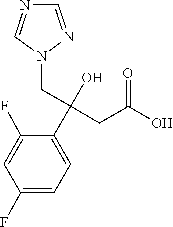 [SMILES: O=C(O)CC(O)(Cn1cncn1)c1ccc(F)cc1F]

Methyl 3-(2,4-difluorophenyl)-3-hydroxy-4-(1H-1,2,4-triazol-1-yl)butanoate 10-1 (500 mg, 1.68 mmol) was dissolved in MeOH (10 ml), followed by adding 1 M NaOH (8.42 ml, 8.42 mmol) and the mixture was stirred at room temperature for 2 hours. Most MeOH was concentrated and the pH was adjusted to 4 with 1 N HCl. The filtered solid was concentrated and afford desired product 10-2 (450 mg, 1.59 mmol) as a white solid with 94.7% yield. LCMS: Calculated Exact Mass=283, Found [M+H]+ (ESI+)=284.

### 3-(2,4-difluorophenyl)-N-((2-(4-fluorophenyl)-3-(pyridin-4-yl)pyrazolo[1,5-a]pyridin-6-yl)methyl)-3-hydroxy-4-(1H-1,2,4-triazol-1-yl)butanamide (I-255)

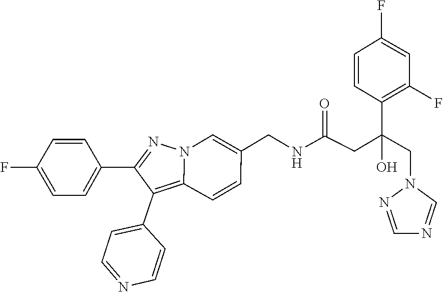 [SMILES: O=C(CC(O)(Cn1cncn1)c1ccc(F)cc1F)NCc1ccc2c(-c3ccncc3)c(-c3ccc(F)cc3)nn2c1]

A solution of (2-(4-fluorophenyl)-3-(pyridin-4-yl)pyrazolo[1,5-a]pyridin-6-yl)methanamine 10-3 (280 mg, 0.88 mmol), 3-(2,4-difluorophenyl)-3-hydroxy-4-(1H-1,2,4-triazol-1-yl)butanoic acid 10-2 (25 mg, 0.88 mmol) HATU (501 mg, 1.32 mmol) and Et₃N (0.25 mL, 1.76 mmol) in DCM (10 mL) was stirred at room temperature for 4 hours. The reaction solution was quenched with water (50 ml) and extracted with DCM (20 mL*3). The organic layer was washed with brine (20 mL), dried over Na₂SO₄, filtered, concentrated under reduced pressure and purified by flash column with DCM/MeOH 0-10% to afford 3-(2,4-difluorophenyl)-N-((2-(4-fluorophenyl)-3-(pyridin-4-yl)pyrazolo[1,5-a]pyridin-6-yl)methyl)-3-hydroxy-4-(1H-1,2,4-triazol-1-yl)butanamide 1-255 (300 mg, 0.514 mmol) as a yellow solid with 58.4% yield.

LCMS: Calculated Exact Mass=583.1, Found [M+H]+ (ESI+)=584.2. ¹H NMR (400 MHz, DMSO) δ 8.72 (t, J=5.9 Hz, 1H), 8.57 (dd, J=4.5, 1.5 Hz, 2H), 8.46 (s, 1H), 8.36 (s, 1H), 7.81 (s, 1H), 7.55 (ddd, J=10.5, 8.7, 5.7 Hz, 3H), 7.42 (dd, J=15.9, 9.0 Hz, 1H), 7.31-7.25 (m, 4H), 7.18 (ddd, J=11.7, 9.1, 2.5 Hz, 1H), 6.94 (dd, J=9.2, 1.2 Hz, 1H), 6.92-6.87 (m, 1H), 6.49 (s, 1H), 4.56 (q, J=14.3 Hz, 2H), 4.30 (dd, J=15.2, 6.1 Hz, 1H), 4.15 (dd, J=15.4, 5.5 Hz, 1H), 2.97 (d, J=14.9 Hz, 1H), 2.62 (d, J=14.7 Hz, 1H).

### Example 13—Synthesis of Compound 2-(2,4-difluorophenyl)-4-(((2-(4-fluorophenyl)-3-(pyridin-4-yl)pyrazolo[1,5-a]pyridin-6-yl)methyl)amino)-1-(1H-1,2,4-triazol-1-yl)butan-2-ol (I-258)

Procedures for the synthesis of compound 2-(2,4-difluorophenyl)-4-(((2-(4-fluorophenyl)-3-(pyridin-4-yl)pyrazolo[1,5-a]pyridin-6-yl)methyl)amino)-1-(1H-1,2,4-triazol-1-yl)butan-2-ol (I-258) is shown below in Scheme 27:

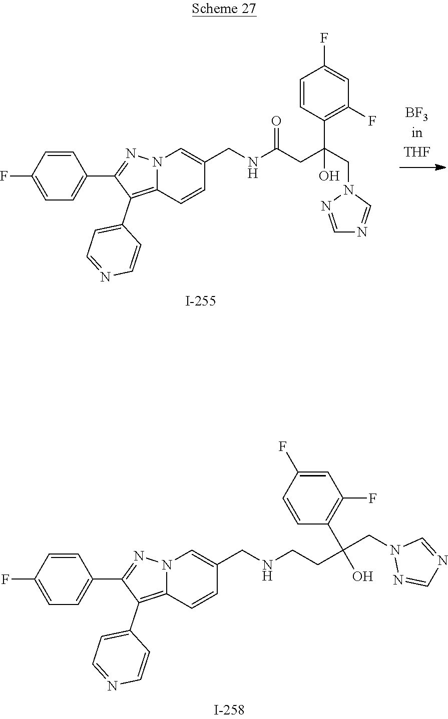 [SMILES: O=C(CC(O)(Cn1cncn1)c1ccc(F)cc1F)NCc1ccc2c(-c3ccncc3)c(-c3ccc(F)cc3)nn2c1 ; OC(CCNCc1ccc2c(-c3ccncc3)c(-c3ccc(F)cc3)nn2c1)(Cn1cncn1)c1ccc(F)cc1F]

### 2-(2,4-difluorophenyl)-4-(((2-(4-fluorophenyl)-3-(pyridin-4-yl)pyrazolo[1,5-a]pyridin-6-yl)methyl)amino)-1-(1H-1,2,4-triazol-1-yl)butan-2-ol (I-258)

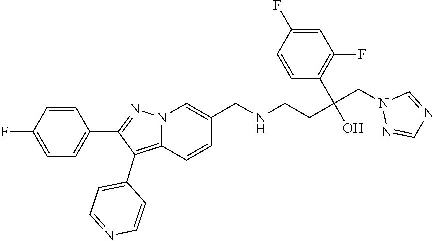 [SMILES: OC(CCNCc1ccc2c(-c3ccncc3)c(-c3ccc(F)cc3)nn2c1)(Cn1cncn1)c1ccc(F)cc1F]

Borane-tetrahydrofuran (20 ml) was added in 3-(2,4-difluorophenyl)-N-((2-(4-fluorophenyl)-3-(pyridin-4-yl)pyrazolo[1,5-a]pyridin-6-yl)methyl)-3-hydroxy-4-(1H-1,2,4-triazol-1-yl)butanamide 1-255 (250 mg, 0.429 mmol) at 0° C. under N₂. The reaction was stirred at 80° C. for 3 hours. The reaction solution was quenched with saturated ammonium chloride solution (50 mL) and extracted with AcOEt (30 mL*3). The organic layer was washed with brine (30 mL), dried over Na₂SO₄, filtered, concentrated under reduced pressure and purified by Prep-HPLC (ACN/H₂O with 0.05% NH₃) to afford 2-(2,4-difluorophenyl)-4-(((2-(4-fluorophenyl)-3-(pyridin-4-yl)pyrazolo[1,5-a]pyridin-6-yl)methyl)amino)-1-(1H-1,2,4-triazol-1-yl)butan-2-ol I-258 (3.7 mg) as a white solid with 1.52% yield.

LCMS: Calculated Exact Mass=569.2, Found [M+H]+ (ESI+)=570.2 ¹H NMR (400 MHz, DMSO) δ 8.59 (s, 1H), 8.57 (dd, J=4.5, 1.6 Hz, 2H), 8.28 (s, 1H), 7.77-7.69 (m, 2H), 7.56-7.50 (m, 2H), 7.34-7.29 (m, 4H), 7.27 (d, J=8.9 Hz, 3H), 7.12 (ddd, J=11.8, 9.1, 2.5 Hz, 1H), 6.66 (td, J=8.5, 2.4 Hz, 1H), 4.49-4.39 (m, 2H), 3.69 (d, J=13.3 Hz, 1H), 3.50 (d, J=13.5 Hz, 1H), 2.91 (s, 1H), 2.33 (d, J=1.8 Hz, 1H), 2.12 (d, J=15.6 Hz, 1H), 1.89-1.80 (m, 1H).

### Example 14—Synthesis of 1-(trans-5-((1H-1,2,4-triazol-1-yl)methyl)-5-(2,4-difluorophenyl)tetrahydrofuran-3-yl)-N-((2-(4-fluorophenyl)-3-(pyridin-4-yl)pyrazolo[1,5-a]pyridin-6-yl)methyl)methanamine (I-256) and 1-(cis-5-((1H-1,2,4-triazol-1-yl)methyl)-5-(2,4-difluorophenyl)tetrahydrofuran-3-yl)-N-((2-(4-fluorophenyl)-3-(pyridin-4-yl)pyrazolo[1,5-a]pyridin-6-yl)methyl)methanamine (I-257)

Procedures for the synthesis of compounds 1-256 and 1-257 is shown below in Scheme 28:

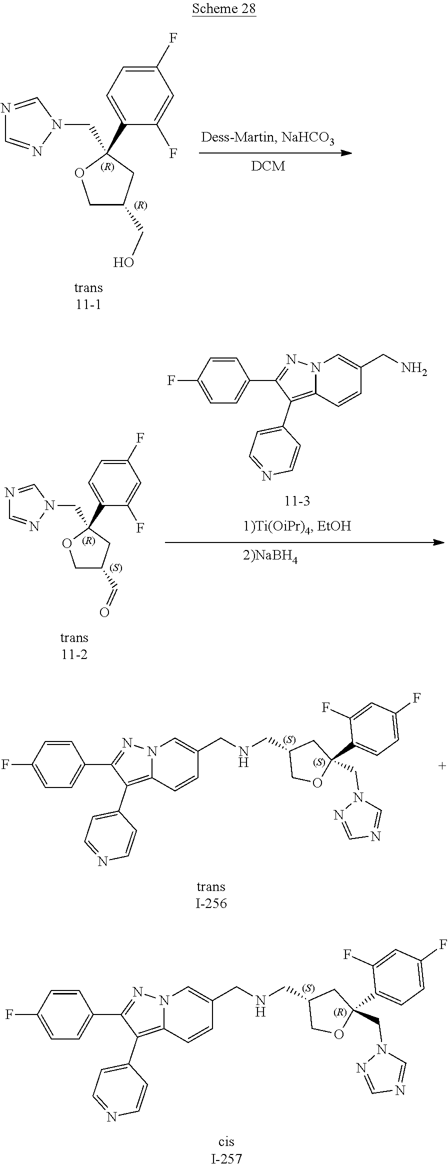

### Trans-5-((1H-1,2,4-triazol-1-yl)methyl)-5-(2,4-difluorophenyl)tetrahydrofuran-3-carbaldehyde (11-2)

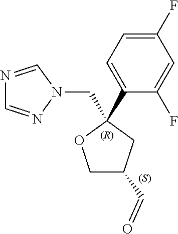

(trans-5-((1H-1,2,4-triazol-1-yl)methyl)-5-(2,4-difluorophenyl)tetrahydrofuran-3-yl)methanol 11-1 (500 mg, 1.69 mmol) and NaHCO₃ (710 mg, 8.45 mmol) were dissolved in DCM (20 ml), followed by adding Dess-Martin periodinane (1.433 g, 3.38 mmol) at 0° C. The reaction was stirred at room temperature for 2 hours. Then the reaction was filtered and rinsed with DCM (50 mL). The filtrate was washed with saturated sodium thiosulfate solution (50 mL*3), dried over Na₂SO₄, filtered, concentrated under reduced pressure and purified by flash column with DCM/MeOH 0-10% to afford desired product 11-2 (350 mg, 1.19 mmol) as a white solid with 70.7% yield. LCMS: Calculated Exact Mass=583.1, Found [M+H]+ (ESI+)=584.2.

### 1-(cis-5-((1H-1,2,4-triazol-1-yl)methyl)-5-(2,4-difluorophenyl)tetrahydrofuran-3-yl)-N-((2-(4-fluorophenyl)-3-(pyridin-4-yl)pyrazolo[1,5-a]pyridin-6-yl)methyl)methanamine (I-256) and 1-(trans-5-((1H-1,2,4-triazol-1-yl)methyl)-5-(2,4-difluorophenyl)tetrahydrofuran-3-yl)-N-((2-(4-fluorophenyl)-3-(pyridin-4-yl)pyrazolo[1,5-a]pyridin-6-yl)methyl)methanamine (I-257)

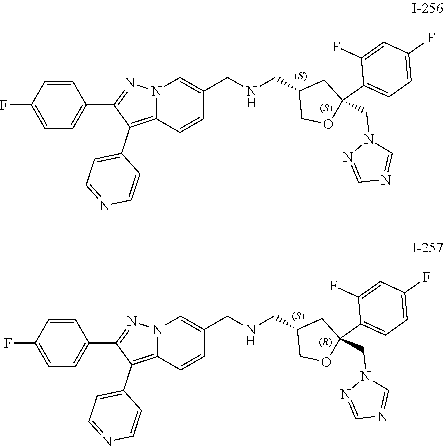

(2-(4-fluorophenyl)-3-(pyridin-4-yl)pyrazolo[1,5-a]pyridin-6-yl)methanamine 11-3 (271 mg, 0.853 mmol) and trans-5-((1H-1,2,4-triazol-1-yl)methyl)-5-(2,4-difluorophenyl)tetrahydrofuran-3-carbaldehyde 11-2 (250 mg, 0.853 mmol) were dissolved in 15 mL EtOH, followed by adding titanium tetraisopropanolate (4 drops). The reaction was stirred at 90° C. by microwave for 2 hours. Then NaBH₄ (162 mg, 4.26 mmol) was added and stirred at room temperature for 30 minutes. The reaction solution was quenched with water (50 mL) and extracted with AcOEt (20 mL*3). The organic layer was washed with brine (30 mL), dried over Na₂SO₄, filtered, concentrated under reduced pressure and purified by Prep-HPLC (ACN/H₂O with 0.05% NH₃) to afford 1-(trans-5-((1H-1,2,4-triazol-1-yl)methyl)-5-(2,4-difluorophenyl)tetrahydrofuran-3-yl)-N-((2-(4-fluorophenyl)-3-(pyridin-4-yl)pyrazolo[1,5-a]pyridin-6-yl)methyl)methanamine 1-256 (60 mg) as a white solid with 11.8% yield, and 1-(cis-5-((1H-1,2,4-triazol-1-yl)methyl)-5-(2,4-difluorophenyl)tetrahydrofuran-3-yl)-N-((2-(4-fluorophenyl)-3-(pyridin-4-yl)pyrazolo[1,5-a]pyridin-6-yl)methyl)methanamine 1-257 (53 mg) as a white solid with 10.4% yield.

1-(trans-5-((1H-1,2,4-triazol-1-yl)methyl)-5-(2,4-difluorophenyl)tetrahydrofuran-3-yl)-N-((2-(4-fluorophenyl)-3-(pyridin-4-yl)pyrazolo[1,5-a]pyridin-6-yl)methyl)methanamine 1-256 LCMS: Calculated Exact Mass=595.2, Found [M+H]+ (ESI+)=596.2

¹H NMR (400 MHz, DMSO) δ 8.59 (s, 1H), 8.56 (dd, J=4.5, 1.6 Hz, 2H), 8.32 (s, 1H), 7.80 (s, 1H), 7.69 (d, J=9.1 Hz, 1H), 7.56-7.50 (m, 2H), 7.34-7.23 (m, 6H), 7.19 (ddd, J=11.6, 9.2, 2.5 Hz, 1H), 6.87 (td, J=8.4, 2.3 Hz, 1H), 4.43 (dd, J=43.5, 14.3 Hz, 2H), 4.08-4.02 (m, 1H), 3.64 (s, 2H), 3.53 (t, J=8.0 Hz, 1H), 2.69-2.60 (m, 1H), 2.39-2.25 (m, 3H), 2.16-2.04 (m, 1H), 1.80 (dd, J=11.4, 7.9 Hz, 1H).

1-(cis-5-((1H-1,2,4-triazol-1-yl)methyl)-5-(2,4-difluorophenyl)tetrahydrofuran-3-yl)-N-((2-(4-fluorophenyl)-3-(pyridin-4-yl)pyrazolo[1,5-a]pyridin-6-yl)methyl)methanamine I-257. LCMS: Calculated Exact Mass=595.2, Found [M+H]+ (ESI+)=596.2.

¹H NMR (400 MHz, DMSO) δ 8.66 (s, 1H), 8.56 (dd, J=4.5, 1.6 Hz, 2H), 8.30 (s, 1H), 7.75 (s, 1H), 7.71 (d, J=9.2 Hz, 1H), 7.56-7.51 (m, 2H), 7.37 (dd, J=9.2, 1.3 Hz, 1H), 7.32-7.21 (m, 6H), 6.96 (td, J=8.5, 2.4 Hz, 1H), 4.53 (s, 2H), 4.02 (t, J=7.9 Hz, 1H), 3.69 (s, 2H), 3.54 (t, J=8.2 Hz, 1H), 2.45-2.30 (m, 4H), 2.22 (d, J=7.5 Hz, 1H), 1.91 (dd, J=12.7, 9.0 Hz, 1H).

### Example 14—Synthesis of Compound 2-(2,4-difluorophenyl)-1-(((2-(4-fluorophenyl)-3-(pyridin-4-yl)pyrazolo[1,5-a]pyridin-6-yl)methyl)(methyl)amino)-3-(1H-1,2,4-triazol-1-yl)propan-2-ol I-259

Procedures for the synthesis of compound 2-(2,4-difluorophenyl)-1-(((2-(4-fluorophenyl)-3-(pyridin-4-yl)pyrazolo[1,5-a]pyridin-6-yl)methyl)(methyl)amino)-3-(1H-1,2,4-triazol-1-yl)propan-2-ol I-259 is shown below in Scheme 29:

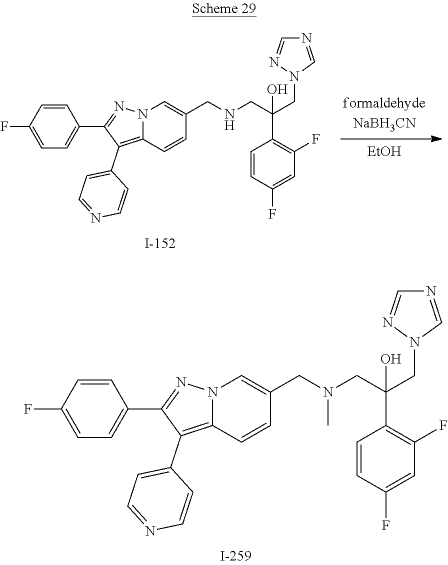 [SMILES: "EtOH" ; OC(CNCc1ccc2c(-c3ccncc3)c(-c3ccc(F)cc3)nn2c1)(Cn1cncn1)c1ccc(F)cc1F ; CN(Cc1ccc2c(-c3ccncc3)c(-c3ccc(F)cc3)nn2c1)CC(O)(Cn1cncn1)c1ccc(F)cc1F]

### 2-(2,4-difluorophenyl)-1-(((2-(4-fluorophenyl)-3-(pyridin-4-yl)pyrazolo[1,5-a]pyridin-6-yl)methyl)(methyl)amino)-3-(1H-1,2,4-triazol-1-yl)propan-2-ol (I-259)

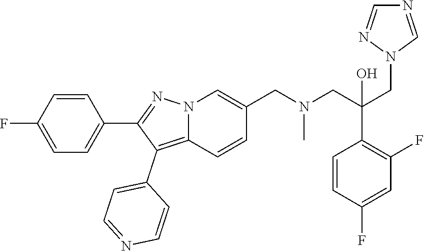 [SMILES: CN(Cc1ccc2c(-c3ccncc3)c(-c3ccc(F)cc3)nn2c1)CC(O)(Cn1cncn1)c1ccc(F)cc1F]

2-(2,4-difluorophenyl)-1-(((2-(4-fluorophenyl)-3-(pyridin-4-yl)pyrazolo[1,5-a]pyridin-6-yl)methyl)amino)-3-(1H-1,2,4-triazol-1-yl)propan-2-ol 1-152 (140 mg, 0.25 mmol) was dissolved in EtOH (5 mL), followed by adding formaldehyde (0.46 mL, 1.25 mmol) and the reaction was stirred at room temperature for 8 hours. Then NaBH₄ (95 mg, 2.5 mmol) was added and the reaction was stirred at 60° C. for 24 hours. The reaction solution was quenched with water (30 mL) and extracted with AcOEt (20 mL*3). The organic layer was washed with brine (30 mL), dried over Na₂SO₄, filtered, concentrated under reduced pressure and purified by Prep-HPLC (ACN/H₂O with 0.05% NH₃) to afford 2-(2,4-difluorophenyl)-1-(((2-(4-fluorophenyl)-3-(pyridin-4-yl)pyrazolo[1,5-a]pyridin-6-yl)methyl)(methyl)amino)-3-(1H-1,2,4-triazol-1-yl)propan-2-ol 1-259 (20 mg, 0.035 mmol) as a white solid with 13.9% yield.

LCMS: Calculated Exact Mass=569.2, Found [M+H]+ (ESI+)=570.2. ¹H NMR (400 MHz, DMSO) δ 8.59-8.52 (m, 3H), 8.29 (s, 1H), 7.73 (s, 1H), 7.61 (d, J=9.2 Hz, 1H), 7.56-7.50 (m, 2H), 7.43 (dd, J=15.9, 8.9 Hz, 1H), 7.28 (dd, J=16.7, 7.5 Hz, 4H), 7.12 (ddd, J=11.8, 9.1, 2.5 Hz, 1H), 7.04 (d, J=9.3 Hz, 1H), 6.96 (td, J=8.5, 2.5 Hz, 1H), 5.82 (s, 1H), 4.54 (q, J=14.3 Hz, 2H), 3.66 (d, J=13.5 Hz, 1H), 3.46 (d, J=13.5 Hz, 1H), 3.10 (d, J=15.0 Hz, 1H), 2.80 (d, J=13.7 Hz, 1H), 2.15 (s, 3H).

### Example 15—Synthesis of Compound 2-(2,4-difluorophenyl)-1-(((2-(4-fluorophenyl)-3-(pyridin-4-yl)pyrazolo[1,5-a]pyridin-6-yl)methyl)amino)-3-(1H-tetrazol-1-yl)propan-2-ol (I-260)

Procedures for the synthesis of compound 2-(2,4-difluorophenyl)-1-(((2-(4-fluorophenyl)-3-(pyridin-4-yl)pyrazolo[1,5-a]pyridin-6-yl)methyl)amino)-3-(1H-tetrazol-1-yl)propan-2-ol (I-260) is shown below in Scheme 30:

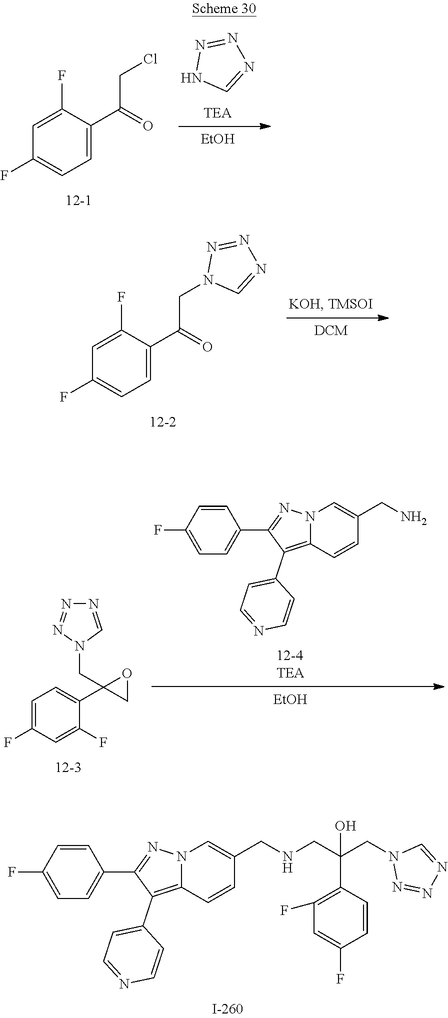

### 1-(2,4-difluorophenyl)-2-(1H-tetrazol-1-yl)ethan-1-one (12-2)

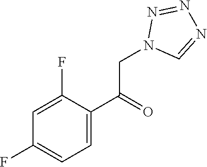 [SMILES: O=C(Cn1cnnn1)c1ccc(F)cc1F]

A solution of 2-chloro-1-(2,4-difluorophenyl)ethan-1-one 12-1 (4.5 g, 23.7 mmol), 1H-tetrazole (1.66 g, 23.7 mmol), Et₃N (6.6 ml, 47.4 mmol) in EtOH (50 mL) was stirred at 70° C. for 8 hours. The reaction solution was quenched with water (100 ml) and extracted with AcOEt (50 mL*3). The organic layer was washed with brine (100 mL), dried over Na₂SO₄, filtered, concentrated under reduced pressure and purified by flash column with DCM/MeOH 0-5% to afford desired product 12-2 (2.1 g, 9.37 mmol) as a yellow solid with 39.6% yield. LCMS: Calculated Exact Mass=224, Found [M+H]+ (ESI+)=225.

### 1-((2-(2,4-difluorophenyl)oxiran-2-yl)methyl)-1H-tetrazole (12-3)

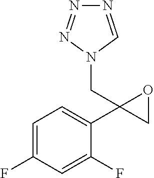 [SMILES: Fc1ccc(C2(Cn3cnnn3)CO2)c(F)c1]

A solution 1-(2,4-difluorophenyl)-2-(1H-tetrazol-1-yl)ethan-1-one 12-2 (2.1 g, 9.3 mmol), TMSOI (8.25 g, 37.5 mmol), KOH (2.13 g, 37.5 mmol) and CTAB (339 mg, 0.93 mmol) in DCM (20 mL) and H₂O (2 mL) was stirred at 50° C. for 24 hours. The reaction solution was quenched with water (150 mL) and extracted with AcOEt (50 mL*3). The organic layer was washed with brine (100 mL), dried over Na₂SO₄, filtered, concentrated under reduced pressure and purified by Prep-HPLC (ACN/H₂O with 0.05% NH₃) to afford desired product 12-3 (1.1 g, 4.62 mmol) as a white solid with 50% yield. LCMS: Calculated Exact Mass=238, Found [M+H]+ (ESI+)=239.

### 2-(2,4-difluorophenyl)-1-(((2-(4-fluorophenyl)-3-(pyridin-4-yl)pyrazolo[1,5-a]pyridin-6-yl)methyl)amino)-3-(1H-tetrazol-1-yl)propan-2-ol (I-260)

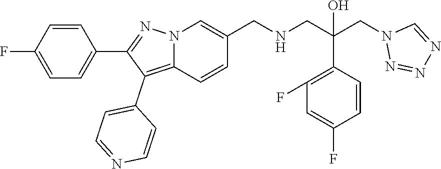 [SMILES: OC(CNCc1ccc2c(-c3ccncc3)c(-c3ccc(F)cc3)nn2c1)(Cn1cnnn1)c1ccc(F)cc1F]

1-((2-(2,4-difluorophenyl)oxiran-2-yl)methyl)-1H-tetrazole 12-3 (224 mg, 0.94 mmol) and (2-(4-fluorophenyl)-3-(pyridin-4-yl)pyrazolo[1,5-a]pyridin-6-yl)methanamine 12-4 (150 mg, 0.47 mmol) were dissolved in 10 mL EtOH, followed by adding Et₃N (0.13 mL, 0.94 mmol). The reaction was stirred at 100° C. under microwave for 3 hours. Then the reaction mixture was concentrated, purified by Prep-HPLC (ACN/H₂O with 0.05% NH₃) to afford 2-(2,4-difluorophenyl)-1-(((2-(4-fluorophenyl)-3-(pyridin-4-yl)pyrazolo[1,5-a]pyridin-6-yl)methyl)amino)-3-(1H-tetrazol-1-yl)propan-2-ol 1-260 (31 mg) as a white solid with 11.8% yield.

LCMS: Calculated Exact Mass=556.2, Found [M+H]+ (ESI+)=557.2. ¹H NMR (400 MHz,) δ 9.12 (s, 1H), 8.67 (s, 1H), 8.57 (d, J=6.0 Hz, 2H), 7.70 (d, J=9.2 Hz, 1H), 7.54 (dd, J=8.7, 5.6 Hz, 2H), 7.30 (dt, J=17.8, 9.6 Hz, 6H), 7.18 (s, 1H), 6.94 (s, 1H), 6.02 (s, 1H), 4.88 (s, 2H), 3.77 (s, 2H), 2.99 (s, 2H), 2.49-2.43 (m, 1H).

### Example 16—Synthesis of Compound 2-(2,4-difluorophenyl)-1-(((2-(4-fluorophenyl)-3-(pyridin-4-yl)pyrazolo[1,5-a]pyridin-6-yl)methyl-d2)amino)-3-(1H-1,2,4-triazol-1-yl)propan-2-ol (1261)

Procedures for the synthesis of compound 2-(2,4-difluorophenyl)-1-(((2-(4-fluorophenyl)-3-(pyridin-4-yl)pyrazolo[1,5-a]pyridin-6-yl)methyl-d2)amino)-3-(1H-1,2,4-triazol-1-yl)propan-2-ol (I-261) is shown below in Scheme 31:

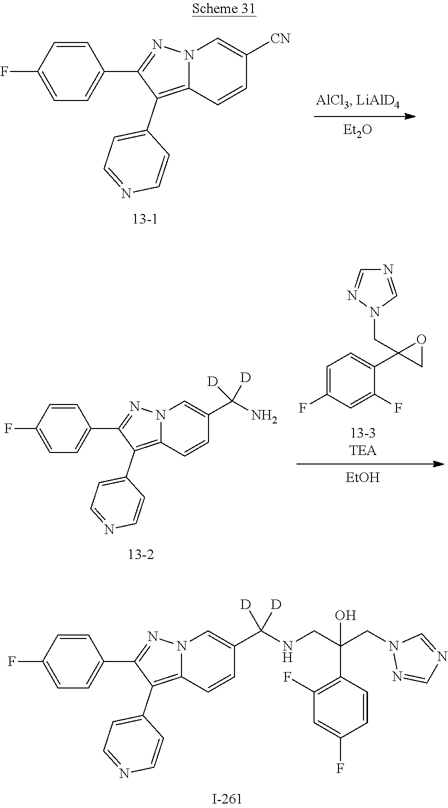 [SMILES: "Et2O" ; N#Cc1ccc2c(-c3ccncc3)c(-c3ccc(F)cc3)nn2c1 ; "EtOH" ; [2H]C([2H])(N)c1ccc2c(-c3ccncc3)c(-c3ccc(F)cc3)nn2c1 ; Fc1ccc(C2(Cn3cncn3)CO2)c(F)c1 ; [2H]C([2H])(NCC(O)(Cn1cncn1)c1ccc(F)cc1F)c1ccc2c(-c3ccncc3)c(-c3ccc(F)cc3)nn2c1]

### (2-(4-fluorophenyl)-3-(pyridin-4-yl)pyrazolo[1,5-a]pyridin-6-yl)methan-d2-amine 13-2

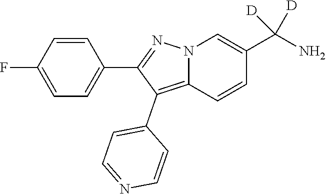 [SMILES: [2H]C([2H])(N)c1ccc2c(-c3ccncc3)c(-c3ccc(F)cc3)nn2c1]

2-(4-fluorophenyl)-3-(pyridin-4-yl)pyrazolo[1,5-a]pyridine-6-carbonitrile 13-1 (250 mg, 0.79 mmol) and AlCl₃ (525 mg, 3.95 mmol) were dissolved in Et₂O (10 mL), followed by adding lithium aluminum deuteride (165.8 mg, 3.95 mmol) at 0° C. The reaction was stirred at room temperature for 16 hours. Then the reaction mixture was diluted with water (50 mL), extracted with EA (30 mL*3). The organic layer was washed with brine (30 mL), dried over Na₂SO₄, filtered, concentrated under reduced pressure and purified by flash column with DCM/MeOH 0-20% to afford desired product 13-2 (100 mg, 0.31 mmol) as a white solid with 39.3% yield. LCMS: Calculated Exact Mass=320, Found [M+H]+ (ESI+)=321.

### 2-(2,4-difluorophenyl)-1-(((2-(4-fluorophenyl)-3-(pyridin-4-yl)pyrazolo[1,5-a]pyridin-6-yl)methyl-d2)amino)-3-(1H-1,2,4-triazol-1-yl)propan-2-ol (I-261)

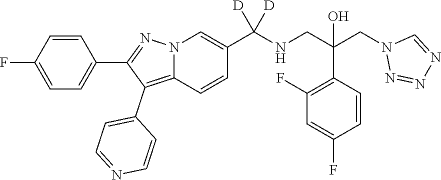 [SMILES: [2H]C([2H])(NCC(O)(Cn1cnnn1)c1ccc(F)cc1F)c1ccc2c(-c3ccncc3)c(-c3ccc(F)cc3)nn2c1]

(2-(4-fluorophenyl)-3-(pyridin-4-yl)pyrazolo[1,5-a]pyridin-6-yl)methan-d2-amine 13-2 (70 mg, 0.218 mmol) and 1-((2-(2,4-difluorophenyl)oxiran-2-yl)methyl)-1H-1,2,4-triazole 13-3 (78 mg, 0.328 mmol) were dissolved in 5 mL EtOH, followed by adding Et₃N (0.06 mL, 0.436 mmol). The reaction was stirred at 90° C. microwave for 2 hours. Then the reaction mixture was concentrated, purified by Prep-HPLC (ACN/H₂O with 0.05% NH₃) to 2-(2,4-difluorophenyl)-1-(((2-(4-fluorophenyl)-3-(pyridin-4-yl)pyrazolo[1,5-a]pyridin-6-yl)methyl-d2)amino)-3-(1H-1,2,4-triazol-1-yl)propan-2-ol 1-261 (19.5 mg) as a white solid with 16.1% yield.

¹H NMR (400 MHz, DMSO) δ 8.64 (s, 1H), 8.56 (dd, J=4.5, 1.5 Hz, 2H), 8.28 (s, 1H), 7.74-7.67 (m, 2H), 7.57-7.51 (m, 2H), 7.38 (dd, J=15.9, 9.0 Hz, 1H), 7.30 (ddd, J=20.7, 11.4, 6.2 Hz, 5H), 7.12 (ddd, J=11.9, 9.2, 2.5 Hz, 1H), 6.94 (td, J=8.5, 2.5 Hz, 1H), 5.80 (s, 1H), 4.63-4.52 (m, 2H), 2.94 (dd, J=27.7, 12.5 Hz, 2H), 2.36 (s, 1H).

### Example 16—Synthesis of Compound 2-(2,4-difluorophenyl)-1,1-difluoro-1-(5-(4-((((2-(4-fluorophenyl)-3-(pyridin-4-yl)pyrazolo[1,5-a]pyridin-6-yl)methyl)amino)methyl)phenyl)pyridin-2-yl)-3-(1H-1,2,4-triazol-1-yl)propan-2-ol (I-262)

Procedures for the synthesis of compound 2-(2,4-difluorophenyl)-1,1-difluoro-1-(5-(4-((((2-(4-fluorophenyl)-3-(pyridin-4-yl)pyrazolo[1,5-a]pyridin-6-yl)methyl)amino)methyl)phenyl)pyridin-2-yl)-3-(1H-1,2,4-triazol-1-yl)propan-2-ol (I-262) is shown below in Scheme 32:

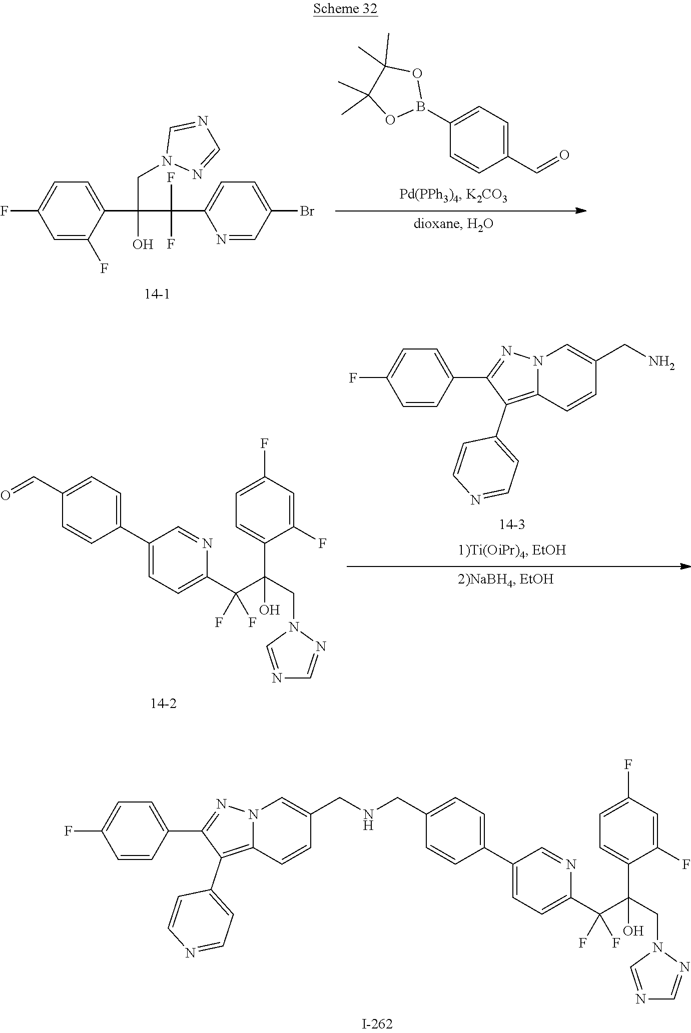 [SMILES: OC(Cn1cncn1)(c1ccc(F)cc1F)C(F)(F)c1ccc(Br)cn1 ; CC1(C)OB(c2ccc(C=O)cc2)OC1(C)C ; O=Cc1ccc(-c2ccc(C(F)(F)C(O)(Cn3cncn3)c3ccc(F)cc3F)nc2)cc1 ; NCc1ccc2c(-c3ccncc3)c(-c3ccc(F)cc3)nn2c1 ; OC(Cn1cncn1)(c1ccc(F)cc1F)C(F)(F)c1ccc(-c2ccc(CNCc3ccc4c(-c5ccncc5)c(-c5ccc(F)cc5)nn4c3)cc2)cn1]

### 4-(6-(2-(2,4-difluorophenyl)-1,1-difluoro-2-hydroxy-3-(1H-1,2,4-triazol-1-yl)propyl)pyridin-3-yl)benzaldehyde (14-2)

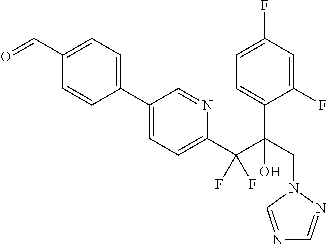 [SMILES: O=Cc1ccc(-c2ccc(C(F)(F)C(O)(Cn3cncn3)c3ccc(F)cc3F)nc2)cc1]

A solution of 1-(5-bromopyridin-2-yl)-2-(2,4-difluorophenyl)-1,1-difluoro-3-(1H-1,2,4-triazol-1-yl)propan-2-ol 14-1 (synthesized according to patent: WO2015/143172, A1) (550 mg, 1.28 mmol), 4-(4,4,5,5-tetramethyl-1,3,2-dioxaborolan-2-yl)benzaldehyde (594 mg, 2.56 mmol), tetrakis(triphenylphosphine)palladium(0) (148 mg, 0.13 mmol) and potassium carbonate (353.5 mg, 9.51 mmol) in dioxane (10 mL) and H₂O (2.5 mL) was stirred at 100° C. by microwave for 2 hours. Then the reaction was cooled to room temperature and filtered through a pad of Celite and rinsed with EtOAc (50 mL). The filtrate was washed with water (30 mL*2), brine (30 mL), dried over Na₂SO₄, filtered, concentrated under reduced pressure and purified by flash column with DCM/MeOH 0-5% to afford desired product 14-2 (450 mg, 0.98 mmol) as a yellow solid with 77.1% yield. LCMS: Calculated Exact Mass=456.1, Found [M+H]+ (ESI+)=457.1.

### 2-(2,4-difluorophenyl)-1,1-difluoro-1-(5-(4-((((2-(4-fluorophenyl)-3-(pyridin-4-yl)pyrazolo[1,5-a]pyridin-6-yl)methyl)amino)methyl)phenyl)pyridin-2-yl)-3-(1H-1,2,4-triazol-1-yl)propan-2-ol (I-262)

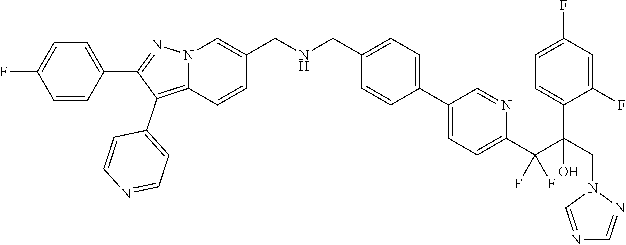 [SMILES: OC(Cn1cncn1)(c1ccc(F)cc1F)C(F)(F)c1ccc(-c2ccc(CNCc3ccc4c(-c5ccncc5)c(-c5ccc(F)cc5)nn4c3)cc2)cn1]

A solution of 4-(6-(2-(2,4-difluorophenyl)-1,1-difluoro-2-hydroxy-3-(1H-1,2,4-triazol-1-yl)propyl)pyridin-3-yl)benzaldehyde 14-2 (150 mg, 0.328 mmol), (2-(4-fluorophenyl)-3-(pyridin-4-yl)pyrazolo[1,5-a]pyridin-6-yl)methanamine 14-3 (156 mg, 0.482 mmol), and titanium tetraisopropanolate (2 drops) in EtOH (10 mL) was stirred at 90° C. by microwave for 2 hours. Then the reaction was cooled to room temperature and sodium borohydride (62 mg, 1.64 mmol) was added into the mixture and stirred at room temperature for 30 minutes. The mixture was concentrated and then purified by Prep-HPLC (ACN/H₂O with 0.05% NH₃) to afford 2-(2,4-difluorophenyl)-1,1-difluoro-1-(5-(4-((((2-(4-fluorophenyl)-3-(pyridin-4-yl)pyrazolo[1,5-a]pyridin-6-yl)methyl)amino)methyl)phenyl)pyridin-2-yl)-3-(1H-1,2,4-triazol-1-yl)propan-2-ol I-262 (54.3 mg) as a white solid with 21.8% yield.

LCMS: Calculated Exact Mass=758.2, Found [M+H]+ (ESI+)=759.1. ¹H NMR (400 MHz, DMSO) δ 8.91 (d, J=1.9 Hz, 1H), 8.70 (s, 1H), 8.59-8.52 (m, 2H), 8.36 (s, 1H), 8.20 (d, J=8.3 Hz, 1H), 7.77-7.69 (m, 4H), 7.56-7.49 (m, 5H), 7.45 (d, J=9.2 Hz, 1H), 7.33-7.23 (m, 5H), 7.13 (ddd, J=11.9, 9.2, 2.5 Hz, 1H), 7.05 (s, 1H), 6.86 (td, J=8.6, 2.4 Hz, 1H), 5.39 (d, J=14.7 Hz, 1H), 4.86 (d, J=14.6 Hz, 1H), 3.79 (s, 4H), 2.98 (s, 1H).

### Example 17—Synthesis of (R)-2-(2,4-difluorophenyl)-1-(((S)-1-(2-(4-fluorophenyl)-3-(pyridin-4-yl)pyrazolo[1,5-a]pyridin-6-yl)ethyl)amino)-3-(1H-1,2,4-triazol-1-yl)propan-2-ol (relative stereochemistry) (I-263) and (S)-2-(2,4-difluorophenyl)-1-(((S)-1-(2-(4-fluorophenyl)-3-(pyridin-4-yl)pyrazolo[1,5-a]pyridin-6-yl)ethyl)amino)-3-(1H-1,2,4-triazol-1-yl)propan-2-ol (relative stereochemistry) (I-264)

Procedures for the synthesis of compounds 1-263 and 1-264 is shown below in Scheme 33:

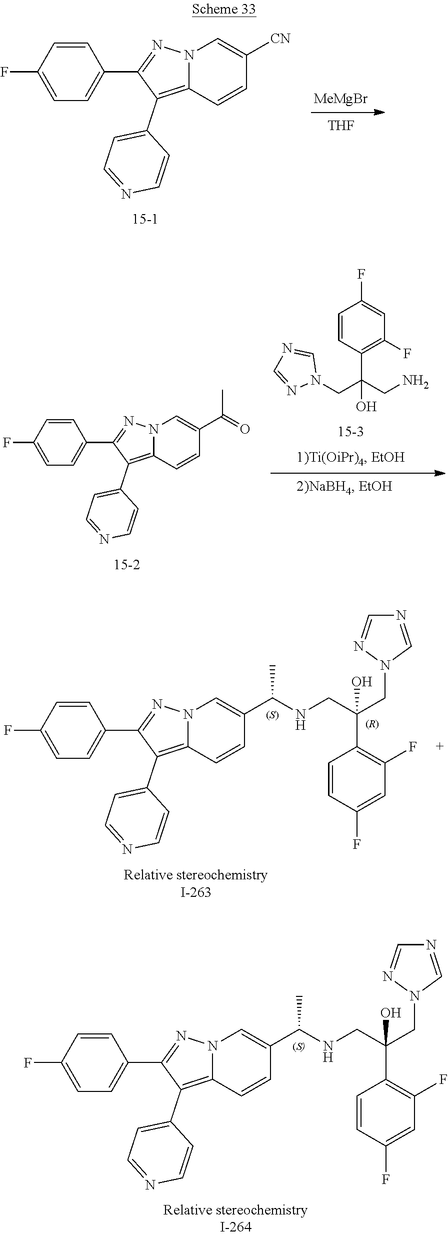

### 1-(2-(4-fluorophenyl)-3-(pyridin-4-yl)pyrazolo[1,5-a]pyridin-6-yl)ethan-1-one (15-2)

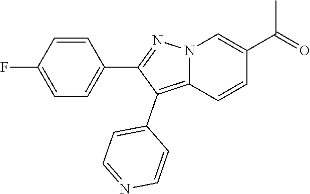 [SMILES: CC(=O)c1ccc2c(-c3ccncc3)c(-c3ccc(F)cc3)nn2c1]

2-(4-fluorophenyl)-3-(pyridin-4-yl)pyrazolo[1,5-a]pyridine-6-carbonitrile 15-1 (350 mg, 1.11 mmol) was dissolved in THF (3 mL), followed by adding MeMgBr (11.1 ml, 11.1 mmol) at 0° C. The reaction was stirred at 70° C. for 16 hours. Then the reaction mixture was diluted with saturated ammonium chloride solution (50 mL), extracted with EA (30 mL*3). The organic layer was washed with brine (30 mL), dried over Na₂SO₄, filtered, concentrated under reduced pressure and purified by flash column with DCM/MeOH 0-10% to afford desired product 15-2 (40 mg) as a yellow solid with 10.8% yield. LCMS: Calculated Exact Mass=331, Found [M+H]+ (ESI+)=332.

### (R)-2-(2,4-difluorophenyl)-1-(((S)-1-(2-(4-fluorophenyl)-3-(pyridin-4-yl)pyrazolo[1,5-a]pyridin-6-yl)ethyl)amino)-3-(1H-1,2,4-triazol-1-yl)propan-2-ol (relative stereochemistry) (I-263) and (S)-2-(2,4-difluorophenyl)-1-(((S)-1-(2-(4-fluorophenyl)-3-(pyridin-4-yl)pyrazolo[1,5-a]pyridin-6-yl)ethyl)amino)-3-(1H-1,2,4-triazol-1-yl)propan-2-ol (relative stereochemistry) (I-264)

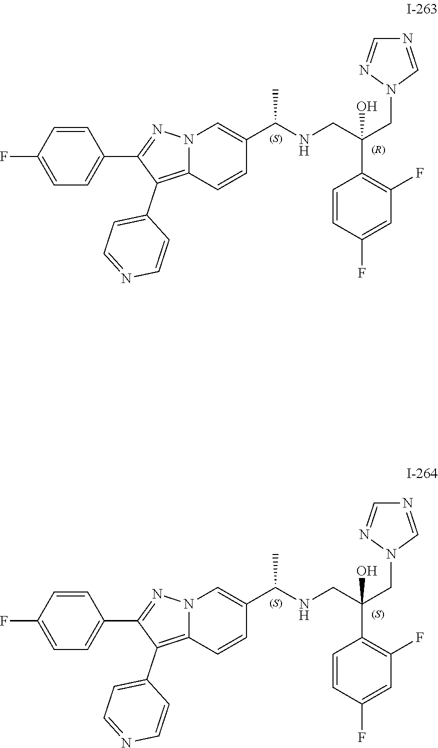

A solution of 1-(2-(4-fluorophenyl)-3-(pyridin-4-yl)pyrazolo[1,5-a]pyridin-6-yl)ethan-1-one 15-2 (40 mg, 0.12 mmol), 1-amino-2-(2,4-difluorophenyl)-3-(1H-1,2,4-triazol-1-yl)propan-2-ol 15-3 (46 mg, 0.18 mmol), and titanium tetraisopropanolate (2 drops) in EtOH (10 mL) was stirred at 90° C. in microwave for 8 hours. Then the reaction was cooled to room temperature and sodium borohydride (46 mg, 1.2 mmol) was added into the mixture and stirred at 50° C. for 16 hours. The mixture was filtered and concentrated then purified by Prep-HPLC (ACN/H₂O with 0.05% NH₃) to afford (R)-2-(2,4-difluorophenyl)-1-(((S)-1-(2-(4-fluorophenyl)-3-(pyridin-4-yl)pyrazolo[1,5-a]pyridin-6-yl)ethyl)amino)-3-(1H-1,2,4-triazol-1-yl)propan-2-ol (relative stereochemistry) 1-263 (1.8 mg) as a white solid with 2.6% yield and (S)-2-(2,4-difluorophenyl)-1-(((S)-1-(2-(4-fluorophenyl)-3-(pyridin-4-yl)pyrazolo[1,5-a]pyridin-6-yl)ethyl)amino)-3-(1H-1,2,4-triazol-1-yl)propan-2-ol (relative stereochemistry) 1-264 (1.5 mg) as a white solid with 2.2% yield.

(R)-2-(2,4-difluorophenyl)-1-(((S)-1-(2-(4-fluorophenyl)-3-(pyridin-4-yl)pyrazolo[1,5-a]pyridin-6-yl)ethyl)amino)-3-(1H-1,2,4-triazol-1-yl)propan-2-ol (relative stereochemistry) (I-263) LCMS: Calculated Exact Mass=569.2, Found [M+H]+ (ESI+)=570.2. ¹H NMR (400 MHz, DMSO) δ 8.59-8.54 (m, 3H), 8.28 (s, 1H), 7.73 (s, 1H), 7.66 (d, J=9.2 Hz, 1H), 7.54 (dd, J=8.7, 5.6 Hz, 2H), 7.37 (dd, J=16.0, 9.0 Hz, 1H), 7.32-7.24 (m, 5H), 7.07 (ddd, J=11.8, 9.2, 2.5 Hz, 1H), 6.94 (td, J=8.5, 2.5 Hz, 1H), 5.80 (s, 1H), 4.57-4.48 (m, 2H), 3.83-3.74 (m, 1H), 2.84 (d, J=6.8 Hz, 2H), 2.23 (s, 1H), 1.28 (d, J=6.5 Hz, 3H).

(S)-2-(2,4-difluorophenyl)-1-(((S)-1-(2-(4-fluorophenyl)-3-(pyridin-4-yl)pyrazolo[1,5-a]pyridin-6-yl)ethyl)amino)-3-(1H-1,2,4-triazol-1-yl)propan-2-ol (relative stereochemistry) (I-264) LCMS: Calculated Exact Mass=569.2, Found [M+H]+ (ESI+)=570.2. ¹H NMR (400 MHz, DMSO) δ 8.64 (s, 1H), 8.57 (d, J=6.0 Hz, 2H), 8.26 (s, 1H), 7.73 (d, J=9.3 Hz, 1H), 7.67 (s, 1H), 7.54 (dd, J=8.7, 5.6 Hz, 2H), 7.41 (d, J=9.4 Hz, 1H), 7.36 (d, J=7.0 Hz, 1H), 7.33-7.25 (m, 4H), 7.11 (ddd, J=11.9, 9.3, 2.4 Hz, 1H), 6.94 (td, J=8.6, 2.5 Hz, 1H), 5.79 (s, 1H), 4.62 (d, J=14.3 Hz, 1H), 4.51 (d, J=14.3 Hz, 1H), 3.76-3.68 (m, 1H), 2.84 (dd, J=14.6, 6.6 Hz, 2H), 2.38 (s, 1H), 1.30 (d, J=6.6 Hz, 3H).

### Example 18—Synthesis of Compound 2-(2,4-difluorophenyl)-1,1-difluoro-1-(5-(4-(((2-(4-fluorophenyl)-3-(pyridin-4-yl)pyrazolo[1,5-a]pyridin-6-yl)methyl)amino)phenyl)pyridin-2-yl)-3-(1H-1,2,4-triazol-1-yl)propan-2-ol (I-265)

Procedures for the synthesis of compound 2-(2,4-difluorophenyl)-1,1-difluoro-1-(5-(4-(((2-(4-fluorophenyl)-3-(pyridin-4-yl)pyrazolo[1,5-a]pyridin-6-yl)methyl)amino)phenyl)pyridin-2-yl)-3-(1H-1,2,4-triazol-1-yl)propan-2-ol (I-265) is shown below in Scheme 34:

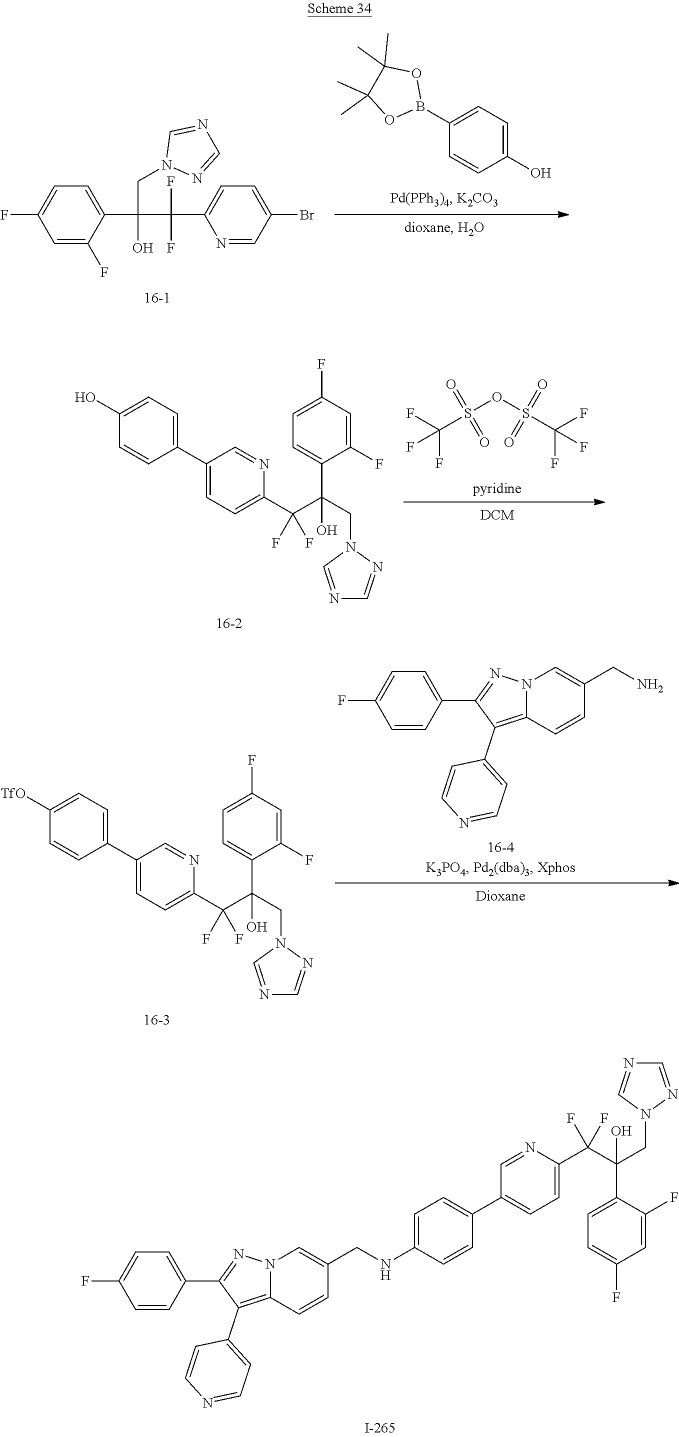

### 4-(6-(2-(2,4-difluorophenyl)-1,1-difluoro-2-hydroxy-3-(1H-1,2,4-triazol-1-yl)propyl)pyridin-3-yl)phenol (16-2)

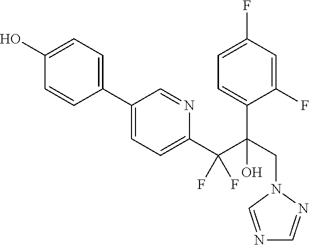 [SMILES: Oc1ccc(-c2ccc(C(F)(F)C(O)(Cn3cncn3)c3ccc(F)cc3F)nc2)cc1]

A solution of 1-(5-bromopyridin-2-yl)-2-(2,4-difluorophenyl)-1,1-difluoro-3-(1H-1,2,4-triazol-1-yl)propan-2-ol 16-1 (550 mg, 1.28 mmol), 4-(4,4,5,5-tetramethyl-1,3,2-dioxaborolan-2-yl)phenol (563 mg, 2.56 mmol), tetrakis(triphenylphosphine)palladium(0) (148 mg, 0.13 mmol) and potassium carbonate (353.5 mg, 9.51 mmol) in dioxane (10 mL) and H₂O (2.5 mL) was stirred at 100° C. in microwave for 2 hours. Then the reaction was cooled to room temperature and filtered through a pad of Celite and rinsed with EtOAc (50 mL). The filtrate was washed with water (30 mL*2), brine (30 mL), dried over Na₂SO₄, filtered, concentrated under reduced pressure and purified by flash column with DCM/MeOH 0-5% to afford desired product 16-2 (450 mg) as a yellow solid with 79% yield. LCMS: Calculated Exact Mass=444.1, Found [M+H]+ (ESI+)=445.1.

### 4-(6-(2-(2,4-difluorophenyl)-1,1-difluoro-2-hydroxy-3-(1H-1,2,4-triazol-1-yl)propyl)pyridin-3-yl)phenyl trifluoromethanesulfonate (16-3)

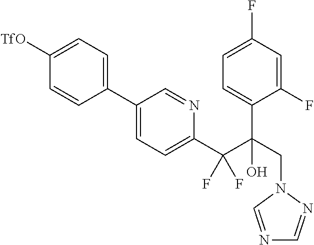 [SMILES: O=S(=O)(Oc1ccc(-c2ccc(C(F)(F)C(O)(Cn3cncn3)c3ccc(F)cc3F)nc2)cc1)C(F)(F)F]

4-(6-(2-(2,4-difluorophenyl)-1,1-difluoro-2-hydroxy-3-(1H-1,2,4-triazol-1-yl)propyl)pyridin-3-yl)phenol 16-2 (250 mg, 0.56 mmol) and pyridine (222.5 mg, 2.81 mmol) were dissolved in DCM (5 mL), followed by adding trifluoromethanesulfonic anhydride (317.4 mg, 1.13 mmol) at 0° C. The reaction was stirred at room temperature for 16 hours. Then the reaction mixture was diluted with water (30 mL), extracted with DCM (20 mL*2). The organic layer was washed with brine (20 mL), dried over Na₂SO₄, filtered, concentrated under reduced pressure and purified by flash column with DCM/MeOH 0-5% to afford desired product 16-3 (200 mg) as a yellow solid with 62% yield. LCMS: Calculated Exact Mass=576, Found [M+H]+ (ESI+)=577.

### 2-(2,4-difluorophenyl)-1,1-difluoro-1-(5-(4-(((2-(4-fluorophenyl)-3-(pyridin-4-yl)pyrazolo[1,5-a]pyridin-6-yl)methyl)amino)phenyl)pyridin-2-yl)-3-(1H-1,2,4-triazol-1-yl)propan-2-ol (I-265)

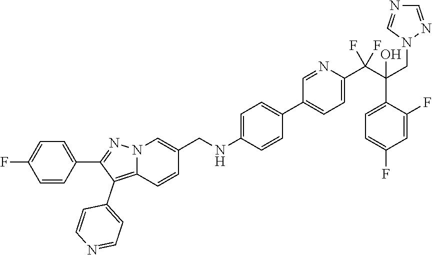 [SMILES: OC(Cn1cncn1)(c1ccc(F)cc1F)C(F)(F)c1ccc(-c2ccc(NCc3ccc4c(-c5ccncc5)c(-c5ccc(F)cc5)nn4c3)cc2)cn1]

A solution of 4-(6-(2-(2,4-difluorophenyl)-1,1-difluoro-2-hydroxy-3-(1H-1,2,4-triazol-1-yl)propyl)pyridin-3-yl)phenyl trifluoromethanesulfonate 16-3 (145 mg, 0.25 mmol), (2-(4-fluorophenyl)-3-(pyridin-4-yl)pyrazolo[1,5-a]pyridin-6-yl)methanamine 16-4 (80 mg, 0.25 mmol), tris(dibenzylideneacetone)dipalladium (46 mg, 0.05 mmol), potassium phosphate tribasic (106.8 mg, 0.5 mmol) and X-Phos (24 mg, 0.05 mmol) in dioxane (10 mL) was stirred at 100° C. by microwave for 6 hours. Then the reaction was cooled to room temperature and filtered through a pad of Celite and rinsed with EtOAc (30 mL). The organic layer was washed with brine (20 ml), dried over Na₂SO₄, filtered, concentrated under reduced pressure and purified by Prep-HPLC (ACN/H₂O with 0.05% NH₃) to afford 2-(2,4-difluorophenyl)-1,1-difluoro-1-(5-(4-(((2-(4-fluorophenyl)-3-(pyridin-4-yl)pyrazolo[1,5-a]pyridin-6-yl)methyl)amino)phenyl)pyridin-2-yl)-3-(1H-1,2,4-triazol-1-yl)propan-2-ol 1-265 (11.3 mg) as a white solid with 6.1% yield.

LCMS: Calculated Exact Mass=744.2, Found [M+H]+ (ESI+)=745.1 ¹H NMR (400 MHz, DMSO) δ 8.82-8.76 (m, 2H), 8.56 (dd, J=4.5, 1.5 Hz, 2H), 8.35 (s, 1H), 8.04 (dd, J=8.3, 2.2 Hz, 1H), 7.76 (d, J=9.2 Hz, 1H), 7.69 (s, 1H), 7.57-7.49 (m, 4H), 7.44 (dd, J=9.2, 1.2 Hz, 1H), 7.39 (d, J=8.3 Hz, 1H), 7.30 (dd, J=4.5, 1.6 Hz, 2H), 7.29-7.22 (m, 3H), 7.11 (ddd, J=12.0, 9.2, 2.6 Hz, 1H), 7.01 (s, 1H), 6.88-6.82 (m, 1H), 6.80 (d, J=8.7 Hz, 3H), 5.36 (d, J=14.7 Hz, 1H), 4.83 (d, J=14.7 Hz, 1H), 4.44 (d, J=6.0 Hz, 2H).

### Example 19—Synthesis of Compound (2R,3R)-2-(2,4-difluorophenyl)-3-(((2-(4-fluorophenyl)-3-(pyridin-4-yl)pyrazolo[1,5-a]pyridin-6-yl)methyl)amino)-1-(1H-1,2,4-triazol-1-yl)butan-2-ol (I-266)

Procedures for the synthesis of compound (2R,3R)-2-(2,4-difluorophenyl)-3-(((2-(4-fluorophenyl)-3-(pyridin-4-yl)pyrazolo[1,5-a]pyridin-6-yl)methyl)amino)-1-(1H-1,2,4-triazol-1-yl)butan-2-ol (I-266) is shown below in Scheme 35:

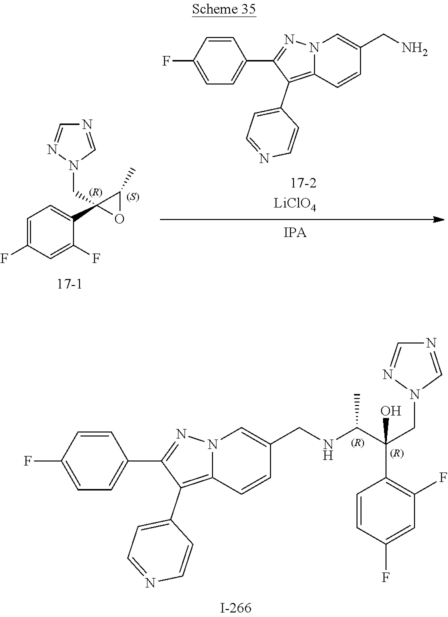

### (2E,3R)-2-(2,4-difluorophenyl)-3-(((2-(4-fluorphenyl)-3-(pyridin-4-yl)pyrazolo[1,5-a]pyridin-6-yl)methyl)amino)-1-(1H-1,2,4-triazol-1-yl)butan-2-ol (I-266)

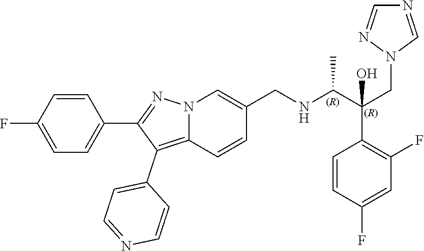

A solution of 1-(((2R,3S)-2-(2,4-difluorophenyl)-3-methyloxiran-2-yl)methyl)-1H-1,2,4-triazole 17-1 (200 mg, 0.796 mmol), (2-(4-fluorophenyl)-3-(pyridin-4-yl)pyrazolo[1,5-a]pyridin-6-yl)methanamine 17-2 (506 mg, 1.593 mmol), and LiClO₄ (338 mg, 3.184 mmol) in IPA (20 mL) was stirred at 120° C. microwave for 20 hours. The mixture was concentrated then purified by Prep-HPLC (ACN/H₂O with 0.05% NH₃) to afford (2R,3R)-2-(2,4-difluorophenyl)-3-(((2-(4-fluorophenyl)-3-(pyridin-4-yl)pyrazolo[1,5-a]pyridin-6-yl)methyl)amino)-1-(1H-1,2,4-triazol-1-yl)butan-2-ol 1-266 (26.5 mg) as a white solid with 5.84% yield.

LCMS: Calculated Exact Mass=569.2, Found [M+H]+ (ESI+)=570.2 ¹H NMR (400 MHz, DMSO) δ 8.88 (s, 1H), 8.57 (dd, J=4.5, 1.6 Hz, 2H), 8.25 (s, 1H), 7.77 (d, J=9.2 Hz, 1H), 7.61 (s, 1H), 7.59-7.50 (m, 3H), 7.33 (dd, J=4.5, 1.6 Hz, 2H), 7.31-7.22 (m, 3H), 7.08 (ddd, J=11.9, 9.2, 2.5 Hz, 1H), 6.86 (td, J=8.5, 2.5 Hz, 1H), 5.53 (s, 1H), 4.97 (d, J=14.4 Hz, 1H), 4.63 (d, J=14.7 Hz, 1H), 4.05 (d, J=13.3 Hz, 1H), 3.83 (d, J=11.7 Hz, 1H), 3.21 (s, 1H), 2.11 (s, 1H), 0.81 (d, J=6.5 Hz, 3H).

### Example 20—Synthesis of Compound 2-(2,4-difluorophenyl)-1,1-difluoro-1-(5-((4-((((2-(4-fluorophenyl)-3-(pyridin-4-yl)pyrazolo[1,5-a]pyridin-6-yl)methyl)amino)methyl)phenyl)ethynyl)pyridin-2-yl)-3-(1H-1,2,4-triazol-1-yl)propan-2-ol (I-267)

Procedures for the synthesis of compound 2-(2,4-difluorophenyl)-1,1-difluoro-1-(5-((4-((((2-(4-fluorophenyl)-3-(pyridin-4-yl)pyrazolo[1,5-a]pyridin-6-yl)methyl)amino)methyl)phenyl)ethynyl)pyridin-2-yl)-3-(1H-1,2,4-triazol-1-yl)propan-2-ol (I-267) is shown below in Scheme 36:

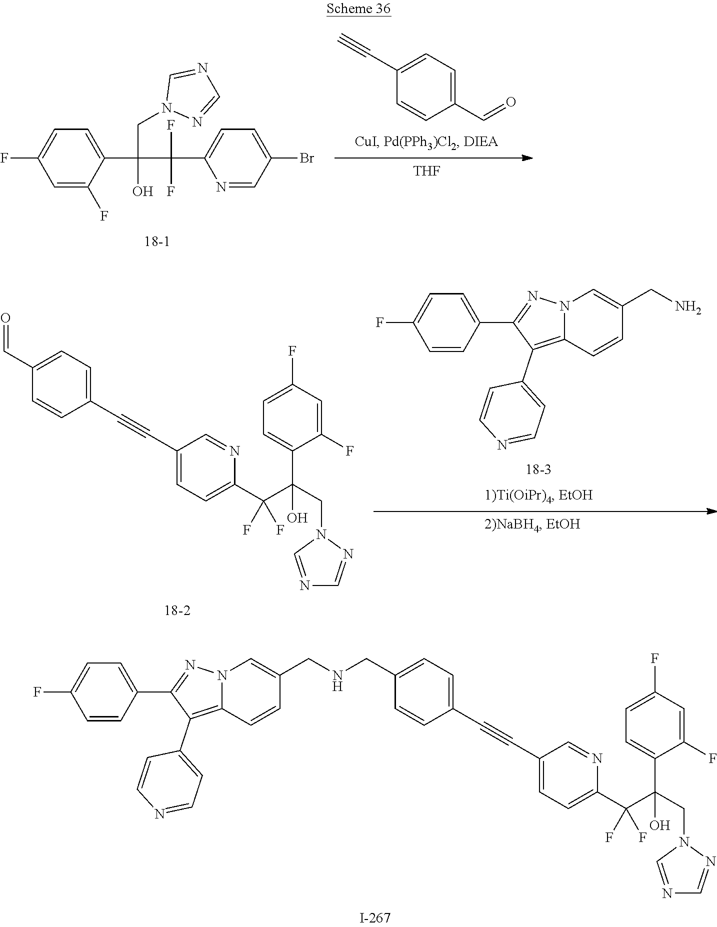

### 4-(6-(2-(2,4-difluorophenyl)-1,1-difluoro-2-hydroxy-3-(1H-1,2,4-triazol-1-yl) propyl)pyridin-3-yl)phenol-(18-2)

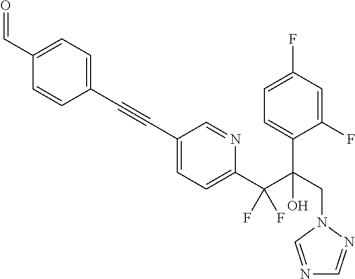 [SMILES: O=Cc1ccc(C#Cc2ccc(C(F)(F)C(O)(Cn3cncn3)c3ccc(F)cc3F)nc2)cc1]

A solution of 1-(5-bromopyridin-2-yl)-2-(2,4-difluorophenyl)-1,1-difluoro-3-(1H-1,2,4-triazol-1-yl)propan-2-ol 18-1 (270 mg, 0.63 mmol), 4-ethynylbenzaldehyde (163.3 mg, 1.26 mmol), bis(triphenylphosphine)palladium(II) chloride (44 mg, 0.06 mmol) and N,N-diisopropylethylamine (162.3 mg, 1.26 mmol) in THF (10 mL) was stirred at 80° C. microwave for 2 hours. Then the reaction was cooled to room temperature and filtered through a pad of Celite and rinsed with EtOAc (30 mL). The organic layer was washed with brine (20 mL), dried over Na₂SO₄, filtered, concentrated under reduced pressure and purified by flash column with DCM/MeOH 0-5% to afford desired product 18-2 (200 mg) as a brown solid with 66% yield. LCMS: Calculated Exact Mass=480.1, Found [M+H]+ (ESI+)=481.1.

### 2-(2,4-difluorophenyl)-1,1-difluoro-1-(5-((4-((((2-(4-fluorophenyl)-3-(pyridin-4-yl)pyrazolo[1,5-a]pyridin-6-yl)methyl)amino)methyl)phenyl)ethynyl)pyridin-2-yl)-3-(1H-1,2,4-triazol-1-yl)propan-2-ol (I-267)

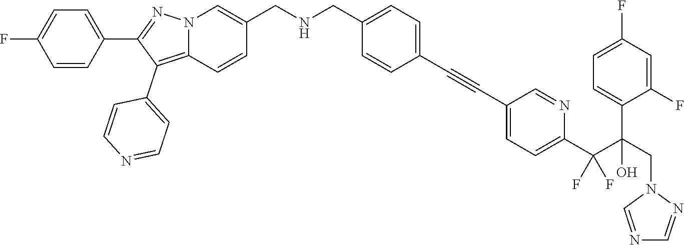 [SMILES: OC(Cn1cncn1)(c1ccc(F)cc1F)C(F)(F)c1ccc(C#Cc2ccc(CNCc3ccc4c(-c5ccncc5)c(-c5ccc(F)cc5)nn4c3)cc2)cn1]

A solution of 4-(6-(2-(2,4-difluorophenyl)-1,1-difluoro-2-hydroxy-3-(1H-1,2,4-triazol-1-yl)propyl)pyridin-3-yl)phenol 18-2 (200 mg, 0.42 mmol), (2-(4-fluorophenyl)-3-(pyridin-4-yl)pyrazolo[1,5-a]pyridin-6-yl)methanamine 18-3 (100 mg, 0.31 mmol), and titanium tetraisopropanolate (2 drops) in EtOH (10 mL) was stirred at 90° C. in microwave for 2 hours. Then the reaction was cooled to room temperature and sodium borohydride (60 mg, 1.57 mmol) was added into the mixture and stirred at room temperature for 30 minutes. The mixture was concentrated and then purified by Prep-HPLC (ACN/H₂O with 0.05% NH₃) to afford 2-(2,4-difluorophenyl)-1,1-difluoro-1-(5-((4-((((2-(4-fluorophenyl)-3-(pyridin-4-yl)pyrazolo[1,5-a]pyridin-6-yl)methyl)amino)methyl)phenyl)ethynyl)pyridin-2-yl)-3-(1H-1,2,4-triazol-1-yl)propan-2-ol 1-267 (19 mg) as a white solid with 7.8% yield.

LCMS: Calculated Exact Mass=782.2, Found [M+H]+ (ESI+)=783.1 ¹H NMR (400 MHz, DMSO) δ 8.70 (d, J=5.1 Hz, 2H), 8.58-8.54 (m, 2H), 8.36 (s, 1H), 8.07 (dd, J=8.2, 2.0 Hz, 1H), 7.76-7.70 (m, 2H), 7.59-7.52 (m, 4H), 7.50-7.42 (m, 4H), 7.32 (dd, J=4.5, 1.5 Hz, 2H), 7.27 (t, J=8.9 Hz, 2H), 7.19 (d, J=7.0 Hz, 1H), 7.17-7.11 (m, 1H), 7.08 (s, 1H), 6.85 (dd, J=13.7, 5.5 Hz, 1H), 5.35 (d, J=14.7 Hz, 1H), 4.85 (d, J=14.8 Hz, 1H), 3.77 (s, 4H), 2.97 (s, 1H).

### Example 21—Synthesis of Compound 2-(2,4-difluorophenyl)-1,1-difluoro-1-(5-((4-((((2-(4-fluorophenyl)-3-(pyridin-4-yl)pyrazolo[1,5-a]pyridin-6-yl)methyl)amino)methyl)phenyl)ethynyl)pyridin-2-yl)-3-(1H-1,2,4-triazol-1-yl)propan-2-ol (I-268)

Procedures for the synthesis of compound 2-(2,4-difluorophenyl)-1,1-difluoro-1-(5-((4-((((2-(4-fluorophenyl)-3-(pyridin-4-yl)pyrazolo[1,5-a]pyridin-6-yl)methyl)amino)methyl)phenyl)ethynyl)pyridin-2-yl)-3-(1H-1,2,4-triazol-1-yl)propan-2-ol (I-268) is shown below in Scheme 37:

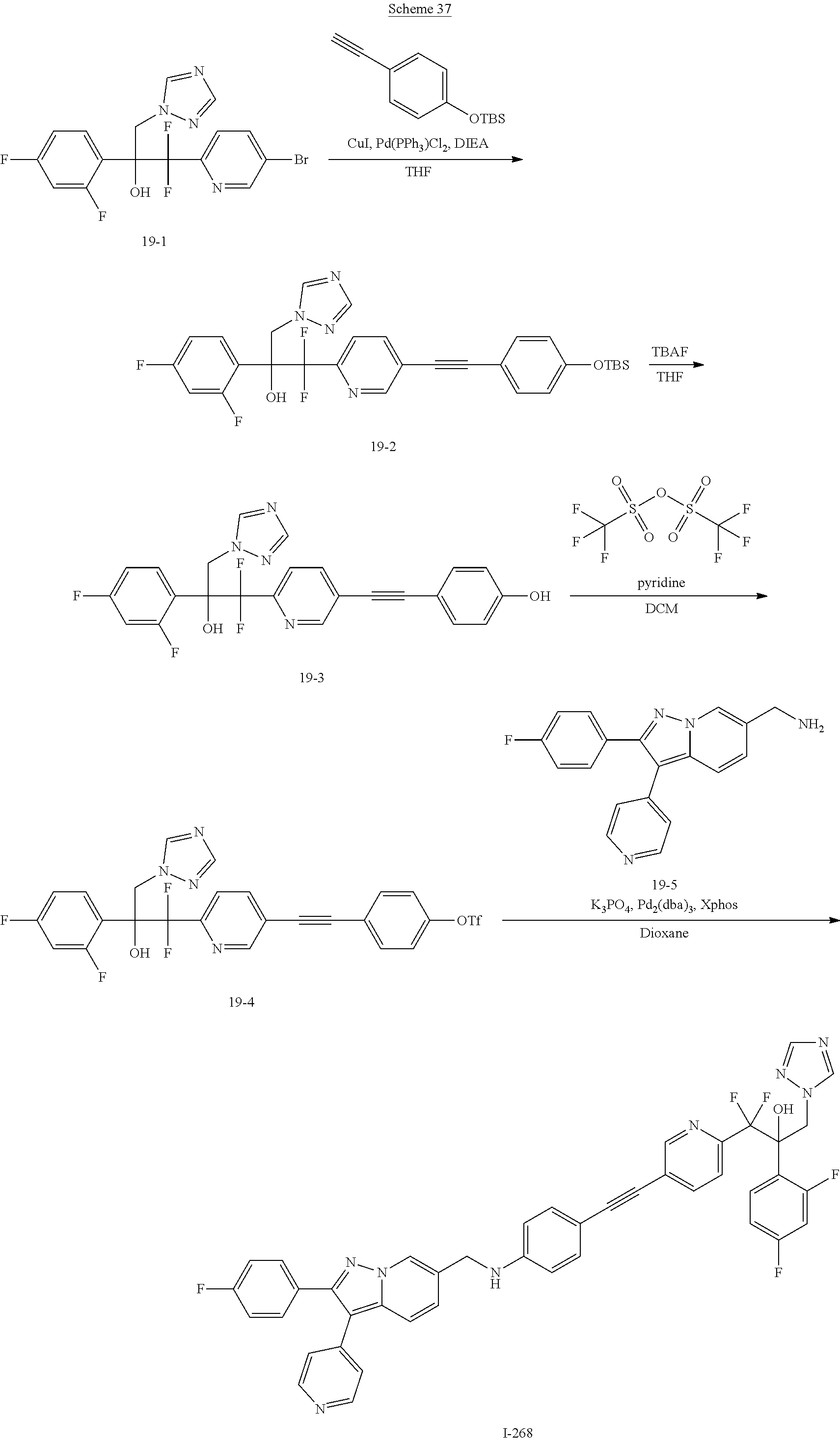

### 1-(5-((4-((tert-butyldimethylsilyl)oxy)phenyl)ethynyl)pyridin-2-yl)-2-(2,4-difluorophenyl)-1,1-difluoro-3-(1H-1,2,4-triazol-1-yl)propan-2-ol (19-2)

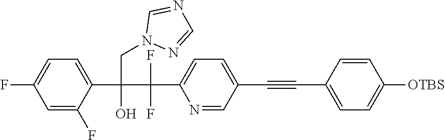 [SMILES: CC(C)(C)[Si](C)(C)Oc1ccc(C#Cc2ccc(C(F)(F)C(O)(Cn3cncn3)c3ccc(F)cc3F)nc2)cc1]

A solution of 1-(5-bromopyridin-2-yl)-2-(2,4-difluorophenyl)-1,1-difluoro-3-(1H-1,2,4-triazol-1-yl)propan-2-ol (850 mg, 1.98 mmol), tert-butyl(4-ethynylphenoxy)dimethylsilane 19-1 (688.3 mg, 2.97 mmol), bis(triphenylphosphine)palladium(II) chloride (138.7 mg, 0.2 mmol) and N,N-diisopropylethylamine (511.6 mg, 3.95 mmol) in THF (10 mL) was stirred at 80° C. by microwave for 2 hours. Then the reaction was cooled to room temperature and filtered through a pad of Celite and rinsed with EtOAc (50 mL). The organic layer was washed with brine (20 mL), dried over Na₂SO₄, filtered, concentrated under reduced pressure and purified by flash column with DCM/MeOH 0-5% to afford desired product 19-2 (1.05 g) as a white solid with 91% yield. LCMS: Calculated Exact Mass=582.2, Found [M+H]+ (ESI+)=583.2.

### 4-((6-(2-(2,4-difluorophenyl)-1,1-difluoro-2-hydroxy-3-(1H-1,2,4-triazol-1-yl)propyl)pyridin-3-yl)ethynyl)phenol (19-3)

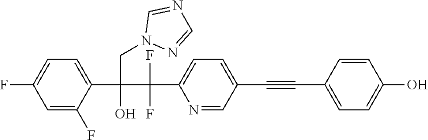 [SMILES: Oc1ccc(C#Cc2ccc(C(F)(F)C(O)(Cn3cncn3)c3ccc(F)cc3F)nc2)cc1]

A solution of 1-(5-((4-((tert-butyldimethylsilyl)oxy)phenyl)ethynyl)pyridin-2-yl)-2-(2,4-difluorophenyl)-1,1-difluoro-3-(1H-1,2,4-triazol-1-yl)propan-2-ol 19-2 (1.05 g, 1.8 mmol) and tetrabutylammonium fluoride (2.7 mL, 1M in THF, 2.7 mmol) in THF (10 mL) was stirred at room temperature for 2 hours. Then the reaction was diluted with water (100 mL), extracted with EtOAc (50 mL*3). The organic layer was washed with brine (20 mL), dried over Na₂SO₄, filtered, concentrated under reduced pressure and purified by flash column with DCM/MeOH 0-10% to afford desired product 19-3 (800 mg) as a white solid with 95% yield. LCMS: Calculated Exact Mass=468.1, Found [M+H]+ (ESI+)=469.1.

### 4-((6-(2-(2,4-difluorophenyl)-1,1-difluoro-2-hydroxy-3-(1H-1,2,4-triazol-1-yl)propyl)pyridin-3-yl)ethynyl)phenyl trifluoromethanesulfonate (19-4)

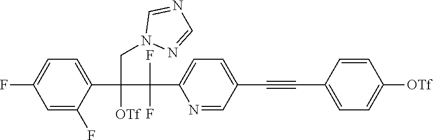 [SMILES: O=S(=O)(Oc1ccc(C#Cc2ccc(C(F)(F)C(Cn3cncn3)(OS(=O)(=O)C(F)(F)F)c3ccc(F)cc3F)nc2)cc1)C(F)(F)F]

4-(6-(2-(2,4-difluorophenyl)-1,1-difluoro-2-hydroxy-3-(1H-1,2,4-triazol-1-yl)propyl)pyridin-3-yl)phenol 19-3 (270 mg, 0.58 mmol) and pyridine (228 mg, 2.88 mmol) were dissolved in DCM (3 mL), followed by adding trifluoromethanesulfonic anhydride (179 mg, 0.63 mmol) at 0° C. The reaction was stirred at 0° C. for 1 hour. Then the reaction mixture was diluted with water (30 mL), extracted with DCM (20 mL*2). The organic layer was washed with brine (20 mL), dried over Na₂SO₄, filtered, concentrated under reduced pressure and purified by flash column with DCM/MeOH 0-5% to afford desired product 19-4 (200 mg) as a white solid with 57% yield. LCMS: Calculated Exact Mass=732, Found [M+H]+ (ESI+)=733.

### 2-(2,4-difluorophenyl)-1,1-difluoro-1-(5-((4-((((2-(4-fluorophenyl)-3-(pyridin-4-yl)pyrazolo[1,5-a]pyridin-6-yl)methyl)amino)methyl)phenyl)ethynyl)pyridin-2-yl)-3-(1H-1,2,4-triazol-1-yl)propan-2-ol (I-268)

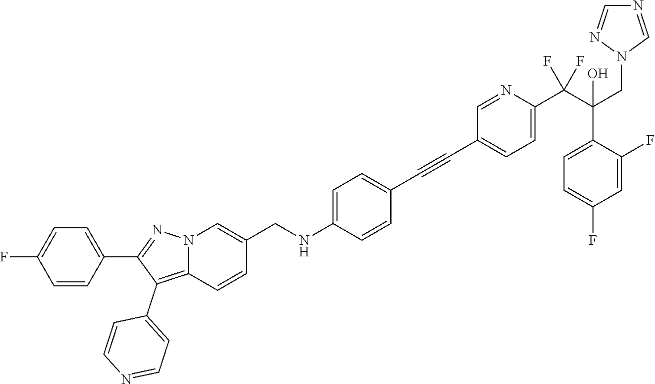 [SMILES: OC(Cn1cncn1)(c1ccc(F)cc1F)C(F)(F)c1ccc(C#Cc2ccc(NCc3ccc4c(-c5ccncc5)c(-c5ccc(F)cc5)nn4c3)cc2)cn1]

A solution of 4-((6-(2-(2,4-difluorophenyl)-1,1-difluoro-2-hydroxy-3-(1H-1,2,4-triazol-1-yl)propyl)pyridin-3-yl)ethynyl)phenyl trifluoromethanesulfonate 19-4 (170 mg, 0.28 mmol), (2-(4-fluorophenyl)-3-(pyridin-4-yl)pyrazolo[1,5-a]pyridin-6-yl)methanamine 19-5 (90 mg, 0.28 mmol), tris(dibenzylideneacetone)dipalladium (52 mg, 0.06 mmol), potassium phosphate tribasic (120 mg, 0.57 mmol) and X-Phos (27 mg, 0.06 mmol) in dioxane (10 mL) was stirred at 100° C. by microwave for 6 hours. Then the reaction was cooled to room temperature and filtered through a pad of Celite and rinsed with EtOAc (30 mL). The organic layer was washed with brine (20 mL), dried over Na₂SO₄, filtered, concentrated under reduced pressure and purified by Prep-HPLC (ACN/H₂O with 0.05% NH₃) to afford 2-(2,4-difluorophenyl)-1,1-difluoro-1-(5-(4-(((2-(4-fluorophenyl)-3-(pyridin-4-yl)pyrazolo[1,5-a]pyridin-6-yl)methyl)amino)phenyl)pyridin-2-yl)-3-(1H-1,2,4-triazol-1-yl)propan-2-ol 1-268 (29.2 mg) as a white solid with 13.6% yield.

LCMS: Calculated Exact Mass=768.2, Found [M+H]+ (ESI+)=769.1 ¹H NMR (400 MHz, DMSO) δ 8.78 (s, 1H), 8.61 (d, J=1.8 Hz, 1H), 8.56 (dd, J=4.5, 1.6 Hz, 2H), 8.35 (s, 1H), 7.95 (dd, J=8.2, 2.1 Hz, 1H), 7.76 (d, J=9.2 Hz, 1H), 7.70 (s, 1H), 7.55-7.50 (m, 2H), 7.41 (d, J=8.0 Hz, 2H), 7.31 (dd, J=9.1, 4.8 Hz, 4H), 7.29-7.24 (m, 2H), 7.22-7.16 (m, 1H), 7.13 (s, 1H), 7.04 (s, 1H), 6.98 (s, 1H), 6.85 (d, J=2.5 Hz, 1H), 6.72 (d, J=8.8 Hz, 2H), 5.34 (d, J=14.7 Hz, 1H), 4.83 (d, J=14.7 Hz, 1H), 4.42 (d, J=6.0 Hz, 2H).

### Example 22—Synthesis of Compound 2-(2,4-difluorophenyl)-1,1-difluoro-1-(5-(4-((((2-(4-fluorophenyl)-3-(pyridin-4-yl)pyrazolo[1,5-a]pyridin-6-yl)methyl)amino)methyl)phenyl)pyridin-2-yl)-3-(1H-tetrazol-1-yl)propan-2-ol (I-269)

Procedures for the synthesis of compound 2-(2,4-difluorophenyl)-1,1-difluoro-1-(5-(4-((((2-(4-fluorophenyl)-3-(pyridin-4-yl)pyrazolo[1,5-a]pyridin-6-yl)methyl)amino)methyl)phenyl)pyridin-2-yl)-3-(1H-tetrazol-1-yl)propan-2-ol (I-269) is shown below in Scheme 38:

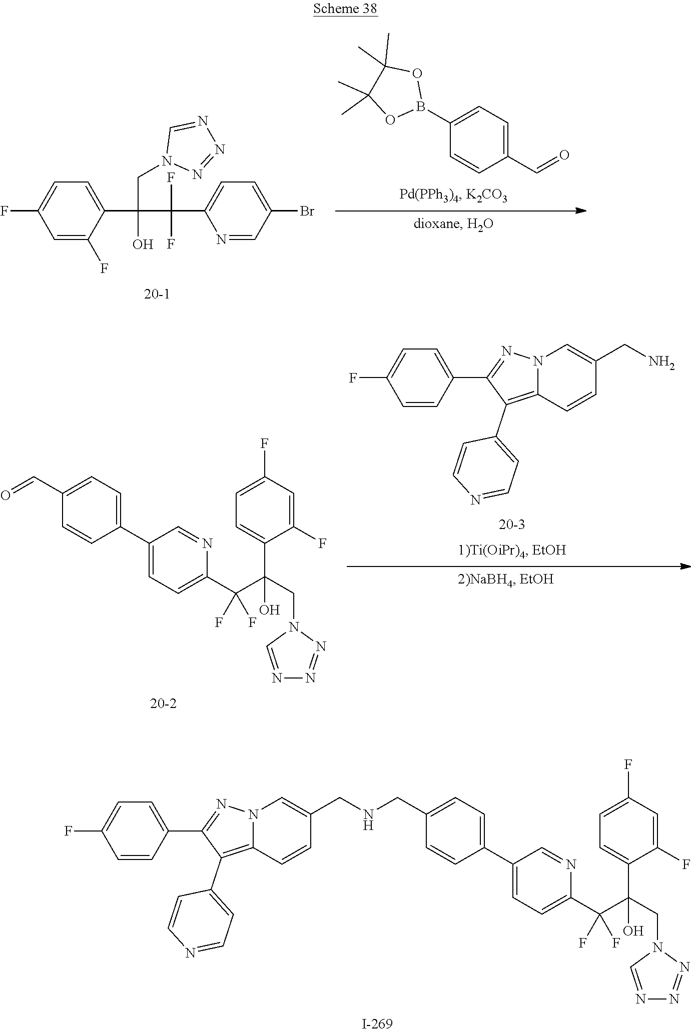 [SMILES: OC(Cn1cnnn1)(c1ccc(F)cc1F)C(F)(F)c1ccc(Br)cn1 ; CC1(C)OB(c2ccc(C=O)cc2)OC1(C)C ; O=Cc1ccc(-c2ccc(C(F)(F)C(O)(Cn3cnnn3)c3ccc(F)cc3F)nc2)cc1 ; NCc1ccc2c(-c3ccncc3)c(-c3ccc(F)cc3)nn2c1 ; OC(Cn1cnnn1)(c1ccc(F)cc1F)C(F)(F)c1ccc(-c2ccc(CNCc3ccc4c(-c5ccncc5)c(-c5ccc(F)cc5)nn4c3)cc2)cn1]

### 4-(6-(2-(2,4-difluorophenyl)-1,1-difluoro-2-hydroxy-3-(1H-tetrazol-1-yl)propyl)pyridin-3-yl)benzaldehyde (20-2)

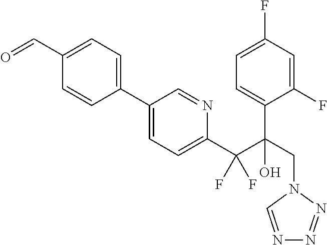 [SMILES: O=Cc1ccc(-c2ccc(C(F)(F)C(O)(Cn3cnnn3)c3ccc(F)cc3F)nc2)cc1]

A solution of 1-(5-bromopyridin-2-yl)-2-(2,4-difluorophenyl)-1,1-difluoro-3-(1H-1,2,4-triazol-1-yl)propan-2-ol 20-1 (300 mg, 0.7 mmol), 4-(4,4,5,5-tetramethyl-1,3,2-dioxaborolan-2-yl)benzaldehyde (323 mg, 1.4 mmol), tetrakis(triphenylphosphine)palladium(0) (148 mg, 0.7 mmol) and potassium carbonate (192 mg, 1.4 mmol) in dioxane (10 mL) and H₂O (2.5 mL) was stirred at 100° C. in microwave for 2 hours. Then the reaction was cooled to room temperature and filtered through a pad of Celite and rinsed with EtOAc (30 mL). The organic layer was washed with brine (20 mL), dried over Na₂SO₄, filtered, concentrated under reduced pressure and purified by flash column with DCM/MeOH 0-5% to afford desired product 20-2 (250 mg) as a brown solid with 78% yield. LCMS: Calculated Exact Mass=457.1, Found [M+H]+ (ESI+)=458.1.

### 2-(2,4-difluorophenyl)-1,1-difluoro-1-(5-(4-((((2-(4-fluorophenyl)-3-(pyridin-4-yl)pyrazolo[1,5-a]pyridin-6-yl)methyl)amino)methyl)phenyl)pyridin-2-yl)-3-(1H-tetrazol-1-yl)propan-2-ol (I-269)

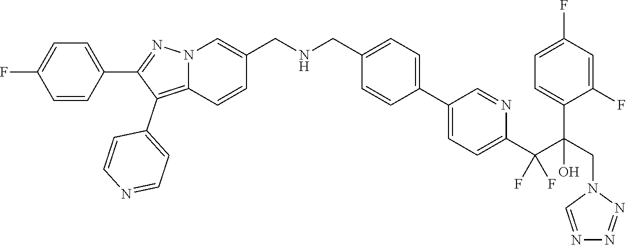 [SMILES: OC(Cn1cnnn1)(c1ccc(F)cc1F)C(F)(F)c1ccc(-c2ccc(CNCc3ccc4c(-c5ccncc5)c(-c5ccc(F)cc5)nn4c3)cc2)cn1]

A solution of 4-(6-(2-(2,4-difluorophenyl)-1,1-difluoro-hydroxy-3-(1H-tetrazol-1-yl)propyl)pyridin-3-yl)benzaldehyde 20-2 (158 mg, 0.35 mmol), (2-(4-fluorophenyl)-3-(pyridin-4-yl)pyrazolo[1,5-a]pyridin-6-yl)methanamine 20-3 (110 mg, 0.35 mmol), and titanium tetraisopropanolate (2 drops) in EtOH (10 mL) was stirred at 90° C. in microwave for 2 hours. Then the reaction was cooled to room temperature and sodium borohydride (65.7 mg, 1.73 mmol) was added into the mixture and stirred at room temperature for 1 hour. The mixture was concentrated then purified by Prep-HPLC (ACN/H₂O with 0.05% NH₃) to afford 2-(2,4-difluorophenyl)-1,1-difluoro-1-(5-(4-((((2-(4-fluorophenyl)-3-(pyridin-4-yl)pyrazolo[1,5-a]pyridin-6-yl)methyl)amino)methyl)phenyl)pyridin-2-yl)-3-(1H-tetrazol-1-yl)propan-2-ol 1-269 (113 mg) as a white solid with 40% yield.

LCMS: Calculated Exact Mass=759.2, Found [M+H]+ (ESI+)=760.1 ¹H NMR (400 MHz, DMSO) δ 9.16 (s, 1H), 8.94 (d, J=1.9 Hz, 1H), 8.71 (s, 1H), 8.56 (d, J=6.0 Hz, 2H), 8.23 (dd, J=8.3, 2.0 Hz, 1H), 7.75 (t, J=6.5 Hz, 3H), 7.56-7.50 (m, 5H), 7.45 (d, J=9.2 Hz, 1H), 7.35-7.25 (m, 6H), 7.24-7.17 (m, 1H), 6.95-6.86 (m, 1H), 5.68 (d, J=14.7 Hz, 1H), 5.12 (d, J=14.8 Hz, 1H), 3.80 (s, 4H), 3.03 (s, 1H).

### Example 23—Synthesis of Compound 2-(2,4-difluorophenyl)-1,1-difluoro-1-(5-(4-((((2-(4-fluorophenyl)-3-(pyridin-4-yl)pyrazolo[1,5-a]pyridin-6-yl)methyl)amino)methyl)phenyl)pyridin-2-yl)-3-(1H-tetrazol-1-yl)propan-2-ol (I-270)

Procedures for the synthesis of compound 2-(2,4-difluorophenyl)-1,1-difluoro-1-(5-(4-((((2-(4-fluorophenyl)-3-(pyridin-4-yl)pyrazolo[1,5-a]pyridin-6-yl)methyl)amino)methyl)phenyl)pyridin-2-yl)-3-(1H-tetrazol-1-yl)propan-2-ol (I-270) is shown below in Scheme 39:

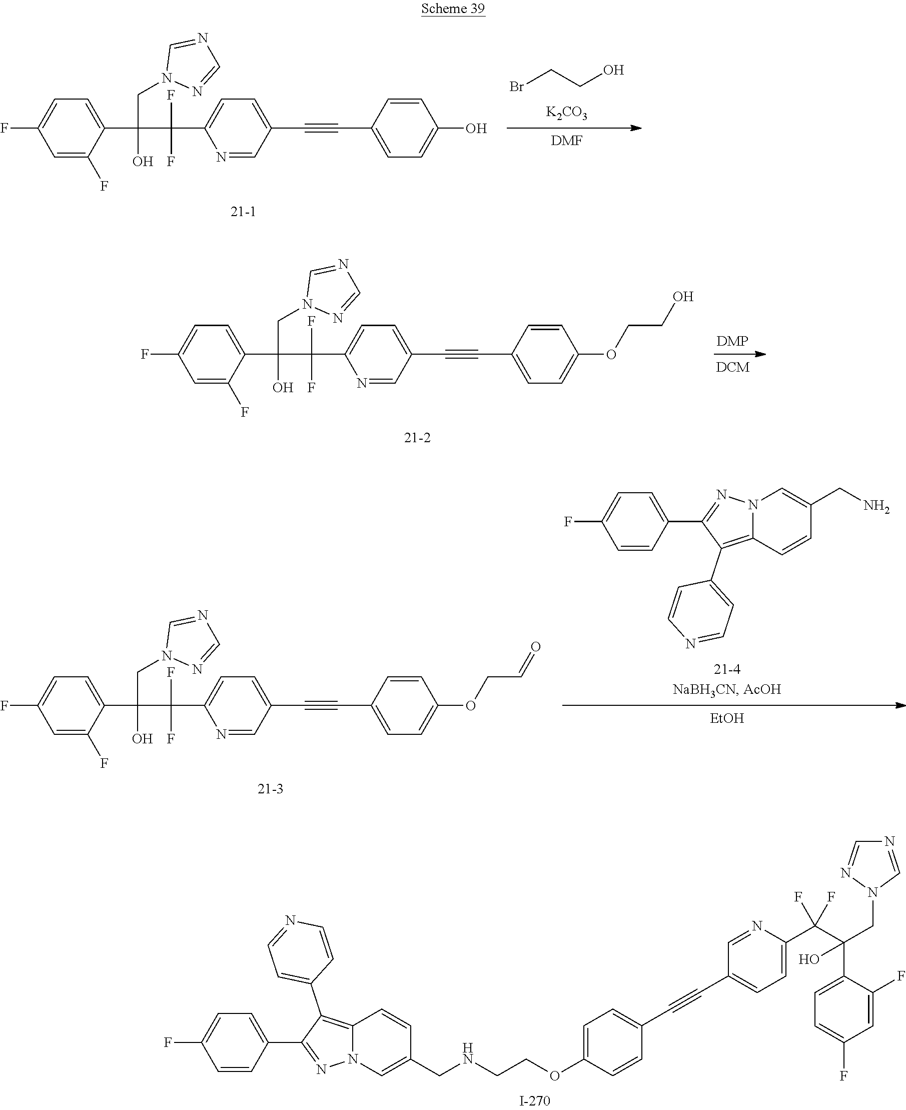

### 2-(2,4-difluorophenyl)-1,1-difluoro-1-(5-((4-(2-hydroxyethoxy)phenyl)ethynyl)pyridin-2-yl)-3-(1H-1,2,4-triazol-1-yl)propan-2-ol (21-2)

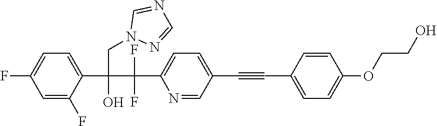 [SMILES: OCCOc1ccc(C#Cc2ccc(C(F)(F)C(O)(Cn3cncn3)c3ccc(F)cc3F)nc2)cc1]

4-((6-(2-(2,4-difluorophenyl)-1,1-difluoro-2-hydroxy-3-(1H-1,2,4-triazol-1-yl)propyl)pyridin-3-yl)ethynyl)phenol 21-1 (500 mg, 1.07 mmol) and 2-bromoethan-1-ol (265 mg, 2.14 mmol) were dissolved in 10 mL DMF, followed by adding potassium carbonate (295 mg, 2.14 mmol). The reaction was stirred at 70° C. for 48 hours. Then the reaction mixture was diluted with water (100 mL), extracted with EtOAc (50 mL*2). The organic layer was washed with brine (100 mL*3), dried over Na₂SO₄, filtered, concentrated under reduced pressure and purified by flash column with DCM/MeOH 0-5% to afford desired product 21-2 (400 mg) as a white solid with 73% yield. LCMS: Calculated Exact Mass=512.1, Found [M+H]+ (ESI+)=513.1.

### 2-(4-((6-(2-(2,4-difluorophenyl)-1,1-difluoro-2-hydroxy-3-(1H-1,2,4-triazol-1-yl)propyl)pyridin-3-yl)ethynyl)phenoxy)acetaldehyde (21-3)

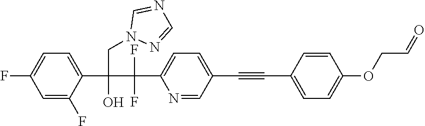 [SMILES: O=CCOc1ccc(C#Cc2ccc(C(F)(F)C(O)(Cn3cncn3)c3ccc(F)cc3F)nc2)cc1]

2-(2,4-difluorophenyl)-1,1-difluoro-1-(5-((4-(2-hydroxyethoxy)phenyl)ethynyl)pyridin-2-yl)-3-(1H-1,2,4-triazol-1-yl)propan-2-ol 21-2 (250 mg, 0.49 mmol) was dissolved in 10 mL DCM, followed by adding Dess-Martin periodinane (414 mg, 0.98 mmol). The reaction was stirred at room temperature for 48 hours. Then the reaction was filtered and rinsed with DCM (20 mL). The organic layer was washed with saturated sodium thiosulfate (30 mL*3), dried over Na₂SO₄, filtered, concentrated under reduced pressure to afford crude product 21-3 (140 mg). LCMS: Calculated Exact Mass=510, Found [M+H]+ (ESI+)=543.

### 2-(2,4-difluorophenyl)-1,1-difluoro-1-(5-(4-((((2-(4-fluorophenyl)-3-(pyridin-4-yl)pyrazolo[1,5-a]pyridin-6-yl)methyl)amino)methyl)phenyl)pyridin-2-yl)-3-(1H-tetrazol-1-yl)propan-2-ol (I-270)

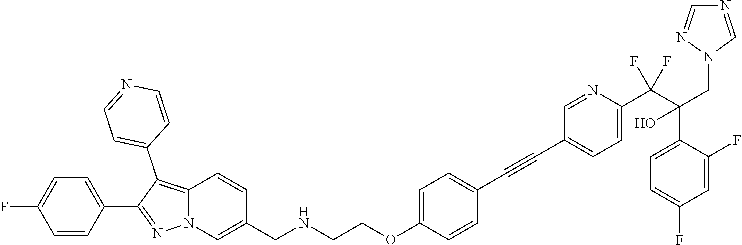 [SMILES: OC(Cn1cncn1)(c1ccc(F)cc1F)C(F)(F)c1ccc(C#Cc2ccc(OCCNCc3ccc4c(-c5ccncc5)c(-c5ccc(F)cc5)nn4c3)cc2)cn1]

2-(4-((6-(2-(2,4-difluorophenyl)-1,1-difluoro-2-hydroxy-3-(1H-1,2,4-triazol-1-yl)propyl)pyridin-3-yl)ethynyl)phenoxy)acetaldehyde 21-3 (165 mg, 0.32 mmol) and (2-(4-fluorophenyl)-3-(pyridin-4-yl)pyrazolo[1,5-a]pyridin-6-yl)methanamine 21-4 (103 mg, 0.32 mmol) were dissolved in 10 mL EtOH, followed by adding sodium cyanoborohydride (61.5 mg, 0.98 mmol) and acetic acid (2 drops). The reaction was stirred at 70° C. in microwave for 2 hours. Then the reaction was concentrated and then purified by Prep-HPLC (ACN/H₂O with 0.05% NH₃) to afford 2-(2,4-difluorophenyl)-1,1-difluoro-1-(5-(4-((((2-(4-fluorophenyl)-3-(pyridin-4-yl)pyrazolo[1,5-a]pyridin-6-yl)methyl)amino)methyl)phenyl)pyridin-2-yl)-3-(1H-tetrazol-1-yl)propan-2-ol 1-270 (62.6 mg) as a white solid with 24% yield.

LCMS: Calculated Exact Mass=812.2, Found [M+H]+ (ESI+)=813.2 ¹H NMR (400 MHz, DMSO) δ 8.74 (s, 1H), 8.68 (s, 1H), 8.56 (d, J=5.4 Hz, 2H), 8.36 (s, 1H), 8.03 (d, J=8.5 Hz, 1H), 7.72 (d, J=10.0 Hz, 2H), 7.54 (t, J=8.1 Hz, 4H), 7.44 (dd, J=13.2, 8.7 Hz, 2H), 7.31 (d, J=5.7 Hz, 2H), 7.27 (t, J=8.9 Hz, 2H), 7.22-7.11 (m, 2H), 7.08 (s, 1H), 7.02 (d, J=8.7 Hz, 2H), 6.85 (t, J=8.5 Hz, 1H), 5.35 (d, J=14.5 Hz, 1H), 4.85 (d, J=14.6 Hz, 1H), 4.11 (t, J=5.3 Hz, 2H), 3.85 (s, 2H), 2.91 (t, J=5.3 Hz, 2H), 2.60 (s, 1H).

### Example 24—Synthesis of Compound 2-(2,4-difluorophenyl)-5-(((2-(4-fluorophenyl)-3-(pyridin-4-yl)pyrazolo[1,5-a]pyridin-6-yl)methyl)amino)-1-(1H-1,2,4-triazol-1-yl)pentan-2-ol (I-279)

Procedures for the synthesis of compound 2-(2,4-difluorophenyl)-5-(((2-(4-fluorophenyl)-3-(pyridin-4-yl)pyrazolo[1,5-a]pyridin-6-yl)methyl)amino)-1-(1H-1,2,4-triazol-1-yl)pentan-2-ol (I-279) is shown below in Scheme 40:

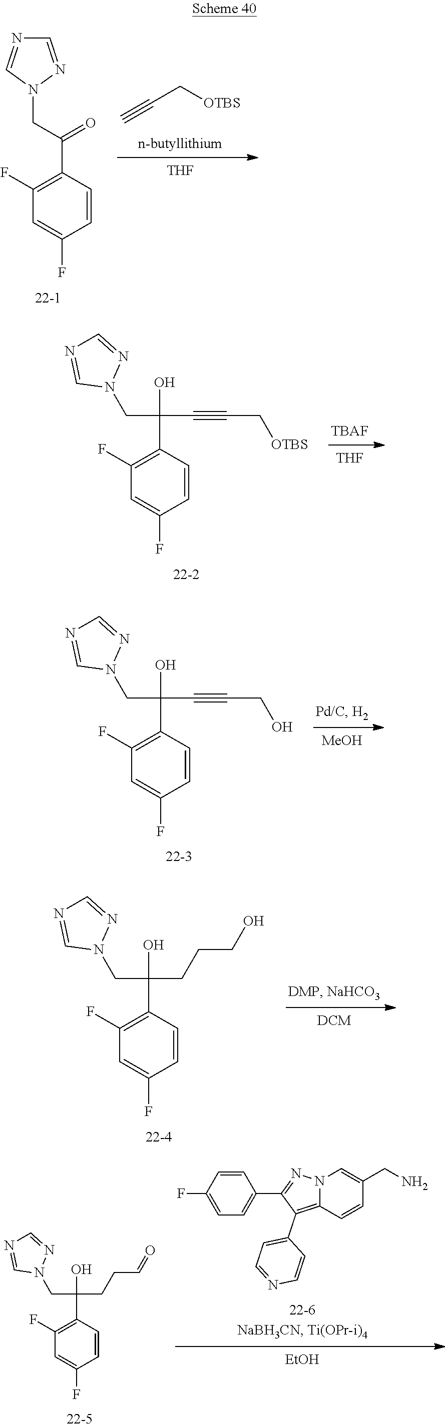

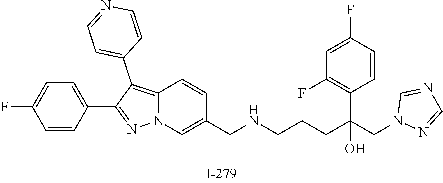 [SMILES: OC(CCCNCc1ccc2c(-c3ccncc3)c(-c3ccc(F)cc3)nn2c1)(Cn1cncn1)c1ccc(F)cc1F]

### 5-((tert-butyldimethylsilyl)oxy)-2-(2,4-difluorophenyl)-1-(1H-1,2,4-triazol-1-yl pent-3-yn-2-ol (22-2)

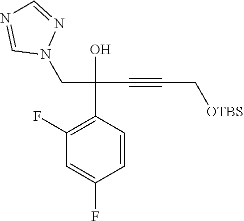 [SMILES: CC(C)(C)[Si](C)(C)OCC#CC(O)(Cn1cncn1)c1ccc(F)cc1F]

Tert-butyldimethyl (prop-2-yn-1-yloxy)silane (11.4 g, 67.2 mmol) was dissolved in 50 mL THF and n-butyllithium (2.5 M, 30.5 mL, 76.1 mmol) in THF was added with dropwise at −78° C. with a dry ice acetone bath. The reaction was stirred at −78° C. for 1 hour. Then a solution of 1-(2,4-difluorophenyl)-2-(1H-1,2,4-triazol-1-yl)ethan-1-one 22-1 (10 g, 44.8 mmol) in THF (10 mL) was added with dropwise at −78° C. The mixture was stirred at −78° C. to room temperature for 6 hours. The mixture was quenched with NH₄Cl (100 mL) and extracted with EtOAc (100 mL*2), washed with brine (100 mL). The organic layer was dried with Na₂SO₄, concentrated and purified by flash (PE/EA=10/1 to 1:1) to afford 5-((tert-butyldimethylsilyl)oxy)-2-(2,4-difluorophenyl)-1-(1H-1,2,4-triazol-1-yl)pent-3-yn-2-ol 22-2 (6 g) as a white solid with 34% yield. LCMS: Calculated Exact Mass=393.2, Found [M+H]+ (ESI+)=394.1.

### 4-(2,4-difluorophenyl)-5-(1H-1,2,4-triazol-1-yl)pent-2-yne-1,4-diol (22-3)

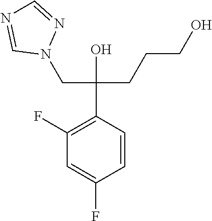 [SMILES: OCCCC(O)(Cn1cncn1)c1ccc(F)cc1F]

To a solution of 5-((tert-butyldimethylsilyl)oxy)-2-(2,4-difluorophenyl)-1-(1H-1,2,4-triazol-1-yl)pent-3-yn-2-ol 22-2 (6 g, 15.2 mmol) in 20 mL THF was added TBAF (1 M, 45.6 mL, 45.6 mmol) in THF at room temperature. The reaction was stirred for 6 hours. Then the reaction was concentrated, diluted with 100 mL EtOAc, washed with 100 mL brine. The organic layer was dried with Na₂SO₄, concentrated and purified by flash (DCM/MeOH=10/1) to afford 4-(2,4-difluorophenyl)-5-(1H-1,2,4-triazol-1-yl)pent-2-yne-1,4-diol 22-3 (4 g) as a white solid with 95% yield. LCMS: Calculated Exact Mass=279.1, Found [M+H]+ (ESI+)=280.2.

### 4-(2,4-difluorophenyl)-5-(1H-1,2,4-triazol-1-yl)pentane-1,4-diol (22-4)

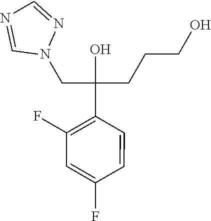 [SMILES: OCCCC(O)(Cn1cncn1)c1ccc(F)cc1F]

To a solution of 4-(2,4-difluorophenyl)-5-(1H-1,2,4-triazol-1-yl)pent-2-yne-1,4-diol 22-3 (2 g, 7.1 mmol) in 20 mL MeOH was added 10% Pd/C (400 mg, 20% wt) at room temperature. The reaction was stirred for 4 hours. Then the reaction was concentrated to afford 4-(2,4-difluorophenyl)-5-(1H-1,2,4-triazol-1-yl)pentane-1,4-diol 22-4 (2 g) as a white solid with 98% yield. LCMS: Calculated Exact Mass=283.1, Found [M+H]+ (ESI+)=284.2.

### 4-(2,4-difluorophenyl)-4-hydroxy-5-(1H-1,2,4-triazol-1-yl)pentanal (22-5)

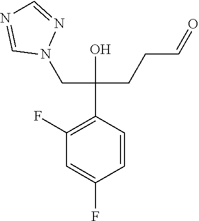 [SMILES: O=CCCC(O)(Cn1cncn1)c1ccc(F)cc1F]

To a solution of 4-(2,4-difluorophenyl)-5-(1H-1,2,4-triazol-1-yl)pentane-1,4-diol 22-4 (2 g, 7.0 mmol) in 20 mL DCM was added NaHCO₃ (0.8 g, 10.5 mmol) and Dess-Martin periodinane (4.4 g, 10.5 mmol) at room temperature. The reaction was stirred for 16 hours. Then the reaction was filtered and washed with solution of Na₂SO₃ (30 mL). The organic layer was concentrated to afford crude 4-(2,4-difluorophenyl)-4-hydroxy-5-(1H-1,2,4-triazol-1-yl)pentanal 22-5 (1.8 g).

### 2-(2,4-difluorophenyl)-5-(((2-(4-fluorophenyl)-3-(pyridin-4-yl)pyrazolo[1,5-a]pyridin-6-yl)methyl)amino)-1-(1H-1,2,4-triazol-1-yl)pentan-2-ol (I-279)

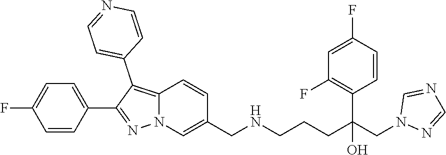 [SMILES: OC(CCCNCc1ccc2c(-c3ccncc3)c(-c3ccc(F)cc3)nn2c1)(Cn1cncn1)c1ccc(F)cc1F]

A solution of 4-(2,4-difluorophenyl)-4-hydroxy-5-(1H-1,2,4-triazol-1-yl)pentanal 22-5 (176 mg, 0.62 mmol) and (2-(4-fluorophenyl)-3-(pyridin-4-yl)pyrazolo[1,5-a]pyridin-6-yl)methanamine 22-6 (100 mg, 0.31 mmol) in 8 mL EtOH was added NaBH₃CN (98 mg, 1.5 mmol) and one drop of Titanium tetraisopropanolate. The reaction was stirred at 70° C. in a microwave for 2 hours. The mixture was concentrated in vacuum and purified by flash column with DCM/MeOH 0-5% and Prep-HPLC (ACN/H₂O with 0.05% NH₃) to afford 2-(2,4-difluorophenyl)-5-(((2-(4-fluorophenyl)-3-(pyridin-4-yl)pyrazolo[1,5-a]pyridin-6-yl)methyl)amino)-1-(1H-1,2,4-triazol-1-yl)pentan-2-ol 1-280 (66.3 mg) as a white solid with 36% yield.

LCMS: Calculated Exact Mass=583.2, Found [M+H]+ (ESI+)=584.2. ¹H NMR (400 MHz, DMSO) δ 8.66 (s, 1H), 8.57 (dd, J=4.5, 1.6 Hz, 2H), 8.28 (s, 1H), 7.77-7.69 (m, 2H), 7.58-7.50 (m, 2H), 7.38 (ddd, J=13.2, 6.6, 4.0 Hz, 2H), 7.33-7.24 (m, 4H), 7.15 (ddd, J=11.8, 9.1, 2.5 Hz, 1H), 6.95 (td, J=8.5, 2.5 Hz, 1H), 6.77 (s, 1H), 4.47 (q, J=14.1 Hz, 2H), 3.72-3.60 (m, 2H), 2.51 (s, 1H), 2.45-2.32 (m, 2H), 2.23-2.12 (m, 1H), 1.75-1.59 (m, 1H), 1.43-1.12 (m, 2H).

### Example 25—Synthesis of Compound 2-(2,4-difluorophenyl)-5-(((2-(4-fluorophenyl)-3-(pyridin-4-yl)pyrazolo[1,5-a]pyridin-6-yl)methyl)amino)-1-(1H-1,2,4-triazol-1-yl)pent-3-yn-2-ol (I-280)

Procedures for the synthesis of compound 2-(2,4-difluorophenyl)-5-(((2-(4-fluorophenyl)-3-(pyridin-4-yl)pyrazolo[1,5-a]pyridin-6-yl)methyl)amino)-1-(1H-1,2,4-triazol-1-yl)pent-3-yn-2-ol (I-280) is shown below in Scheme 41:

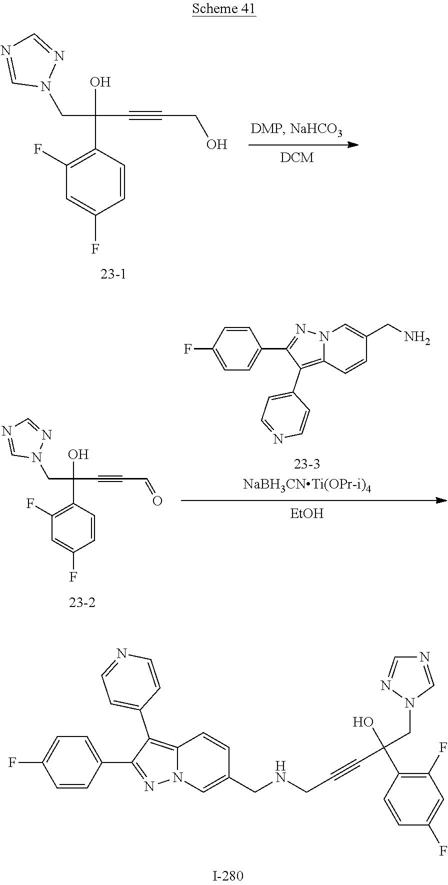

### 4-(2,4-difluorophenyl)-4-hydroxy-5-(1H-1,2,4-triazol-1-yl)pent-2-ynal (23-2)

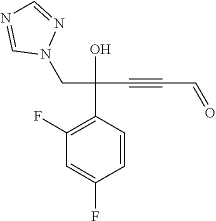 [SMILES: O=CC#CC(O)(Cn1cncn1)c1ccc(F)cc1F]

To a solution of 4-(2,4-difluorophenyl)-5-(1H-1,2,4-triazol-1-yl)pent-2-yne-1,4-diol 23-1 (500 mg, 1.8 mmol) in 10 mL DCM was added NaHCO₃ (226 mg, 2.7 mmol) and Dess-Martin periodinane (1.1 g, 2.7 mmol) at room temperature. The reaction was stirred for 16 hours. Then the reaction was filtered and washed with solution of Na₂SO₃ (30 mL). The organic layer was concentrated to afford crude 4-(2,4-difluorophenyl)-4-hydroxy-5-(1H-1,2,4-triazol-1-yl)pent-2-ynal 23-2 (300 mg).

### 2-(2,4-difluorophenyl)-5-(((2-(4-fluorophenyl)-3-(pyridin-4-yl)pyrazolo[1,5-a]pyridin-6-yl)methyl)amino)-1-(1H-1,2,4-triazol-1-yl)pent-3-yn-2-ol (I-280)

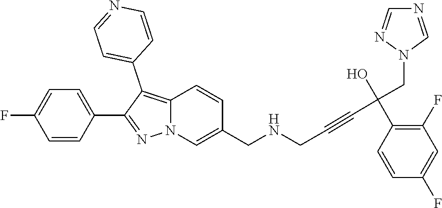 [SMILES: OC(C#CCNCc1ccc2c(-c3ccncc3)c(-c3ccc(F)cc3)nn2c1)(Cn1cncn1)c1ccc(F)cc1F]

A solution of 4-(2,4-difluorophenyl)-4-hydroxy-5-(1H-1,2,4-triazol-1-yl)pent-2-ynal 23-2 (171 mg, 0.62 mmol) and (2-(4-fluorophenyl)-3-(pyridin-4-yl)pyrazolo[1,5-a]pyridin-6-yl)methanamine 23-3 (100 mg, 0.31 mmol) in 8 mL EtOH was added NaBH₃CN (98 mg, 1.5 mmol) and one drop of titanium tetraisopropanolate. The reaction was stirred at 70° C. in a microwave for 2 hours. The mixture was concentrated in vacuum and purified by flash column with DCM/MeOH 0-5% and Prep-HPLC (ACN/H2O with 0.05% NH3) to afford 2-(2,4-difluorophenyl)-5-(((2-(4-fluorophenyl)-3-(pyridin-4-yl)pyrazolo[1,5-a]pyridin-6-yl)methyl)amino)-1-(1H-1,2,4-triazol-1-yl)pent-3-yn-2-ol 1-280 (21.6 mg) as a white solid with 11% yield.

LCMS: Calculated Exact Mass=579.2, Found [M+H]+ (ESI+)=580.2. ¹H NMR (400 MHz, DMSO) δ 8.67 (s, 1H), 8.57 (d, J=6.0 Hz, 2H), 8.38 (s, 1H), 7.78 (s, 1H), 7.73 (d, J=9.2 Hz, 1H), 7.54 (dd, J=8.6, 5.6 Hz, 2H), 7.41 (dd, J=16.7, 9.1 Hz, 2H), 7.33-7.23 (m, 5H), 7.02-6.93 (m, 2H), 4.72-4.58 (m, 2H), 3.80 (s, 2H), 3.40 (s, 2H), 2.71 (s, 1H).

### Example 26—Synthesis of Compound 4-(3-(((2-(4-fluorophenyl)-3-(pyridin-4-yl)pyrazolo[1,5-a]pyridin-6-yl)methyl)amino)propoxy)benzimidamide HCl salt (I-281)

Procedures for the synthesis of compound 4-(3-(((2-(4-fluorophenyl)-3-(pyridin-4-yl)pyrazolo[1,5-a]pyridin-6-yl)methyl)amino)propoxy)benzimidamide HCl salt (I-281) is shown below in Scheme 42:

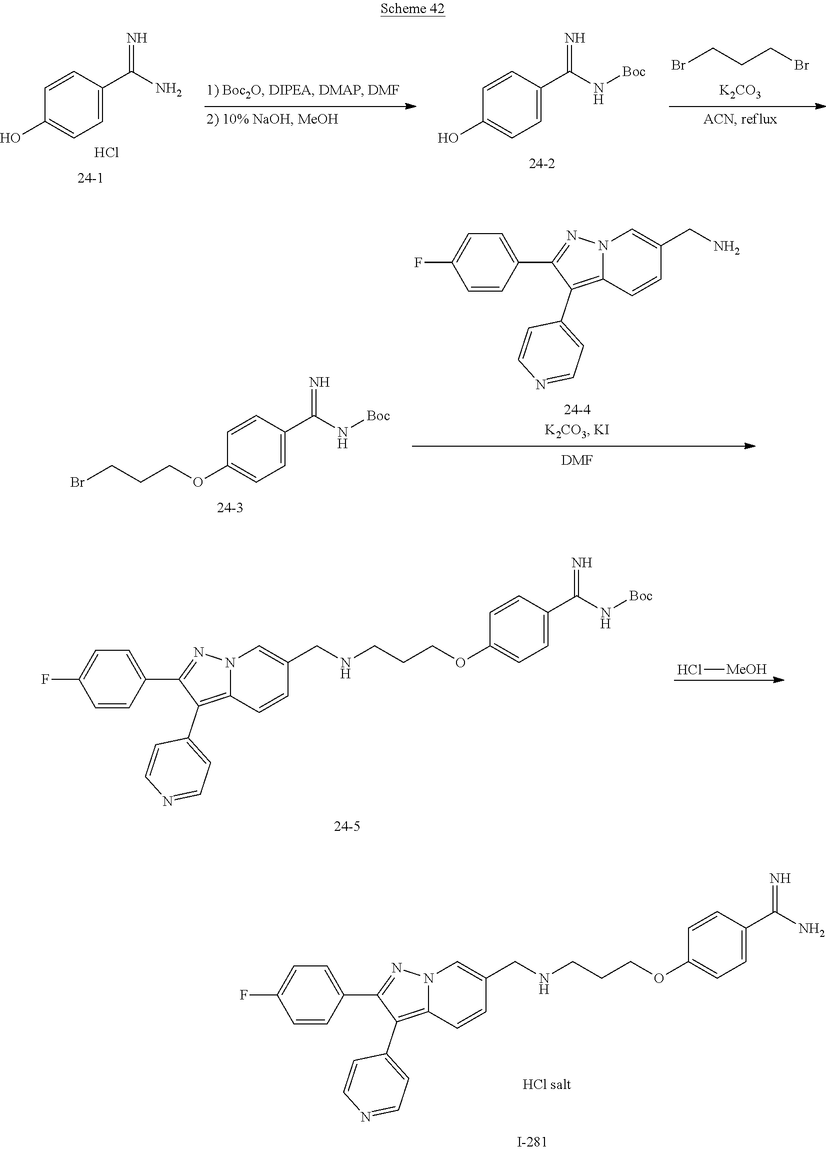

### tert-butyl ((4-hydroxyphenyl)(imino)methyl)carbamate (24-2)

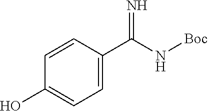 [SMILES: CC(C)(C)OC(=O)NC(=N)c1ccc(O)cc1]

To a solution of 4-hydroxybenzamidine hydrochloride 24-1 (9.0 g, 52.2 mmol), DMAP (630 mg, 5.2 mmol), and DIPEA (34.2 mL, 208.8 mmol) in dry DMF (100 mL) was added Boc₂O (22.5 g, 104.4 mmol) at 0° C., and the resulting suspension was stirred overnight at room temperature. The reaction mixture was diluted with water (200 mL) to form an off-white precipitate and filtered. The solid was dissolved into 200 mL of MeOH with an additional 200 mL of 10% NaOH solution, and the reaction mixture was stirred for 1.5 h at room temperature. The mixture was concentrated and diluted with water (200 mL) and extracted with AcOEt (200 mL*3); Then, the combined organic layer was washed with brine (200 mL) and dried over anhydrous Na₂SO₄. The mixture was concentrated under reduced pressure to afford tert-butyl ((4-hydroxyphenyl)(imino)methyl)carbamate 24-2 (9 g), which was used directly without further purification. LCMS: Calculated Exact Mass=236.1, Found [M+H]+ (ESI+)=237.2.

### tert-butyl ((4-(3-bromopropoxy)phenyl)(imino)methyl)carbamate (24-3)

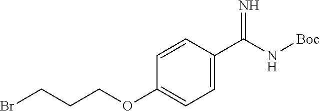 [SMILES: CC(C)(C)OC(=O)NC(=N)c1ccc(OCCCBr)cc1]

Tert-butyl ((4-hydroxyphenyl)(imino)methyl)carbamate 24-2 (1 g, 4.2 mmol) and K₂CO₃ (2.3 g, 16.8 mmol) were dissolved in 12 mL of MeCN. 1,3-dibromopropane (2.31 mL, 21 mmol) was added in portions, and the mixture was stirred under reflux for 6 h. After cooling to room temperature, the resulting mixture was quenched with water (20 mL) and extracted with AcOEt (20 mL*3). The combined organic layer was washed with brine (20 mL), dried over Na₂SO₄, and evaporated under vacuum to obtain the crude product, which was purified by flash chromatography (DCM/MeOH=10:1) to afford tert-butyl ((4-(3-bromopropoxy)phenyl)(imino)methyl)carbamate 24-3 (1.0 g) as white solid with 66% yield. LCMS: Calculated Exact Mass=356.1, Found [M+H]+ (ESI+)=357.2.

### tert-butyl ((4-(3-(((2-(4-fluorophenyl)-3-(pyridin-4-yl)pyrazolo[1,5-a]pyridin-6-yl)methyl)amino)propoxy)phenyl)(imino)methyl)carbamate (24-5)

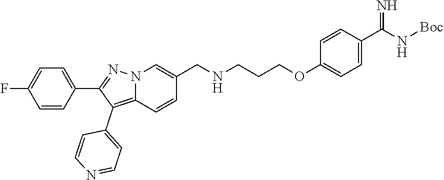 [SMILES: CC(C)(C)OC(=O)NC(=N)c1ccc(OCCCNCc2ccc3c(-c4ccncc4)c(-c4ccc(F)cc4)nn3c2)cc1]

To a solution of tert-butyl ((4-(3-bromopropoxy)phenyl)(imino)methyl)carbamate 24-3 (400 mg, 1.1 mmol) and (2-(4-fluorophenyl)-3-(pyridin-4-yl)pyrazolo[1,5-a]pyridin-6-yl)methanamine 24-4 (357 mg, 1.1 mmol) in 10 mL DMF was added K₂CO₃ (607 mg, 4.4 mmol) and KI (90 mg, 0.55 mmol). The reaction was stirred at room temperature for 16 hours. The mixture was diluted with EtOAc (100 mL) and washed with brine (100 mL) and concentrated. The crude was purified by silica gel column (DCM/MeOH=10:1) to afford tert-butyl ((4-(3-(((2-(4-fluorophenyl)-3-(pyridin-4-yl)pyrazolo[1,5-a]pyridin-6-yl)methyl)amino)propoxy)phenyl)(imino)methyl)carbamate 24-5 (200 mg) as white solid with 30% yield.

LCMS: Calculated Exact Mass=579.2, Found [M+H]+ (ESI+)=580.2. ¹H NMR (400 MHz, DMSO) δ 8.94 (s, 2H), 8.70 (s, 1H), 8.59-8.51 (m, 2H), 7.93 (d, J=8.8 Hz, 2H), 7.69 (d, J=9.2 Hz, 1H), 7.58-7.49 (m, 2H), 7.40 (d, J=9.2 Hz, 1H), 7.28 (dd, J=16.8, 7.6 Hz, 4H), 6.97 (d, J=8.9 Hz, 2H), 4.12 (t, J=6.3 Hz, 2H), 3.78 (s, 2H), 2.67 (t, J=6.7 Hz, 2H), 2.44 (s, 1H), 1.90 (p, J=6.4 Hz, 2H), 1.44 (s, 9H).

### 4-(3-(((2-(4-fluorophenyl)-3-(pyridin-4-yl)pyrazolo[1,5-a]pyridin-6-yl)methyl)amino)propoxy)benzimidamide HCl salt (I-281)

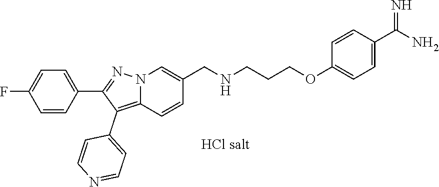 [SMILES: N=C(N)c1ccc(OCCCNCc2ccc3c(-c4ccncc4)c(-c4ccc(F)cc4)nn3c2)cc1]

To a solution of tert-butyl ((4-(3-(((2-(4-fluorophenyl)-3-(pyridin-4-yl)pyrazolo[1,5-a]pyridin-6-yl)methyl)amino)propoxy)phenyl)(imino)methyl)carbamate 24-5 (100 mg, 0.16 mmol) in 2 mL MeOH was added 4 M HCl in MeOH (10 mL). The reaction was stirred at 50° C. for 16 hours. The mixture was concentrated and alkalized with NaHCO₃ solution (20 mL). The precipitate was collected and the crude was purified by Prep-HPLC (ACN/H₂O with 0.05% HCl) to afford 4-(3-(((2-(4-fluorophenyl)-3-(pyridin-4-yl)pyrazolo[1,5-a]pyridin-6-yl)methyl)amino)propoxy)benzimidamide HCl salt 1-281 (9.4 mg) as white solid with 10% yield.

LCMS: Calculated Exact Mass=494.2, Found [M+H]+ (ESI+)=495.2. ¹H NMR (400 MHz, MeOD) δ 9.02 (s, 1H), 8.69 (d, J=5.8 Hz, 2H), 8.13 (d, J=9.2 Hz, 1H), 7.94 (d, J=5.9 Hz, 2H), 7.80 (t, J=5.9 Hz, 3H), 7.62 (dd, J=8.5, 5.4 Hz, 2H), 7.24 (t, J=8.7 Hz, 2H), 7.18 (d, J=8.9 Hz, 2H), 4.45 (s, 2H), 4.27 (t, J=5.7 Hz, 2H), 3.47-3.34 (m, 2H), 2.33 (s, 2H). ¹H NMR (400 MHz, DMSO) δ 9.91 (d, J=55.9 Hz, 2H), 9.21 (dd, J=25.6, 11.1 Hz, 3H), 8.97 (d, J=32.9 Hz, 2H), 8.76 (d, J=5.6 Hz, 2H), 8.05 (t, J=8.0 Hz, 1H), 7.95-7.69 (m, 5H), 7.67-7.54 (m, 2H), 7.34 (t, J=8.8 Hz, 2H), 7.15 (d, J=8.8 Hz, 2H), 4.33 (s, 2H), 4.23 (t, J=5.8 Hz, 2H), 3.12 (s, 2H), 2.24 (s, 2H).

### B: Biological Assays

### (a) HotSpot Kinase Assay

IC50s were determined using the in vitro HotSpot kinase assay (purified enzymes, ³³P-ATP, an appropriate substrate and 1 μM ATP). For enzyme inhibition assays, compounds were tested in range of ten concentrations from 10 uM to 0.0005 uM using purified recombinant Yck2. Reaction conditions were 1 uM ATP, one hour incubation with inhibitor, and kinase activity detected using 33-ATP phosphorylation of an appropriately selected peptide substrate.

### Recombinant Yck2 Protein

Yck2 residues 37-345 were PCR amplified from C. albicans genomic DNA and subcloned into the vector pMCSG53, which codes for a N-terminal His6 tag, TEV protease site, followed by the Yck2 protein. E. coli BL21(DE3)-Gold competent cells were transformed with this plasmid and the Yck2 protein was purified using methodology previously described (Stogios et al., ACS Chem. Biol. 13, 1322-1332 (2018)).

TABLE 3
Hotspot kinase assay results

| Compound | IC50 |
|---|---|
| ID | (IC50-nM) |
| I-77 | 0.4 |
| I-237 | 1.14 |
| I-148 | 1.61 |
| I-232 | 2.61 |
| I-230 | 2.93 |
| I-58 | 3.19 |
| I-80 | 3.33 |
| I-152 | 3.37 |
| I-115 | 3.67 |
| I-231 | 4.13 |
| I-240 | 4.15 |
| I-117 | 5.08 |
| I-35 | 6.8 |
| I-238 | 7.84 |
| I-116 | 7.91 |
| I-54 | 8.12 |
| I-234 | 8.12 |
| I-55 | 8.41 |
| I-71 | 9.33 |
| I-53 | 9.52 |
| I-33 | 12.29 |
| I-242 | 15.34 |
| I-229 | 16.38 |
| I-100 | 32.94 |
| I-147 | 36.8 |
| I-1 | 46.26 |
| I-102 | 46.57 |
| I-90 | 47.59 |
| I-153 | 48.69 |
| I-103 | 49.48 |
| I-233 | 65.93 |
| I-224 | 72.26 |
| I-6 | 72.71 |
| I-2 | 106.8 |
| I-235 | 107.4 |
| I-236 | 168.9 |
| I-154 | 176.3 |
| I-4 | 177.5 |
| I-3 | 179.2 |
| I-239 | 184.8 |
| I-5 | 334.8 |
| I-151 | 764.6 |
| I-248 | 4.69 |
| I-252 | 2.07 |
| I-253 | 5.95 |
| I-254 | 3.09 |

### (b) Antifungal Sensitivity Testing

Minimum inhibitory concentrations (MICs) were determined in flat bottom, 96-well plate format using a modified broth microdilution protocol as previously described (Singh et al., PLoS Pathog. 5, e1000532 (2009); LaFayette et al., PLoS Pathog. 6, 79-80 (2010)). Compounds were formulated in dimethyl sulfoxide (DMSO, Sigma-Aldrich Co.). Each compound was tested in duplicate in at least two independent experiments.

TABLE 4
Antifungal Sensitivity Testing Results

| Compound | CaCi2 MIC |
|---|---|
| ID | (MIC50-uM) |
| I-103 | 0.0488 |
| I-100 | 0.0651 |
| I-147 | 0.0977 |
| I-117 | 0.195 |
| I-239 | 0.195 |
| I-154 | 0.195 |
| I-90 | 0.586 |
| I-238 | 0.781 |
| I-155 | 0.781 |
| I-115 | 1.27 |
| I-153 | 1.56 |
| I-230 | 1.88 |
| I-236 | 2.34 |
| I-102 | 3.12 |
| I-116 | 6.25 |
| I-148 | 6.25 |
| I-224 | 25 |
| I-231 | >25 |
| I-232 | >25 |
| I-58 | >25 |
| I-32 | >50 |
| I-253 | 0.137 |
| I-257 | 0.295 |
| I-261 | 0.195 |
| I-262 | 0.293 |
| I-266 | 0.195 |
| I-270 | 0.586 |
| I-268 | 1.17 |
| I-255 | 1.56 |

### (c) Mammalian Cell Toxicity.

HepG2 cells were seeded overnight at 500 cells/well in 384-well plate, then incubated with compounds (10-point concentration titration) for 48 h. Cell Titer-Glo® reagent (ThermoFisher Scientific) was added to each well and after 10-minute incubation at room temperature the luminescent signal was measured using an Envision plate reader (Perkin Elmer).

TABLE 5
Mammalian Cell Toxicity Results

| Compound | HepG2 |
|---|---|
| ID | (IC50-uM) |
| I-231 | 1.84 |
| I-102 | 2.24 |
| I-229 | 5.59 |
| I-103 | 6.38 |
| I-155 | 10.06 |
| I-244 | 18.4 |
| I-239 | >20 |
| I-153 | >20 |
| I-115 | >20 |
| I-80 | >20 |
| I-242 | >20 |
| I-139 | >20 |
| I-249 | >50 |
| I-250 | >50 |
| I-255 | 22.5 |
| I-256 | 11.79 |
| I-257 | 5.57 |
| I-266 | 19.48 |
| I-267 | 8.03 |
| I-268 | >50 |
| I-269 | 3.67 |
| I-270 | 1.54 |

While the present application has been described with reference to what are presently considered to be the preferred examples, it is to be understood that the application is not limited to the disclosed examples. To the contrary, the present application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1. A conjugate compound of Formula (I) or an enantiomer thereof, or a pharmaceutically acceptable salt, solvate and/or prodrug thereof, 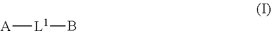
wherein:
A is a moiety that increases fungal cell uptake and/or fungal cell permeability;
B is a CK1 inhibiting moiety; and
L¹ is a linker comprising at least one first complimentary functional group to covalently link to a functional group in A and at least one second complimentary functional group to covalently link to a functional group in B, to form the conjugate of Formula (I),
wherein the compound of Formula I optionally comprises one or more atoms that are isotopic variants.

2. The conjugate compound of claim 1, wherein A is an antifungal moiety, comprising one or more of an alkylene moiety, an azole moiety, an indole moiety, a phenyl moiety, a carbazole moiety, pyridoindole moiety, a tetrahydropyridoindole moiety, a polyamine moiety, a fatty acid alkyl chain moiety, a flucytosine (5-FC) moiety and a triphenylphosphonium moiety, each of which are optionally substituted with one or more groups independently selected from halo, OH, C₁₋₄alkyl, C₁₋₄haloalkyl, OC₁₋₄alkyl, C(O)OH, C(O)NH₂, C(O)C₁₋₄alkyl, C(O)OC₁₋₄alkyl, C(O)NHC₁₋₄alkyl, C(O)N(C₁₋₄alkyl)C₁₋₄alkyl), C(NH)NH₂, C(NC₁₋₄alkyl)NH₂, C(NC₁₋₄ alkyl)NHC₁₋₄alkyl, C(NC₁₋₄alkyl)N(C₁₋₄alkyl)(C₁₋₄alkyl), C(NH)NHC₁₋₄alkyl and C(NH)N(C₁₋₄ alkyl)(C₁₋₄alkyl), wherein each alkyl group is optionally halosubstituted, suitably fluorosubstituted.

3. (canceled)

4. The conjugate compound of claim 1, wherein the azole moiety is selected from an imidazole moiety, a triazole moiety and a tetrazole moiety.

5. The conjugate compound of claim 1, wherein A is selected from fluconazole, itraconazole, clotrimazole, ketoconazole, voriconazole, posaconazole, isavuconazonium, miconazole, flucytosine, olorofim, manogepix, ibrexafungerp, caspofungin, micafungin, anidulafungin, rezafungin, amphotericin B and VT-1161.

6. The conjugate compound of claim 1, wherein A is selected from: 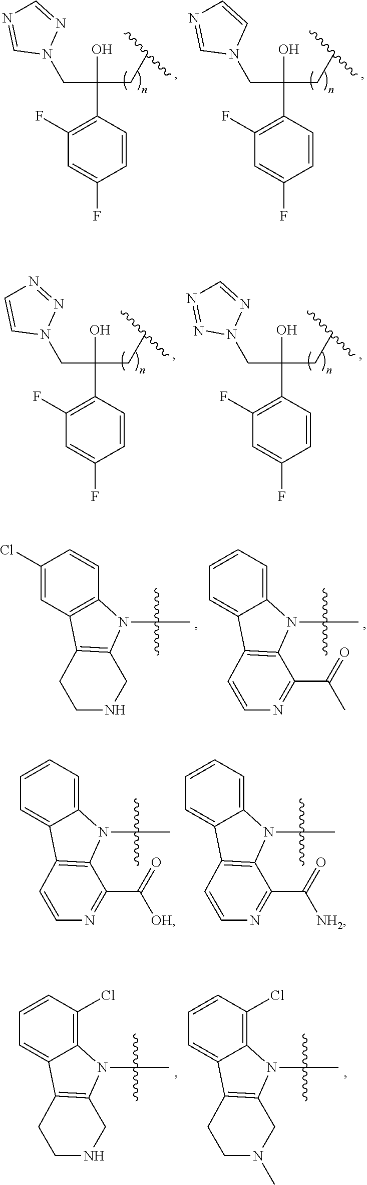 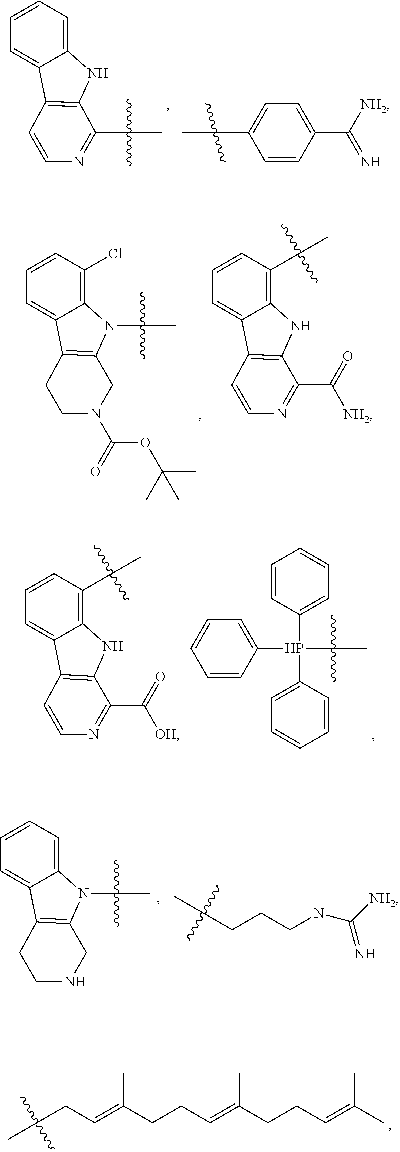 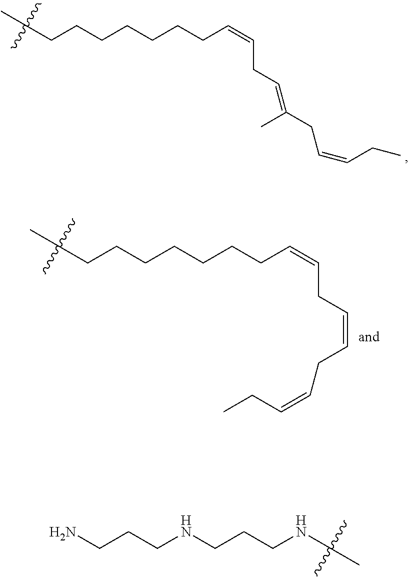
wherein each n is independently an integer from 0 to 4, and 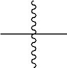
represents the point of attachment to L¹, including all stereoisomers thereof.

7. The conjugate compound of claim 1, wherein A is selected from: 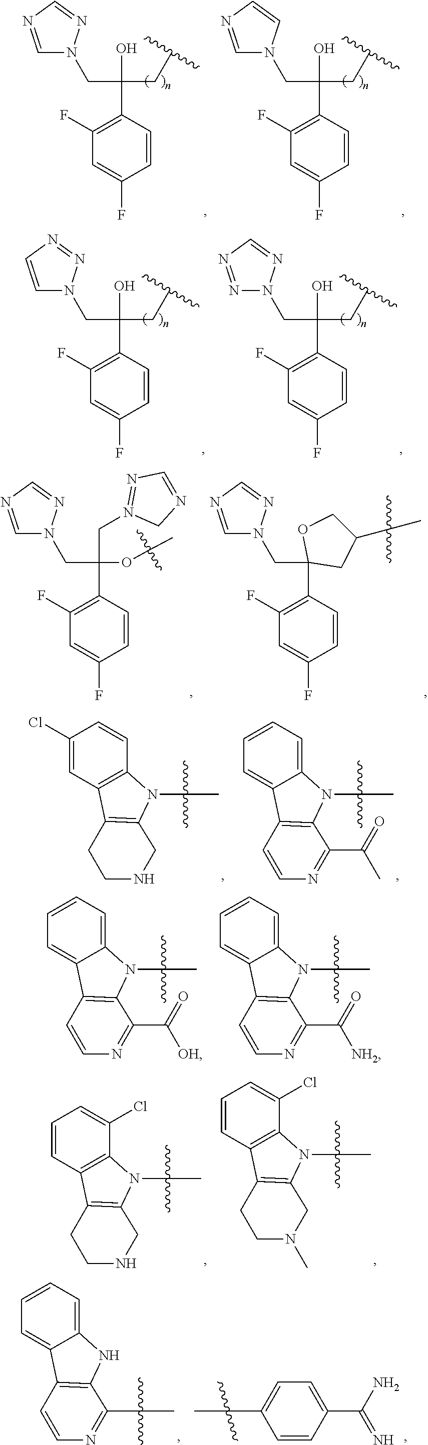 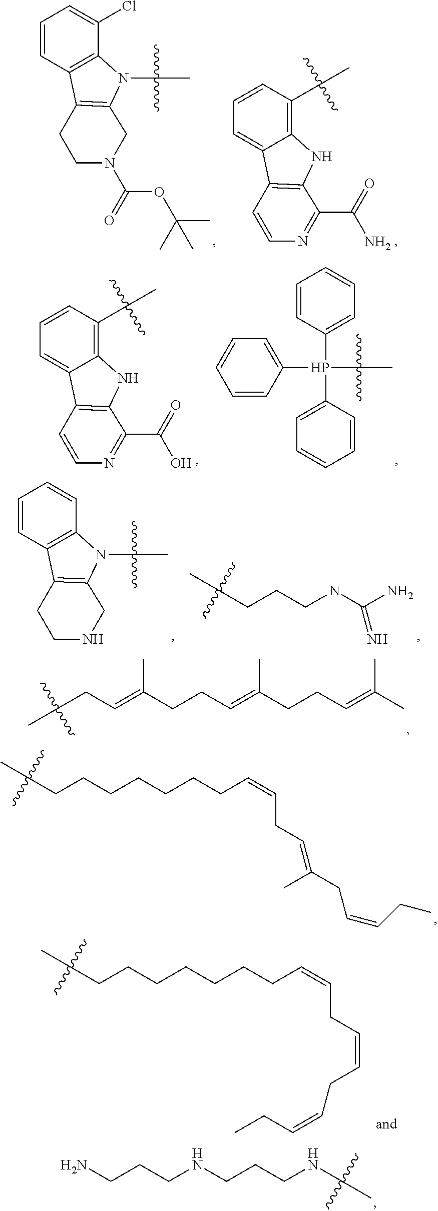
wherein each n is independently an integer from 0 to 4, and 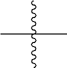
represents the point of attachment to L¹, including all stereoisomers thereof; and
wherein A optionally comprises one or more atoms that are isotopic variants, for example, wherein the isotopic variant is deuterium and A comprises one or more deuterium atoms.

8. The conjugate compound of claim 1, wherein B is a substituted C₄₋₂₀heteroaryl moiety comprising from 2 to 6 N heteroatoms.

9. The conjugate compound of claim 1, wherein B comprises at least one of: a fluorophenyl, a pyridine moiety, a pyrimidine moiety, a pyrazole moiety, a piperazine moiety and an imidazole moiety.

10. (canceled)

11. (canceled)

12. The conjugate compound of claim 1, wherein B comprises a compound listed in Table 1.

13. The conjugate compound of claim 1, wherein B is selected from: 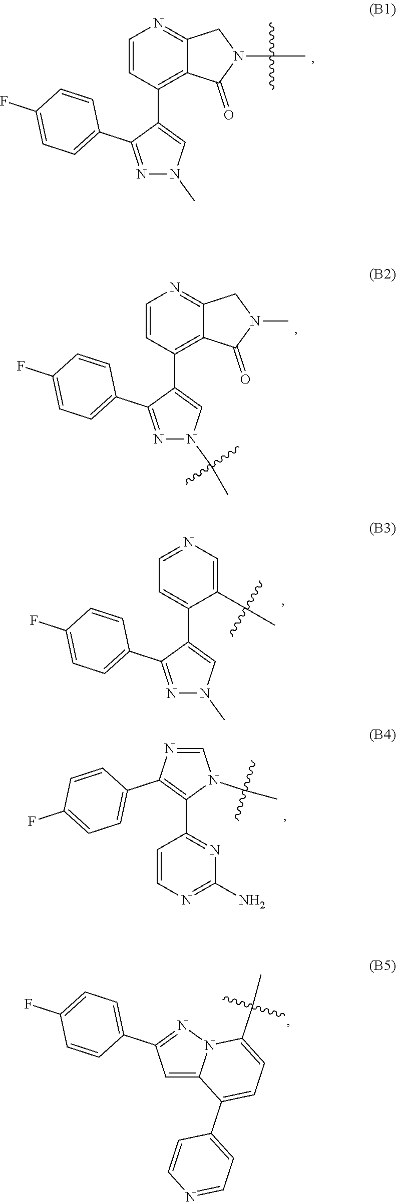 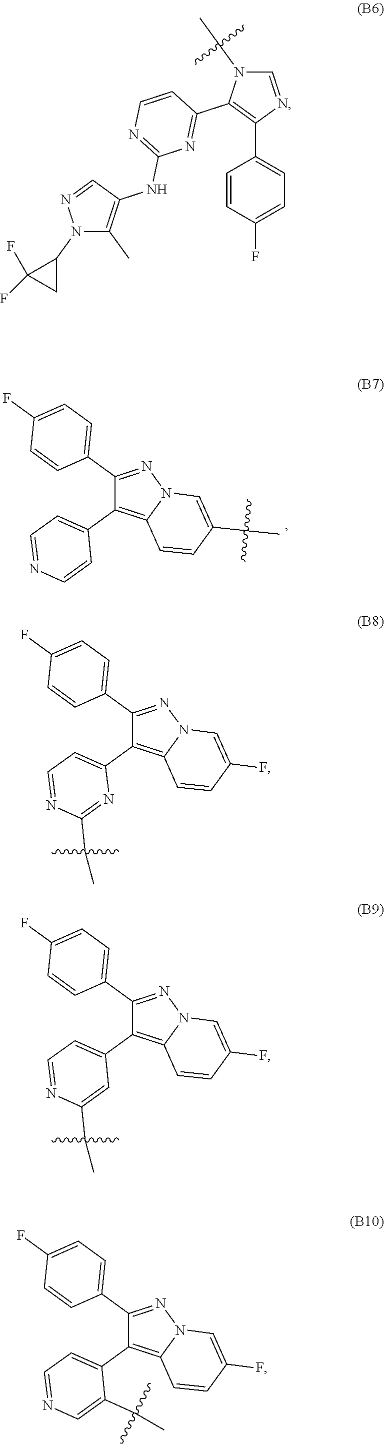 [SMILES: *n1cnc(-c2ccc(F)cc2)c1-c1ccnc(Nc2cnn(C3CC3(F)F)c2C)n1 ; *c1ccc2c(-c3ccncc3)c(-c3ccc(F)cc3)nn2c1 ; *c1nccc(-c2c(-c3ccc(F)cc3)nn3cc(F)ccc23)n1 ; *c1cc(-c2c(-c3ccc(F)cc3)nn3cc(F)ccc23)ccn1 ; *c1cnccc1-c1c(-c2ccc(F)cc2)nn2cc(F)ccc12] 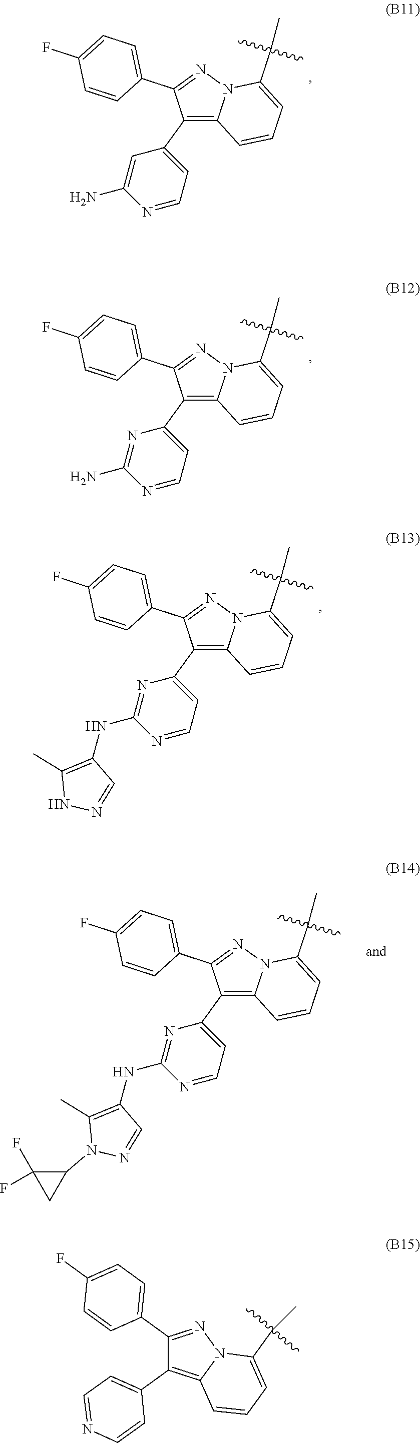 [SMILES: *c1cccc2c(-c3ccnc(N)c3)c(-c3ccc(F)cc3)nn12 ; *c1cccc2c(-c3ccnc(N)n3)c(-c3ccc(F)cc3)nn12 ; *c1cccc2c(-c3ccnc(Nc4cn[nH]c4C)n3)c(-c3ccc(F)cc3)nn12 ; *c1cccc2c(-c3ccnc(Nc4cnn(C5CC5(F)F)c4C)n3)c(-c3ccc(F)cc3)nn12 ; *c1cccc2c(-c3ccncc3)c(-c3ccc(F)cc3)nn12]
wherein 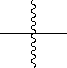
represents the point of attachment to L¹.

14. The conjugate compound of claim 1, wherein L¹ comprises one or more independently selected groups selected from C(O), C(═NR¹), O, NR¹, C₁₋₁₂alkylene, C₆₋₂₀arylene, C₃₋₁₀cycloalkylene, C₂₋₂₀heteroarylene and C₃₋₁₀heterocycloalkylene, wherein each of the alkylene, arylene, cycloalkylene, heteroarylene and heterocycloalkylene is optionally substituted with one or more R², and each R² is independently selected from C₁₋₁₂alkyl, NR³C(O)R⁴, NR³R⁴, halo, C(O)NR³R⁴, and OR³; and R¹, R³ and R⁴ are independently selected from H and C₁₋₁₂alkyl, or L¹ is selected from C₁₋₁₂alkylene-NR¹—C(O), C₁₋₆ alkylene-C₆₋₁₀arylene-NR¹—C(O), C₁₋₆alkylene-NR¹—C(O)—C₂₋₁₀heteroarylene, C₁₋₆alkylene-C₂₋₁₀heteroarylene-NR¹—C(O), C₂₋₁₀heteroarylene-NR¹—C(O), C₁₋₆alkylene-C₃₋₁₀heterocycloalkylene-NR¹—C(O)—C₂₋₁₀heteroarylene, C₁₋₆alkylene-C₃₋₁₀heterocycloalkylene-C₂₋₁₀heteroarylene, C₁₋₆ alkylene-C₃₋₁₀heterocycloalkylene-NR¹—C(O), C₁₋₆alkylene-C₃₋₁₀heterocycloalkylene-C₁₋₆ alkylene-NR¹—C(O), C₁₋₆alkylene-C₃₋₁₀heterocycloalkylene-C₆₋₁₀arylene-NR¹—C(O), C₁₋₆alkylene-C₃₋₁₀heterocycloalkylene-C₁₋₆alkylene-C₆₋₁₀arylene-NR¹—C(O), C₁₋₆alkylene-C(O)—C₃₋₁₀heterocycloalkylene-NR¹—C(O), C₁₋₆alkylene-C(O)—C₃₋₁₀heterocycloalkylene-NR¹—C₁₋₆-alkylene, C₁₋₆alkylene-O—C(O)—C₁₋₆alkylene-C₃₋₁₀heterocycloalkylene-NR¹—C(O), C₁₋₆alkylene-C₃₋₁₀heterocycloalkylene-NR¹—C₁₋₆alkylene, C₁₋₆alkylene-C₃₋₁₀heterocycloalkylene-C₁₋₆alkylene-NR¹—C₁₋₆alkylene, C₁₋₆alkylene-C₃₋₁₀heterocycloalkylene-C₁₋₆alkylene, C₁₋₆alkylene-NR¹—C₁₋₆alkylene, O—C₁₋₁₂alkylene, C₁₋₆alkylene-C₃₋₁₀heterocycloalkylene-O—C₁₋₆alkylene-C₆₋₁₀arylene-NR¹—C(O), C₁₋₆ alkylene-C₃₋₁₀heterocycloalkylene-O—C₁₋₆alkylene-O—C₆₋₁₀arylene-NR¹—C(O), and NR¹—C₁₋₆ alkylene-O—C₁₋₆alkylene-O—C₁₋₆alkylene-NR¹, each L¹ is optionally substituted with one or more R² and A and B are covalently bonded to either end of L¹.

15. (canceled)

16. The conjugate compound of claim 14, wherein each R² is independently selected from C₁₋₆alkyl, NR³C(O)R⁴, NR³R⁴, C(O)NR³R⁴, and OR³; and R³ and R⁴ are independently selected from H and C₁₋₆alkyl.

17. The conjugate compound of claim 14, wherein each R² is independently selected from methyl, ethyl, propyl, F, Cl, NH₂, NHC(O)C₁₋₆alkyl, C(O)NH₂ and OH, and R³ and R⁴ are independently selected from H, methyl, ethyl and propyl.

18. (canceled)

19. The conjugate compound of claim 1, wherein L¹ is selected from: 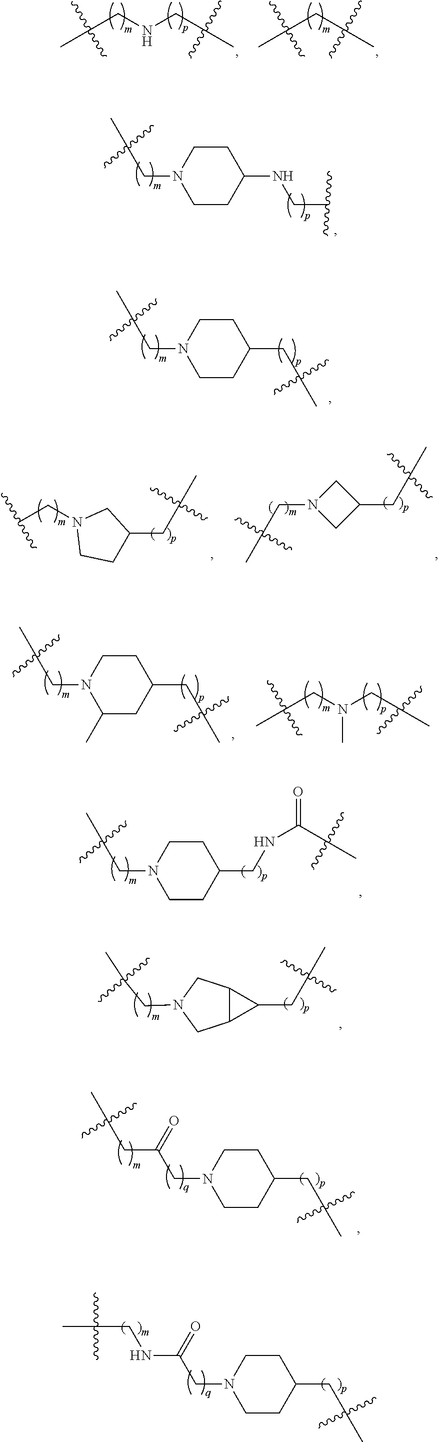 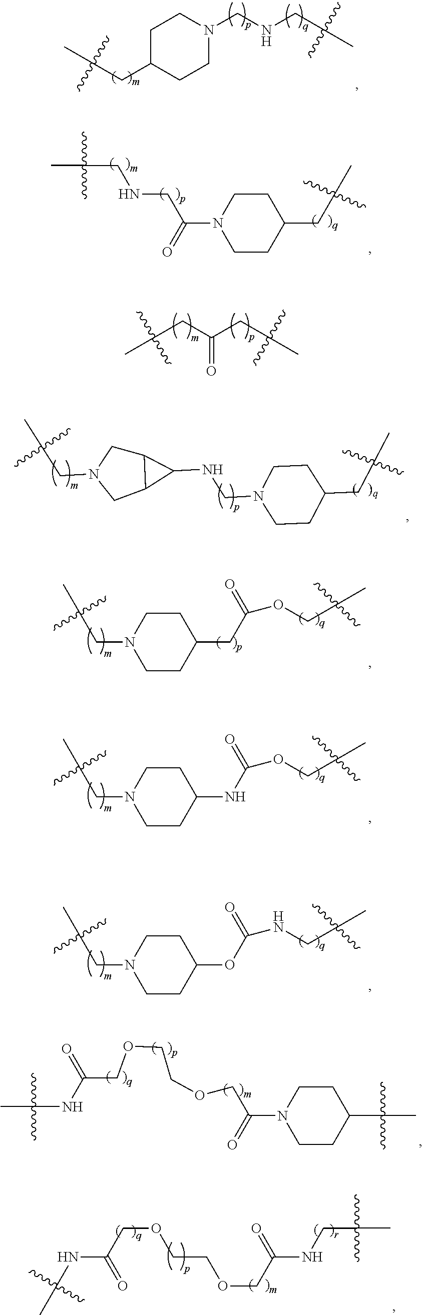 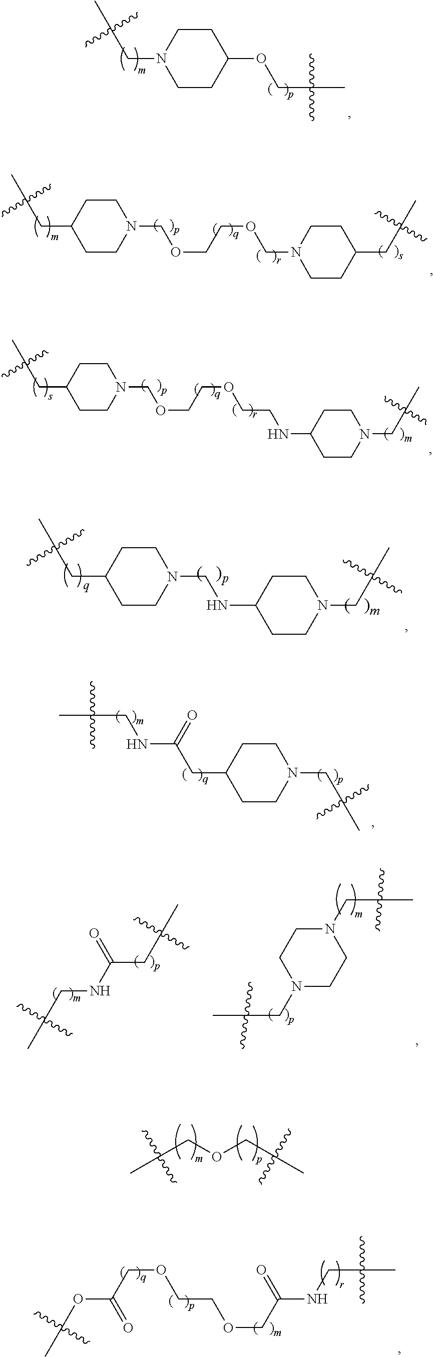 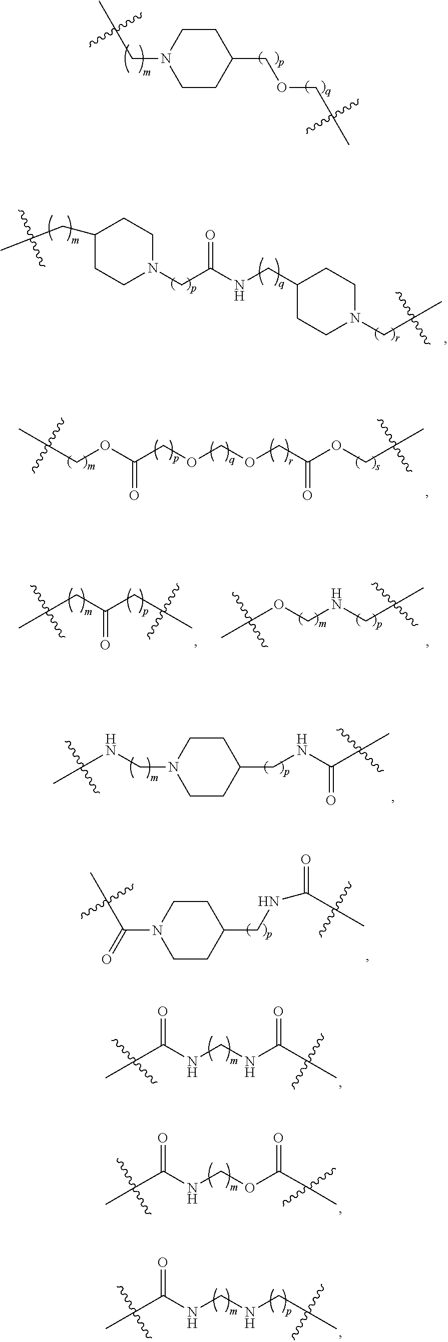 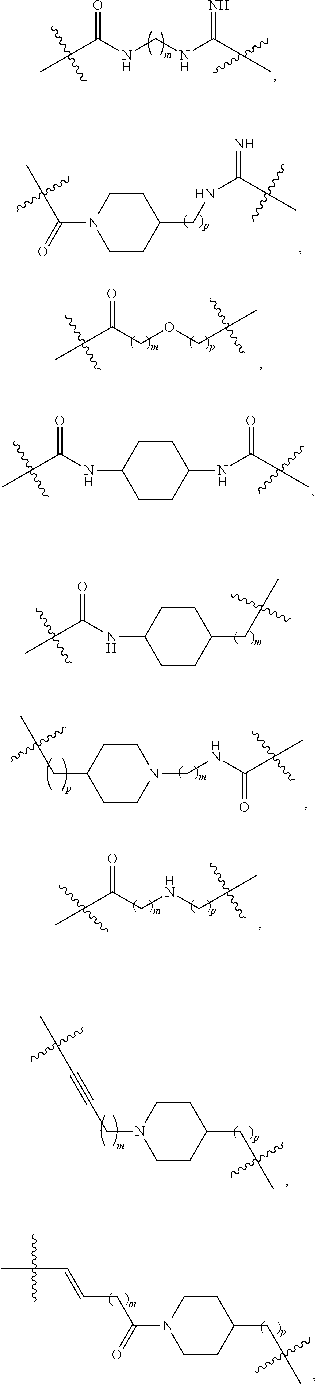 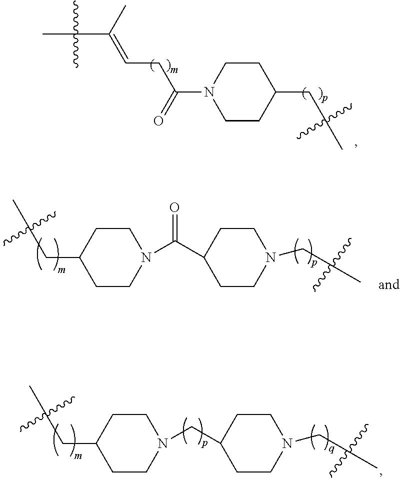
wherein each m, p, q, r and s is independently an integer from 0 to 6, and 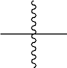
represents the points of attachment to either A or B, including all stereoisomers thereof.

20. The conjugate compound of claim 1, wherein L¹ is selected from: 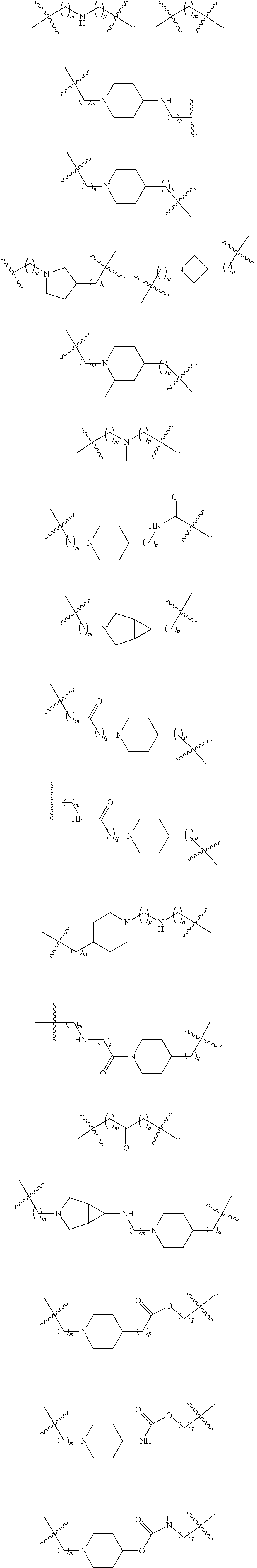 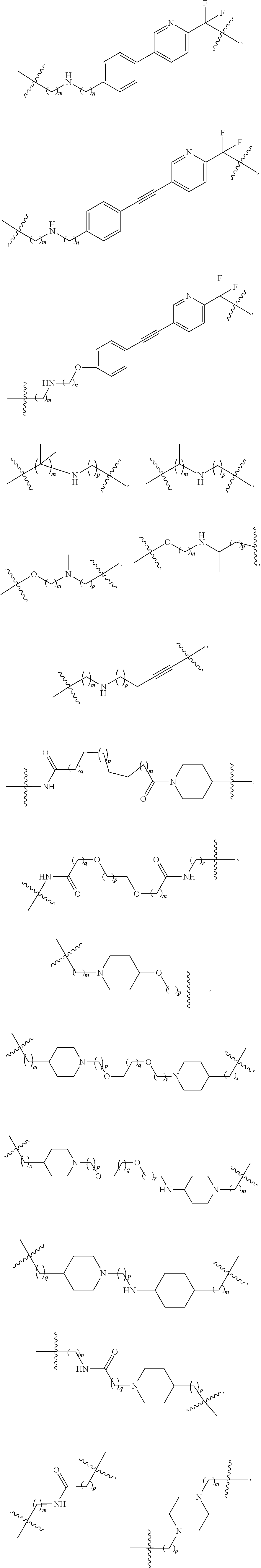 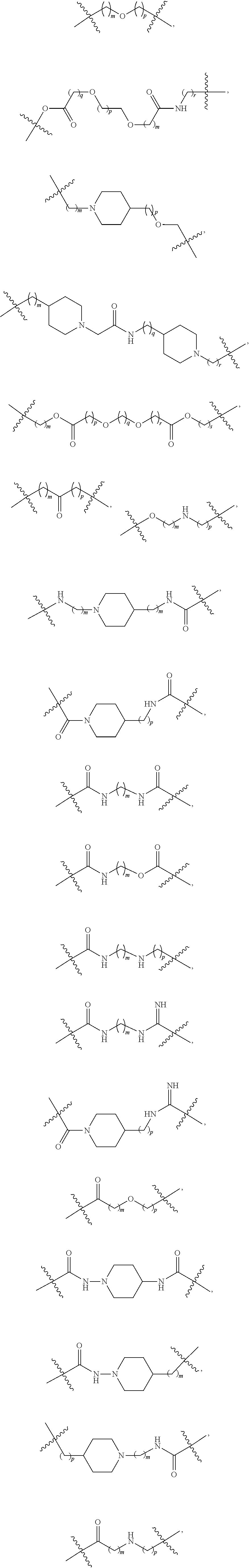 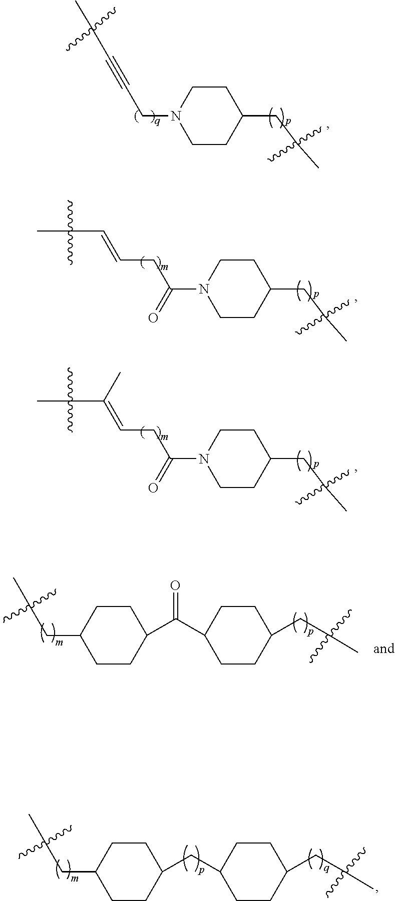
wherein each m, p, q, r and s is independently an integer from 0 to 6, and 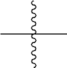
represents the points of attachment to either A or B, including all stereoisomers thereof, and
wherein L¹ optionally comprises one or more atoms that are isotopic variants, for example, wherein the isotopic variant is deuterium and L¹ comprises one or more deuterium atoms.

21. The conjugate compound of claim 1, wherein the compound of Formula (I) is selected from the compounds listed in Table 2 or a salt, and/or solvate thereof.

22. A method of treating or preventing a fungal-related disease, disorder or condition, inhibiting or preventing fungal growth, inhibiting fungal CK1 activity, selectively inhibiting fungal CK1 activity or treating or preventing mycosis, comprising administering a therapeutically effective amount of one or more conjugate compounds of claim 1 to a subject in need thereof.

23.-26. (canceled)

27. A method of treating or preventing a fungal-related disease, disorder or condition, inhibiting or preventing fungal growth, inhibiting fungal CK1 activity, selectively inhibiting fungal CK1 activity or treating or preventing mycosis, comprising administering a therapeutically effective amount of one or more conjugate compounds of claim 1 in combination with another known agent useful for treatment or prevention of a fungal-related disease, disorder or condition, for inhibition or prevention of fungal growth, for inhibition of fungal CK1 activity, for selective inhibition of fungal CK1 activity, or for treatment or prevention of mycosis, to a subject in need thereof.

28. A pharmaceutical composition comprising one or more conjugate compounds of claim 1, or a pharmaceutically acceptable salt, and/or solvate thereof, and a pharmaceutically acceptable carrier and/or diluent.

29. The pharmaceutical composition of claim 28 further comprising an additional therapeutic agent.

30. (canceled)
